(19) United States
(12) Patent Application Publication
(10) Pub. No.: US 2026/0098072 A1
(43) Pub. Date: Apr. 9, 2026

(54) METHODS OF STIMULATING APPETITE AND/OR INCREASING BODY WEIGHT USING NON-NATURALLY OCCURRING MELANOCORTIN RECEPTOR ANTAGONIST ANALOGS

(71) Applicant: Endevica Bio, Inc., Northbrook, IL (US)

(72) Inventor: Russell Potterfield, Winnetka, IL (US)

(21) Appl. No.: 19/183,258

(22) Filed: Apr. 18, 2025

Related U.S. Application Data
(63) Continuation of application No. PCT/US23/35743, filed on Oct. 23, 2023.
(60) Provisional application No. 63/418,468, filed on Oct. 21, 2022.
(60) Provisional application No. 63/486,169, filed on Feb. 21, 2023.
(60) Provisional application No. 63/497,597, filed on Apr. 21, 2023.

(51) Int. Cl. C07K 14/665; A61K 38/00; A61P 3/00

(52) U.S. Cl. CPC C07K 14/665; A61P 3/00; A61K 38/00

(57) ABSTRACT

Provided are methods of increasing body weight of and/or increasing food consumption in a metabolically unchallenged subject using non-naturally occurring melanocortin analogs. Also provided are methods of stimulating appetite in a subject via administration of non-naturally occurring melanocortin analogs. The non-naturally occurring melanocortin analog can be present in a pharmaceutical composition and delivered via parenteral administration (e.g., subcutaneous injection). The methods improve appetite, increase food consumption, increase body weight, muscle mass, and/or fat mass of the subject.

### CROSS-REFERENCE TO RELATED APPLICATION(S)

This application is a U.S. national stage entry under 37 U.S.C. 371 of International Application No. PCT/US2023/035743, filed Oct. 23, 2023, which claims priority to U.S. Provisional Patent Application No. 63/418,468 filed Oct. 21, 2022; U.S. Provisional Patent Application No. 63/486,169 filed Feb. 21, 2023; and U.S. Provisional Patent Application No. 63/497,597 filed Apr. 21, 2023, all of which applications are incorporated herein by reference in their entirety.

### SEQUENCE LISTING INCORPORATED BY REFERENCE

This application contains an ST.26 compliant Sequence Listing which is hereby incorporated by reference in its entirety. The .xml copy, created on Nov. 29, 2023, is named 146316_8008_WO00_SL.xml and is 1,129,627 bytes.

### TECHNICAL FIELD

Described herein are non-naturally occurring melanocortin receptor antagonist analogs and compositions comprising the same.

### SUMMARY

In some embodiments, the present technology generally relates to a method of stimulating appetite of a subject in need thereof, the method including: administering to the subject a therapeutically effective amount of a non-naturally occurring melanocortin analog including a sequence according to Formula (I),

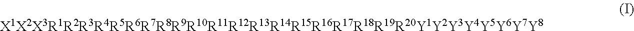

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF3)dPhe);;
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine; and
wherein the non-naturally occurring melanocortin analog comprises one or more of the following features: (i) X² or X³ is present; (ii) R¹ is absent or is selected from the group consisting of dArg, dMet, dIIe, dLeu, dVal, dAla, Ala, Tle, dTle, DNle, Nva, Gly, dPro, dCys, dPhe, dTyr, dGln, dAsn, transPro(guan), cisPro(guan), dTyr, Tyr, and Dmt; (iii) R² is absent; (iv) R³ is selected from the group consisting of dPro, Pro-Gly, and dAla; (v) R⁴ is selected from the group consisting of His, Trp, and Phe; (vi) R⁵ is Phe; (vii) R⁷ is absent; (viii) R⁸ is present; (ix) R⁹ and/or R¹⁰ is present; (x) one or more of R¹¹—R²⁰ are present; and (xi) Y⁴ is present; and
the non-naturally occurring melanocortin analog is cyclized through a moiety selected from the group consisting of:
   a disulfide bond between R¹ or R² and R⁷ or X¹ when R¹ or R² is cysteine and R⁷ or X¹ is cysteine;
   a disulfide bond between R² and any one of R⁵—R²⁰ when R² and the any one of R⁵—R²⁰ are selected from the group consisting of cysteine, Pen, and dPen;
   a lactam bridge between R¹ and R⁷ when R¹ is norleucine and R⁷ is glutamic acid;
   a side-chain lactam bridge between R² and R⁷ when R² is glutamic acid, aspartic acid, or CO-cis-CH—CH—CO, and R⁷ is lysine or ornithine;
   a side-chain lactam bridge between R² and R⁸ when R² is glutamic acid or aspartic acid, R⁸ is lysine, and R⁷ is proline or tryptophan;
   a lactam closure between R¹ and R⁷ when R¹ is succinic acid or o-phthalic acid and R⁷ is lysine; and
   a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

In some embodiments, the present technology generally relates to use of a non-naturally occurring melanocortin analog for stimulating appetite of a subject in need thereof, the use including: administering to the subject a therapeutically effective amount of the non-naturally occurring melanocortin analog, wherein the non-naturally occurring melanocortin analog includes a sequence according to Formula (I),

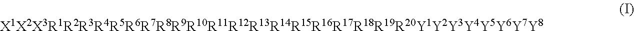

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF3)dPhe);
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine; and
wherein the non-naturally occurring melanocortin analog comprises one or more of the following features: (i) X² or X³ is present; (ii) R¹ is absent or is selected from the group consisting of dArg, dMet, dIIe, dLeu, dVal, dAla, Ala, Tle, dTle, DNle, Nva, Gly, dPro, dCys, dPhe, dTyr, dGIn, dAsn, transPro(guan), cisPro(guan), dTyr, Tyr, and Dmt; (iii) R² is absent; (iv) R³ is selected from the group consisting of dPro, Pro-Gly, and dAla; (v) R⁴ is selected from the group consisting of His, Trp, and Phe; (vi) R⁵ is Phe; (vii) R⁷ is absent; (viii) R⁸ is present; (ix) R⁹ and/or R¹⁰ is present; (x) one or more of R¹¹—R²⁰ are present; and (xi) Y⁴ is present; and
the non-naturally occurring melanocortin analog is cyclized through a moiety selected from the group consisting of:
   a disulfide bond between R¹ or R² and R⁷ or X¹ when R¹ or R² is cysteine and R⁷ or X¹ is cysteine;
   a disulfide bond between R² and any one of R⁵—R²⁰ when R² and the any one of R⁵—R²⁰ are selected from the group consisting of cysteine, Pen, and dPen;
   a lactam bridge between R¹ and R⁷ when R¹ is norleucine and R⁷ is glutamic acid;
   a side-chain lactam bridge between R² and R⁷ when R² is glutamic acid, aspartic acid, or CO-cis-CH═CH—CO, and R⁷ is lysine or ornithine;
   a side-chain lactam bridge between R² and R⁸ when R² is glutamic acid or aspartic acid, R⁸ is lysine, and R⁷ is proline or tryptophan;
   a lactam closure between R¹ and R⁷ when R¹ is succinic acid or o-phthalic acid and R⁷ is lysine; and
   a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

In some embodiments, the present technology generally relates to a use of a non-naturally occurring melanocortin analog in the manufacture of a medicament for stimulating appetite of a subject in need thereof, the use including: administering to the subject a therapeutically effective amount of the non-naturally occurring melanocortin analog, wherein the non-naturally occurring melanocortin analog includes a sequence according to Formula (I),

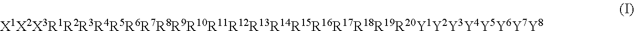

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF3)dPhe);;
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
   wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine; and
wherein the non-naturally occurring melanocortin analog comprises one or more of the following features: (i) X² or X³ is present; (ii) R¹ is absent or is selected from the group consisting of dArg, dMet, dIIe, dLeu, dVal, dAla, Ala, Tle, dTle, DNle, Nva, Gly, dPro, dCys, dPhe, dTyr, dGln, dAsn, transPro(guan), cisPro(guan), dTyr, Tyr, and Dmt; (iii) R² is absent; (iv) R³ is selected from the group consisting of dPro, Pro-Gly, and dAla; (v) R⁴ is selected from the group consisting of His, Trp, and Phe; (vi) R⁵ is Phe; (vii) R⁷ is absent; (viii) R⁸ is present; (ix) R⁹ and/or R¹⁰ is present; (x) one or more of R¹¹—R²⁰ are present; and (xi) Y⁴ is present; and
the non-naturally occurring melanocortin analog is cyclized through a moiety selected from the group consisting of:
   a disulfide bond between R¹ or R² and R⁷ or X¹ when R¹ or R² is cysteine and R⁷ or X¹ is cysteine;
   a disulfide bond between R² and any one of R⁵—R²⁰ when R² and the any one of R⁵—R²⁰ are selected from the group consisting of cysteine, Pen, and dPen;
   a lactam bridge between R¹ and R⁷ when R¹ is norleucine and R⁷ is glutamic acid;
   a side-chain lactam bridge between R² and R⁷ when R² is glutamic acid, aspartic acid, or CO-cis-CH═CH—CO, and R⁷ is lysine or ornithine;
   a side-chain lactam bridge between R² and R⁸ when R² is glutamic acid or aspartic acid, R⁸ is lysine, and R⁷ is proline or tryptophan;
   a lactam closure between R¹ and R⁷ when R¹ is succinic acid or o-phthalic acid and R⁷ is lysine; and
   a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

In some aspects, the N-terminus, if present, is modified by a functional group selected from the group consisting of an acyl group, an imine group, an amide group, a urea group, a carbamate group, a sulfonamide group, and an alkylamine group. In some aspects, the N-terminus, if present, is modified by an acyl group. In some aspects, the acyl group is acetyl group. In some aspects, the acyl group is formyl group. In some aspects, the N-terminus, if present, is modified by an imine group. In some aspects, the N-terminus, if present, is modified by an amide group. In some aspects, the amide group is a pyroglutamyl (pGlu) group. In some aspects, the amide group is derived from a fatty acid. In some aspects, the N-terminus, if present, is not modified. In some aspects, the C-terminus is modified by a functional group selected from the group consisting of an amide group, an ester group, and an aldehyde group. In some aspects, the C-terminus is modified by an amide group. In some aspects, the amide group is an —NHalkyl amide group or an —NHaryl amide group. In some aspects, the—NHaryl amide group is p-nitroanilide group or 7-amino-4-methylcoumarin. In some aspects, the C-terminus is modified by an ester group. In some aspects, the C-terminus is not modified.

In some aspects, R¹ is absent, and R² is D-aspartic acid. In some aspects, X¹, X², and X³ are absent. In some aspects, R⁴ is D-Nal(2′). In some aspects, Y³-Y⁸ are absent. In some aspects, Y¹ is D-valine and Y² is D-proline; or Y¹ is D-proline and Y² is D-valine. In some aspects, Y³ is present and Y⁴-Y⁸ are absent. In some aspects, Y¹ is D-valine or D-proline; Y² is D-valine or D-proline; and/or Y³ is D-valine or D-proline. In some aspects, Y¹ is D-valine, Y² is D-valine, and Y³ is D-proline; Y¹ is D-proline, Y² is D-valine, and Y³ is D-valine; Y¹ is D-valine, Y² is D-proline, and Y³ is D-valine; or Y¹ is D-proline, Y² is D-valine, and Y³ is D-proline. In some aspects, Y³ and Y⁴ are present, and Y⁵-Y⁸ are absent. In some aspects, Y¹ is D-valine or D-proline; Y² is D-valine or D-proline; Y³ is D-valine or D-proline; and/or Y⁴ is D-valine or D-proline. In some aspects, Y¹ is D-valine, Y² is D-valine, Y³ is D-valine, and Y⁴ is D-proline; Y¹ is D-proline, Y² is D-valine, Y³ is D-valine, and Y⁴ is D-valine; Y¹ is D-valine, Y² is D-proline, Y³ is D-valine, and Y⁴ is D-valine; Y¹ is D-valine, Y² is D-valine, Y³ is D-proline, and Y⁴ is D-valine; or Y¹ is D-valine, Y² is D-proline, Y³ is D-valine, and Y⁴ is D-proline. In some aspects, R¹, R², and R⁷ are present and R⁸—R²⁰ are absent, and the sequence of Formula (I) is cyclized through R² and R⁷ via a lactam bond. In some aspects, R¹ is acetylated norleucine; R² is aspartic acid; R³ is selected from the group consisting of proline, hydroxyproline, and D-hydroxyproline; R⁴ is dNal(2′); R⁵ is arginine; R⁶ is D-tryptophan or L-tryptophan; R⁷ is lysine; Y¹ is D-valine; and/or Y² is D-proline.

In some aspects, the sequence of Formula (I) is: Ac-Nle-c(Asp-Pro-dNal2′-Arg-Trp-Lys)-dVal-dPro-NH2 (SEQ ID NO: 3); or Ac-Nle-c(Asp-Hyp-dNal2′-Arg-Trp-Lys)-dVal-dPro-NH2 (SEQ ID NO: 4), wherein c represents cyclization through R² and R⁷ via a lactam bond.

In some aspects, R¹, R², R⁷, and R⁸ are present and R⁹—R²⁰ are absent, and the sequence of Formula (I) is cyclized through R² and R⁸ via a lactam bond.

In some aspects, R¹ is acetylated norleucine; R² is aspartic acid; R³ is selected from the group consisting of proline, hydroxyproline, D-hydroxyproline, phenylalanine, and histidine; R⁴ is histidine or dNal(2′); R⁵ is dNal(2′) or arginine; R⁶ is selected from the group consisting of arginine, D-tryptophan, and L-tryptophan; R⁷ is tryptophan or proline; R⁸ is lysine; Y¹ is selected from the group consisting of D-valine, D-leucine, and D-isoleucine; and/or Y² is D-proline.

In some aspects, the sequence of Formula (I) is selected from the group consisting of: Ac-Nle-c(Asp-Pro-His-dNal(2′)-Arg-Trp-Lys)-dVal-dPro-NH2 (SEQ ID NO: 6); Ac-Nle-c(Asp-Phe-His-dNal(2′)-Arg-Trp-Lys)-dLeu-dPro-NH2 (SEQ ID NO: 7); Ac-Nle-c(Asp-Phe-His-dNal(2′)-Arg-Trp-Lys)-dIIe-dPro-NH2 (SEQ ID NO: 8); Ac-Nle-c(Asp-His-dNal(2′)-Arg-Trp-Pro-Lys)-dVal-dPro-NH2 (SEQ ID NO: 9); Ac-Nle-c(Asp-Hyp-dNal(2′)-Arg-Trp-Pro-Lys)-dVal-dPro-NH2 (SEQ ID NO: 10); and Ac-Nle-c(Asp-Pro-His-dNal(2′)-Arg-Trp-Lys)-dPro-dVal-NH2 (SEQ ID NO: 11; D5), wherein c represents cyclization through R² and R⁸ via a lactam bond.

In some aspects, R¹—R² and R⁷—R¹⁰ are present and R¹¹—R²⁰ are absent, and the sequence of Formula (I) is cyclized through R² and R¹⁰ via a lactam bond.

In some aspects, the sequence of Formula (I) is Ac-Nle-c(Asp-Phe-Phe-Pro-His-dNal(2′)-Arg-Trp-Lys)-dVal-dPro-NH2 (SEQ ID NO: 12), wherein c represents cyclization through R² and R¹⁰ via a lactam bond. In some aspects, R¹—R² and R⁷—R¹⁰ are present and R¹¹—R²⁰ are absent, and the sequence of Formula (I) is cyclized through R⁴ and R¹⁰ via a lactam bond.

In some aspects, the sequence of Formula (I) is Ac-Nle-Phe-Phe-c(Asp-Phe-His-dNal(2′)-Arg-Trp-Lys)-dVal-Dpro-NH2 (SEQ ID NO: 13), wherein c represents cyclization through R⁴ and R¹⁰ via a lactam bond.

In some aspects, the sequence of Formula (I) is Ac-Nle-c(Asp-Pro-dNal(2′)-Arg-Trp-Lys)-dVal-dPro(SEQ ID NO: 2).

In some aspects, the sequence of Formula (I) is linear.

In some aspects, the sequence of Formula (I) is Ac-Nle-Asp-Pro-dNal(2′)-Arg-Trp-Lys-dVal-dPro-NH2 (SEQ ID NO: 14) or Ac-Nle-Pro-dNal(2′)-Arg-Trp-dVal-dPro-NH2 (SEQ ID NO: 15).

In some aspects, the sequence of Formula (I) is selected from the group consisting of: Ac-dArg-c(Asp-Pro-dNal(2′)-Arg-Trp-Lys)-dVal-dPro-NH2 (SEQ ID NO: 16); Ac-dMet-c(Asp-Pro-dNal(2′)-Arg-Trp-Lys)-dVal-dPro-NH2 (SEQ ID NO: 17); Ac-dIIe-c(Asp-Pro-dNal(2′)-Arg-Trp-Lys)-dVal-dPro-NH2 (SEQ ID NO: 18); Ac-dLeu-c(Asp-Pro-dNal(2′)-Arg-Trp-Lys)-dVal-dPro-NH2 (SEQ ID NO: 19); Ac-dVal-c(Asp-Pro-dNal(2′)-Arg-Trp-Lys)-dVal-dPro-NH2 (SEQ ID NO: 20); Ac-dAla-c(Asp-Pro-dNal(2′)-Arg-Trp-Lys)-dVal-dPro-NH2 (SEQ ID NO: 21); Ac-Ala-c(Asp-Pro-dNal(2′)-Arg-Trp-Lys)-dVal-dPro-NH2 (SEQ ID NO: 22); Ac-Tle-c(Asp-Pro-dNal(2′)-Arg-Trp-Lys)-dVal-dPro-NH2 (SEQ ID NO: 23); Ac-dTle-c(Asp-Pro-dNal(2′)-Arg-Trp-Lys)-dVal-dPro-NH2 (SEQ ID NO: 24); Ac-dNle-c(Asp-Pro-dNal(2′)-Arg-Trp-Lys)-dVal-dPro-NH2 (SEQ ID NO: 25); Ac-Nva-c(Asp-Pro-dNal(2′)-Arg-Trp-Lys)-dVal-dPro-NH2 (SEQ ID NO: 26); Ac-Gly-c(Asp-Pro-dNal(2′)-Arg-Trp-Lys)-dVal-dPro-NH2 (SEQ ID NO: 27); Ac-dPro-c(Asp-Pro-dNal(2′)-Arg-Trp-Lys)-dVal-dPro-NH2 (SEQ ID NO: 28); Ac-dCys-c(Asp-Pro-dNal(2′)-Arg-Trp-Lys)-dVal-dPro-NH2 (SEQ ID NO: 29); Ac-dPhe-c(Asp-Pro-dNal(2′)-Arg-Trp-Lys)-dVal-dPro-NH2 (SEQ ID NO: 30); Ac-dTyr-c(Asp-Pro-dNal(2′)-Arg-Trp-Lys)-dVal-dPro-NH2 (SEQ ID NO: 31); Ac-dGIn-c(Asp-Pro-dNal(2′)-Arg-Trp-Lys)-dVal-dPro-NH2 (SEQ ID NO: 32); Ac-dAsn-c(Asp-Pro-dNal(2′)-Arg-Trp-Lys)-dVal-dPro-NH2 (SEQ ID NO: 33); Ac-transPro(guan)-c(Asp-Pro-dNal(2′)-Arg-Trp-Lys)-dVal-dPro-NH2 (SEQ ID NO: 34); Ac-cisPro(guan)-c(Asp-Pro-dNal(2′)-Arg-Trp-Lys)-dVal-dPro-NH2 (SEQ ID NO: 35); dTyr-c(Asp-Pro-dNal(2′)-Arg-Trp-Lys)-dVal-dPro-NH2 (SEQ ID NO: 36); Tyr-c(Asp-Pro-dNal(2′)-Arg-Trp-Lys)-dVal-dPro-NH2 (SEQ ID NO: 37); and Dmt-c(Asp-Pro-dNal(2′)-Arg-Trp-Lys)-dVal-dPro-NH2 (SEQ ID NO: 38), wherein c represents cyclization through R² and R⁷ via a lactam bond.

In some aspects, the sequence of Formula (I) is selected from the group consisting of: Ac-Nle-c(dAsp-Pro-dNal(2′)-Arg-Trp-Lys)-dVal-dPro-NH2 (SEQ ID NO: 39); Ac-Nle-c(Asp-Pro-dNal(2′)-Arg-Trp-dLys)-dVal-dPro-NH2 (SEQ ID NO: 40); Ac-Nle-c(Cys-Pro-dNal(2′)-Arg-Trp-Cys)-dVal-dPro-NH2 (SEQ ID NO: 41); Ac-Nle-c(dCys-Pro-dNal(2′)-Arg-Trp-Cys)-dVal-dPro-NH2 (SEQ ID NO: 42); Ac-Nle-c(Cys-Pro-dNal(2′)-Arg-Trp-dCys)-dVal-dPro-NH2 (SEQ ID NO: 43); Ac-Nle-c(dCys-Pro-dNal(2′)-Arg-Trp-dCys)-dVal-dPro-NH2 (SEQ ID NO: 44); Ac-Nle-c(Cys-dNal(2′)-Arg-Trp-Cys)-dVal-dPro-NH2 (SEQ ID NO: 45); Ac-Nle-c(dCys-dNal(2′)-Arg-Trp-Cys)-dVal-dPro-NH2 (SEQ ID NO: 46); Ac-Nle-c(Cys-dNal(2′)-Arg-Trp-dCys)-dVal-dPro-NH2 (SEQ ID NO: 47); Ac-Nle-c(dCys-dNal(2′)-Arg-Trp-dCys)-dVal-dPro-NH2 (SEQ ID NO: 48); Ac-Nle-c(Cys-Pro-dNal(2′)-Arg-Trp-Pen)-dVal-dPro-NH2 (SEQ ID NO: 49); Ac-Nle-c(dCys-Pro-dNal(2′)-Arg-Trp-Pen)-dVal-dPro-NH2 (SEQ ID NO: 50); Ac-Nle-c(Pen-Pro-dNal(2′)-Arg-Trp-Cys)-dVal-dPro-NH2 (SEQ ID NO: 51); Ac-Nle-c(Pen-Pro-dNal(2′)-Arg-Trp-dCys)-dVal-dPro-NH2 (SEQ ID NO: 52); Ac-Nle-c (Pen-Pro-dNal(2′)-Arg-Trp-Pen)-dVal-dPro-NH2 (SEQ ID NO: 53); Ac-Nle-c(dPen-Pro-dNal(2′)-Arg-Trp-Pen)-dVal-dPro-NH2 (SEQ ID NO: 54); Ac-Nle-c(dPen-Pro-dNal(2′)-Arg-Trp-dPen)-dVal-dPro-NH2 (SEQ ID NO: 55); Ac-Nle-c(Pen-Pro-dNal(2′)-Arg-Trp-dPen)-dVal-dPro-NH2 (SEQ ID NO: 56); Ac-Nle-c(Cys-dNal(2′)-Arg-Trp-Pen)-dVal-dPro-NH2 (SEQ ID NO: 57); Ac-Nle-c(dCys-dNal(2′)-Arg-Trp-Pen)-dVal-dPro-NH2 (SEQ ID NO: 58); Ac-Nle-c(Pen-dNal(2′)-Arg-Trp-Cys)-dVal-dPro-NH2 (SEQ ID NO: 59); Ac-Nle-c(Pen-dNal(2′)-Arg-Trp-dCys)-dVal-dPro-NH2 (SEQ ID NO: 60); Ac-Nle-c(Pen-dNal(2′)-Arg-Trp-Pen)-dVal-dPro-NH2 (SEQ ID NO: 61); Ac-Nle-c(dPen-dNal(2′)-Arg-Trp-Pen)-dVal-dPro-NH2 (SEQ ID NO: 62); Ac-Nle-c(dPen-dNal(2′)-Arg-Trp-dPen)-dVal-dPro-NH2 (SEQ ID NO: 63); Ac-Nle-c(Pen-dNal(2′)-Arg-Trp-dPen)-dVal-dPro-NH2 (SEQ ID NO: 64); Ac-Nle-c(Asp-Pro-dNal(2′)-Arg-Trp-Orn)-dVal-dPro-NH2 (SEQ ID NO: 65); Ac-Nle-c(Asp-Pro-dNal(2′)-Arg-Trp-dOrn)-dVal-dPro-NH2 (SEQ ID NO: 66); Ac-Nle-c(Glu-Pro-dNal(2′)-Arg-Trp-Orn)-dVal-dPro-NH2 (SEQ ID NO: 67); and Ac-Nle-c(Glu-Pro-dNal(2′)-Arg-Trp-dOrn)-dVal-dPro-NH2 (SEQ ID NO: 68), wherein c represents cyclization through R² and R⁶ or R⁷ via a lactam bond or a disulfide bond.

In some aspects, the sequence of Formula (I) is selected from the group consisting of: Ac-Nle-c[Asp-dNal(2′)-Arg-Trp-Lys]-dVal-dPro-NH2 (SEQ ID NO: 69); Ac-Nle-c[Asp-Ala-dNal(2′)-Arg-Trp-Lys]-dVal-dPro-NH2 (SEQ ID NO: 70); Ac-Nle-c(Asp-dPro-dNal(2′)-Arg-Trp-Lys)-dVal-dPro-NH2 (SEQ ID NO: 71); Ac-Nle-c(Asp-dAla-dNal(2′)-Arg-Trp-Lys)-dVal-dPro-NH2 (SEQ ID NO: 72); Ac-Nle-c(Asp-dMet-dNal(2′)-Arg-Trp-Lys)-dVal-dPro-NH2 (SEQ ID NO: 73); Ac-Nle-c(Asp-Pro-Gly-dNal(2′)-Arg-Trp-Lys)-dVal-dPro-NH2 (SEQ ID NO: 74); Ac-Nle-c(Asp-Gly-Gly-dNal(2′)-Arg-Trp-Lys)-dVal-dPro-NH2 (SEQ ID NO: 75); Ac-Nle-c[Asp-Gly-Dnal(2′)-Arg-Trp-Lys]-dVal-dPro-NH2 (SEQ ID NO: 76); Ac-Nle-c[Asp-Leu-Dnal(2′)-Arg-Trp-Lys]-dVal-dPro-NH2 (SEQ ID NO: 77); Ac-Nle-c[Asp-Ile-Dnal(2′)-Arg-Trp-Lys]-dVal-dPro-NH2 (SEQ ID NO: 78); Ac-Nle-c[Asp-Val-Dnal(2′)-Arg-Trp-Lys]-dVal-dPro-NH2 (SEQ ID NO: 79); Ac-Nle-c[Asp-dLeu-dNal(2′)-Arg-Trp-Lys]-dVal-dPro-NH2 (SEQ ID NO: 80); Ac-Nle-c[Asp-dIIe-dNal(2′)-Arg-Trp-Lys]-dVal-dPro-NH2 (SEQ ID NO: 81); Ac-Nle-c[Asp-dVal-dNal(2′)-Arg-Trp-Lys]-dVal-dPro-NH2 (SEQ ID NO: 82); Ac-Nle-c[Asp-Trp-dNal(2′)-Arg-Trp-Lys]-dVal-dPro-NH2 (SEQ ID NO: 83); Ac-Nle-c[Asp-dTrp-dNal(2′)-Arg-Trp-Lys]-dVal-dPro-NH2 (SEQ ID NO: 84); Ac-Nle-c[Asp-transPro(guan)-dNal(2′)-Arg-Trp-Lys]-dVal-dPro-NH2 (SEQ ID NO: 85); and Ac-Nle-c[Asp-cisPro(guan)-dNal(2′)-Arg-Trp-Lys]-dVal-dPro-NH2 (SEQ ID NO: 86), wherein c represents cyclization through R² and R⁷ via a lactam bond.

In some aspects, the sequence of Formula (I) is selected from the group consisting of: Ac-Nle-c(Asp-Pro-dNal(2′)-Trp-Lys)-dVal-dPro-NH2 (SEQ ID NO: 87); Ac-Nle-c(Asp-Pro-dNal(2′)-Lys-Trp-Lys)-dVal-dPro-NH2 (SEQ ID NO: 88); Ac-Nle-c(Asp-Pro-dNal(2′)-dLys-Trp-Lys)-dVal-dPro-NH2 (SEQ ID NO: 89); Ac-Nle-c(Asp-Pro-dNal(2′)-dArg-Trp-Lys)-dVal-dPro-NH2 (SEQ ID NO: 90); Ac-Nle-c(Asp-Pro-dNal(2′)-Orn-Trp-Lys)-dVal-dPro-NH2 (SEQ ID NO: 91); Ac-Nle-c(Asp-Pro-dNal(2′)-dOrn-Trp-Lys)-dVal-dPro-NH2 (SEQ ID NO: 92); Ac-Nle-c(Asp-Pro-dNal(2′)-His-Trp-Lys)-dVal-dPro-NH2 (SEQ ID NO: 93); Ac-Nle-c(Asp-Pro-dNal(2′)-Ala-Trp-Lys)-dVal-dPro-NH2 (SEQ ID NO: 94); Ac-Nle-c(Asp-Pro-dNal(2′)-Gly-Trp-Lys)-dVal-dPro-NH2 (SEQ ID NO: 95); Ac-Nle-c(Asp-Pro-dNal(2′)-Asp-Trp-Lys)-dVal-dPro-NH2 (SEQ ID NO: 96); Ac-Nle-c(Asp-Pro-dNal(2′)-Glu-Trp-Lys)-dVal-dPro-NH2 (SEQ ID NO: 97); Ac-Nle-c(Asp-Pro-dNal(2′)-dHis-Trp-Lys)-dVal-dPro-NH2 (SEQ ID NO: 98); Ac-Nle-c(Asp-Pro-dNal(2′)-dAla-Trp-Lys)-dVal-dPro-NH2 (SEQ ID NO: 99); Ac-Nle-c(Asp-Pro-dNal(2′)-dAsp-Trp-Lys)-dVal-dPro-NH2 (SEQ ID NO: 100); and Ac-Nle-c(Asp-Pro-dNal(2′)-dGlu-Trp-Lys)-dVal-dPro-NH2 (SEQ ID NO: 101), wherein c represents cyclization through R² and R⁷ via a lactam bond.

In some aspects, the sequence of Formula (I) is selected from the group consisting of: Ac-Nle-c(Asp-Pro-dNal(2′)-Arg-Lys)-dVal-dPro-NH2 (SEQ ID NO: 102); Ac-Nle-c(Asp-Pro-dNal(2′)-Arg-Nal(1′)-Lys)-dVal-dPro-NH2 (SEQ ID NO: 103); Ac-Nle-c(Asp-Pro-dNal(2′)-Arg-Aia-Lys)-dVal-dPro-NH2 (SEQ ID NO: 104); Ac-Nle-c(Asp-Pro-dNal(2′)-Arg-Phe-Lys)-dVal-dPro-NH2 (SEQ ID NO: 105); Ac-Nle-c(Asp-Pro-dNal(2′)-Arg-Tyr-Lys)-dVal-dPro-NH2 (SEQ ID NO: 106); Ac-Nle-c(Asp-Pro-dNal(2′)-Arg-His-Lys)-dVal-dPro-NH2 (SEQ ID NO: 107); Ac-Nle-c(Asp-Pro-dNal(2′)-Arg-Ala-Lys)-dVal-dPro-NH2 (SEQ ID NO: 108); Ac-Nle-c(Asp-Pro-dNal(2′)-Arg-dNal(1′)-Lys)-dVal-dPro-NH2 (SEQ ID NO: 109); Ac-Nle-c(Asp-Pro-dNal(2′)-Arg-dPhe-Lys)-dVal-dPro-NH2 (SEQ ID NO: 110); Ac-Nle-c(Asp-Pro-dNal(2′)-Arg-dNal(2′)-Lys)-dVal-dPro-NH2 (SEQ ID NO: 111); Ac-Nle-c(Asp-Pro-dNal(2′)-Arg-dTyr-Lys)-dVal-dPro-NH2 (SEQ ID NO: 112); Ac-Nle-c(Asp-Pro-dNal(2′)-Arg-dHis-Lys)-dVal-dPro-NH2 (SEQ ID NO: 113); and Ac-Nle-c(Asp-Pro-dNal(2′)-Arg-dAla-Lys)-dVal-dPro-NH2 (SEQ ID NO: 114), wherein c represents cyclization through R² and R⁷ via a lactam bond.

In some aspects, the sequence of Formula (I) is Ac-Nle-c(Asp-Pro-Bip-Arg-Trp-Lys)-dVal-dPro-NH2 (SEQ ID NO: 115), wherein c represents cyclization through R² and R⁷ via a lactam bond.

In some aspects, the sequence of Formula (I) is selected from the group consisting of: Ac-Nle-c[Asp-Pro-dNal(2′)-Arg-Trp-Lys]-Val-Pro-NH2 (SEQ ID NO: 116); Ac-Nle-c[Asp-Pro-dNal(2′)-Arg-Trp-Lys]-dVal-dPro-OH (SEQ ID NO: 117); Ac-Nle-c[Asp-Pro-dNal(2′)-Arg-Trp-Lys]-dPro-dVal-OH (SEQ ID NO: 118); Ac-Nle-c[Asp-Pro-dNal(2′)-Arg-Trp-Lys]-dVal-Hyp-NH2 (SEQ ID NO: 119); Ac-Nle-c[Asp-Pro-dNal(2′)-Arg-Trp-Lys]-dVal-dHyp-NH2 (SEQ ID NO: 120); Ac-Nle-c[Asp-Pro-dNal(2′)-Arg-Trp-Lys]-Val-Hyp-NH2 (SEQ ID NO: 121); Ac-Nle-c[Asp-Pro-dNal(2′)-Arg-Trp-Lys]-Val-dHyp-NH2 (SEQ ID NO: 122); Ac-Nle-c[Asp-Pro-dNal(2′)-Arg-Trp-Lys]-Hyp-dVal-NH2 (SEQ ID NO: 123); Ac-Nle-c[Asp-Pro-dNal(2′)-Arg-Trp-Lys]-dHyp-dVal-NH2 (SEQ ID NO: 124); Ac-Nle-c[Asp-Pro-dNal(2′)-Arg-Trp-Lys]-Hyp-Val-NH2 (SEQ ID NO: 125); Ac-Nle-c[Asp-Pro-dNal(2′)-Arg-Trp-Lys]-dHyp-Val-NH2 (SEQ ID NO: 126); Ac-Nle-c[Asp-Pro-dNal(2′)-Arg-Trp-Lys]-dVal-dVal-NH2 (SEQ ID NO: 127); Ac-Nle-c[Asp-Pro-dNal(2′)-Arg-Trp-Lys]-dPro-dPro-NH2 (SEQ ID NO: 128); Ac-Nle-c[Asp-Pro-dNal(2′)-Arg-Trp-Lys]-dVal-NH2 (SEQ ID NO: 129); Ac-Nle-c[Asp-Pro-dNal(2′)-Arg-Trp-Lys]-dPro-NH2 (SEQ ID NO: 130); Ac-Nle-c[Asp-Pro-dNal(2′)-Arg-Trp-Lys]-Val-NH2 (SEQ ID NO: 131); Ac-Nle-c[Asp-Pro-dNal(2′)-Arg-Trp-Lys]-Pro-NH2 (SEQ ID NO: 132); Ac-Nle-c[Asp-Pro-dNal(2′)-Arg-Trp-Lys]-Ala-NH2 (SEQ ID NO: 133); Ac-Nle-c[Asp-Pro-dNal(2′)-Arg-Trp-Lys]-dAla-NH2 (SEQ ID NO: 134); Ac-Nle-c[Asp-Pro-dNal(2′)-Arg-Trp-Lys]-dHyp-NH2 (SEQ ID NO: 135); Ac-Nle-c[Asp-Pro-dNal(2′)-Arg-Trp-Lys]-Hyp-NH2 (SEQ ID NO: 136); Ac-Nle-c[Asp-Pro-dNal(2′)-Arg-Trp-Lys]-dAla-dAla-NH2 (SEQ ID NO: 137); Ac-Nle-c[Asp-Pro-dNal(2′)-Arg-Trp-Lys]-Ala-Ala-NH2 (SEQ ID NO: 138); Ac-Nle-c[Asp-Pro-dNal(2′)-Arg-Trp-Lys]-Gly-Gly-NH2 (SEQ ID NO: 139); Ac-Nle-c[Asp-Pro-dNal(2′)-Arg-Trp-Lys]-dVal-Asp-NH2 (SEQ ID NO: 140); Ac-Nle-c[Asp-Pro-dNal(2′)-Arg-Trp-Lys]-dVal-Arg-NH2 (SEQ ID NO: 141); Ac-Nle-c[Asp-Pro-dNal(2′)-Arg-Trp-Lys]-dVal-Asn-NH2 (SEQ ID NO: 142); Ac-Nle-c[Asp-Pro-dNal(2′)-Arg-Trp-Lys]-dVal-dAsp-NH2 (SEQ ID NO: 143); Ac-Nle-c[Asp-Pro-dNal(2′)-Arg-Trp-Lys]-dVal-dArg-NH2 (SEQ ID NO: 144); Ac-Nle-c[Asp-Pro-dNal(2′)-Arg-Trp-Lys]-dVal-dAsn-NH2 (SEQ ID NO: 145); Ac-Nle-c[Asp-Pro-dNal(2′)-Arg-Trp-Lys]-Asp-dPro-NH2 (SEQ ID NO: 146); Ac-Nle-c[Asp-Pro-dNal(2′)-Arg-Trp-Lys]-Arg-dPro-NH2 (SEQ ID NO: 147); Ac-Nle-c[Asp-Pro-dNal(2′)-Arg-Trp-Lys]-Asn-dPro-NH2 (SEQ ID NO: 148); Ac-Nle-c[Asp-Pro-dNal(2′)-Arg-Trp-Lys]-dAsp-dPro-NH2 (SEQ ID NO: 149); Ac-Nle-c[Asp-Pro-dNal(2′)-Arg-Trp-Lys]-dArg-dPro-NH2 (SEQ ID NO: 150); Ac-Nle-c[Asp-Pro-dNal(2′)-Arg-Trp-Lys]-dAsn-dPro-NH2 (SEQ ID NO: 151); Ac-Nle-c[Asp-Pro-dNal(2′)-Arg-Trp-Lys]-Asp-NH2 (SEQ ID NO: 152); Ac-Nle-c[Asp-Pro-dNal(2′)-Arg-Trp-Lys]-Arg-NH2 (SEQ ID NO: 153); Ac-Nle-c[Asp-Pro-dNal(2′)-Arg-Trp-Lys]-Asn-NH2 (SEQ ID NO: 154); Ac-Nle-c[Asp-Pro-dNal(2′)-Arg-Trp-Lys]-dAsp-NH2 (SEQ ID NO: 155); Ac-Nle-c[Asp-Pro-dNal(2′)-Arg-Trp-Lys]-dArg-NH2 (SEQ ID NO: 156); Ac-Nle-c[Asp-Pro-dNal(2′)-Arg-Trp-Lys]-dAsn-NH2 (SEQ ID NO: 157); Ac-Nle-c[Asp-Pro-dNal(2′)-Arg-Trp-Lys]-Lys-Pro-Val-NH2 (SEQ ID NO: 158); Ac-Nle-c[Asp-Pro-dNal(2′)-Arg-Trp-Lys]-Lys-dPro-dVal-NH2 (SEQ ID NO: 159); Ac-Nle-c[Asp-Pro-dNal(2′)-Arg-Trp-Lys]-dLys-dPro-dVal-NH2 (SEQ ID NO: 160); Ac-Nle-c[Asp-Pro-dNal(2′)-Arg-Trp-Lys]-Lys-dPro-NH2 (SEQ ID NO: 161); Ac-Nle-c[Asp-Pro-dNal(2′)-Arg-Trp-Lys]-dLys-dPro-NH2 (SEQ ID NO: 162); Ac-Nle-c[Asp-Pro-dNal(2′)-Arg-Trp-Lys]-Lys-Val-Pro-NH2 (SEQ ID NO: 163); Ac-Nle-c[Asp-Pro-dNal(2′)-Arg-Trp-Lys]-Lys-dVal-dPro-NH2 (SEQ ID NO: 164); Ac-Nle-c[Asp-Pro-dNal(2′)-Arg-Trp-Lys]-dLys-dVal-dPro-NH2 (SEQ ID NO: 165); Ac-Nle-c[Asp-Pro-dNal(2′)-Arg-Trp-Lys]-Arg-Pro-Val-NH2 (SEQ ID NO: 166); Ac-Nle-c[Asp-Pro-dNal(2′)-Arg-Trp-Lys]-Arg-dPro-dVal-NH2 (SEQ ID NO: 167); Ac-Nle-c[Asp-Pro-dNal(2′)-Arg-Trp-Lys]-dArg-dPro-dVal-NH2 (SEQ ID NO: 168); Ac-Nle-c[Asp-Pro-dNal(2′)-Arg-Trp-Lys]-Arg-Val-Pro-NH2 (SEQ ID NO: 169); Ac-Nle-c[Asp-Pro-dNal(2′)-Arg-Trp-Lys]-Arg-dVal-dPro-NH2 (SEQ ID NO: 170); Ac-Nle-c[Asp-Pro-dNal(2′)-Arg-Trp-Lys]-dArg-dVal-dPro-NH2 (SEQ ID NO: 171); Ac-Nle-c[Asp-Pro-dNal(2′)-Arg-Trp-Lys]-dTle-dVal-dPro-NH2 (SEQ ID NO: 172); Ac-Nle-c[Asp-Pro-dNal(2′)-Arg-Trp-Lys]-dTle-dPro-NH2 (SEQ ID NO: 173); Ac-Nle-c[Asp-Pro-dNal(2′)-Arg-Trp-Lys]-dTle-dVal-NH2 (SEQ ID NO: 174); Ac-Nle-c[Asp-Pro-dNal(2′)-Arg-Trp-Lys]-dTle-dTle-NH2 (SEQ ID NO: 175); Ac-Nle-c[Asp-Pro-dNal(2′)-Arg-Trp-Lys]-dTle-dTle-dPro-NH2 (SEQ ID NO: 176); Ac-Nle-c[Asp-Pro-dNal(2′)-Arg-Trp-Lys]-dTle-dTle-dVal-NH2 (SEQ ID NO: 177); Ac-Nle-c[Asp-Pro-dNal(2′)-Arg-Trp-Lys]-dPro-dTle-NH2 (SEQ ID NO: 178); and Ac-Nle-c[Asp-Pro-dNal(2′)-Arg-Trp-Lys]-dVal-dTle-NH2 (SEQ ID NO: 179), wherein c represents cyclization through R² and R⁷ via a lactam bond.

In some aspects, the sequence of Formula (I) is selected from the group consisting of: Ac-Nle-c(Asp-Pro-dNal(2′)-Arg-Trp-Lys)-dVal-dVal-dPro-NH2 (SEQ ID NO: 180); Ac-Nle-c(Asp-Pro-dNal(2′)-Arg-Trp-Lys)-dPro-dVal-dPro-NH2 (SEQ ID NO: 181); Ac-Nle-c(Asp-Pro-dNal(2′)-Arg-Trp-Lys)-dVal-dVal-dVal-dPro-NH2 (SEQ ID NO: 182); Ac-Nle-c(Asp-Pro-dNal(2′)-Arg-Trp-Lys)-dVal-dPro-dVal-dPro-NH2 (SEQ ID NO: 183); and Ac-Nle-c(Asp-Pro-dNal(2′)-Arg-Trp-Lys)-dVal-dVal-dVal-dVal-dVal-dVal-dPro-NH2 (SEQ ID NO: 184), wherein c represents cyclization through R² and R⁷ via a lactam bond.

In some aspects, X¹ is present and is acetylated norleucine, and R¹ is present and is norleucine. In some aspects, X² is present and is norleucine. In some aspects, X³ is present and is norleucine.

In some aspects, the sequence of Formula (I) is selected from the group consisting of: Ac-Nle-Nle-c(Asp-Pro-dNal(2′)-Arg-Trp-Lys)-dVal-dPro-NH2 (SEQ ID NO: 185); Ac-Nle-Nle-Nle-c(Asp-Pro-dNal(2′)-Arg-Trp-Lys)-dVal-dPro-NH2 (SEQ ID NO: 186); and Ac-Nle-Nle-Nle-Nle-c(Asp-Pro-dNal(2′)-Arg-Trp-Lys)-dVal-dPro-NH2 (SEQ ID NO: 187), wherein c represents cyclization through R² and R⁷ via a lactam bond.

In some aspects, the sequence of Formula (I) is selected from the group consisting of: Ac-Nle-c[Asp-Pro-Pro-dNal(2′)-Arg-Trp-Lys]-dVal-dPro-NH2 (SEQ ID NO: 195); Ac-Nle-c[Asp-Trp-Pro-dNal(2′)-Arg-Trp-Lys]-dVal-dPro-NH2 (SEQ ID NO: 196); Ac-Nle-c[Asp-Pro-dNal(2′)-Arg-dTrp-Lys]-dVal-dPro-NH2 (SEQ ID NO: 197); c[CO-cis-CH═CH—CO-Pro-D-Nal(2′)-Arg-Trp-Lys]-dVal-dPro-NH2 (SEQ ID NO: 198); Ac-Nle-c[Asp-Aba-D-Phe-Arg-Trp-Lys]-dVal-dPro-NH2 (SEQ ID NO: 199); Ac-Nle-c[Asp-β-Ala-D-Nal(2′)-Arg-Trp-Lys]-dVal-dPro-NH2 (SEQ ID NO: 200); Ac-Nle-c[Asp-Mamb-D-Nal(2′)-Arg-Trp-Lys]-dVal-dPro-NH2 (SEQ ID NO: 201); Ac-Nle-c[Asp-Acpc-D-Nal(2′)-Arg-Trp-Lys]-dVal-dPro-NH2 (SEQ ID NO: 202); Ac-c[Cys-Arg-D-Phe-Cys]-Trp-dVal-dPro-NH2 (SEQ ID NO: 203); Ac-Nle-c[Asp-Pro-dNal(2′)-Arg-Trp-Lys]-Trp-NH2 (SEQ ID NO: 204); Ac-Nle-c(Asp-Aba-dNal(2′)-Arg-Trp-Lys)-dVal-dPro-NH2 (SEQ ID NO: 205); Ac-Nle-c(Asp-Aia-dNal(2′)-Arg-Trp-Lys)-dVal-dPro-NH2 (SEQ ID NO: 206); Ac-Nle-c(Asp-Ata-dNal(2′)-Arg-Trp-Lys)-dVal-dPro-NH2 (SEQ ID NO: 207); Ac-Nle-c(Asp-Aia-dPhe-Arg-Trp-Lys)-dVal-dPro-NH2 (SEQ ID NO: 208); Ac-Nle-c(Asp-Ata-dPhe-Arg-Trp-Lys)-dVal-dPro-NH2 (SEQ ID NO: 209); Ac-Nle-c(Asp-Aba-p(CI)dPhe-Arg-Trp-Lys)-dVal-dPro-NH2 (SEQ ID NO: 210); Ac-Nle-c(Asp-Aia-p(CI)dPhe-Arg-Trp-Lys)-dVal-dPro-NH2 (SEQ ID NO: 211); and Ac-Nle-c(Asp-Ata-p(CI)dPhe-Arg-Trp-Lys)-dVal-dPro-NH2 (SEQ ID NO: 212), wherein c represents cyclization through R² and R⁷ or R⁸ via a lactam bond or through R² and R⁵ via a disulfide bond.

In some aspects, the sequence of Formula (I) is selected from the group consisting of: Ac-Nle-c[Asp-Aic-dNal(2′)-Arg-Trp-Lys]-dVal-dPro-NH2 (SEQ ID NO: 240); Ac-Nle-c[Asp-Cpe-dNal(2′)-Arg-Trp-Lys]-dVal-dPro-NH2 (SEQ ID NO: 241); Ac-Nle-c[Asp-Che-dNal(2′)-Arg-Trp-Lys]-dVal-dPro-NH2 (SEQ ID NO: 242); Ac-Nle-c[Asp-Oic-dNal(2′)-Arg-Trp-Lys]-dVal-dPro-NH2 (SEQ ID NO: 243); Ac-Nle-c[Asp-loc-dNal(2′)-Arg-Trp-Lys]-dVal-dPro-NH2 (SEQ ID NO: 244); Ac-Nle-c[Asp-Tic-dNal(2′)-Arg-Trp-Lys]-dVal-dPro-NH2 (SEQ ID NO: 245); Ac-Nle-c[Asp-Pro-dNal(2′)-Arg-Trp-Pro-Lys]-dVal-dPro-NH2 (SEQ ID NO: 246); Ac-Nle-c[Asp-His-dNal(2′)-Pro-Trp-Lys]-dVal-dPro-NH2 (SEQ ID NO: 247); Ac-Nle-c[Asp-His-dNal(2′)-transPro(guan)-Trp-Lys]-dVal-dPro-NH2 (SEQ ID NO: 248); Ac-Nle-c[Asp-His-dNal(2′)-cisPro(guan)-Trp-Lys]-dVal-dPro-NH2 (SEQ ID NO: 249); Ac-Nle-c[Asp-Pro-dNal(2′)-Pro-Trp-Lys]-dVal-dPro-NH2 (SEQ ID NO: 250); Ac-Nle-c[Asp-Pro-dNal(2′)-transPro(guan)-Trp-Lys]-dVal-dPro-NH2 (SEQ ID NO: 251); Ac-Nle-c[Asp-Pro-dNal(2′)-cisPro(guan)-Trp-Lys]-dVal-dPro-NH2 (SEQ ID NO: 252); Ac-Nle-c(Asp-Pro-dNal(2′)-Arg-Aia-Lys)-dVal-dPro-NH2 (SEQ ID NO: 253); Ac-Nle-c(Asp-Pro-dNal(2′)-Arg-Aba-Lys)-dVal-dPro-NH2 (SEQ ID NO: 254); Ac-Nle-c(Asp-Pro-dNal(2′)-Arg-Ata-Lys)-dVal-dPro-NH2 (SEQ ID NO: 255); Ac-Nle-c[Asp-Glu-dNal(2′)-Arg-Trp-Lys]-dVal-dPro-NH2 (SEQ ID NO: 256); Ac-Nle-c[Asp-Glu-dNal(2′)-Arg-Trp-Gly-Lys]-dVal-dPro-NH2 (SEQ ID NO: 257); Ac-Nle-c[Asp-Pro-Glu-dNal(2′)-Arg-Trp-Lys]-dVal-dPro-NH2 (SEQ ID NO: 258); Ac-Nle-c[Asp-Pro-Glu-dNal(2′)-Arg-Trp-Gly-Lys]-dVal-dPro-NH2 (SEQ ID NO: 259); Ac-Nle-c[Asp-Pro-dNal(2′)-Arg-Trp-Gly-Lys]-dVal-dPro-NH2 (SEQ ID NO: 260); Ac-Nle-c[Asp-Pro-dNal(2′)-Arg-Trp-Gly-Lys]-dPro-dPro-Lys-Asp-NH2 (SEQ ID NO: 261); Ac-Nle-c[Asp-Pro-dNal(2′)-Arg-Trp-Gly-Lys]-dPro-dPro-dLys-dAsp-NH2 (SEQ ID NO: 262); Ac-Glu-c[Asp-Pro-dNal(2′)-Arg-Trp-Gly-Lys]-dPro-dPro-Lys-Asp-NH2 (SEQ ID NO: 263); and Ac-Nle-c[Asp-Pro-dNal(2′)-Arg-Trp-Lys]-dPro-dPro-dLys-dAsp-NH2 (SEQ ID NO: 264), wherein c represents cyclization through R² and R⁷ via a lactam bond.

In some aspects, the sequence of Formula (I) is selected from the group consisting of: Ac-Nle-c[Asp-Glu-His-dNal(2′)-Arg-Trp-Lys]-dVal-dPro-NH2 (SEQ ID NO: 265); Ac-Nle-c[Asp-Glu-His-dNal(2′)-Arg-Trp-Gly-Lys]-dVal-dPro-NH2 (SEQ ID NO: 266); Ac-Nle-c(Asp-dAla-His-dNal(2′)-Arg-Trp-Lys)-dVal-dPro-NH2 (SEQ ID NO: 267); Ac-Arg-c(Asp-dAla-His-dNal(2′)-Arg-Trp-Lys)-dVal-dPro-NH2 (SEQ ID NO: 268); Ac-Arg-c(Asp-dAla-His-dPhe-Arg-Trp-Lys)-dVal-dPro-NH2 (SEQ ID NO: 269); Ac-Arg-c (Cys-dAla-His-dNal(2′)-Arg-Trp-Cys)-dVal-dPro-NH2 (SEQ ID NO: 270); Ac-dArg-c(Asp-dAla-His-dNal(2′)-Arg-Trp-Lys)-dVal-dPro-NH2 (SEQ ID NO: 271); Ac-Arg-c(Asp-dAla-dNal(2′)-Arg-Trp-Lys)-dVal-dPro-NH2 (SEQ ID NO: 272); Ac-dArg-c(Asp-dAla-dNal(2′)-Arg-Trp-Lys)-dVal-dPro-NH2 (SEQ ID NO: 273); Ac-Nle-c(Asp-Ala-His-dNal(2′)-Arg-Trp-Lys)-dVal-dPro-NH2 (SEQ ID NO: 274); Ac-Arg-c(Asp-Ala-His-dNal(2′)-Arg-Trp-Lys)-dVal-dPro-NH2 (SEQ ID NO: 275); Ac-dArg-c(Asp-Ala-His-dNal(2′)-Arg-Trp-Lys)-dVal-dPro-NH2 (SEQ ID NO: 276); Ac-Nle-c(Asp-Trp-Arg-dNal(2′)-Pro-Lys)-dVal-dPro-NH2 (SEQ ID NO: 277); Ac-Nle-c(Asp-Pro-Trp-dNal(2′)-Lys)-dVal-dPro-NH2 (SEQ ID NO: 278); Ac-Nle-c(Asp-Pro-Trp-Arg-dNal(2′)-Pro-Lys)-dVal-dPro-NH2 (SEQ ID NO: 279); Ac-Nle-c(Asp-Pro-Trp-Arg-dNal(2′)-Lys)-dVal-dPro-NH2 (SEQ ID NO: 280); Ac-Nle-c(Lys-Trp-Arg-dNal(2′)-Pro-Asp)-dVal-dPro-NH2 (SEQ ID NO: 281) Ac-Arg-c(Asp-dAla-His-dPhe-Arg-Trp-Lys)-NH2 (SEQ ID NO: 282); Ac-Arg-c (Cys-dAla-His-dNal(2′)-Arg-Trp-Cys)-NH2 (SEQ ID NO: 283); and Ac-Arg-c(Asp-dAla-His-dNal(2′)-Arg-Trp-Lys)-NH2 (SEQ ID NO: 284), wherein c represents cyclization through R² and any of R⁵—R⁸ via a lactam bond or a disulfide bond.

In some aspects, the sequence of Formula (I) is: Ac-dVal-dPro-c(Asp-Trp-Arg-dNal(2′)-Pro-Lys)-Nle-NH2 (SEQ ID NO: 285); Ac-dVal-dPro-c (Lys-Trp-Arg-dNal(2′)-Pro-Asp)-Nle-NH2 (SEQ ID NO: 286); Ac-dVal-dPro-c(Asp-Trp-Arg-dNal(2′)-Pro-Lys)-Nle-Nle-NH2 (SEQ ID NO: 287); Ac-dVal-dPro-c (Lys-Trp-Arg-dNal(2′)-Pro-Asp)-Nle-Nle-NH2 (SEQ ID NO: 288); Ac-dVal-dPro-c(Asp-Trp-Arg-dNal(2′)-Pro-Lys)-dVal-dPro-NH2 (SEQ ID NO: 289); Ac-dVal-dPro-c (Lys-Trp-Arg-dNal(2′)-Pro-Asp)-dVal-dPro-NH2 (SEQ ID NO: 290), wherein c represents cyclization through R¹ and any one of R⁶ via a lactam bond.

In some aspects, the non-naturally occurring melanocortin analog includes any one of the sequences of SEQ ID NOs: 2-290. In some aspects, the non-naturally occurring melanocortin analog includes any one of the sequences of SEQ ID NOs: 2-187, 196-212, and 240-290. In some aspects, the non-naturally occurring melanocortin analog includes a sequence of SEQ ID NO: 3.

In some aspects, the non-naturally occurring melanocortin analog is present in a composition. In some aspects, the composition further includes a pharmaceutical salt. In some aspects, the composition further includes a pharmaceutical carrier.

In some aspects, the non-naturally occurring melanocortin analog is present in the composition in a concentration of 0.1 mg/mL to 500 mg/mL, relative to a total volume of the composition. In some aspects, the non-naturally occurring melanocortin analog includes a sequence of SEQ ID NO: 3, and wherein the non-naturally occurring melanocortin analog is present in the composition in a concentration of 5 mg/mL to 100 mg/mL, relative to a total volume of the composition. In some aspects, the non-naturally occurring melanocortin analog is present in the composition in a concentration of about 50 mg/mL, relative to a total volume of the composition.

In some aspects, the non-naturally occurring melanocortin analog is administered via intraperitoneal, intravenous, parenteral, subcutaneous, intramuscular, intracerebroventricular, intranasal, or oral administration. In some aspects, the composition including the non-naturally occurring melanocortin analog is administered to the subject parenterally. In some aspects, the composition including the non-naturally occurring melanocortin analog is administered to the subject subcutaneously.

In some aspects, the non-naturally occurring melanocortin analog crosses the blood-brain-barrier of the subject.

In some aspects, the therapeutically effective amount of the non-naturally occurring melanocortin analog is from 0.001 mg/kg to 25 mg/kg per body weight of the subject. In some aspects, the therapeutically effective amount of the non-naturally occurring melanocortin analog is from 0.5 mg/kg to 10 mg/kg per body weight of the subject. In some aspects, the non-naturally occurring melanocortin analog is administered at least once daily in an amount ranging from 0.001 mg/kg to 25 mg/kg per body weight of the subject. In some aspects, the non-naturally occurring melanocortin analog is administered at least once daily in an amount ranging from about 0.5 mg/kg to about 10 mg/kg per body weight of the subject.

In some aspects, the non-naturally occurring melanocortin analog includes a sequence of SEQ ID NO: 3, and the non-naturally occurring melanocortin analog is administered at least once daily in an amount ranging from 0.001 mg/kg to 25 mg/kg per body weight of the subject. In some aspects, the non-naturally occurring melanocortin analog includes a sequence of SEQ ID NO: 3, and the non-naturally occurring melanocortin analog is administered at least once daily in an amount ranging from 0.5 mg/kg to 10 mg/kg per body weight of the subject.

In some aspects, the non-naturally occurring melanocortin analog is administered to the subject for at least 1 day, 1 week, 1 month, 3 months, 6 months, 1 year, or 5 years. In some aspects, the non-naturally occurring melanocortin analog is administered to the subject for 1 day, 5 days, 7 days, 14 days, 21 days, 28 days, 35 days, 40 days, 45 days, 50 days, 60 days, 75 days, 90 days, 100 days, 110 days, or 120 days. In some aspects, the pharmaceutical carrier includes water.

In some aspects, the subject experiences loss of appetite, reduced appetite, decreased food consumption, and/or weight loss prior to the administration.

In some aspects, the loss of appetite, reduced appetite, decreased food consumption, and/or weight loss is caused by cachexia.

In some aspects, the method (i) stimulates appetite of the subject; (ii) increases food consumption by the subject; (iii) prevents or alleviates nausea, emesis, and/or anorexia in the subject; (iv) increases or maintains body weight or BMI of the subject; (v) prevents or reduces weight loss of the subject; (vi) increases or maintains muscle mass of the subject; (vii) prevents or reduces muscle mass loss of the subject; (viii) increases or maintains fat mass of the subject; and/or (ix) prevents or reduces fat mass loss of the subject.

In some aspects, the method increases body weight or BMI of and/or increases food consumption by the subject.

In some aspects, appetite of the subject is increased by at least 10% to 200% after the administration. In some aspects, appetite of the subject is increased by at least 25% to 100% after the administration.

In some aspects, appetite is increased for at least 1 day, 1 week, 1 month, 3 months, 6 months, 1 year, or 5 years during the administration. In some aspects, appetite is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration. In some aspects, the increased appetite is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

In some aspects, the appetite is assessed by a scale for measuring desire to eat, feeling of hunger, and/or satiety. In some aspects, the appetite is assessed between meals.

In some aspects, the subject includes two or more subjects, and wherein the appetite is an average appetite of the two or more subjects.

In some aspects, food consumption of the subject is increased by at least 25% to 2,000% after the administration. In some aspects, food consumption of the subject is increased by at least 50% to 500% after the administration.

In some aspects, food consumption is increased for at least 1 day, 1 week, 1 month, 3 months, 6 months, 1 year, or 5 years during the administration. In some aspects, food consumption is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration. In some aspects, the increased food consumption is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

In some aspects, the food consumption is determined by total calories (kcal) consumed in 1 day or mass of food (grams or kilograms) consumed in 1 day.

In some aspects, anorexia is determined by food consumption.

In some aspects, body weight of the subject is increased by at least 5% to 200% after the administration. In some aspects, body weight of the subject is increased by at least 5% to 25% after the administration. In some aspects, body weight of the subject is increased by at least 20% to 100% after the administration.

In some aspects, the increased body weight is maintained for at least 1 week, 2 weeks, 3 weeks, 1 month, 3 months, 6 months, 1 year, or 5 years, after the administration is stopped.

In some aspects, muscle mass of the subject is increased by at least 1% to 100% after the administration. In some aspects, muscle mass of the subject is increased by at least 5% to 15% after the administration. In some aspects, muscle mass of the subject is increased by at least 25% to 50% after the administration.

In some aspects, the increased muscle mass is maintained for at least 1 week, 2 weeks, 3 weeks, 1 month, 3 months, 6 months, 1 year, or 5 years, after the administration is stopped.

In some aspects, the muscle mass is cardiac muscle mass, skeletal muscle mass, or both.

In some aspects, fat mass of the subject is increased by at least 1% to 100% after the administration. In some aspects, fat mass of the subject is increased by at least 5% to 15% after the administration. In some aspects, fat mass of the subject is increased by at least 25% to 50% after the administration.

In some aspects, the increased fat mass is maintained for at least 1 week, 2 weeks, 3 weeks, 1 month, 3 months, 6 months, 1 year, or 5 years, after the administration is stopped.

In some aspects, the method stimulates appetite in the subject compared to a control subject, wherein the control subject is not administered the non-naturally occurring melanocortin analog. In some aspects, the control subject experiences loss of appetite, reduced appetite, decreased food consumption, and/or weight loss. In some aspects, the loss of appetite, reduced appetite, decreased food consumption, and/or weight loss is caused by cachexia.

In some aspects, appetite of the subject is increased after the administration by at least 10% to 200%, relative to appetite of the control subject. In some aspects, appetite of the subject is increased after the administration by at least 25% to 100%, relative to appetite of the control subject.

In some aspects, appetite is increased for at least 1 day, 1 week, 1 month, 3 months, 6 months, 1 year, or 5 years during the administration.

In some aspects, appetite is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration. In some aspects, the increased appetite is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

In some aspects, the appetite is assessed by a scale for measuring desire to eat, feeling of hunger, and/or level of satiety. In some aspects, the appetite is assessed between meals.

In some aspects, the subject includes two or more subjects, and wherein the appetite of the subject is an average appetite of the two or more subjects. In some aspects, the control subject includes two or more control subjects, and wherein the appetite of the control subject is an average appetite of the two or more control subjects.

In some aspects, food consumption of the subject is increased after the administration by at least 25% to 2,000%, relative to food consumption of the control subject. In some aspects, food consumption of the subject is increased after the administration by at least 50% to 500%, relative to food consumption of the control subject.

In some aspects, food consumption is increased for at least 1 day, 1 week, 1 month, 3 months, 6 months, 1 year, or 5 years during the administration. In some aspects, food consumption is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration. In some aspects, the increased food consumption is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

In some aspects, the food consumption is determined by total calories (kcal) consumed in 1 day or mass of food (grams or kilograms) consumed in 1 day.

In some aspects, body weight of the subject is increased after the administration, and wherein weight gain of the subject after the administration is at least 5% to 200% greater than that of the control subject. In some aspects, body weight of the subject is increased after the administration, and wherein weight gain of the subject after the administration is at least 5% to 25% greater than that of the control subject. In some aspects, body weight of the subject is increased after the administration, and wherein weight gain of the subject after the administration is at least 20% to 100% greater than that of the control subject.

In some aspects, the increased body weight is maintained for at least 1 week, 2 weeks, 3 weeks, 1 month, 3 months, 6 months, 1 year, or 5 years, after the administration is stopped.

In some aspects, muscle mass of the subject is increased after the administration, and wherein muscle mass gain of the subject after the administration is at least 1% to 100% greater than that of the control subject. In some aspects, muscle mass of the subject is increased after the administration, and wherein muscle mass gain of the subject after the administration is at least 5% to 15% greater than that of the control subject. In some aspects, muscle mass of the subject is increased after the administration, and wherein muscle mass gain of the subject after the administration is at least 25% to 50% greater than that of the control subject.

In some aspects, the increased muscle mass is maintained for at least 1 week, 2 weeks, 3 weeks, 1 month, 3 months, 6 months, 1 year, or 5 years, after the administration is stopped.

In some aspects, fat mass of the subject is increased after the administration, and wherein fat mass gain of the subject after the administration is at least 1% to 100% greater than that of the control subject. In some aspects, fat mass of the subject is increased after the administration, and wherein fat mass gain of the subject after the administration is at least 5% to 15% greater than that of the control subject. In some aspects, fat mass of the subject is increased after the administration, and wherein fat mass gain of the subject after the administration is at least 25% to 50% greater than that of the control subject.

In some aspects, the increased fat mass is maintained for at least 1 week, 2 weeks, 3 weeks, 1 month, 3 months, 6 months, 1 year, or 5 years, after the administration is stopped.

In some aspects, the subject is a human. In some aspects, the subject is an animal.

In some aspects, the subject has a body mass index (BMI) of 18.5 kg/m2 to 25 kg/m2. In some aspects, the subject has a BMI of less than 20 kg/m2. In some aspects, the subject has a BMI of less than 18.5 kg/m2.

In some aspects, the subject is a metabolically unchallenged subject, and wherein the method increases body weight of and/or increases food consumption by the metabolically unchallenged subject compared to a control subject. In some aspects, the metabolically unchallenged subject and the control subject are the same subject. In some aspects, the metabolically unchallenged subject and the control subject are different subjects. In some aspects, the metabolically unchallenged subject experiences loss of appetite, reduced appetite, a loss of food consumption, and/or weight loss prior to the administration.

In some aspects, the non-naturally occurring melanocortin analog is administered to the at least once daily in an amount ranging from 0.001 mg/kg to 25 mg/kg per body weight of the metabolically unchallenged subject. In some aspects, the non-naturally occurring melanocortin analog is administered at least once daily in an amount ranging from about 0.5 mg/kg to about 10 mg/kg per body weight of the metabolically unchallenged subject.

In some aspects, the non-naturally occurring melanocortin analog is administered to the metabolically unchallenged subject for at least 1 day, 1 week, 1 month, 3 months, 6 months, 1 year, or 5 years. In some aspects, the non-naturally occurring melanocortin analog is administered to the metabolically unchallenged subject for 1 day, 7 days, 14 days, 21 days, 28 days, 35 days, 40 days, 45 days, 50 days, 60 days, 75 days, 90 days, 100 days, 110 days, or 120 days.

In some aspects, body weight of the metabolically unchallenged subject is increased after the administration by at least 5% to 200%, relative to body weight of the control subject before the administration, wherein the control subject is a metabolically unchallenged subject. In some aspects, body weight of the metabolically unchallenged subject is increased after the administration by at least about 5% to about 25%, relative to body weight of the control subject before the administration, wherein the control subject is a metabolically unchallenged subject. In some aspects, body weight of the metabolically unchallenged subjects increased after the administration by at least about 20% to about 100%, relative to body weight of the control subject before the administration, wherein the control subject is a metabolically unchallenged subject. In some aspects, body weight of the metabolically unchallenged subject is increased after the administration by at least 5% to 200%, relative to body weight of the control subject, wherein the control subject is a metabolically unchallenged subject who experiences loss of appetite, reduced appetite, and/or weight loss. In some aspects, body weight of the subject is increased after the administration by at least about 5% to about 25%, relative to body weight of the control subject, wherein the control subject is a metabolically unchallenged subject who experiences loss of appetite, reduced appetite, and/or weight loss. In some aspects, body weight of the subject is increased after the administration by at least about 20% to about 100%, relative to body weight of the control subject, wherein the control subject is a metabolically unchallenged subject who experiences loss of appetite, reduced appetite, and/or weight loss.

In some aspects, the increased body weight is maintained for at least 1 week, 2 weeks, 3 weeks, 1 month, 3 months, 6 months, 1 year, or 5 years, after the administration is stopped.

In some aspects, food consumption of the metabolically unchallenged subject is increased after the administration by at least 50% to 2,000%, relative to food consumption of the metabolically unchallenged subject before the administration. In some aspects, food consumption of the metabolically unchallenged subject is increased after the administration by at least about 100%, relative to food consumption of the metabolically unchallenged subject before the administration. In some aspects, food consumption of the metabolically unchallenged subject is increased after the administration by at least about 1,000%, relative to food consumption of the metabolically unchallenged subject before the administration. In some aspects, food consumption of the metabolically unchallenged subject is increased after the administration by at least 50% to 2,000%, relative to food consumption of the control subject, wherein the control subject is a metabolically unchallenged subject who experiences loss of appetite, reduced appetite, and/or weight loss.

In some aspects, food consumption is increased for at least 1 day, 1 week, 1 month, 3 months, 6 months, 1 year, or 5 years during the administration. In some aspects, food consumption is increased for at least 1 day, 7 days, 14 days, 21 days, 28 days, 35 days, 40 days, 45 days, 50 days, 60 days, 75 days, 90 days, 100 days, 110 days, or 120 days during the administration. In some aspects, the increased food consumption is maintained for at least 1 week, 2 weeks, 3 weeks, 1 month, 3 months, 6 months, 1 year, or 5 years, after the administration is stopped.

In some aspects, food consumption is determined by total calories (kcal) consumed in 1 day or mass of food (grams or kilograms) consumed in 1 day.

In some aspects, one or more side effects exhibited by the metabolically unchallenged subject after administration of the non-naturally occurring melanocortin analog are reduced compared to one or more side effects exhibited by a control subject that was administered a natural melanocortin.

In some aspects, the control subject is a metabolically unchallenged subject.

In some aspects, the metabolically unchallenged subject has a disease or condition selected from the group consisting of a psychological disease or condition, an allergy, an intolerance, a gastrointestinal disease or condition, a side-effect from a medication, substance use, a viral infection, a bacterial infection, food poisoning, dehydration, fatigue, hormonal imbalance, pain, a cardiovascular disease or condition, anemia, an autoimmune disease or condition, a respiratory disease or condition, and an inflammatory disease or condition.

In some aspects, the psychological disease or condition is selected from the group consisting of depression, bipolar disorder, schizophrenia, anorexia nervosa, anxiety, grief, stress, bulimia, post-traumatic stress disorder, phobia, aversions such as smell, taste, sight, texture, social anxiety, shock, obsessive-compulsive disorder, eating disorders dementia, Alzheimer's, Parkinson's, multiple sclerosis.

In some aspects, the allergy or intolerance is selected from the group consisting of gluten, dairy, soy, nut and seed.

In some aspects, the gastrointestinal disease or condition is selected from the group consisting of irritable bowel syndrome (IBS), celiac disease, and Crohn's disease.

In some aspects, the disease or condition is advanced age.

In some aspects, the medication is selected from the group consisting of antibiotics, codeine, morphine, sleeping pills, blood pressure medications, diuretics, anabolic steroids, cardiovascular medications, including digoxin, fluoxetine and hydralazine. In some aspects, the substance use is selected from the group consisting of cocaine, methamphetamines, heroin, alcohol.

In some aspects, the disease or condition is substance withdrawal. In some aspects, the disease or condition is food poisoning. In some aspects, the disease or condition is dehydration. In some aspects, the viral infection, bacterial infection, or disease or condition caused by a viral infection or a bacterial infection is selected from the group consisting of upper respiratory infection, COVID-19, pneumonia, gastroenteritis, skin, meningitis, HIV, hepatitis, flu, common cold, urine infection. In some aspects, the disease or condition is selected from the group consisting of sleep deprivation, fatigue, chronic fatigue syndrome, nausea, loss of taste, smell, sight, and satiety. In some aspects, the disease or condition is pregnancy or hormonal therapy.

In some aspects, the pain is caused by a condition or disease selected from the group consisting of fibromyalgia, migraines, nerve damage, postural orthostatic tachycardia syndrome, post-surgical, oral pain, and dental pain.

In some aspects, the disease or condition is selected from the group consisting of heart disease, high blood pressure, anemia, lupus, rheumatoid arthritis, chronic lung disease, inflammatory conditions, and menopause.

In some aspects, the metabolically unchallenged subject is a human. In some aspects, the metabolically unchallenged subject is an animal.

In some aspects, the increased body weight or BMI includes increased muscle mass, increased fat mass, or both. In some aspects, the muscle mass is skeletal muscle mass, cardiac muscle mass, or both. In some aspects, the increased body weight or BMI includes increased lean mass.

In some aspects, the subject has a cancer. In some aspects, the cancer is at least one selected from the group consisting of bone cancer, testicular cancer, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colorectal cancer, oral cancer, skin cancer, and melanoma. In some aspects, the cancer is at least one selected from the group consisting of bone cancer, lung cancer, testicular cancer, breast cancer, ovarian cancer, cervical cancer, bladder cancer, and head and neck cancer. In some aspects, the cancer is at least one selected from the group consisting of non-small-cell lung cancer, colorectal cancer, stomach cancer, ovarian cancer, and pancreatic cancer.

In some aspects, the present technology further includes administering an anticancer agent to the subject. In some aspects, the anticancer agent is at least one chemotherapeutic agent.

In some aspects, the at least one chemotherapeutic agent includes one or more chemotherapeutic agents selected from the group consisting of a platinum-coordination complex, an antimetabolite, a tubulin binding agent, an alkylating antineoplastic agent, and a cytotoxic antibiotic.

In some aspects, the present technology further includes administering an appetite-regulating agent to the subject.

In some aspects, the subject does not experience an adverse event following administration of the non-naturally occurring melanocortin analog. In some aspects, the subject does not experience an adverse event attributable to the non-naturally occurring melanocortin analog following administration.

In some aspects, appetite of the subject is assessed by a scale for measuring satiety.

In some aspects, satiety decreased by at least 40% to 150% within 8 days after the administration. In some aspects, satiety decreased by at least 7 points within 8 days after the administration. In some aspects, satiety decreased below 15 within 8 days after the administration. In some aspects, body weight of the subject is increased by at least 2% to 10% between day 2 after the administration and day 7 after the administration. In some aspects, body weight of the subject is increased by at least 1.5 kg between day 2 after the administration and day 7 after the administration. In some aspects, BMI of the subject is increased by at least 1% to 10% between day 2 after the administration and day 7 after the administration. In some aspects, BMI of the subject is increased by at least 0.5 between day 2 after administration and day 7 after administration.

In some aspects, appetite of the subject is assessed by a scale for measuring satiety.

In some aspects, satiety of the subject is decreased after the administration by at least 40% to 150%, relative to satiety of the control subject.

In some aspects, satiety of the subject is decreased after the administration by at least 14 points relative to satiety of the control subject.

In some aspects, body weight of the subject is increased between day 2 after the administration and day 7 after the administration, and wherein weight gain of the subject between day 2 after the administration and day 7 after the administration is at least 20% to 150% greater than that of the control subject. In some aspects, body weight of the subject is increased between day 2 after the administration and day 7 after the administration, and wherein weight gain of the subject between day 2 after the administration and day 7 after administration is at least 0.5 kg greater than that of the control subject. In some aspects, BMI of the subject is increased between day 2 after the administration and day 7 after the administration, and wherein BMI increase of the subject between day 2 after the administration and day 7 after administration is at least 20% to 100% greater than that of the control subject. In some aspects, BMI of the subject is increased between day 2 after the administration and day 7 after the administration, and wherein BMI increase of the subject between day 2 after the administration and day 7 after administration is at least 0.25 greater than that of the control subject.

### BRIEF DESCRIPTION OF THE DRAWINGS

FIGS. 1A and 1B illustrate percent activation of the melanocortin 3 receptor (MC3[R]) (FIG. 1A) and the melanocortin 4 receptor (MC4[R]) (FIG. 1B) following stimulation with a control receptor antagonist (HS024 (TFA)) at different concentrations, as measured by cyclic adenosine monophosphate (CAMP) levels in accordance with embodiments of the present technology.

FIGS. 2A-AA illustrate percent activation of the MC3 receptor following stimulation with non-naturally occurring melanocortin analog peptides at different concentrations as measured by CAMP levels in accordance with embodiments of the present technology. Example synthetic peptides included A1 (SEQ ID NO: 14; FIG. 2A), A2 (SEQ ID NO: 15; FIG. 2B), B1 (SEQ ID NO: 291; FIG. 2C), B2 (SEQ ID NO: 292; FIG. 2D), B3 (SEQ ID NO: 293; FIG. 2E), B4 (SEQ ID NO: 294; FIG. 2F), B5 (SEQ ID NO: 295; FIG. 2G), B6 (SEQ ID NO: 296; FIG. 2H), B7 (SEQ ID NO: 297; FIG. 21), B8 (SEQ ID NO: 298; FIG. 2J), B9 (SEQ ID NO: 299; FIG. 2K), C1 (SEQ ID NO: 300; FIG. 2L), C2 (SEQ ID NO: 301; FIG. 2M), C3 (SEQ ID NO: 302; FIG. 2N), C4 (SEQ ID NO: 303; FIG. 20), C5 (SEQ ID NO: 304; FIG. 2P), C6 (SEQ ID NO: 305; FIG. 2Q), C7 (SEQ ID NO: 306; FIG. 2R), C8 (SEQ ID NO: 307; FIG. 2S), C9 (SEQ ID NO: 308; FIG. 2T), D1 (SEQ ID NO: 6; FIG. 2U), D2 (SEQ ID NO: 9; FIG. 2V), D3 (SEQ ID NO: 4; FIG. 2W), D4 (SEQ ID NO: 10; FIG. 2X), D5 (SEQ ID NO: 11; FIG. 2Y), TCMCB02 (SEQ ID NO: 309; FIG. 2Z), and TCMCB07 (SEQ ID NO: 3; FIG. 2AA).

FIGS. 3A-AA illustrate percent activation of the MC4 receptor following stimulation with non-naturally occurring melanocortin analog peptides at different concentrations as measured by CAMP levels in accordance with embodiments of the present technology. Example synthetic peptides included A1 (FIG. 3A), A2 (FIG. 3B), B1 (FIG. 3C), B2 (FIG. 3D), B3 (FIG. 3E), B4 (FIG. 3F), B5 (FIG. 3G), B6 (FIG. 3H), B7 (FIG. 31), B8 (FIG. 3J), B9 (FIG. 3K), C1 (FIG. 3L), C2 (FIG. 3M), C3 (FIG. 3N), C4 (FIG. 30), C5 (FIG. 3P), C6 (FIG. 3Q), C7 (FIG. 3R), C8 (FIG. 3S), C9 (FIG. 3T), D1 (FIG. 3U), D2 (FIG. 3V), D3 (FIG. 3W), D4 (FIG. 3X), D5 (FIG. 3Y), TCMCB02 (FIG. 3Z), and TCMCB07 (FIG. 3AA).

FIG. 4A-4E show cardiovascular effects of non-naturally occurring melanocortin analog peptides (MCs) in rats. (FIG. 4A) An ECG tracing (lead 2) from a rat given IV PT-141 (600 nmol/kg). Note the inverted P-wave (arrows). (FIG. 4B) An ECG tracing (lead 2) from a rat given IV MT-II (600 nmol/kg). Note the inconsistent presence of a P-wave (arrows). (FIG. 4C) The MAP (upper panel) and HR (lower panel) effects of a lethal dose of PT-141 (900 nmol/kg IV at arrow). Each y-axis block is 20 mm Hg or 60 BPM, while each x-axis block is 15 seconds. Baseline MAP was 80 mmHg, rising to 140, before falling. Baseline heart rate was 312 and fell. (FIG. 4D) A second by second tracing (1 block=1 sec) of the arterial pressure (upper panel), mean arterial pressure (MAP) (middle panel) and heart rate (bottom panel) produced by γ-melanocortin (150 nmol/kg IV dose at arrow). Bursts of tachycardia (′50 BPM) appear during a period of bradycardia, as MAP increases, a characteristic of tachybradycardia syndrome. Joung et al., Am. J. Physiol. 299 (3): H634-642 (2010). (FIG. 4E) Arterial pressure response (upper panel, horizontal dashed line is MAP) to γ-melanocortin (150 nmol/kg, point of administration off screen to left), with a lead 2 ECG tracing (lower panel). γ-melanocortin produced ECGs with an intermittently suppressed P-wave (examples at arrows) during a bradycardia.

FIG. 5 shows total food intake over days 0-8 of the study in saline control rats and rats in treated with a non-naturally occurring melanocortin analog (TCMCB01) prior to tumor implantation and prior to treatment.

FIGS. 6A-6F shows the inhibition of cancer cachexia by: (i) an endogenous melanocortin system inhibitor (agouti-related protein; AgRP); (ii) SHU9119 (“SHU” SEQ ID NO: 313); and (iii) TCMCB01 (SEQ ID NO: 315). Rats were administered once per day at a dose of 2 nmol. FIGS. 6A-6C show the daily food intake of rats bearing a Lewis sarcoma with and without treatment. FIGS. 6D-6F show total food intake for the treatment period. A sham operation or implantation of tumor cells was on day 1, and significant cancer anorexia was present by days 6-7. The SHU9119-induced reversal of anorexia is about 70% of that observed with AGRP. TCMCB01 produces a significantly greater response, equivalent to AGRP.

FIG. 7 shows the daily body weight of tumor-bearing rats in the saline control group and the TCMCB01-treated group.

FIG. 8. shows tumor weights of rats in the saline control group and the TCMCB01-treated group.

FIG. 9. shows lean body mass gain (% of initial) of tumor-bearing rats in the saline control group and TCMCB01-treated group.

FIGS. 10A-10B show data from treatment with the non-naturally occurring melanocortin analog, TCMCB02. (FIG. 10A) TCMCB02 (2 mg/kg, IP) was administered to rats previously injected with lipopolysaccharide (LPS), in an attempt to reverse endotoxin-induced cachexia. Treatment with TCMCB02 stimulated feeding in 12-hours overnight compared to a previous 4-day average (baseline). Saline-administered control rats ate 40% of their baseline and TCMCB02-treated rats ate 65%. (FIG. 10B) Saline-treated rats gained little weight overnight while the TCMCB02-treated rats gained almost 5%. The TCMCB02-treated animals approached normal weight gain during baseline.

FIGS. 11A-11B show the effect of a LPS injection in rats before a dark cycl, on (FIG. 11A) food intake and (FIG. 11B) body weight (as percent of pre-LPS baseline). Measurements were made for 24-hours following the challenge, and non-naturally occurring melanocortin analogs were given intraperitoneally at 0.2 mg/kg. The saline-treated control rats were anorexic and lost weight. Rats treated with TCMCB05 (“B05” SEQ ID NO: 318) and TCMCB07 (“B07” SEQ ID NO: 3) had stimulated appetites and gained weight. The TCMCB06 (“B06” SEQ ID NO: 319)-treated rats ate less food than the other non-naturally occurring melanocortin analogs, and had no weight gain. While the weight gain in rats treated with TCMCB05 or TCMCB07 appear less than that observed with TCMCB01 (see FIG. 8B), the TCMCB05 and TCMCB07 data were recorded at the end of the 12 hour dark cycle when eating occurred. These data are 24 hour data, including the dark/feeding phase and the subsequent 12 hours of light cycle, when weight loss occurs. * p<0.05, ***p<0.001

FIG. 12 shows the effects of peripherally administered TCMCB04 on food intake in a bacterial toxin-(LPS)-induced model of cachexia. No difference was observed between the non-naturally occurring melanocortin analog and saline (i.e., no transport across the blood-brain barrier), even though the C-terminal di-peptide is the same as TCMCB02, which had peripheral LPS-anti-cachexia activity.

FIG. 13 shows a comparison of the peripheral activity of TCMCB05 (“B05”) and TCMCB07 (“B07”) on food intake during LPS-induced cachexia. Only TCMCB07 produced a significant increase in food intake.

FIG. 14 shows the effects of TCMCB07 (“B07”) and TCMCB08 (“B08”) on food intake 24 hours following gavage (intra-gastric) administration in a rat model of LPS cachexia.

FIG. 15 shows the effects of TCMCB07 (“B07”) and TCMCB07A (“B07A”) on food intake 24 hours following gavage (intra-gastric) administration in a rat model of LPS cachexia.

FIG. 16 shows high performance liquid chromatograms of rat plasma. Time points are pre-intravenous administration and 2 minutes post administration of 750 nmol/kg TCMCB07. A naphthylalanine (Nal residue)-positive fluorescence peak appears at the retention time (14.5 minutes) of TCMCB07. Concentration of TCMCB07 in plasma 2 minutes post-intravenous administration is 7 nmol/mL.

FIGS. 17A-17B show high performance liquid chromatograms of rat cerebrospinal fluid. (FIG. 17A) Time points: pre administration, 5 and 30 minutes post administration of intravenous TCMCB07 (750 nmol/kg). A naphthylalanine (Nal residue)-positive fluorescence peak appears at a retention time of 12.9 minutes, slightly earlier than TCMCB07 (14.5 minutes). The small peak at the 12.9-minute retention time in the pre-administration sample represents a co-eluting component of cerebrospinal fluid. FIG. 17B shows the increased cerebrospinal fluid concentration (represented as peak height) of the proposed TCMCB07 metabolite over time.

FIG. 18A-18C shows mass spectrometry analysis of cerebrospinal fluid pre-injection (FIG. 18A), 5 min post-injection (FIG. 18B), or 30 min post-injection (FIG. 18C) of the collected eluent from the peak with a retention time of 12.9 minutes. The arrows indicate the occurrence of a peptide in the post-injection samples, but not in the pre-injection sample. This peak has an atomic weight of 1,209 Daltons, 21 Daltons less than cyclized TCMCB07, at 1,230 Da.

FIGS. 19A-19B depict depicts plots comparing the mean body weights in rats administered with 0 mg/kg/day TCMCB07 (Group 1), 0.5 mg/kg/day TCMCB07 (Group 2), 3 mg/kg/day TCMCB07 (Group 3), and 10 mg/kg/day TCMCB07 (Group 4), respectively, for 28 days. The body weights are monitored from 3 days prior to the administration until 56 days after administration of the first dose of TCMCB07. FIG. 19A shows the results of male rats, and FIG. 19B shows the results of female rats.

FIGS. 20A-20B depict plots comparing the mean food consumptions in rats administered with 0 mg/kg/day TCMCB07 (Group 1), 0.5 mg/kg/day TCMCB07 (Group 2), 3 mg/kg/day TCMCB07 (Group 3), and 10 mg/kg/day TCMCB07 (Group 4), respectively, for 28 days. The body weights are monitored from 3 days prior to the administration until 56 days after administration of the first dose of TCMCB07. FIG. 20A shows the results of male rats, and FIG. 20B shows the results of female rats.

FIGS. 21A-21B depict plots comparing the mean body weights in dogs administered with 0 mg/kg/day TCMCB07 (Group 1), 0.5 mg/kg/day TCMCB07 (Group 2), 3 mg/kg/day TCMCB07 (Group 3), and 10 mg/kg/day TCMCB07 (Group 4), respectively, for 28 days. The body weights are monitored from 3 days prior to the administration until 56 days after administration of the first dose of TCMCB07. FIG. 21A shows results of male dogs, and FIG. 21B shows the results of female dogs.

FIGS. 22A-22B depict plots comparing the mean food consumptions in dogs administered with 0 mg/kg/day TCMCB07 (Group 1), 0.5 mg/kg/day TCMCB07 (Group 2), 3 mg/kg/day TCMCB07 (Group 3), and 10 mg/kg/day TCMCB07 (Group 4), respectively, for 28 days. The body weights are monitored from 3 days prior to the administration until 56 days after administration of the first dose of TCMCB07. FIG. 22A shows results of male dogs, and FIG. 22B shows results of female dogs.

FIG. 23A-23B shows weight change in KPC mouse model (pancreatic ductal adenocarcinoma) treated with TCMCB07 and control. FIG. 23A depicts plots comparing overall lean mass gains in saline (control group) and TCMCB07 (experimental group), respectively. FIG. 23B depicts plots comparing heart weights in saline-treated mice (control group, left) and two cohorts of TCMCB07-treated mice (experimental group, center and right), respectively.

FIG. 24 depicts plots comparing gastrocnemius weights in Lewis Sarcoma rat model administered with saline (control group), low dose of TCMCB07 (experimental group), and high dose of TCMCB07 (experimental group), respectively.

FIG. 25 summarizes safety readouts of a phase 1 clinical trial utilizing TCMCB07.

FIG. 26 shows a boxplot of the differences in BMI between day 2 and days 3, 4, 5, 6, 7, or 8 in patients treated with TCMCB07 or placebo. Time+02 is time point time point-2 hrs but coded to +2 to make the program generate tables and plots in the order of early time points to later time points. For example, Day2+02 is day2 at time-02.

FIG. 27 shows a boxplot of the differences in weight between day 2 and days 3, 4, 5, 6, 7, or 8 in patients treated with TCMCB07 or placebo. Time+02 is time point time point-2 hrs but coded to +2 to make the program generate tables and plots in the order of early time points to later time points. For example, Day2+02 is day2 at time-02.

FIG. 28 shows appetite, as measured by ease of eating over time for patients treated with TCMCB07. Time+02 is time point time point-2 hrs but coded to +2 to make the program generate tables and plots in the order of early time points to later time points. For example, Day2+02 is day2 at time-02.

### DETAILED DESCRIPTION

Described herein are methods of promoting appetite of a subject in need thereof by administering a non-naturally occurring melanocortin analog to the subject. The non-naturally occurring melanocortin analog can be present in a pharmaceutical composition and delivered via parenteral administration (e.g., subcutaneous injection). The appetite promoting methods can lead to increased food consumption, increased body weight, increased muscle mass, and/or increased fat mass of the subject. The appetite promoting methods can also lead to preventing or reducing risk of appetite loss or loss of appetite, reducing or preventing body weight loss, reducing or preventing loss of muscle mass, and/or reducing or preventing loss of fat mass of the subject. The methods can also prevent or alleviate nausea, emesis, and/or anorexia in the subject. The subject can be a healthy subject and/or a metabolically unchallenged subject. Alternatively, the subject can be a cancer patient including those suffering from cancer cachexia.

The methods disclosed herein use non-naturally occurring melanocortin receptor analogs, or a pharmaceutically acceptable salt, solvate, or stereoisomer thereof, referred to herein collectively as non-naturally occurring melanocortin analogs. Some of the non-naturally occurring melanocortin analogs can bind only the melanocortin 3 receptor or the melanocortin 4 receptor. Other non-naturally occurring melanocortin analogs can bind the melanocortin 3 receptor with greater affinity than the melanocortin 4 receptor, whereas other non-naturally occurring melanocortin analogs can bind the melanocortin 4 receptor with greater affinity than the melanocortin 3 receptor. Certain non-naturally occurring melanocortin analogs can bind the melanocortin 3 receptor with the same or generally similar affinity as the melanocortin 4 receptor.

Each of the synthetic peptides can have one or more beta hairpin (β-hairpin) and/or beta turn (β-turn) structures. The presence of amino acids that are structurally rigid, such as, for example, Aia, Aba, Ata, Hyp, D-Hyp, Pro, D-Pro, transPro(guan), and cisPro(guan), can lead to formation of β-hairpin and/or β-turn structures in the peptide. Additionally, D-Met and disulfide bridges (cyclization via a disulfide bridge) can induce and/or stabilize beta-turn structures of the synthetic peptides. Pro-Gly and Gly-Gly linkers can also induce and/or stabilize beta-turns. In general, cyclization can stabilize beta-turns, and D-amino acids can induce and/or stabilize beta-turns. The synthetic peptides include D-valine-D-proline (dV-dP) or D-proline-D-valine (dP-dV) chain as their C-terminus, which can provide enhanced transport and resistance to degradation. The synthetic peptides can be linear or optionally cyclized via a disulfide bridge or a lactam bond at various positions of the peptide chain. Each of the synthetic peptides can include one or more of the foregoing features.

The following description is merely exemplary in nature and is not intended to limit the present technology, its applications, or its uses. It should be understood that throughout the drawings, corresponding reference numerals indicate like or corresponding parts and features. The description of specific examples indicated in various embodiments of the present technology are intended for purposes of illustration only and are not intended to limit the scope of the present technology disclosed herein. Moreover, recitation of multiple embodiments having stated features is not intended to exclude other embodiments having additional features or other embodiments incorporating different combinations of the stated features.

Furthermore, the detailed description of various embodiments herein makes reference to the accompanying drawing/FIGS, which show various embodiments by way of illustration. While the embodiments are described in sufficient detail to enable those skilled in the art to practice the present technology, it should be understood that other embodiments may be realized and that logical and mechanical changes may be made without departing from the spirit and scope of the present technology. Thus, the detailed description herein is presented for purposes of illustration only and not of limitation. For example, steps or functions recited in descriptions, any method, system, or process, may be executed in any order and are not limited to the order presented. Moreover, any of the step or functions thereof may be outsourced to or performed by one or more third parties.

### Definitions

Unless defined otherwise, all technical and scientific terms used herein have the same meaning as commonly understood by those of ordinary skill in the art to which the present technology belongs. For the purposes of the present technology, the following terms are defined below.

The articles “a” and “an” are used herein to refer to one or to more than one (i.e., to at least one) of the grammatical object of the article. By way of example, “an element” means one element or more than one element.

The term “about” means a quantity, level, value, number, frequency, percentage, dimension, size, amount, weight or length that varies by acceptable levels in the art. In some embodiments, such variation may be as much as 30, 25, 20, 15, 10, 9, 8, 7, 6, 5, 4, 3, 2 or 1% to a reference quantity, level, value, number, frequency, percentage, dimension, size, amount, weight or length. When the term “about” is used in conjunction with a numerical range, it modifies that range by extending the boundaries above and below the numerical values set forth.

The terms “administering” or “administer” include delivery of non-naturally occurring melanocortin analogs (also referred to herein as peptides) of the present technology to a subject either by local or systemic administration. Administration may be topical (including ophthalmic and to mucous membranes including vaginal and rectal delivery), pulmonary (e.g., by inhalation or insufflation of powders or aerosols, including by nebulizer), intratracheal, intranasal, epidermal and transdermal, oral or parenteral. Parenteral administration includes intravenous, intraarterial, subcutaneous, intraperitoneal or intramuscular injection or infusion; or intracranial, e.g., intrathecal or intraventricular, administration.

“Melanocortin analogs,” “melanocortin peptides,” or “melanocortins,” are used interchangeably and refer to melanocortin-receptor ligands, which are macromolecules containing at least one melanocortin pharmacophore. Melanocortin analogs are typically peptides that bind melanocortin receptors under physiological conditions. Melanocortin analogs include non-naturally occurring melanocortin peptides and truncated and/or modified versions of melanocortin full-length protein or peptides. For example, the full-length pro-opiomelanocortin protein (POMC), prior to proteolytic cleavage of “sub-peptides,” consists of 241 amino acids. Tissue-specific proteolytic cleavage of POMC yields peptides ranging in size from 13 amino acids to 76 amino acids. See Bicknell and Lawry, Encyclopedia of Stress, vol. 3, 257-265, Academic Press (2000). Synthesized, non-naturally occurring melanocortin analogs having increased melanocortin receptor activity as discussed herein are approximately 7-12 amino acids in size. Melanocortin analogs exhibit binding functionality with melanocortin receptors. The binding to the melanocortin receptor is inhibitory (antagonist). In addition to peptides, the melanocortin analogs include small molecule analogs of melanocortin or portions thereof comprised of organic compounds, inorganic compounds, or combinations of peptide and small molecule—i.e., peptide mimetics, or various combinations thereof. “Melanocortin peptides” can be structurally similar and/or functionally similar to biological melanocortin proteins in their ability to bind melanocortin receptors. Further, the melanocortin analogs generally contain the pharmacophore: His-Phe-Arg-Trp(SEQ ID NO: 1) or a modified version thereof, or a structural or functional peptide mimetic thereof.

A “pharmacophore” is the minimum set of amino acid residues necessary to achieve a physiological effect; or a small molecule that is (with respect to a receptor) a structural mimic of the amino acid residues required for binding to and activation of a receptor. His-Phe-Arg-Trp(SEQ ID NO: 1) and their analogs are the pharmacophore of melanocortin for the regulated physiological effect. Therefore, non-naturally occurring melanocortin pharmacophore analogs can be small peptides or organic molecules designed to mimic the appearance or function (including activation or deactivation of receptor activity) of the melanocortin pharmacophore core sequence peptide.

A melanocortin receptor “antagonist” is a naturally occurring substance or manufactured drug substance or composition that opposes the melanocortin receptor-associated responses normally induced by a melanocortin receptor agonist agent.

“Potentiated therapeutic activity” refers to an increase in melanocortin activity in a non-naturally occurring melanocortin analog that has undergone derivatization at the N- and/or C-terminus. Such derivatizations do not necessarily involve the pharmacophore, but do imply a relative increase in in vivo biological half-life.

“Substantial degradation” refers to the degradation of the N-terminal extension, the C-terminal extension, both N- and C-terminal degradation or degradation to other regions of the non-naturally occurring melanocortin analog by physiological enzymes and other factors, in such a manner or to a degree that side effects appear. According to one aspect, a melanocortin analog having a C-terminal extension that resists substantial degradation is one where no more than 50% of the administered peptide causes side effects and/or displays a low half-life. In some aspects, no more than 25% of the administered peptide causes side effects and/or displays a low half-life. More preferably, in some aspects, less than 10% of the administered peptide causes side effects and/or displays a low half-life, as compared to a melanocortin analog that lacks a C-terminal extension.

The terms “bind,” “binding,” “complex,” and “complexing,” refer to all types of physical and chemical binding, reactions, complexing, attraction, chelating and the like.

The “peptides” described herein can be (a) naturally-occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides.

The term “peptide” as used herein includes any structure comprised of two or more amino acids, including chemical modifications and derivatives of amino acids. The amino acids forming all or a part of a peptide may be naturally occurring amino acids, stereoisomers and modifications of such amino acids, non-protein amino acids, post-translationally modified amino acids, enzymatically modified amino acids, constructs or structures designed to mimic amino acids, and the like, so that the term “peptide” includes pseudopeptides and peptidomimetics, including structures which have a non-peptidic backbone. The term “peptide” also includes dimers or multimers of peptides. A “manufactured” peptide includes a peptide produced by chemical synthesis, recombinant DNA technology, biochemical, or enzymatic fragmentation of larger molecules, combinations of the foregoing or, in general, made by any other method. The term “peptide” includes peptides containing a variable number of amino acid residues, optionally with non-amino acid residue groups at the N- and C-termini, such groups including acyl, acetyl, alkenyl, alkyl, N-alkyl, amine, or amide groups, among others.

By employing chemical synthesis, a useful means of production, it is possible to introduce various amino acids which do not naturally occur along the chain, modify the N- or C-terminus, and the like, thereby providing for improved stability and formulation, resistance to protease degradation, and the like.

“Amino acids” are molecules containing an amine group, a carboxylic acid group, and a side-chain that is specific to each amino acid. The key elements of an amino acid are carbon, hydrogen, oxygen, and nitrogen and have the generic formula H₂N—CHR—COOH, wherein R represents a side chain group. The various α-amino acids differ in the side-chain moiety that is attached to the α-carbon. The “amino acids” of the present technology include the known naturally occurring protein amino acids, which are referred to by both their common three letter abbreviation and single letter abbreviation. See generally Synthetic Peptides: A User's Guide, G. A. Grant, editor, W. H. Freeman & Co., New York (1992), the teachings of which are incorporated herein by reference, including the text and table set forth at pages 11 through 24. As set forth above, the term “amino acid” also includes stereoisomers and modifications of naturally occurring protein amino acids, non-protein amino acids, post-translationally modified amino acids, enzymatically synthesized amino acids, derivatized amino acids, constructs or structures designed to mimic amino acids, and the like. Modified and unusual amino acids are described generally in Synthetic Peptides: A User's Guide, supra; Hruby et al., Biochem. J. 268:249-262 (1990); and Toniolo, Int. J. Peptide Protein Res. 35:287-300 (1990); the teachings of all of which are incorporated herein by reference.

The phrase “amino acid side chain moiety” used herein, including as used in the specification and claims, includes any side chain of any amino acid, as the term “amino acid” is defined herein. This thus includes the side chain moiety present in naturally occurring amino acids. It further includes side chain moieties in modified naturally occurring amino acids, such as glycosylated amino acids. It further includes side chain moieties in stereoisomers and modifications of naturally occurring protein amino acids, non-protein amino acids, post-translationally modified amino acids, enzymatically synthesized amino acids, derivatized amino acids, constructs, or structures designed to mimic amino acids, and the like. For example, the side chain moiety of any amino acid disclosed herein is included within the definition. A “derivative” of an amino acid side chain moiety is included within the definition of an amino acid side chain moiety.

The “derivative” of an amino acid side chain moiety includes any modification to or variation in any amino acid side chain moieties, including a modification of naturally occurring amino acid side chain moieties. By way of example, derivatives of amino acid side chain moieties include straight chain or branched, cyclic or noncyclic, substituted or unsubstituted, saturated or unsaturated, alkyl, aryl or aralkyl moieties.

In the peptides described herein, conventional amino acid residues have their conventional meaning as given in Chapter 2400, of the Manual of Patent Examining Procedure, 8th Ed. Thus, “Ala” is alanine; “Arg” is arginine; “Asn” is asparagine; “Asp” is aspartic acid; “Cys” is cysteine; “Gln” is glutamine; “Glu” is glutamic acid; “His” is histidine; “11e” is isoleucine; “Leu” is leucine; “Lys” is lysine; “Met” is methionine; “Phe” is phenylalanine; “Pro” is proline; “Ser” is serine; Thr is threonine; “Trp” is tryptophan; “Tyr” is tryosine; and “Val” is valine. Unless otherwise indicated, all amino acids abbreviations represent either isomer, i.e., the L-isomer, the D-isomer, or combinations thereof can be used. Thus, for example, “L-Phe” or “IPhe” is L-phenylalanine; “D-Phe” or “dPhe” is D-phenylalanine; dVal is D-valine; dPro is D-proline; “D-/L-Phe” or “d/IPhe” is D-phenylalanine, L-phenylalanine, or combinations thereof; “Phe” is also D-phenylalanine, L-phenylalanine, or combinations thereof, and so on. Non-standard amino acids are “Nle” is norleucine; “Nal” is naphthylalanine; “D-Nal” is D-naphthylalanine; D-Nal(2′) or DNal(2′) is D-2′-naphthylalanine; L-Nal(2′) or LNal(2′) is L-2′-naphthylalanine; L-Nal(1′) is L-1′-naphthylalanine; D-Nal(1′) or DNal(1′) is D-1′-naphthylalanine; Tle is tert-Leucine; Nva is norvaline; Orn is ornithine; Bip is biphenyl amino acid; and so on.

An alpha (α)-amino acid has the generic formula H₂N—C_{α}HR—COOH, where R is a side chain moiety and the amino group is attached to the carbon atom immediately adjacent to the carboxylate group(i.e., the α-carbon). Other types of amino acids exist when the amino group is attached to a different carbon atom. For example, beta (B)-amino acids, the carbon atom to which the amino group is attached is separated from the carboxylate group by one carbon atom, Cp. For example, α-alanine has the formula H₂N—C_{α}H(CH₃)—COOH. In contrast, β-alanine has the general formula H₂N—C_{β}H₂—C_{α}H₂—COOH (i.e., 3-aminopropanoic acid).

When β-amino acids are incorporated into peptides, two main types of β-peptides exist: those with the side chain residue, R, on the carbon next to the amine are called 3 peptides and those with the side chain residue on the carbon next to the carbonyl group are called 32 amino acids. As a non-limiting example, “β-valine” can refer to:
—NH-C_{β}H2-C_{α}H(CH₃)₂—CO—, i.e., 32-valine (R on carboxy side);
—NH—C_{β}H(CH₃)₂—C_{α}H₂—CO—, i.e., 33-valine (R on amino side); or
—NH—C_{β}H(CH₃)₂—C_{α}H(CH₃)₂—CO—, i.e., B2.3-valine (R at both positions).

Gamma (γ)-amino acids are amino acids with the carbon atom to which the amino group attaches is separated from the carboxylate moiety by two carbon atoms. For example, γ-amino butyric acid has the formula, H₂N-C_{γ}H2-C_{β}H2-C_{α}H₂—COOH.

For additional modified and unusual amino acids, see § 2422 of the MPEP, particularly Table 4 at 2400-24. Additionally, “Ac” indicates N-acetyl and “cyclo” refers to a cyclic structure, which is also shown in the literature as “c” or referred to as a “lactam.” “NH₂” indicates an amine group, typically added on the C-terminus of a polypeptide. Accordingly, as used herein, an —NH₂ moiety on the C-terminus of a peptide indicates an amidated C-terminus. In addition, the following abbreviations are used herein: Harg is Homo arginine; Hlys is Homo lysine; Nal(2′) is D-(2′-naphthyl) alanine.

Additional abbreviations are used as follows: tBu is tert-butyl; Hyp(Bzl) is benzyl-L-hydroxy-proline; Mamb is 3-aminomethyl-benzoic acid; glutaric acid linker is CO—(CH₂) 3—CO; Pen is L-Penicillamine; Aib is 2-Aminoisobutyric acid; Tic is 1,2,3,4-Tetrahydroisoquinoline-3-carboxylic Acid; Aba is 4-amino-1,2,4,5-tetra-hydro-2-benzazepin-3-one; Pip is piperidine-2-carboxylic acid; Nip is piperidine-3-carboxylic acid; Tic is tetrahydroquinoline-3-carboxylic acid; Bipisbiphenylalanine; Phg is α-Phenyl-glycine; Sar is Sarcosine; Azt is 3′-azido-3′-deoxythymidine; Oic is Octohydroindole-2-carboxylic acid; Aia is 7-amino-7,8-dihydro4H-[1,2,3]triazolo[1,5-a][1,4]diazepin-6 (5H)-one; Mamb is 3-aminomethylbenzoic acid; Atc is 2-Aminotetraline-2-carboxylic acid; APC is 1-Amino-4-phenylcyclohexane-carboxylic acid; ACC is 4-Aminophenylpiperidine-4-carboxylic acid (APPC); Acpc is 1-aminocyclo-propane-1-carboxylic acid; Aic is 2-aminoindone-2-carboxylic acid; pCIPhe is para-chloro-phenylalanine (I-iodo, Br-bromo); and Tic is Tetrahydro-isoquinoline-3-carboxylic Acid.

The term “alkene” includes unsaturated hydrocarbons that contain one or more double carbon-carbon bonds. Examples of such alkene groups include ethylene, propene, and the like.

The term “alkenyl” includes a linear monovalent hydrocarbon radical of two to six carbon atoms or a branched monovalent hydrocarbon radical of three to six carbon atoms containing at least one double bond; examples thereof include ethenyl, 2-propenyl, and the like.

The “alkyl” groups specified herein include those alkyl radicals of the designated length in either a straight or branched configuration. Examples of such alkyl radicals include methyl, ethyl, propyl, isopropyl, butyl, sec-butyl, tertiary butyl, pentyl, isopentyl, hexyl, isohexyl, and the like.

The term “alkynyl” includes a linear monovalent hydrocarbon radical of two to six carbon atoms or a branched monovalent hydrocarbon radical of three to six carbon atoms containing at least one triple bond; examples thereof include ethynyl, propynyl, butynyl, and the like.

The term “aryl” includes a monovalent or bicyclic aromatic hydrocarbon radical of 6-to-12 ring atoms, and optionally substituted independently with one or more substituents selected from alkyl, haloalkyl, cycloalkyl, alkoxy, alkythio, halo, nitro, acyl, cyano, amino, monosubstituted amino, disubstituted amino, hydroxy, carboxy, or alkoxy-carbonyl. Examples of an aryl group include phenyl, biphenyl, naphthyl, 1-naphthyl, and 2-naphthyl, derivatives thereof, and the like.

The term “aliphatic” includes compounds with hydrocarbon chains, such as for example alkanes, alkenes, alkynes, and derivatives thereof.

The term “acyl” includes a group RCO—, where R is an organic group. An example is the acetyl group CH₃CO—, referred to herein as “Ac.”

The term “fatty acid” describes a carboxylic acid with an aliphatic chain, which can be fully saturated or partially unsaturated, and optionally attached to a functional group such as a hydroxyl group or a carboxyl group. The aliphatic chain can contain e.g., from 6 to 26 carbon atoms and hydrogen atoms.

A peptide or aliphatic moiety is “acylated” when an alkyl or substituted alkyl group as defined above is bonded through one or more carbonyl {—(C═O)-} groups. A peptide is most usually acylated at the N-terminus.

An “omega amino derivative” includes an aliphatic moiety with a terminal amino group. Examples of omega amino derivatives include aminoheptanoyl and the amino acid side chain moieties of ornithine and lysine.

The term “heteroaryl” includes mono- and bicyclic aromatic rings containing from 1 to 4 heteroatoms selected from nitrogen, oxygen, and sulfur. Five- or six-membered heteroaryl are monocyclic heteroaromatic rings; examples thereof include thiazole, oxazole, thiophene, furan, pyrrole, imidazole, isoxazole, pyrazole, triazole, thiadiazole, tetrazole, oxadiazole, pyridine, pyridazine, pyrimidine, pyrazine, and the like. Bicyclic heteroaromatic rings include, but are not limited to, benzothiadiazole, indole, benzothiophene, benzofuran, benzimidazole, benzisoxazole, benzothiazole, quinoline, benzotriazole, benzoxazole, isoquinoline, purine, furopyridine, and thienopyridine.

An “amine” includes compounds that contain an amine group(—NH₂).

An “amide” includes compounds that have a trivalent nitrogen attached to a carbonyl group(i.e., —CO—NH₂), such as for example methylamide, ethylamide, propylamide, and the like. A peptide is most usually amidated at the C-terminus by the addition of an amine (—NH₂) moiety to the C-terminal carboxyl group.

An “imine” includes compounds that have a carbon-nitrogen double bond, with the nitrogen also attached to a hydrogen (NH═CH—R).

An “imide” includes compounds containing an imido group(—OC—NH—CO—).

A “nitrile” includes compounds that are carboxylic acid derivatives and contain a (—CN) group bound to an organic group.

The term “halogen” is intended to include the halogen atoms fluorine, chlorine, bromine and iodine, and groups including one or more halogen atoms, such as —CF3 and the like.

Amino acids, including stereoisomers and modifications of naturally occurring amino acids, protein amino acids, non-protein amino acids, post-translationally modified amino acids, enzymatically synthesized amino acids, derivatized amino acids, constructs, or structures designed to mimic amino acids (peptide mimetics), and the like, including all of the foregoing, are sometimes referred to herein as “residues.”

A peptide or amino acid “mimetic” is a non-amino acid molecule that mimics a peptide (a chain of amino acids) or one amino acid residue.

“Substantial degradation” refers to the degradation of the N-terminal extension, the C-terminal extension, both N- and C-terminal degradation or degradation to other regions of the non-naturally occurring melanocortin analogs by physiological enzymes and other factors, in such a manner or to a degree that side effects appear. According to one aspect, a melanocortin analog having a C-terminal extension that resists substantial degradation is one where no more than 50% of the administered peptide causes side effects and/or displays a low half-life. In some aspects, no more than 25% of the administered peptide causes side effects and/or displays a low half-life. More preferably, in some aspects, less than 10% of the administered peptide causes side effects and/or displays a low half-life, as compared to a melanocortin analog that lacks a C-terminal extension.

As used herein, a “composition” refers to a mixture of the active ingredient with other chemical components.

A melanocortin receptor “agonist” is a naturally occurring substance or manufactured drug substance or composition that can interact with a melanocortin receptor and initiate a pharmacological response characteristic of the melanocortin receptor. By a melanocortin receptor “antagonist” is a naturally occurring substance or manufactured drug substance or composition that opposes the melanocortin receptor-associated responses normally induced by a melanocortin receptor agonist agent. A melanocortin receptor “inverse agonist” is a drug or a compound that stabilizes the inactive conformation of the melanocortin receptor and inhibits basal activity.

As used herein, a “pharmaceutically acceptable carrier and/or excipient” of the pharmaceutical composition refers to a carrier or diluent that does not cause significant irritation to an organism, does not abrogate the biological activity and properties of the administered active ingredient, and/or does not interact in a deleterious manner with the other components of the composition in which it is contained. The term “carrier” encompasses any binder, diluent, filler, salt, buffer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005, which is incorporated herein by reference in its entirety. An “excipient” of the pharmaceutical composition refers to an inert substance added to a composition to further facilitate administration of a compound.

As used herein, the terms “effective amount” or “therapeutically effective amount”, refer to that amount of the active ingredient being administered which will relieve to some extent one or more of the symptoms of the disease being treated. The result can be a reduction and/or alleviation of the signs, symptoms, or causes of a disease, or any other desired alteration of a biological system. An appropriate “effective amount” can differ from one individual to another. An appropriate “effective amount” in any individual case can be determined using techniques, such as a dose escalation study.

The term “after administration” refers to any duration of time after the non-naturally occurring melanocortin analog or pharmaceutical composition thereof has been administered to a subject. “After administration” can also refer to the duration of time after one dose has been completed or after more than one dose has been completed, such as two doses, three doses, four doses, and the like. In some embodiments, “after administration” refers to completion of dosing regimen that includes one or more doses. Unless otherwise specified, durations of time encompassed by “after administration” can include seconds, minutes, hours, days, weeks, months, and years.

“Appetite” in a subject and/or patient is typically assessed by their desire to eat and/or the amount of food they consume. As used herein, appetite can be assessed by various appetite assessment methods, tests, and techniques such as using a visual analogue scales (VAS), levels of one or more biomarkers of satiety, such as Ghrelin, cholecystokinin (CCK), glucagon-like peptide 1 (GLP-1), or peptide YY (PYY) in a sample taken from the subject or patient, through a daily questionnaire given at specified or random times of the day, or is inferred from one or more of the subject's characteristics and/or behaviors.

In the questionnaire, subjects or patients rate their hunger, ease/difficulty of eating ranging from 0 (extremely easy to eat) to 100 (extremely difficult to eat) and/or desire to eat greater varieties of food using scales ranging from 0 (not at all) to 100 (extremely). Inferring the change in appetite may correlate to a change in the subject's weight or a change in the subject or patient's eating habits compared to a control subject or patient, or the same subject or patient at baseline. Additional methods, tests, and techniques for assessing “appetite” include those known to one of skill in the art, for example those described in Gibbons et al. 8 (2) Curr. Obes. Rep. 77 (2019), the entirety of which is incorporated by reference.

“Cachexia” refers to a state of general ill health and malnutrition characterized by loss of body mass including loss of weight, loss of muscle mass (skeletal, smooth, and/or cardiac muscle), loss of fat mass, or a combination thereof, and wasting. It is often associated with and induced by certain diseases or conditions such as, but not limited to, cancer, cystic fibrosis, or AIDS. The term “cancer cachexia” refers to cachexia induced by cancer.

“Anorexia” refers to a loss of appetite, whether brought on by medical, physiological, or psychological factors. Anorexia is often closely associated with, and generally contributes to, cachexia seen in patients with advanced cancers and other conditions.

The term “Body Mass Index” or “BMI” refers to a value derived from an individual's body weight and height. Specifically, BMI is determined by body weight (kilograms) divided by the square of height (m²) and is expressed in units of “kg/m²”. “Normal” BMI ranges are known to a person of ordinary skill in the art and consider factors such as patient sex, age, height, race, and body type. Typically, a normal BMI range is about 18.5 kg/m² to about 25 kg/m².

The terms “treat,” “treatment,” and “treating” refer to a manner of providing a pharmaceutical composition and/or melanocortin analog to alleviate disease outcomes. This includes utilizing administration techniques as described in the context of the present technology. Efficacy of treatment can be determined by various assessment methods as described in the context of the present technology (e.g., assessment of appetite, food consumption, body weight or BMI, muscle mass, fat mass, and measurement of biomarkers).

The term “biomarker” refers to a biological output that is used as a measure of cellular response, whether that be to assess response to therapeutics, disease status, such as cachexia, or as a predictor of clinical outcomes. Biomarkers evaluated in the context of cells, tissue, or whole organisms. The term “disease” herein refers to any disorder adversely affecting biological status. This includes weight-related disorders, such as cachexia. Disease also can be in the context of human and animal health.

The terms “subject” and “patient” refer to anyone being evaluated for disease or condition or being administered a therapeutic or pharmaceutical composition. This includes people without diagnosed or confirmed disease or condition. This also includes people with diagnosed or confirmed disease or condition, such as loss of appetite, nausea, emesis, anorexia, or cachexia. This also includes people who are generally healthy, but at risk of developing a disease or condition in the future.

For instance, subjects in accordance with the present technology can include both metabolically unchallenged and metabolically challenged subjects.

A “metabolically unchallenged subject” refers to a subject having a physiologic metabolism that is not impacted or altered by a disease or condition. Metabolically unchallenged subjects do not have increased metabolisms, decreased metabolisms, or metabolisms that are otherwise impaired or different from a healthy, normal metabolism. A metabolically unchallenged subject has a normal, healthy, physiologic metabolism. This includes those without metabolic or weight-related disease, such as cachexia. In contrast, a “metabolically challenged subject” refers to a subject who experiences abnormal physiologic metabolism and/or abnormal metabolic response under either the basal or stressed state. Metabolically challenged subjects can have increased metabolisms, decreased metabolisms, or metabolisms that are otherwise impaired or different from a healthy, normal metabolism.

The term “control subject,” as used herein, refers to any subject used as a basis for comparison to the subject (e.g., test subject). A control subject includes, but is not limited to, any subject who has not been administered the therapeutic or pharmaceutical composition (e.g., the non-naturally occurring melanocortin analog, a therapeutically effective amount of the non-naturally occurring melanocortin analog or a pharmaceutical composition thereof) or administered a placebo.

The term “after administration” refers to any duration of time after the non-naturally occurring melanocortin analog or pharmaceutical composition thereof has been administered to a subject. Unless otherwise specified, durations of time encompassed by “after administration” can include seconds, minutes, hours, days, weeks, months, and years.

The disclosure of all publications, patents, and published patent applications listed herein are hereby incorporated by reference in their entireties, including but not limited to U.S. Pat. Nos. 8,541,545 and 9,534,018.

### Non-naturally Occurring Melanocortin Analogs

In some aspects, the present technology provides a non-naturally occurring melanocortin analog. In some embodiments, the non-naturally occurring melanocortin analog comprises a sequence according to Formula (I),

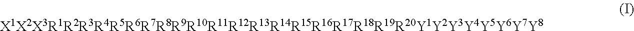

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, D-tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-blindol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), D-Nal(1′), 7-amino-7,8-dihydro4H-[1,2,3]triazolo[1,5-a][1,4]diazepin-6 (5H)-one (Aia), phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine;
the non-naturally occurring melanocortin analog is optionally cyclized through a moiety selected from the group consisting of:
a disulfide bond between R¹ or R² and R⁷ or X¹ when R¹ or R² is cysteine and R⁷ or X¹ is cysteine;
a disulfide bond between R² and any one of R⁵—R²⁰ when R² and the any one of R⁵—R²⁰ are selected from the group consisting of cysteine, Pen, and dPen;
a lactam bridge between R¹ and R⁷ when R¹ is norleucine and R⁷ is glutamic acid;
a side-chain lactam bridge between R² and R⁷ when R² is glutamic acid, aspartic acid, or CO-cis-CH—CH—CO, and R⁷ is lysine or ornithine;
a side-chain lactam bridge between R² and R⁸ when R² is glutamic acid or aspartic acid, R⁸ is lysine, and R⁷ is proline or tryptophan;
a lactam closure between R¹ and R⁷ when R¹ is succinic acid or o-phthalic acid and R⁷ is lysine; and
a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

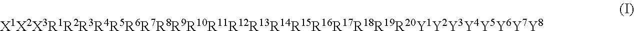

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl (Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);;
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(l)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
   wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
   Y⁸ is absent or is D-proline or D-valine;
the non-naturally occurring melanocortin analog is optionally cyclized through a moiety selected from the group consisting of:
   a disulfide bond between R¹ or R² and R⁷ or X¹ when R¹ or R² is cysteine and R⁷ or X¹ is cysteine;
   a disulfide bond between R² and any one of R⁵—R²⁰ when R² and the any one of R⁵—R²⁰ are selected from the group consisting of cysteine, Pen, and dPen;
   a lactam bridge between R¹ and R⁷ when R¹ is norleucine and R⁷ is glutamic acid;
   a side-chain lactam bridge between R² and R⁷ when R² is glutamic acid, aspartic acid, or CO-cis-CH═CH—CO, and R⁷ is lysine or ornithine;
   a side-chain lactam bridge between R² and R⁸ when R² is glutamic acid or aspartic acid, R⁸ is lysine, and R⁷ is proline or tryptophan;
   a lactam closure between R¹ and R⁷ when R¹ is succinic acid or o-phthalic acid and R⁷ is lysine; and
   a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

In some aspects, the present technology provides a non-naturally occurring melanocortin analog. In some embodiments, the non-naturally occurring melanocortin analog comprises a sequence according to Formula (I),

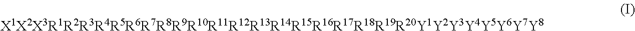

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, D-tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-blindol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), D-Nal(1′), 7-amino-7,8-dihydro4H-[1,2,3]triazolo[1,5-a][1,4]diazepin-6 (5H)-one (Aia), phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine;
the non-naturally occurring melanocortin analog is optionally cyclized through a moiety selected from the group consisting of:
a disulfide bond between R¹ or R² and R⁷ or X¹ when R¹ or R² is cysteine and R⁷ or X¹ is cysteine;
a disulfide bond between R² and any one of R⁵—R²⁰ when R² and the any one of R⁵—R²⁰ are selected from the group consisting of cysteine, Pen, and dPen;
a lactam bridge between R¹ and R⁷ when R¹ is norleucine and R⁷ is glutamic acid;
a side-chain lactam bridge between R² and R⁷ when R² is glutamic acid, aspartic acid, or CO-cis-CH═CH—CO, and R⁷ is lysine or ornithine;
a side-chain lactam bridge between R² and R⁸ when R² is glutamic acid or aspartic acid, R⁸ is lysine, and R⁷ is proline or tryptophan;
a lactam closure between R¹ and R⁷ when R¹ is succinic acid or o-phthalic acid and R⁷ is lysine; and
a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

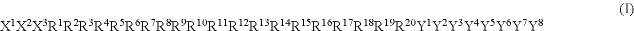

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);;
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
   wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine; and
wherein the non-naturally occurring melanocortin analog comprises one or more of the following features: (i) X² or X³ is present; (ii) R¹ is absent or is selected from the group consisting of dArg, dMet, dIIe, dLeu, dVal, dAla, Ala, Tle, dTle, DNle, Nva, Gly, dPro, dCys, dPhe, dTyr, dGIn, dAsn, transPro(guan), cisPro(guan), dTyr, Tyr, and Dmt; (iii) R² is absent; (iv) R³ is selected from the group consisting of dPro, Pro-Gly, and dAla; (v) R⁴ is selected from the group consisting of His, Trp, and Phe; (vi) R⁵ is Phe; (vii) R⁷ is absent; (viii) R⁸ is present; (ix) R⁹ and/or R¹⁰ is present; (x) one or more of R¹¹—R²⁰ are present; and (xi) Y⁴ is present; and
the non-naturally occurring melanocortin analog is cyclized through a moiety selected from the group consisting of:
   a disulfide bond between R¹ or R² and R⁷ or X¹ when R¹ or R² is cysteine and R⁷ or X¹ is cysteine;
   a disulfide bond between R² and any one of R⁵—R²⁰ when R² and the any one of R⁵—R²⁰ are selected from the group consisting of cysteine, Pen, and dPen;
   a lactam bridge between R¹ and R⁷ when R¹ is norleucine and R⁷ is glutamic acid;
   a side-chain lactam bridge between R² and R⁷ when R² is glutamic acid, aspartic acid, or CO-cis-CH═CH—CO, and R⁷ is lysine or ornithine;
   a side-chain lactam bridge between R² and R⁸ when R² is glutamic acid or aspartic acid, R⁸ is lysine, and R⁷ is proline or tryptophan;
   a lactam closure between R¹ and R⁷ when R¹ is succinic acid or o-phthalic acid and R⁷ is lysine; and
   a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

In some embodiments, the non-naturally occurring melanocortin analog has one or more beta hairpin (β-hairpin) and/or beta turn (β-turn) structures. In some embodiments, R³ of the sequence according to Formula (I), which can be D-proline, L-proline, hydroxyproline, D-hydroxyproline, D-alanine, D-methionine, D-valine, prolylglycine (Pro-Gly), glycine, transPro(guan), cisPro(guan), provides the β-hairpin and/or β-turn structures of the non-naturally occurring melanocortin analog. In some embodiments, the disulfide bond of the sequence according to Formula (I), if present, provides the β-hairpin and/or β-turn structures of the non-naturally occurring melanocortin analog.

In some embodiments, the N-terminus of the non-naturally occurring melanocortin analog, if present, is modified by a functional group selected from the group consisting of an acyl group, an imine group, an amide group, a urea group, a carbamate group, a sulfonamide group, and an alkylamine group.

In some embodiments, the N-terminus of the non-naturally occurring melanocortin analog, if present, is modified by an acyl group. In some embodiments, the acyl group is acetyl group

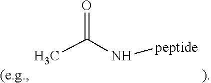

In some embodiments, the acyl group is formyl group

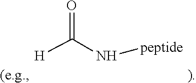

In some embodiments, the N-terminus of the non-naturally occurring melanocortin analog, if present, is modified by an imine group.

In some embodiments, the N-terminus of the non-naturally occurring melanocortin analog, if present, is modified by an amide group. In some embodiments, the amide group is a pyroglutamyl (pGlu) group

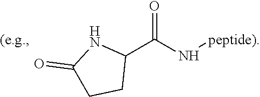

In some embodiments, the amide group is derived from a fatty acid

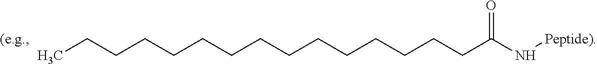

Other examples of the N-terminal modifications include, but are not limited to,

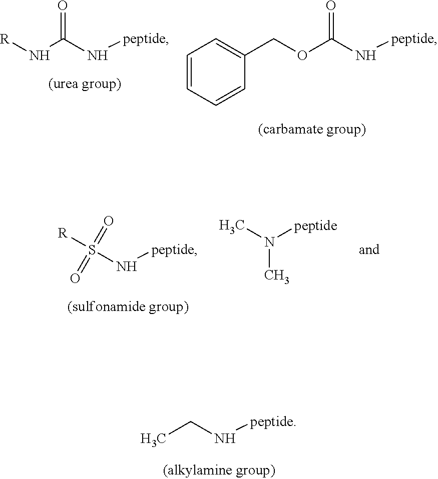

In some embodiments, the N-terminus of the non-naturally occurring melanocortin analog, if present, is not modified.

As discussed above, Y¹Y²Y³Y⁴Y⁵Y⁶Y⁷Y⁸ represents a C-terminus of the non-naturally occurring melanocortin analog. In some embodiments, Y³-Y⁸ are absent.

In some embodiments, Y³-Y⁸ are absent, and Y¹ is D-valine and Y² is D-proline, or Y¹ is D-proline and Y² is D-valine.

In some embodiments, Y³ is present and Y⁴-Y⁸ are absent.

In some embodiments, Y³ is present and Y⁴-Y⁸ are absent, and (i) Y¹ is D-valine or D-proline, (ii) Y² is D-valine or D-proline, and/or (iii) Y³ is D-valine or D-proline. In some embodiments, Y³ is present and Y⁴-Y⁸ are absent, and (i) Y¹ is D-valine, Y² is D-valine, and Y³ is D-proline, (ii) Y¹ is D-proline, Y² is D-valine, and Y³ is D-valine, or (iii) Y¹ is D-valine, Y² is D-proline, and Y³ is D-valine.

In some embodiments, Y³ and Y⁴ are present, and Y⁵-Y⁸ are absent. In some embodiments, Y³ and Y⁴ are present, and Y⁵-Y⁸ are absent, and (i) Y¹ is D-valine or D-proline, (ii) Y² is D-valine or D-proline, (iii) Y³ is D-valine or D-proline, and/or (iv) Y⁴ is D-valine or D-proline. In some embodiments, Y³ and Y⁴ are present, and Y⁵-Y⁸ are absent, and (i) Y¹ is D-valine, Y² is D-valine, Y³ is D-valine, and Y⁴ is D-proline, (ii) Y¹ is D-proline, Y² is D-valine, Y³ is D-valine, and Y⁴ is D-valine, (iii) Y¹ is D-valine, Y² is D-proline, Y³ is D-valine, and Y⁴ is D-valine, or (iv) Y¹ is D-valine, Y² is D-valine, Y³ is D-proline, and Y⁴ is D-valine.

In some embodiments, the C-terminus includes additional derivatives to extend the length of C-terminus of the non-naturally occurring melanocortin analog. Exemplary additional C-terminus include, but are not limited to, Y¹Y²Y³Y⁴Y⁵Y⁶Y⁷Y⁸Y⁹, Y¹Y²Y³Y⁴Y⁵Y⁶Y⁷Y⁸Y⁹Y¹⁰Y¹¹Y¹²Y¹³, Y¹Y²Y³Y⁴Y⁵Y⁶Y⁷Y⁸Y⁹Y¹⁰Y¹¹Y¹²Y¹³Y¹⁴, Y¹Y²Y³Y⁴Y⁵Y⁶Y⁷Y⁸Y⁹Y¹⁰Y¹¹Y¹²Y¹³Y¹⁴Y¹⁵, Y¹Y²Y³Y⁴Y⁵Y⁶Y⁷Y⁸Y⁹Y¹⁰Y¹¹Y¹²Y¹³Y¹⁴Y¹⁵Y¹⁶, Y¹Y²Y³Y⁴Y⁵Y⁶Y⁷Y⁸Y⁹Y¹⁰Y¹¹Y¹²Y¹³Y¹⁴Y¹⁵Y¹⁶Y¹⁷, Y¹Y²Y³Y⁴Y⁵Y⁶Y⁷Y⁸Y⁹Y¹⁰Y¹¹Y¹²Y¹³Y¹⁴Y¹⁵Y¹⁶Y¹⁷Y¹⁸, Y¹Y²Y³Y⁴Y⁵Y⁶Y⁷Y⁸Y⁹Y¹⁰Y¹¹Y¹²Y¹³Y¹⁴Y¹⁵Y¹⁶Y¹⁷Y¹⁸, Y¹⁹, Y¹Y²Y³Y⁴Y⁵Y⁶Y⁷Y⁸Y⁹Y¹⁰Y¹¹Y¹²Y¹³Y¹⁴Y¹⁵Y¹⁶Y¹⁷Y¹⁸, Y¹⁹, Y²⁰, Y¹Y²Y³Y⁴Y⁵Y⁶Y⁷Y⁸Y⁹Y¹⁰Y¹¹Y¹²Y¹³Y¹⁴Y¹⁵Y¹⁶Y¹⁷Y¹⁸, Y¹⁹, Y²⁰, Y²¹, Y¹Y²Y³Y⁴Y⁵Y⁶Y⁷Y⁸Y⁹Y¹⁰Y¹¹Y¹²Y¹³Y¹⁴Y¹⁵Y¹⁶Y¹⁷Y¹⁸Y¹⁹Y²⁰Y²¹Y²², Y¹Y²Y³Y⁴Y⁵Y⁶Y⁷Y⁸Y⁹Y¹⁰Y¹¹Y¹²Y¹³Y¹⁴Y¹⁵Y¹⁶Y¹⁷Y¹⁸Y¹⁹Y²⁰Y²¹Y²², Y²³ and Y¹Y²Y³Y⁴Y⁵Y⁶Y⁷Y⁸Y⁹Y¹⁰Y¹¹Y¹²Y¹³Y¹⁴Y¹⁵Y¹⁶Y¹⁷Y¹⁸Y¹⁹Y²⁰Y²¹Y²²Y²³, Y²⁴, etc., wherein Y⁹-Y²⁴, if present, are each independently D-proline or D-valine.

In some embodiments, the C-terminus of the non-naturally occurring melanocortin analog is modified by a functional group selected from the group consisting of an amidegroup, an ester group, and an aldehyde group.

In some embodiments, the C-terminus of the non-naturally occurring melanocortin analog is modified by an amide group

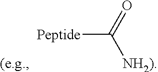

In the sequence of Formula (I), a non-naturally occurring melanocortin analog with a C-terminus modified by an amide may be represented by a terminal-NH₂, such as, for example, in the sequence Ac-Nle-c[Asp-Pro-dNal(2′)-Arg-Trp-Lys]-Val-Pro-NH₂ (SEQ ID NO: 116). In some embodiments, the amide group is an N-alkyl amide group

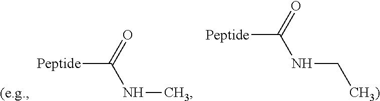

or an N-aryl amide group. In some embodiments, the N-aryl amide group is p-nitroanilide group(pNA)

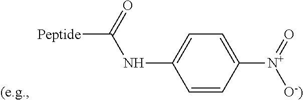

or 7-amino-4-methylcoumarin (AMC)

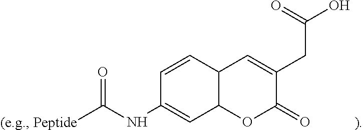

In some embodiments, the C-terminus of the non-naturally occurring melanocortin analog is modified by an ester group

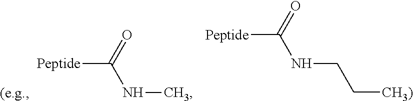

In some embodiments, the C-terminus of the non-naturally occurring melanocortin analog is modified by an aldehyde

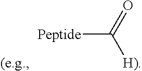

In some embodiments, the C-terminus of the non-naturally occurring melanocortin analog is not modified. In the sequence of Formula (I), a non-naturally occurring melanocortin analog with an unmodified C-terminus may be represented by—OH, such as, for example, in the sequence Ac-Nle-c[Asp-Pro-dNal(2′)-Arg-Trp-Lys]-dVal-dPro-OH (SEQ ID NO: 117) or by the absence of a C-terminal group, such as, for example, in the sequence Ac-Nle-c(Asp-Pro-dNal2′-Arg-Trp-Lys)-dVal-dPro(SEQ ID NO: 2; B07a). In some embodiments, the C-terminus of the non-naturally occurring melanocortin analog is not modified, and the sequence of Formula (I) is Ac-Nle-c(Asp-Pro-dNal2′-Arg-Trp-Lys)-dVal-dPro(SEQ ID NO: 2).

In some embodiments, R¹ is absent, and R² is D-aspartic acid.

In some embodiments, X¹, X², and X³ are absent.

In some embodiments, R⁴ is not D-phenylalanine. In some embodiments, R⁴ is D-Nal(2′). When R⁴ is D-Nal(2′), the non-naturally occurring melanocortin analog disclosed herein can act as a melanocortin receptor antagonist.

In some embodiments, the non-naturally occurring melanocortin analog disclosed herein is cyclized. For example, the non-naturally occurring melanocortin analog can be cyclized through a moiety selected from the group consisting of: a disulfide bond between R¹ or R² and R⁷ or X¹ when R¹ or R² is cysteine and R⁷ or X¹ is cysteine; a disulfide bond between R² and any one of R⁵—R²⁰ when R² and the any one of R⁵—R²⁰ are selected from the group consisting of cysteine, Pen, and dPen; a lactam bridge between R¹ and R⁷ when R¹ is norleucine and R⁷ is glutamic acid; a side-chain lactam bridge between R¹ and any of R⁵⁻⁸ when R¹ is aspartic acid and any of R⁵⁻⁸ are lysine; a side-chain lactam bridge between R¹ and R⁶ when R¹ is lysine and R⁶ is aspartic acid; a side-chain lactam bridge between R² and R⁷ when R² is glutamic acid, aspartic acid, or CO-cis-CH═CH—CO, and R⁷ is lysine or ornithine; a side-chain lactam bridge between R² and R⁸ when R² is glutamic acid or aspartic acid, R⁸ is lysine, and R⁷ is proline; a lactam closure between R¹ and R⁷ when R¹ is succinic acid or o-phthalic acid and R⁷ is lysine; and/or a lactam closure between R² and R⁷ when R² is succinic acid and R⁷ is 2,3-diamino-propionic acid. Cyclization can also occur from any of the X residues (optional stabilizing N-terminus residues). For example, X² can an aspartic acid that can be used for cyclization.

In some embodiments, R¹, R², and R⁷ are present and R⁸—R²⁰ are absent, and the sequence of Formula (I) is cyclized through R² and R⁷ via a lactam bond. In some embodiments, R¹ is acetylated norleucine, R² is aspartic acid, R³ is selected from the group consisting of proline, hydroxyproline, and D-hydroxyproline, R⁴ is D-Nal(2′), R⁵ is arginine, R⁶ is D-tryptophan or L-tryptophan, R⁷ is lysine, Y¹ is D-valine, and/or Y² is D-proline.

In some embodiments, the sequence of Formula (I) is: Ac-Nle-c(Asp-Pro-DNal2′-Arg-Trp-Lys)-dVal-dPro-NH₂ (SEQ ID NO: 3; B07) or Ac-Nle-_{c}(Asp-Hyp-DNal2′-Arg-Trp-Lys)-dVal-dPro-NH₂ (SEQ ID NO: 4; D3), wherein c represents cyclization through R² and R⁷ via a lactam bond.

In some embodiments, R¹, R², R⁷, and R⁸ are present and R⁹—R²⁰ are absent, and the sequence of Formula (I) is cyclized through R² and R⁸ via a lactam bond. In some embodiments, R¹ is acetylated norleucine, R² is aspartic acid, R³ is selected from the group consisting of proline, hydroxyproline, D-hydroxyproline, phenylalanine, and histidine, R⁴ is histidine or dNal(2′), R⁵ is dNal(2′) or arginine, R⁶ is selected from the group consisting of arginine, D-tryptophan, and L-tryptophan, R⁷ is tryptophan or proline, R⁸ is lysine, Y¹ is selected from the group consisting of D-valine, D-leucine, and D-isoleucine, and/or Y² is D-proline.

In some embodiments, the sequence of Formula (I) is selected from the group consisting of:

(SEQ ID NO: 6; D1)
Ac-Nle-_{c}(Asp-Pro-His-dNal(2′)-Arg-Trp-Lys)-dVal-
dPro-NH₂;
(SEQ ID NO: 7; D1γ)
Ac-Nle-_{c}(Asp-Phe-His-dNal(2′)-Arg-Trp-Lys)-dLeu-
dPro-NH₂;
(SEQ ID NO: 8; D1δ)
Ac-Nle-_{c}(Asp-Phe-His-dNal(2′)-Arg-Trp-Lys)-dIle-
dPro-NH₂;
(SEQ ID NO: 9; D2)
Ac-Nle-_{c}(Asp-His-dNal(2′)-Arg-Trp-Pro-Lys)-dVal-
dPro-NH₂;
(SEQ ID NO: 10; D4)
Ac-Nle-_{c}(Asp-Hyp-dNal(2′)-Arg-Trp-Pro-Lys)-dVal-
dPro-NH₂;
and
(SEQ ID NO: 11; D5)
Ac-Nle-_{c}(Asp-Pro-His-DNal(2′)-Arg-Trp-Lys)-DPro-
DVal-NH₂,

wherein c represents cyclization through R² and R⁸ via a lactam bond.

In some embodiments, R¹—R² and R⁷—R¹⁰ are present and R¹¹—R²⁰ are absent, and the sequence of Formula (I) is cyclized through R² and R¹⁰ via a lactam bond. In some embodiments, the sequence of Formula (I) is Ac-Nle-c(Asp-Phe-Phe-Pro-His-dNal(2′)-Arg-Trp-Lys)-dVal-dPro-NH₂ (SEQ ID NO: 12; D1a), wherein c represents cyclization through R² and R¹⁰ via a lactam bond.

In some embodiments, R¹—R² and R⁷—R¹⁰ are present and R¹¹—R²⁰ are absent, and the sequence of Formula (I) is cyclized through R⁴ and R¹⁰ via a lactam bond. In some embodiments, the sequence of Formula (I) is Ac-Nle-Phe-Phe-c(Asp-Phe-His-dNal(2′)-Arg-Trp-Lys)-dVal-dPro-NH₂ (SEQ ID NO: 13; D1B), wherein c represents cyclization through R⁴ and R¹⁰ via a lactam bond.

In some embodiments, the sequence of Formula (I) is linear. In some embodiments, the sequence of Formula (I) is Ac-Nle-Asp-Pro-dNal(2′)-Arg-Trp-Lys-dVal-dPro-NH₂ (SEQ ID NO: 14; A1). In some embodiments, the sequence of Formula (I) is Ac-Nle-Pro-DNal(2′)-Arg-Trp-DVal-DPro-NH₂ (SEQ ID NO: 15; A2).

In some embodiments of the sequence of Formula (I), R¹ is acetylated norleucine. Alternatively, in some embodiments, R¹ is an acetylated amino acid other than acetylated norleucine. In some embodiments, R¹ is a non-acetylated amino acid. In some embodiments, the sequence of Formula (I) is selected from the group consisting of:

(SEQ ID NO: 16)
Ac-dArg-_{c}(Asp-Pro-dNal(2′)-Arg-Trp-Lys)-dVal-dPro-
NH₂;
(SEQ ID NO: 17)
Ac-dMet-_{c}(Asp-Pro-dNal(2′)-Arg-Trp-Lys)-dVal-dPro-
NH₂;
(SEQ ID NO: 18)
Ac-dIle-_{c}(Asp-Pro-dNal(2′)-Arg-Trp-Lys)-dVal-dPro-
NH₂;
(SEQ ID NO: 19)
Ac-dLeu-_{c}(Asp-Pro-dNal(2′)-Arg-Trp-Lys)-dVal-dPro-
NH₂;
(SEQ ID NO: 20)
Ac-dVal-_{c}(Asp-Pro-dNal(2′)-Arg-Trp-Lys)-dVal-dPro-
NH₂;
(SEQ ID NO: 21)
Ac-dAla-_{c}(Asp-Pro-dNal(2′)-Arg-Trp-Lys)-dVal-dPro-
NH₂;
(SEQ ID NO: 22)
Ac-Ala-_{c}(Asp-Pro-dNal(2′)-Arg-Trp-Lys)-dVal-dPro-
NH₂;
(SEQ ID NO: 23)
Ac-Tle-_{c}(Asp-Pro-dNal(2′)-Arg-Trp-Lys)-dVal-dPro-
NH₂;
(SEQ ID NO: 24)
Ac-dTle-_{c}(Asp-Pro-dNal(2′)-Arg-Trp-Lys)-dVal-dPro-
NH₂;
(SEQ ID NO: 25)
Ac-dNle-_{c}(Asp-Pro-dNal(2′)-Arg-Trp-Lys)-dVal-dPro-
NH₂;
(SEQ ID NO: 26)
Ac-Nva-_{c}(Asp-Pro-dNal(2′)-Arg-Trp-Lys)-dVal-dPro-
NH₂;
(SEQ ID NO: 27)
Ac-Gly-_{c}(Asp-Pro-dNal(2′)-Arg-Trp-Lys)-dVal-dPro-
NH₂;
(SEQ ID NO: 28)
Ac-dPro-_{c}(Asp-Pro-dNal(2′)-Arg-Trp-Lys)-dVal-dPro-
NH₂;
(SEQ ID NO: 29)
Ac-dCys-_{c}(Asp-Pro-dNal(2′)-Arg-Trp-Lys)-dVal-dPro-
NH₂;
(SEQ ID NO: 30)
Ac-dPhe-_{c}(Asp-Pro-dNal(2′)-Arg-Trp-Lys)-dVal-dPro-
NH₂;
(SEQ ID NO: 31)
Ac-dTyr-_{c}(Asp-Pro-dNal(2′)-Arg-Trp-Lys)-dVal-dPro-
NH₂;
(SEQ ID NO: 32)
Ac-dGln-_{c}(Asp-Pro-dNal(2′)-Arg-Trp-Lys)-dVal-dPro-
NH₂;
(SEQ ID NO: 33)
Ac-dAsn-_{c}(Asp-Pro-dNal(2′)-Arg-Trp-Lys)-dVal-dPro-
NH₂;
(SEQ ID NO: 34)
Ac-transPro(guan)-c(Asp-Pro-dNal(2′)-Arg-Trp-Lys)-
dVal-dPro-NH₂;
(SEQ ID NO: 35)
Ac-cisPro(guan)-c(Asp-Pro-dNal(2′)-Arg-Trp-Lys)-
dVal-dPro-NH₂;
(SEQ ID NO: 36)
dTyr-c(Asp-Pro-dNal(2′)-Arg-Trp-Lys)-dVal-dPro-
NH₂;
(SEQ ID NO: 37)
Tyr-c(Asp-Pro-dNal(2′)-Arg-Trp-Lys)-dVal-dPro-NH₂;
and
(SEQ ID NO: 38)
Dmt-c(Asp-Pro-dNal(2′)-Arg-Trp-Lys)-dVal-dPro-NH₂,

wherein c represents cyclization through R² and R⁷ via a lactam bond.

In some embodiments, when the sequence of Formula (I) is cyclized through R² and R⁷, then R² is Asp and R⁷ is Lys. Alternatively, in some embodiments, when the sequence of Formula (I) is cyclized through R² and R⁷, then R² is an amino acid capable of forming a linkage to the residue at R⁷ other than Asp and R⁷ is an amino acid capable of forming a linkage to the residue at R² other than Lys. For example, when R² is an amino acid other than Arg and R⁷ is an amino acid other than Lys, the residue at R² may be capable of forming a linkage such as a lactam bond or a disulfide bond with the residue at R⁷. In some embodiments, the sequence of Formula (I) is selected from the group consisting of:

(SEQ ID NO: 39)
Ac-Nle-c(dAsp-Pro-dNal(2′)-Arg-Trp-Lys)-dVal-dPro-
NH₂;
(SEQ ID NO: 40)
Ac-Nle-c(Asp-Pro-dNal(2′)-Arg-Trp-dLys)-dVal-dPro-
NH₂;
(SEQ ID NO: 41)
Ac-Nle-c(Cys-Pro-dNal(2′)-Arg-Trp-Cys)-dVal-dPro-
NH₂;
(SEQ ID NO: 42)
Ac-Nle-c(dCys-Pro-dNal(2′)-Arg-Trp-Cys)-dVal-
dPro-NH₂;
(SEQ ID NO: 43)
Ac-Nle-c(Cys-Pro-dNal(2′)-Arg-Trp-dCys)-dVal-
dPro-NH₂;
(SEQ ID NO: 44)
Ac-Nle-c(dCys-Pro-dNal(2′)-Arg-Trp-dCys)-dVal-
dPro-NH₂;
(SEQ ID NO: 45)
Ac-Nle-c(Cys-dNal(2′)-Arg-Trp-Cys)-dVal-dPro-
NH₂;
(SEQ ID NO: 46)
Ac-Nle-c(dCys-dNal(2′)-Arg-Trp-Cys)-dVal-dPro-
NH₂;
(SEQ ID NO: 47)
Ac-Nle-c(Cys-dNal(2′)-Arg-Trp-dCys)-dVal-dPro-
NH₂;
(SEQ ID NO: 48)
Ac-Nle-c(dCys-dNal(2′)-Arg-Trp-dCys)-dVal-dPro-
NH₂;
(SEQ ID NO: 49)
Ac-Nle-c(Cys-Pro-dNal(2′)-Arg-Trp-Pen)-dVal-dPro-
NH₂;
(SEQ ID NO: 50)
Ac-Nle-c(dCys-Pro-dNal(2′)-Arg-Trp-Pen)-dVal-
dPro-NH₂;
(SEQ ID NO: 51)
Ac-Nle-c(Pen-Pro-dNal(2′)-Arg-Trp-Cys)-dVal-dPro-
NH₂;
(SEQ ID NO: 52)
Ac-Nle-c(Pen-Pro-dNal(2′)-Arg-Trp-dCys)-dVal-
dPro-NH₂;
(SEQ ID NO: 53)
Ac-Nle-c(Pen-Pro-dNal(2′)-Arg-Trp-Pen)-dVal-dPro-
NH₂;
(SEQ ID NO: 54)
Ac-Nle-c(dPen-Pro-dNal(2′)-Arg-Trp-Pen)-dVal-dPro-
NH₂;
(SEQ ID NO: 55)
Ac-Nle-c(dPen-Pro-dNal(2′)-Arg-Trp-dPen)-dVal-
dPro-NH₂;
(SEQ ID NO: 56)
Ac-Nle-c(Pen-Pro-dNal(2′)-Arg-Trp-dPen)-dVal-
dPro-NH₂;
(SEQ ID NO: 57)
Ac-Nle-c(Cys-dNal(2′)-Arg-Trp-Pen)-dVal-dPro-NH₂;
(SEQ ID NO: 58)
Ac-Nle-c(dCys-dNal(2′)-Arg-Trp-Pen)-dVal-dPro-
NH₂;
(SEQ ID NO: 59)
Ac-Nle-c(Pen-dNal(2′)-Arg-Trp-Cys)-dVal-dPro-NH₂;
(SEQ ID NO: 60)
Ac-Nle-c(Pen-dNal(2′)-Arg-Trp-dCys)-dVal-dPro-NH₂;
(SEQ ID NO: 61)
Ac-Nle-c(Pen-dNal(2′)-Arg-Trp-Pen)-dVal-dPro-NH₂;
(SEQ ID NO: 62)
Ac-Nle-c(dPen-dNal(2′)-Arg-Trp-Pen)-dVal-dPro-NH₂;
(SEQ ID NO: 63)
Ac-Nle-c(dPen-dNal(2′)-Arg-Trp-dPen)-dVal-dPro-
NH₂;
(SEQ ID NO: 64)
Ac-Nle-c(Pen-dNal(2′)-Arg-Trp-dPen)-dVal-dPro-NH₂;
(SEQ ID NO: 65)
Ac-Nle-c(Asp-Pro-dNal(2′)-Arg-Trp-Orn)-dVal-dPro-
NH₂;
(SEQ ID NO: 66)
Ac-Nle-c(Asp-Pro-dNal(2′)-Arg-Trp-dOrn)-dVal-dPro-
NH₂;
(SEQ ID NO: 67)
Ac-Nle-c(Glu-Pro-dNal(2′)-Arg-Trp-Orn)-dVal-dPro-
NH₂;
and
(SEQ ID NO: 68)
Ac-Nle-c(Glu-Pro-dNal(2′)-Arg-Trp-dOrn)-dVal-dPro-
NH₂,

wherein c represents cyclization through R² and R⁶ or R⁷ via a lactam bond or a disulfide bond.

In some embodiments of the sequence of Formula (I), R³ is Pro. Alternatively, in some embodiments, R³ is absent or an amino acid other than Pro. In some embodiments, the sequence of Formula (I) is selected from the group consisting of:

(SEQ ID NO: 69)
Ac-Nle-c[Asp-dNal(2′)-Arg-Trp-Lys]-dVal-dPro-NH₂;
(SEQ ID NO: 70)
Ac-Nle-c[Asp-Ala-dNal(2′)-Arg-Trp-Lys]-dVal-dPro-NH₂;
(SEQ ID NO: 71)
Ac-Nle-c(Asp-dPro-dNal(2′)-Arg-Trp-Lys)-dVal-dPro-NH₂;
(SEQ ID NO: 72)
Ac-Nle-c(Asp-dAla-dNal(2′)-Arg-Trp-Lys)-dVal-dPro-NH₂;
(SEQ ID NO: 73)
Ac-Nle-c(Asp-dMet-dNal(2′)-Arg-Trp-Lys)-dVal-dPro-NH₂;
(SEQ ID NO: 74)
Ac-Nle-c(Asp-Pro-Gly-dNal(2′)-Arg-Trp-Lys)-dVal-dPro-NH₂;
(SEQ ID NO: 75)
Ac-Nle-c(Asp-Gly-Gly-dNal(2′)-Arg-Trp-Lys)-dVal-dPro-NH₂;
(SEQ ID NO: 76)
Ac-Nle-c[Asp-Gly-dNal(2′)-Arg-Trp-Lys]-dVal-dPro-NH₂;
(SEQ ID NO: 77)
Ac-Nle-c[Asp-Leu-dNal(2′)-Arg-Trp-Lys]-dVal-dPro-NH₂;
(SEQ ID NO: 78)
Ac-Nle-c[Asp-Ile-dNal(2′)-Arg-Trp-Lys]-dVal-dPro-NH₂;
(SEQ ID NO: 79)
Ac-Nle-c[Asp-Val-dNal(2′)-Arg-Trp-Lys]-dVal-dPro-NH₂;
(SEQ ID NO: 80)
Ac-Nle-c[Asp-dLeu-dNal(2′)-Arg-Trp-Lys]-dVal-dPro-NH₂;
(SEQ ID NO: 81)
Ac-Nle-c[Asp-dlle-dNal(2′)-Arg-Trp-Lys]-dVal-dPro-NH₂;
(SEQ ID NO: 82)
Ac-Nle-c[Asp-dVal-dNal(2′)-Arg-Trp-Lys]-dVal-dPro-NH₂;
(SEQ ID NO: 83)
Ac-Nle-c[Asp-Trp-dNal(2′)-Arg-Trp-Lys]-dVal-dPro-NH₂;
(SEQ ID NO: 84)
Ac-Nle-c[Asp-dTrp-dNal(2′)-Arg-Trp-Lys]-dVal-dPro-NH₂;
(SEQ ID NO: 85)
Ac-Nle-c[Asp-transPro(guan)-dNal(2′)-Arg-Trp-Lys]-dVal-dPro-NH₂;
and
(SEQ ID NO: 86)
Ac-Nle-c[Asp-cisPro(guan)-dNal(2′)-Arg-Trp-Lys]-dVal-dPro-NH₂,

wherein c represents cyclization through R² and R⁷ via a lactam bond.

In some embodiments of the sequence of Formula (I), R⁵ is Arg. Alternatively, in some embodiments, R⁵ is absent or an amino acid other than Arg. In some embodiments, the sequence of Formula (I) is selected from the group consisting of:

(SEQ ID NO: 87)
Ac-Nle-c(Asp-Pro-dNal(2′)-Trp-Lys)-dVal-dPro-NH₂;
(SEQ ID NO: 88)
Ac-Nle-c(Asp-Pro-dNal(2′)-Lys-Trp-Lys)-dVal-dPro-NH₂;
(SEQ ID NO: 89)
Ac-Nle-c(Asp-Pro-dNal(2′)-dLys-Trp-Lys)-dVal-dPro-NH₂;
(SEQ ID NO: 90)
Ac-Nle-c(Asp-Pro-dNal(2′)-dArg-Trp-Lys)-dVal-dPro-NH₂;
(SEQ ID NO: 91)
Ac-Nle-c(Asp-Pro-dNal(2′)-Orn-Trp-Lys)-dVal-dPro-NH₂;
(SEQ ID NO: 92)
Ac-Nle-c(Asp-Pro-dNal(2′)-dOrn-Trp-Lys)-dVal-dPro-NH₂;
(SEQ ID NO: 93)
Ac-Nle-c(Asp-Pro-dNal(2′)-His-Trp-Lys)-dVal-dPro-NH₂;
(SEQ ID NO: 94)
Ac-Nle-c(Asp-Pro-dNal(2′)-Ala-Trp-Lys)-dVal-dPro-NH₂;
(SEQ ID NO: 95)
Ac-Nle-c(Asp-Pro-dNal(2′)-Gly-Trp-Lys)-dVal-dPro-NH₂;
(SEQ ID NO: 96)
Ac-Nle-c(Asp-Pro-dNal(2′)-Asp-Trp-Lys)-dVal-dPro-NH₂;
(SEQ ID NO: 97)
Ac-Nle-c(Asp-Pro-dNal(2′)-Glu-Trp-Lys)-dVal-dPro-NH₂;
(SEQ ID NO: 98)
Ac-Nle-c(Asp-Pro-dNal(2′)-dHis-Trp-Lys)-dVal-dPro-NH₂;
(SEQ ID NO: 99)
Ac-Nle-c(Asp-Pro-dNal(2′)-dAla-Trp-Lys)-dVal-dPro-NH₂;
(SEQ ID NO: 100)
Ac-Nle-c(Asp-Pro-dNal(2′)-dAsp-Trp-Lys)-dVal-dPro-NH₂;
and
(SEQ ID NO: 101)
Ac-Nle-c(Asp-Pro-dNal(2′)-dGlu-Trp-Lys)-dVal-dPro-NH₂,

wherein c represents cyclization through R² and R⁷ via a lactam bond.

In some embodiments of the sequence of Formula (I), R⁶ is Trp. Alternatively, in some embodiments, R⁶ is absent or an amino acid other than Trp. In some embodiments, the sequence of Formula (I) is selected from the group consisting of:

(SEQ ID NO: 102)
Ac-Nle-c(Asp-Pro-dNal(2′)-Arg-Lys)-dVal-dPro-NH₂;
(SEQ ID NO: 103)
Ac-Nle-c(Asp-Pro-dNal(2′)-Arg-Nal(1′)-Lys)-dVal-dPro-NH₂;
(SEQ ID NO: 104)
Ac-Nle-c(Asp-Pro-dNal(2′)-Arg-Aia-Lys)-dVal-dPro-NH₂;
(SEQ ID NO: 105)
Ac-Nle-c(Asp-Pro-dNal(2′)-Arg-Phe-Lys)-dVal-dPro-NH₂;
(SEQ ID NO: 106)
Ac-Nle-c(Asp-Pro-dNal(2′)-Arg-Tyr-Lys)-dVal-dPro-NH₂;
(SEQ ID NO: 107)
Ac-Nle-c(Asp-Pro-dNal(2′)-Arg-His-Lys)-dVal-dPro-NH₂;
(SEQ ID NO: 108)
Ac-Nle-c(Asp-Pro-dNal(2′)-Arg-Ala-Lys)-dVal-dPro-NH₂;
(SEQ ID NO: 109)
Ac-Nle-c(Asp-Pro-dNal(2′)-Arg-dNal(1′)-Lys)-dVal-dPro-NH₂;
(SEQ ID NO: 110)
Ac-Nle-c(Asp-Pro-dNal(2′)-Arg-dPhe-Lys)-dVal-dPro-NH₂;
(SEQ ID NO: 111)
Ac-Nle-c(Asp-Pro-dNal(2′)-Arg-dNal(2′)-Lys)-dVal-dPro-NH₂;
(SEQ ID NO: 112)
Ac-Nle-c(Asp-Pro-dNal(2′)-Arg-dTyr-Lys)-dVal-dPro-NH₂;
(SEQ ID NO: 113)
Ac-Nle-c(Asp-Pro-dNal(2′)-Arg-dHis-Lys)-dVal-dPro-NH₂;
and
(SEQ ID NO: 114)
Ac-Nle-c(Asp-Pro-dNal(2′)-Arg-dAla-Lys)-dVal-dPro-NH₂,

wherein c represents cyclization through R² and R⁷ via a lactam bond.

In some embodiments of the sequence of Formula (I), R⁴ is dNal(2′). Alternatively, in some embodiments, R⁴ is an amino acid other than dNal(2′). For example, in some embodiments, when R⁴ is an amino acid other than dNal(2′), R⁴ is Bip. In some embodiments, the sequence of Formula (I) is Ac-Nle-c(Asp-Pro-Bip-Arg-Trp-Lys)-dVal-dPro-NH₂ (SEQ ID NO: 115), wherein c represents cyclization through R² and R⁷ via a lactam bond. In some embodiments, when R⁴ is an amino acid other than dNal(2′), R⁴ is dPhe, p(CI)dPhe p(I)dPhe, p(Br)dPhe, p(F)dPhe, or p(CF₃)dPhe.

In some embodiments of the sequence of Formula (I), Y¹ is dVal and Y² is dPro. Alternatively, in some embodiments, Y¹ is an amino acid other than dVal and Y² is an amino acid other than dPro. In some embodiments, Y¹ is dVal and Y² is an amino acid other than dPro. In some embodiments, Y¹ is an amino acid other than dVal and Y² is dPro.

In some embodiments, Y¹ is dVal, Y² is dPro, and the C-terminus is modified by NH₂. In some embodiments, Y¹ is an amino acid other than dVal, Y² is an amino acid other than dPro, and the C-terminus is modified by NH₂. In some embodiments, Y¹ is dVal, Y² is an amino acid other than dPro, and the C-terminus is modified by NH₂. In some embodiments, Y¹ is an amino acid other than dVal, Y² is dPro, and the C-terminus is modified by NH₂. In some embodiments, Y¹ is dVal, Y² is dPro, and the C-terminus is unmodified.

In embodiments of Formula (I) when Y¹ is an amino acid other than dVal, then Y¹ is selected from dPro, Val, Hyp, dHyp, Pro, Ala, dAla, Gly, Asp, Arg, Asn, dAsp, dArg, dAsn, Lys, dLys, and dTle. In embodiments of Formula (I) when Y² is an amino acid other than dPro, then Y² or selected from dVal, Val, Hyp, dHyp, Pro, Ala, dAla, Gly, Asp, Arg, Asn, dAsp, dArg, dAsn, and dTle. In some embodiments, the sequence of Formula (I) is selected from the group consisting of:

(SEQ ID NO: 116)
Ac-Nle-c[Asp-Pro-dNal(2′)-Arg-Trp-Lys]-Val-Pro-NH₂;
(SEQ ID NO: 117)
Ac-Nle-c[Asp-Pro-dNal(2′)-Arg-Trp-Lys]-dVal-dPro-OH;
(SEQ ID NO: 118)
Ac-Nle-c[Asp-Pro-dNal(2′)-Arg-Trp-Lys]-dPro-dVal-OH;
(SEQ ID NO: 119)
Ac-Nle-c[Asp-Pro-dNal(2′)-Arg-Trp-Lys]-dVal-Hyp-NH₂;
(SEQ ID NO: 120)
Ac-Nle-c[Asp-Pro-dNal(2′)-Arg-Trp-Lys]-dVal-dHyp-NH₂;
(SEQ ID NO: 121)
Ac-Nle-c[Asp-Pro-dNal(2′)-Arg-Trp-Lys]-Val-Hyp-NH₂;
(SEQ ID NO: 122)
Ac-Nle-c[Asp-Pro-dNal(2′)-Arg-Trp-Lys]-Val-dHyp-NH₂;
(SEQ ID NO: 123)
Ac-Nle-c[Asp-Pro-dNal(2′)-Arg-Trp-Lys]-Hyp-dVal-NH₂;
(SEQ ID NO: 124)
Ac-Nle-c[Asp-Pro-dNal(2′)-Arg-Trp-Lys]-dHyp-dVal-NH₂;
(SEQ ID NO: 125)
Ac-Nle-c[Asp-Pro-dNal(2′)-Arg-Trp-Lys]-Hyp-Val-NH₂;
(SEQ ID NO: 126)
Ac-Nle-c[Asp-Pro-dNal(2′)-Arg-Trp-Lys]-dHyp-Val-NH₂;
(SEQ ID NO: 127)
Ac-Nle-c[Asp-Pro-dNal(2′)-Arg-Trp-Lys]-dVal-dVal-NH₂;
(SEQ ID NO: 128)
Ac-Nle-c[Asp-Pro-dNal(2′)-Arg-Trp-Lys]-dPro-dPro-NH₂;
(SEQ ID NO: 129)
Ac-Nle-c[Asp-Pro-dNal(2′)-Arg-Trp-Lys]-dVal-NH₂;
(SEQ ID NO: 130)
Ac-Nle-c[Asp-Pro-dNal(2′)-Arg-Trp-Lys]-dPro-NH₂;
(SEQ ID NO: 131)
Ac-Nle-c[Asp-Pro-dNal(2′)-Arg-Trp-Lys]-Val-NH₂;
(SEQ ID NO: 132)
Ac-Nle-c[Asp-Pro-dNal(2′)-Arg-Trp-Lys]-Pro-NH₂;
(SEQ ID NO: 133)
Ac-Nle-c[Asp-Pro-dNal(2′)-Arg-Trp-Lys]-Ala-NH₂;
(SEQ ID NO: 134)
Ac-Nle-c[Asp-Pro-dNal(2′)-Arg-Trp-Lys]-dAla-NH₂;
(SEQ ID NO: 135)
Ac-Nle-c[Asp-Pro-dNal(2′)-Arg-Trp-Lys]-dHyp-NH₂;
(SEQ ID NO: 136);
Ac-Nle-c[Asp-Pro-dNal(2′)-Arg-Trp-Lys]-Hyp-NH₂
(SEQ ID NO: 137)
Ac-Nle-c[Asp-Pro-dNal(2′)-Arg-Trp-Lys]-dAla-dAla-NH₂;
(SEQ ID NO: 138)
Ac-Nle-c[Asp-Pro-dNal(2′)-Arg-Trp-Lys]-Ala-Ala-NH₂;
(SEQ ID NO: 139)
Ac-Nle-c[Asp-Pro-dNal(2′)-Arg-Trp-Lys]-Gly-Gly-NH₂;
(SEQ ID NO: 140)
Ac-Nle-c[Asp-Pro-dNal(2′)-Arg-Trp-Lys]-dVal-Asp-NH₂;
(SEQ ID NO: 141)
Ac-Nle-c[Asp-Pro-dNal(2′)-Arg-Trp-Lys]-dVal-Arg-NH₂;
(SEQ ID NO: 142)
Ac-Nle-c[Asp-Pro-dNal(2′)-Arg-Trp-Lys]-dVal-Asn-NH₂;
(SEQ ID NO: 143)
Ac-Nle-c[Asp-Pro-dNal(2′)-Arg-Trp-Lys]-dVal-dAsp-NH₂;
(SEQ ID NO: 144)
Ac-Nle-c[Asp-Pro-dNal(2′)-Arg-Trp-Lys]-dVal-dArg-NH₂;
(SEQ ID NO: 145)
Ac-Nle-c[Asp-Pro-dNal(2′)-Arg-Trp-Lys]-dVal-dAsn-NH₂;
(SEQ ID NO: 146)
Ac-Nle-c[Asp-Pro-dNal(2′)-Arg-Trp-Lys]-Asp-dPro-NH₂;
(SEQ ID NO: 147)
Ac-Nle-c[Asp-Pro-dNal(2′)-Arg-Trp-Lys]-Arg-dPro-NH₂;
(SEQ ID NO: 148)
Ac-Nle-c[Asp-Pro-dNal(2′)-Arg-Trp-Lys]-Asn-dPro-NH₂;
(SEQ ID NO: 149)
Ac-Nle-c[Asp-Pro-dNal(2′)-Arg-Trp-Lys]-dAsp-dPro-NH₂;
(SEQ ID NO: 150)
Ac-Nle-c[Asp-Pro-dNal(2′)-Arg-Trp-Lys]-dArg-dPro-NH₂;
(SEQ ID NO: 151)
Ac-Nle-c[Asp-Pro-dNal(2′)-Arg-Trp-Lys]-dAsn-dPro-NH₂;
(SEQ ID NO: 152)
Ac-Nle-c[Asp-Pro-dNal(2′)-Arg-Trp-Lys]-Asp-NH₂;
(SEQ ID NO: 153)
Ac-Nle-c[Asp-Pro-dNal(2′)-Arg-Trp-Lys]-Arg-NH₂;
(SEQ ID NO: 154)
Ac-Nle-c[Asp-Pro-dNal(2′)-Arg-Trp-Lys]-Asn-NH₂;
(SEQ ID NO: 155)
Ac-Nle-c[Asp-Pro-dNal(2′)-Arg-Trp-Lys]-dAsp-NH₂;
(SEQ ID NO: 156)
Ac-Nle-c[Asp-Pro-dNal(2′)-Arg-Trp-Lys]-dArg-NH₂;
(SEQ ID NO: 157)
Ac-Nle-c[Asp-Pro-dNal(2′)-Arg-Trp-Lys]-dAsn-NH₂;
(SEQ ID NO: 158)
Ac-Nle-c[Asp-Pro-dNal(2′)-Arg-Trp-Lys]-Lys-Pro-Val-NH₂;
(SEQ ID NO: 159)
Ac-Nle-c[Asp-Pro-dNal(2′)-Arg-Trp-Lys]-Lys-dPro-dVal-NH₂;
(SEQ ID NO: 160)
Ac-Nle-c[Asp-Pro-dNal(2′)-Arg-Trp-Lys]-dLys-dPro-dVal-NH₂;
(SEQ ID NO: 161)
Ac-Nle-c[Asp-Pro-dNal(2′)-Arg-Trp-Lys]-Lys-dPro-NH₂;
(SEQ ID NO: 162)
Ac-Nle-c[Asp-Pro-dNal(2′)-Arg-Trp-Lys]-dLys-dPro-NH₂;
(SEQ ID NO: 163)
Ac-Nle-c[Asp-Pro-dNal(2′)-Arg-Trp-Lys]-Lys-Val-Pro-NH₂;
(SEQ ID NO: 164)
Ac-Nle-c[Asp-Pro-dNal(2′)-Arg-Trp-Lys]-Lys-dVal-dPro-NH₂;
(SEQ ID NO: 165)
Ac-Nle-c[Asp-Pro-dNal(2′)-Arg-Trp-Lys]-dLys-dVal-dPro-NH₂;
(SEQ ID NO: 166)
Ac-Nle-c[Asp-Pro-dNal(2′)-Arg-Trp-Lys]-Arg-Pro-Val-NH₂;
(SEQ ID NO: 167)
Ac-Nle-c[Asp-Pro-dNal(2′)-Arg-Trp-Lys]-Arg-dPro-dVal-NH₂;
(SEQ ID NO: 168)
Ac-Nle-c[Asp-Pro-dNal(2′)-Arg-Trp-Lys]-dArg-dPro-dVal-NH₂;
(SEQ ID NO: 169)
Ac-Nle-c[Asp-Pro-dNal(2′)-Arg-Trp-Lys]-Arg-Val-Pro-NH₂;
(SEQ ID NO: 170)
Ac-Nle-c[Asp-Pro-dNal(2′)-Arg-Trp-Lys]-Arg-dVal-dPro-NH₂;
(SEQ ID NO: 171)
Ac-Nle-c[Asp-Pro-dNal(2′)-Arg-Trp-Lys]-dArg-dVal-dPro-NH₂;
(SEQ ID NO: 172)
Ac-Nle-c[Asp-Pro-dNal(2′)-Arg-Trp-Lys]-dTle-dVal-dPro-NH₂;
(SEQ ID NO: 173)
Ac-Nle-c[Asp-Pro-dNal(2′)-Arg-Trp-Lys]-dTle-dPro-NH₂;
(SEQ ID NO: 174)
Ac-Nle-c[Asp-Pro-dNal(2′)-Arg-Trp-Lys]-dTle-dVal-NH₂;
(SEQ ID NO: 175)
Ac-Nle-c[Asp-Pro-dNal(2′)-Arg-Trp-Lys]-dTle-dTle-NH₂;
(SEQ ID NO: 176)
Ac-Nle-c[Asp-Pro-dNal(2′)-Arg-Trp-Lys]-dTle-dTle-dPro-NH₂;
(SEQ ID NO: 177)
Ac-Nle-c[Asp-Pro-dNal(2′)-Arg-Trp-Lys]-dTle-dTle-dVal-NH₂;
(SEQ ID NO: 178)
Ac-Nle-c[Asp-Pro-dNal(2′)-Arg-Trp-Lys]-dPro-dTle-NH₂;
and
(SEQ ID NO: 179)
Ac-Nle-c[Asp-Pro-dNal(2′)-Arg-Trp-Lys]-dVal-dTle-NH₂,

wherein c represents cyclization through R² and R⁷ via a lactam bond.

In some embodiments of the sequence of Formula (I) when Y³ is present and Y⁴-8 are absent, then each of Y¹, Y², and Y³ are independently selected form dVal and dPro. In some embodiments of the sequence of Formula (I) when Y³ and Y⁴ are present and Y⁵⁻⁸ are absent, then each of Y¹, Y², Y³, and Y⁴ are independently selected form dVal and dPro. In some embodiments of the sequence of Formula (I) when Y³-Y⁵ are present and Y⁶-8 are absent, then each of Y¹-Y⁵ are independently selected form dVal and dPro. In some embodiments of the sequence of Formula (I) when Y³-Y⁶ are present and Y⁷-8 are absent, then each of Y¹-Y⁶ are independently selected form dVal and dPro. In some embodiments of the sequence of Formula (I) when Y³-Y⁷ are present and Y⁸ is absent, then each of Y¹-Y⁷ are independently selected form dVal and dPro. In some embodiments of the sequence of Formula (I) when Y³-Y⁸ are present, then each of Y¹-Y⁸ are independently selected form dVal and dPro. In some embodiments, the sequence of Formula (I) is selected from the group consisting of:

(SEQ ID NO: 180)
Ac-Nle-c(Asp-Pro-dNal(2′)-Arg-Trp-Lys)-dVal-dVal-
dPro-NH₂;
(SEQ ID NO: 181);
Ac-Nle-c(Asp-Pro-dNal(2′)-Arg-Trp-Lys)-dPro-dVal-
dPro-NH₂
(SEQ ID NO: 182)
Ac-Nle-c(Asp-Pro-dNal(2′)-Arg-Trp-Lys)-dVal-dVal-
dVal-dPro-NH₂;
(SEQ ID NO: 183)
Ac-Nle-c(Asp-Pro-dNal(2′)-Arg-Trp-Lys)-dVal-dPro-
dVal-dPro-NH₂;
and
(SEQ ID NO: 184)
Ac-Nle-c(Asp-Pro-dNal(2′)-Arg-Trp-Lys)-dVal-dVal-
dVal-dVal-dVal-dVal-dPro-NH₂,

wherein c represents cyclization through R² and R⁷ via a lactam bond.

In some embodiments of the sequence of Formula (I), X¹ is present and is acetylated norleucine, and R¹ is present and is norleucine. In some embodiments, X² is present and is norleucine. In some embodiments, X³ is present and is norleucine. In some embodiments, the sequence of Formula (I) is selected from the group consisting of:

(SEQ ID NO: 185)
Ac-Nle-Nle-c(Asp-Pro-dNal(2′)-Arg-Trp-Lys)-dVal-
dPro-NH₂;
(SEQ ID NO: 186)
Ac-Nle-Nle-Nle-c(Asp-Pro-dNal(2′)-Arg-Trp-Lys)-
dVal-dPro-NH₂;
and
(SEQ ID NO: 187)
Ac-Nle-Nle-Nle-Nle-c(Asp-Pro-dNal(2′)-Arg-Trp-
Lys)-dVal-dPro-NH₂,

wherein c represents cyclization through R² and R⁷ via a lactam bond.

As described above, some of the non-naturally occurring melanocortin analogs comprising the sequence of Formula (I) may bind the melanocortin 3 receptor and the melanocortin 4 receptor with the same or generally similar affinity. Other non-naturally occurring melanocortin analogs comprising the sequence of Formula (I) may bind the melanocortin 3 receptor with greater affinity than the melanocortin 4 receptor. In some embodiments, when the non-naturally occurring melanocortin analog of the present technology binds the melanocortin 3 receptor with greater affinity than the melanocortin 4 receptor, then the sequence of Formula (I) is selected from the group consisting of:

(SEQ ID NO: 188)
Ac-Nle-c[Asp-Pro-dPhe-Arg-dTrp-Lys]-dVal-dPro-NH₂;
(SEQ ID NO: 189)
Ac-Nle-c[Asp-His-Arg-p(I)dPhe-Arg-Tic-Lys]-dVal-dPro-NH₂;
(SEQ ID NO: 190)
Ac-Nle-c[Asp-His-Arg-dBip-Arg-Tic-Lys]-dVal-dPro-NH₂;
(SEQ ID NO: 191)
Ac-Nle-c[Asp-Pro-Arg-p(I)dPhe-Arg-Tic-Lys]-dVal-dPro-NH₂;
(SEQ ID NO: 192)
Ac-Nle-c[Asp-Pro-Arg-dBip-Arg-Tic-Lys]-dVal-dPro-NH₂;
(SEQ ID NO: 193)
Ac-Nle-c[Asp-Arg-Pro-p(I)dPhe-Arg-Tic-Lys]-dVal-dPro-NH₂;
(SEQ ID NO: 194)
Ac-Nle-c[Asp-Arg-Pro-dBip-Arg-Tic-Lys]-dVal-dPro-NH₂;
(SEQ ID NO: 195)
Ac-Nle-c[Asp-Pro-Pro-dNal(2′)-Arg-Trp-Lys]-dVal-dPro-NH₂;
(SEQ ID NO: 196)
Ac-Nle-c[Asp-Trp-Pro-dNal(2′)-Arg-Trp-Lys]-dVal-dPro-NH₂;
(SEQ ID NO: 197)
Ac-Nle-c[Asp-Pro-dNal(2′)-Arg-dTrp-Lys]-dVal-dPro-NH₂;
(SEQ ID NO: 198)
c[CO-cis-CH = CH-CO-Pro-dNal(2′)-Arg-Trp-Lys]-dVal-dPro-NH₂;
(SEQ ID NO: 199)
Ac-Nle-c[Asp-Aba-dPhe-Arg-Trp-Lys]-dVal-dPro-NH₂;
(SEQ ID NO: 200)
Ac-Nle-c[Asp-β-Ala-dNal(2′)-Arg-Trp-Lys]-dVal-dPro-NH₂;
(SEQ ID NO: 201)
Ac-Nle-c[Asp-Mamb-dNal(2′)-Arg-Trp-Lys]-dVal-dPro-NH₂;
(SEQ ID NO: 202)
Ac-Nle-c[Asp-Acpc-dNal(2′)-Arg-Trp-Lys]-dVal-dPro-NH₂;
(SEQ ID NO: 203)
Ac-c[Cys-Arg-dPhe-Cys]-Trp-dVal-dPro-NH₂;
(SEQ ID NO: 204)
Ac-Nle-c[Asp-Pro-dNal(2′)-Arg-Trp-Lys]-Trp-NH₂;
(SEQ ID NO: 205)
Ac-Nle-c(Asp-Aba-dNal(2′)-Arg-Trp-Lys)-dVal-dPro-NH₂;
(SEQ ID NO: 206)
Ac-Nle-c(Asp-Aia-dNal(2′)-Arg-Trp-Lys)-dVal-dPro-NH₂;
(SEQ ID NO: 207)
Ac-Nle-c(Asp-Ata-dNal(2′)-Arg-Trp-Lys)-dVal-dPro-NH₂;
(SEQ ID NO: 208)
Ac-Nle-c(Asp-Aia-dPhe-Arg-Trp-Lys)-dVal-dPro-NH₂;
(SEQ ID NO: 209)
Ac-Nle-c(Asp-Ata-dPhe-Arg-Trp-Lys)-dVal-dPro-NH₂;
(SEQ ID NO: 210)
Ac-Nle-c(Asp-Aba-p(Cl)dPhe-Arg-Trp-Lys)-dVal-dPro-NH₂;
(SEQ ID NO: 211)
Ac-Nle-c(Asp-Aia-p(Cl)dPhe-Arg-Trp-Lys)-dVal-dPro-NH₂;
and
(SEQ ID NO: 212)
Ac-Nle-c(Asp-Ata-p(Cl)dPhe-Arg-Trp-Lys)-dVal-dPro-NH₂,

wherein c represents cyclization through R¹ or R² and R⁷ or R⁸ via a lactam bond or through R² and R⁵ via a disulfide bond.

Some non-naturally occurring melanocortin analogs comprising the sequence of Formula (I) may bind the melanocortin 4 receptor with greater affinity than the melanocortin 3 receptor. In some embodiments, when the non-naturally occurring melanocortin analog binds the melanocortin 4 receptor with greater affinity than the melanocortin 3 receptor, then the sequence of Formula (I) is selected from the group consisting of:

(SEQ ID NO: 213)
Ac-Nle-c[Asp-Atc-dPhe-Arg-Trp-Lys]-dVal-dPro-NH₂;
(SEQ ID NO: 214)
Ac-Nle-c[Asp-APC-dPhe-Arg-Trp-Lys]-dVal-dPro-NH₂;
(SEQ ID NO: 215)
Ac-Nle-c[Asp-APPC-dPhe-Arg-Trp-Lys]-dVal-dPro-NH₂;
(SEQ ID NO: 216)
Ac-Nle-c[Asp-Pro-p(Cl)dPhe-Arg-Trp-Lys]-dVal-dPro-NH₂;
(SEQ ID NO: 217)
Ac-Nle-c[Asp-Pro-p(I)dPhe-Arg-Trp-Lys]-dVal-dPro-NH₂;
(SEQ ID NO: 218)
Ac-Nle-c[Asp-Pro-p(Br)dPhe-Arg-Trp-Lys]-dVal-dPro-NH₂;
(SEQ ID NO: 219)
Ac-Nle-c[Asp-Pro-p(F)dPhe-Arg-Trp-Lys]-dVal-dPro-NH₂;
(SEQ ID NO: 220)
Ac-Nle-c(Asp-Pro-p(CF₃)dPhe-Arg-Trp-Lys)-dVal-dPro-NH₂;
(SEQ ID NO: 221)
Ac-Nle-c[Asp-His-p(Cl)dPhe-Arg-Trp-Lys]-dVal-dPro-NH₂;
(SEQ ID NO: 222)
Ac-Nle-c[Asp-His-p(I)dPhe-Arg-Trp-Lys]-dVal-dPro-NH₂;
(SEQ ID NO: 223)
Ac-Nle-c[Asp-His-p(Br)dPhe-Arg-Trp-Lys]-dVal-dPro-NH₂;
(SEQ ID NO: 224)
Ac-Nle-c[Asp-His-p(F)dPhe-Arg-Trp-Lys]-dVal-dPro-NH₂;
(SEQ ID NO: 225)
203. Ac-Nle-c(Asp-His-p(CF₃)dPhe-Arg-Trp-Lys)-dVal-dPro-NH₂;
(SEQ ID NO: 226)
Ac-Nle-c[Asp-Pro-Pro-dPhe-Arg-Trp-Lys]-dVal-dPro-NH₂;
(SEQ ID NO: 227)
Ac-Nle-c[Asp-Trp-Pro-dPhe-Arg-Trp-Lys]-dVal-dPro-NNH₂;
(SEQ ID NO: 228)
Ac-Nle-c[Asp-His-dPhe-Pro-Trp-Lys]-dVal-dPro-NH₂;
(SEQ ID NO: 229)
Ac-Nle-c[Asp-His-dPhe-transPro(guan)-Trp-Lys]-dVal-dPro-NH₂;
(SEQ ID NO: 230)
Ac-Nle-c[Asp-His-dPhe-cisPro(guan)-Trp-Lys]-dVal-dPro-NH₂;
(SEQ ID NO: 231)
Ac-Nle-c[Asp-Pro-dPhe-Pro-Trp-Lys]-dVal-dPro-NH₂;
(SEQ ID NO: 232)
Ac-Nle-c[Asp-Pro-dPhe-transPro(guan)-Trp-Lys]-dVal-dPro-NH₂;
(SEQ ID NO: 233)
Ac-Nle-c[Asp-Pro-dPhe-cisPro(guan)-Trp-Lys]-dVal-dPro-NH₂;
(SEQ ID NO: 234)
Ac-Nle-c(Asp-Pro-dPhe-Arg-Aia-Lys)-dVal-dPro-NH₂;
(SEQ ID NO: 235)
Ac-Nle-c(Asp-Pro-dPhe-Arg-Aba-Lys)-dVal-dPro-NH₂;
(SEQ ID NO: 236)
Ac-Nle-c(Asp-Pro-dPhe-Arg-Ata-Lys)-dVal-dPro-NH₂;
(SEQ ID NO: 237)
Ac-Nle-c(Asp-Pro-p(CF₃)dPhe-Arg-Aia-Lys)-dVal-dPro-NH₂;
(SEQ ID NO: 238)
Ac-Nle-c(Asp-Pro-p(CF₃)dPhe-Arg-Aba-Lys)-dVal-dPro-NH₂;
(SEQ ID NO: 239)
Ac-Nle-c(Asp-Pro-p(CF₃)dPhe-Arg-Ata-Lys)-dVal-dPro-NH₂;
(SEQ ID NO: 240)
Ac-Nle-c[Asp-Aic-dNal(2′)-Arg-Trp-Lys]-dVal-dPro-NH₂;
(SEQ ID NO: 241)
Ac-Nle-c[Asp-Cpe-dNal(2′)-Arg-Trp-Lys]-dVal-dPro-NH₂;
(SEQ ID NO: 242)
Ac-Nle-c[Asp-Che-dNal(2′)-Arg-Trp-Lys]-dVal-dPro-NH₂;
(SEQ ID NO: 243)
Ac-Nle-c[Asp-Oic-dNal(2′)-Arg-Trp-Lys]-dVal-dPro-NH₂;
(SEQ ID NO: 244)
Ac-Nle-c[Asp-loc-dNal(2′)-Arg-Trp-Lys]-dVal-dPro-NH₂;
(SEQ ID NO: 245)
Ac-Nle-c[Asp-Tic-dNal(2′)-Arg-Trp-Lys]-dVal-dPro-NH₂;
(SEQ ID NO: 246)
Ac-Nle-c[Asp-Pro-dNal(2′)-Arg-Trp-Pro-Lys]-dVal-dPro-NH₂;
(SEQ ID NO: 247)
Ac-Nle-c[Asp-His-dNal(2′)-Pro-Trp-Lys]-dVal-dPro-NH₂;
(SEQ ID NO: 248)
Ac-Nle-c[Asp-His-dNal(2′)-transPro(guan)-Trp-Lys]-dVal-dPro-NH₂;
(SEQ ID NO: 249)
Ac-Nle-c[Asp-His-dNal(2′)-cisPro(guan)-Trp-Lys]-dVal-dPro-NH₂;
(SEQ ID NO: 250)
Ac-Nle-c[Asp-Pro-dNal(2′)-Pro-Trp-Lys]-dVal-dPro-NH₂;
(SEQ ID NO: 251)
Ac-Nle-c[Asp-Pro-dNal(2′)-transPro(guan)-Trp-Lys]-dVal-dPro-NH₂;
(SEQ ID NO: 252)
Ac-Nle-c[Asp-Pro-dNal(2′)-cisPro(guan)-Trp-Lys]-dVal-dPro-NH₂;
(SEQ ID NO: 253)
Ac-Nle-c(Asp-Pro-dNal(2′)-Arg-Aia-Lys)-dVal-dPro-NH₂;
(SEQ ID NO: 254)
Ac-Nle-c(Asp-Pro-dNal(2′)-Arg-Aba-Lys)-dVal-dPro-NH₂;
(SEQ ID NO: 255)
Ac-Nle-c(Asp-Pro-dNal(2′)-Arg-Ata-Lys)-dVal-dPro-NH₂;
(SEQ ID NO: 256)
Ac-Nle-c[Asp-Glu-dNal(2′)-Arg-Trp-Lys]-dVal-dPro-NH₂;
(SEQ ID NO: 257)
Ac-Nle-c[Asp-Glu-dNal(2′)-Arg-Trp-Gly-Lys]-dVal-dPro-NH₂;
(SEQ ID NO: 258)
Ac-Nle-c[Asp-Pro-Glu-dNal(2′)-Arg-Trp-Lys]-dVal-dPro-NH₂;
(SEQ ID NO: 259)
Ac-Nle-c[Asp-Pro-Glu-dNal(2′)-Arg-Trp-Gly-Lys]-dVal-dPro-NH₂;
(SEQ ID NO: 260)
Ac-Nle-c[Asp-Pro-dNal(2′)-Arg-Trp-Gly-Lys]-dVal-dPro-NH₂;
(SEQ ID NO: 261)
Ac-Nle-c[Asp-Pro-dNal(2′)-Arg-Trp-Gly-Lys]-dPro-dPro-Lys-Asp-NH₂;
(SEQ ID NO: 262)
Ac-Nle-c[Asp-Pro-dNal(2′)-Arg-Trp-Gly-Lys]-dPro-dPro-dLys-dAsp-NH₂;
(SEQ ID NO: 263)
Ac-Glu-c[Asp-Pro-dNal(2′)-Arg-Trp-Gly-Lys]-dPro-dPro-Lys-Asp-NH₂;
and
(SEQ ID NO: 264)
Ac-Nle-c[Asp-Pro-dNal(2′)-Arg-Trp-Lys]-dPro-dPro-dLys-dAsp-NH_{2,}

wherein c represents cyclization through R¹ or R² and R⁷ via a lactam bond.

In some embodiments of the sequence of Formula (I), when X¹ is present and X² and X³ are absent, the sequence of Formula (I) is cyclized through a lactam bond or a disulfide bond between R¹ and any one of R⁵⁻⁸. In some embodiments, when X¹ is present and X² and X³ are absent, the sequence of Formula (I) is cyclized through a lactam bond between R¹ and R⁶. In some embodiments, when X¹ is present and X² and X³ are absent, the sequence of Formula (I) is cyclized through a lactam bond between R¹ and R⁷. In some embodiments, Y¹ and Y² are present and are dVal and dPro, respectively, and Y³-Y⁸ are absent. In some embodiments, Y¹-Y⁸ are absent. In some embodiments, the sequence of Formula (I) is selected from the group consisting of:

(SEQ ID NO: 265)
Ac-Nle-c[Asp-Glu-His-dNal(2′)-Arg-Trp-Lys]-dVal-dPro-NH₂;
(SEQ ID NO: 266)
Ac-Nle-c[Asp-Glu-His-dNal(2′)-Arg-Trp-Gly-Lys]-dVal-dPro-NH₂;
(SEQ ID NO: 267)
Ac-Nle-c(Asp-dAla-His-dNal(2′)-Arg-Trp-Lys)-dVal-dPro-NH₂;
(SEQ ID NO: 268)
Ac-Arg-c(Asp-dAla-His-dNal(2′)-Arg-Trp-Lys)-dVal-dPro-NH₂;
(SEQ ID NO: 269)
Ac-Arg-c(Asp-dAla-His-dPhe-Arg-Trp-Lys)-dVal-dPro-NH₂;
(SEQ ID NO: 270)
Ac-Arg-c(Cys-dAla-His-dNal(2′)-Arg-Trp-Cys)-dVal-dPro-NH₂;
(SEQ ID NO: 271)
Ac-dArg-c(Asp-dAla-His-dNal(2′)-Arg-Trp-Lys)-dVal-dPro-NH₂;
(SEQ ID NO: 272)
Ac-Arg-c(Asp-dAla-dNal(2′)-Arg-Trp-Lys)-dVal-dPro-NH₂;
(SEQ ID NO: 273)
Ac-dArg-c(Asp-dAla-dNal(2′)-Arg-Trp-Lys)-dVal-dPro-NH₂;
(SEQ ID NO: 274)
Ac-Nle-c(Asp-Ala-His-dNal(2′)-Arg-Trp-Lys)-dVal-dPro-NH₂;
(SEQ ID NO: 275)
Ac-Arg-c(Asp-Ala-His-dNal(2′)-Arg-Trp-Lys)-dVal-dPro-NH₂;
(SEQ ID NO: 276)
Ac-dArg-c(Asp-Ala-His-dNal(2′)-Arg-Trp-Lys)-dVal-dPro-NH₂;
(SEQ ID NO: 277)
Ac-Nle-c(Asp-Trp-Arg-dNal(2′)-Pro-Lys)-dVal-dPro-NH₂;
(SEQ ID NO: 278)
Ac-Nle-c(Asp-Pro-Trp-dNal(2′)-Lys)-dVal-dPro-NH₂;
(SEQ ID NO: 279)
Ac-Nle-c(Asp-Pro-Trp-Arg-dNal(2′)-Pro-Lys)-dVal-dPro-NH₂;
(SEQ ID NO: 280)
Ac-Nle-c(Asp-Pro-Trp-Arg-dNal(2′)-Lys)-dVal-dPro-NH₂;
(SEQ ID NO: 281)
Ac-Nle-c(Lys-Trp-Arg-dNal(2′)-Pro-Asp)-dVal-dPro-NH₂;
(SEQ ID NO: 282)
Ac-Arg-c(Asp-dAla-His-dPhe-Arg-Trp-Lys)-NH₂;
(SEQ ID NO: 283)
Ac-Arg-c(Cys-dAla-His-dNal(2′)-Arg-Trp-Cys)-NH₂;
and
(SEQ ID NO: 284)
Ac-Arg-c(Asp-dAla-His-dNal(2′)-Arg-Trp-Lys)-NH_{2,}

wherein c represents cyclization through R¹ and any one of R⁵⁻⁸ via a lactam bond or a disulfide bond.

In some embodiments of the sequence of Formula (I), when X¹ and X² are present and X³ is absent, the sequence of Formula (I) is cyclized through a lactam bond between R¹ and R⁶. In such embodiments, X¹ and X² are each independently selected from dVal and dPro. In some embodiments, the sequence of Formula (I) is selected from the group consisting of:

(SEQ ID NO: 285)
Ac-dVal-dPro-c(Asp-Trp-Arg-dNal(2′)-Pro-Lys)-Nle-NH₂;
(SEQ ID NO: 286)
Ac-dVal-dPro-c(Lys-Trp-Arg-dNal(2′)-Pro-Asp)-Nie-NH₂;
(SEQ ID NO: 287)
Ac-dVal-dPro-c(Asp-Trp-Arg-dNal(2′)-Pro-Lys)-Nle-Nle-NH₂;
(SEQ ID NO: 288)
Ac-dVal-dPro-c(Lys-Trp-Arg-dNal(2′)-Pro-Asp)-Nle-Nle-NH₂;
(SEQ ID NO: 289)
Ac-dVal-dPro-c(Asp-Trp-Arg-dNal(2′)-Pro-Lys)-dVal-dPro-NH₂;
and
(SEQ ID NO: 290)
Ac-dVal-dPro-c(Lys-Trp-Arg-dNal(2′)-Pro-Asp)-dVal-dPro-NH₂;,

wherein c represents cyclization through R¹ and R⁶ via a lactam bond.

In some embodiments, the non-naturally occurring melanocortin analog comprises any one of the sequences of SEQ ID Nos: 2-285. In some embodiments, the non-naturally occurring melanocortin analog comprises any one of the sequences of SEQ ID Nos: 2-187, 196-212, and 240-290.

In some embodiments, the non-naturally occurring melanocortin analog comprises a sequence of SEQ ID NO: 3 (TCMCB07). The structure of TCMCB07 (“B07”), which comprises Ac-Nle-c[Asp-Pro-dNal(2′)-Arg-Trp-Lys]-dVal-dPro-NH₂ (SEQ ID NO: 3), is shown below:

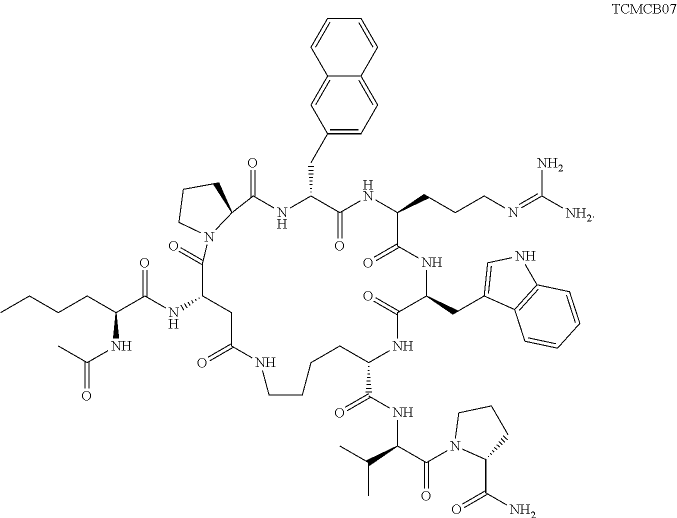 [SMILES: CCCC[C@H](NC(C)=O)C(=O)N[C@H]1CC(=O)NCCCC[C@@H](C(=O)N[C@@H](C(=O)N2CCC[C@@H]2C(N)=O)C(C)C)NC(=O)[C@H](Cc2c[nH]c3ccccc23)NC(=O)[C@H](CCCN=C(N)N)NC(=O)[C@@H](Cc2ccc3ccccc3c2)NC(=O)[C@@H]2CCCN2C1=O]

### Formulations

The compositions comprising the carriers and/or excipients disclosed in the present technology facilitate delivery of the non-naturally occurring melanocortin analog disclosed herein in any of its embodiments to a subject.

In some embodiments, the non-naturally occurring melanocortin analog is present in a composition.

In some embodiments, the non-naturally occurring melanocortin analog is present in the composition in a concentration of 0.1 mg/mL to 500 mg/mL, relative to a total volume of the composition. For example, the non-naturally occurring melanocortin analog is present in the composition in a concentration of 0.1 mg/mL to 500 mg/mL, 0.5 mg/ml to 250 mg/mL, 1 mg/mL to 100 mg/mL, 2.5 mg/mL to 50 mg/mL, or 5 mg/mL to 25 mg/mL, relative to a total volume of the composition. In some embodiments, the non-naturally occurring melanocortin analog is present in the composition in a concentration of about 50 mg/mL, relative to a total volume of the composition.

In some embodiments, the non-naturally occurring melanocortin analog comprises a sequence of SEQ ID NO: 3, and the non-naturally occurring melanocortin analog is present in the composition in a concentration of 5 mg/mL to 100 mg/mL, relative to a total volume of the composition. For example, the non-naturally occurring melanocortin analog comprising a sequence of SEQ ID NO: 3 is present in the composition in a concentration of 5 mg/mL to 100 mg/mL, 10 mg/mL to 75 mg/mL, 15 mg/mL to 50 mg/mL, 20 mg/mL to 40 mg/mL, or 25 mg/mL to 30 mg/mL, relative to a total volume of the composition. In some embodiments, the non-naturally occurring melanocortin analog comprising a sequence of SEQ ID NO: 3 is present in the composition in a concentration of about 50 mg/mL, relative to a total volume of the composition.

In some embodiments, the composition comprising the non-naturally occurring melanocortin analog is administered to the subject parenterally. In some embodiments, the composition comprising the non-naturally occurring melanocortin analog is administered to the subject subcutaneously.

In some embodiments, the composition formulated for parenteral administration (e.g., subcutaneous administration) comprises the non-naturally occurring melanocortin analog of formula (I) at a concentration at about 0.001 nmol, 0.005 nmol, 0.01 nmol, 0.02 nmol, 0.05 nmol, 0.1 nmol, 0.25 nmol, 0.5 nmol, 1 nmol, 2.5 nmol, 5 nmol, 10 nmol, 20 nmol, 25 nmol, 50 nmol, 100 nmol, 250 nmol, 500 nmol, or 1000 nmol, or even more, depending on the specific peptide selected, the desired response, the route of administration, the formulation and other factors known to those of skill in the art. In some embodiments, the non-naturally occurring melanocortin analog comprises a sequence of SEQ ID NO: 3.

In some embodiments, the non-naturally occurring melanocortin analog crosses blood-brain-barrier (BBB) of the subject.

In some embodiments, the subject is a mammal, including but not limited to a human, a non-human primate such as a chimpanzee, a domestic livestock or a farm animal such as a cow, a bison, sheep, a pig, a goat, a horse, a chicken, and a rooster, a domestic pet animal such as a dog, a cat, a rat, a mouse, and a rabbit, and a laboratory subject such as a rodent, including a rat, a mouse, and a guinea pig. In some embodiments, the subject is a human. In some embodiments, the subject is an animal such as a rat or a dog.

In some embodiments, the subject has a body mass index (BMI) of 18.5 kg/m² to 25 kg/m². In some embodiments, the subject has a BMI of less than 20 kg/m². In some embodiments, the subject has a BMI of less than 18.5 kg/m².

In some embodiments, the subject is an underweight subject. Underweight subjects include those having a body weight about 3%, 5% or less, 10% or less, 20% or less, or 30% or less, than the lower end of “normal” BMI (e.g., 18.5 kg/m²).

The compositions comprising the carriers and/or excipients disclosed in the present technology facilitate delivery (e.g., parenteral administration, in particular subcutaneous injection) of the non-naturally occurring melanocortin analog disclosed herein in any of its embodiments to a subject. Other purposes of the compositions comprising the carriers and/or excipients are to enhance dispersion, solubility, and stability of the non-naturally occurring melanocortin analog, and to reduce adverse injection site reactions.

The carriers and/or excipients of the composition can generally include one or more of the following components: a pH buffered aqueous solution comprising (a) sodium acetate, (b) Tris, and (c) water. In some embodiments, all components are compatible with the non-naturally occurring melanocortin analog (i.e., do not react or cause the non-naturally occurring melanocortin analog to react) and are homogeneously dispersed or dissolved uniformly in the composition.

In some embodiments, the carrier and/or excipient is isotonic.

In order to achieve a desirable tonicity, the composition of the present technology can further include a salt such as sodium chloride, sodium succinate, sodium sulfate, potassium chloride, magnesium chloride, magnesium sulfate, and calcium chloride. In some embodiments, the salt is present in the composition in a concentration of 0.1 mg/mL to 50 mg/mL, 1 mg/mL to 25 mg/mL, or 5 mg/mL to 10 mg/mL, relative to a total volume of the composition.

The carriers and/or excipients of the composition also includes a pH buffered aqueous solution which comprises (a) sodium acetate, (b) Tris, and (c) water.

The water used herein can act as a diluent and include, without limitation, water for injection (WFI), sterile water, bacteriostatic water for injection (BWFI), distilled water, bidistilled water, deionized water, deionized distilled water, and reverse osmosis water. In some embodiments, the water present in the pH buffered aqueous solution is water for injection.

In some embodiments, the composition includes water in an amount of about 1 wt % to about 90 wt %, about 10 wt % to about 75 wt %, or about 25 wt % to about 50 wt %, relative to a total weight of the composition.

In some embodiments, sodium acetate is present in the composition in a concentration of 0.5 mg/mL to 50 mg/mL, relative to a total volume of the composition. For example, sodium acetate is present in the composition in a concentration of 0.5 mg/ml to 50 mg/mL, 1 mg/mL to 40 mg/mL, 2 mg/mL to 30 mg/mL, 4 mg/mL to 20 mg/mL, 5 mg/mL to 15 mg/mL, 6 mg/mL to 12 mg/mL, or 8 mg/mL to 10 mg/mL, relative to a total volume of the composition.

In some embodiments, sodium acetate is present in the composition in a concentration of about 6 mg/mL to about 8 mg/mL, relative to a total volume of the composition. For example, sodium acetate is present in the composition in a concentration of 6 mg/mL, 6.5 mg/mL, 7 mg/mL, 7.1 mg/mL, 7.5 mg/mL, or 8 mg/mL, relative to a total volume of the composition.

In some embodiments, sodium acetate is present in the composition in a molar concentration of 5 mM to 700 mM, relative to a total volume of the composition. For example, sodium acetate is present in the composition in a molar concentration of 5 mM to 700 mM, 10 mM to 600 mM, 20 mM to 500 mM, 30 mM to 400 mM, 40 mM to 300 mM, 50 mM to 200 mM, 60 mM to 100 mM, or 70 mM to 80 mM, relative to a total volume of the composition.

In some embodiments, sodium acetate is present in the composition in a molar concentration of about 80 mM to about 100 mM, relative to a total volume of the composition. For example, sodium acetate is present in the composition in a molar concentration of 80 mM, 85 mM, 87 mM, 90 mM, 95 mM, or 100 mM, relative to a total volume of the composition.

The term “Tris” represents tris (hydroxymethyl)aminomethane also known as Tris buffer, Tris base, TRIS, tromethamine, tromethamine buffer, Trizma®, Trisamine, Trometamol, Tromethane, Trisaminol, or THAM. In some embodiments, Tris is present in the composition in a concentration of 0.5 mg/mL to 50 mg/mL, relative to a total volume of the composition. For example, Tris is present in the composition in a concentration of 0.5 mg/mL to 50 mg/mL, 1 mg/mL to 40 mg/mL, 2 mg/mL to 30 mg/mL, 4 mg/mL to 20 mg/mL, 5 mg/mL to 15 mg/mL, 6 mg/mL to 12 mg/mL, or 8 mg/mL to 10 mg/mL, relative to a total volume of the composition.

In some embodiments, Tris is present in the composition in a concentration of about 6 mg/mL to about 8 mg/mL, relative to a total volume of the composition. For example, Tris is present in the composition in a concentration of 6 mg/ml, 6.5 mg/mL, 7 mg/mL, 7.3 mg/mL, 7.6 mg/mL, or 8 mg/mL, relative to a total volume of the composition.

In some embodiments, Tris is present in the composition in a molar concentration of 2 mM to 500 mM, relative to a total volume of the composition. For example, Tris is present in the composition in a molar concentration of 2 mM to 500 mM, 5 mM to 400 mM, 10 mM to 300 mM, 20 mM to 200 mM, 30 mM to 150 mM, 40 mM to 100 mM, 50 mM to 80 mM, or 60 mM to 70 mM, relative to a total volume of the composition.

In some embodiments, Tris is present in the composition in a molar concentration of about 50 mM to about 70 mM, relative to a total volume of the composition. For example, Tris is present in the composition in a molar concentration of 50 mM, 55 mM, 60 mM, 65 mM, or 70 mM, relative to a total volume of the composition.

In some embodiments, the pH buffered aqueous solution provides the composition with a pH equivalent or close to the physiological pH levels. This may reduce adverse injection site reactions and also provide the non-naturally occurring melanocortin analog with enhanced stability and resistance to aggregation and degradation.

In addition to sodium acetate and Tris, the composition can include other buffering agents. Non-limiting examples of additional buffering agents include saline, phosphate, phosphoric acid, citrate, succinate, gluconate, histidine, acetic acid, ascorbate, tartartic acid, maleic acid, glycine, lactate, lactic acid, ascorbic acid, imidazole, bicarbonate, carbonic acid, succinic acid, sodium benzoate, benzoic acid, gluconate, edetate, malate, imidazole, and mixtures thereof. In some embodiments, the composition comprises acetic acid as an additional buffering agent.

In some embodiments, a weight ratio of sodium acetate to Tris is 1:4 to 4:1, 2:7 to 7:2, 1:3 to 3:1, 2:5 to 5:2, 1:2 to 2:1, 2:3 to 3:2, or about 1:1. In some embodiments, the weight ratio of sodium acetate to Tris is about 1:1.

In some embodiments, a weight ratio of the non-naturally occurring melanocortin analog to sodium acetate is 1:1 to 20:1, 3:2 to 15:1, 2:1 to 12:1, 3:1 to 10:1, 4:1 to 9:1, 5:1 to 8:1, or 6:1 to 7:1. In some embodiments, the weight ratio of the non-naturally occurring melanocortin analog to sodium acetate is about 7:1.

In some embodiments, a weight ratio of the non-naturally occurring melanocortin analog to Tris is 1:1 to 20:1, 3:2 to 15:1, 2:1 to 12:1, 3:1 to 10:1, 4:1 to 9:1, 5:1 to 8:1, or 6:1 to 7:1. In some embodiments, the weight ratio of the non-naturally occurring melanocortin analog to Tris is about 7:1.

The composition can also comprise a preservative agent. Exemplary preservative agents include, but are not limited to, ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, propyl gallate, phenol, m-cresol, benzyl alcohol, alpha-tocopherol, citric acid, ethylenediamine tetraacetic acid (EDTA), sorbitol, tartaric acid, phosphoric acid, benzalkonium chloride, phenoxyethanol, and methyl paraben.

If present, the concentration of the preservative agent can range from 0.001 mg/mL to 50 mg/mL, 0.01 mg/mL to 25 mg/mL, 0.1 mg/mL to 10 mg/mL, or 1 mg/mL to 5 mg/mL, relative to a total volume of the composition.

In some embodiments, the composition is in the form of an aqueous solution or a suspension. In some embodiments, the composition is in the form of an emulsion. In some embodiments, the composition is in the form of an aqueous solution. In some embodiments, the composition is in the form of an aqueous solution which is clear, colorless, and/or free of visible foreign matter.

In some embodiments, the composition has a pH ranging from 6.5 to 8.5. In some embodiments, the composition has a pH of about 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

In some embodiments, the composition is basic and has a pH of about 7.1, 7.2, 7.3, 7.4, 7.5, 7.6, 7.7, 7.8, 7.9, 8.0, 8.1, 8.2, 8.3, 8.4, or 8.5. In some embodiments, the composition has a pH ranging from about 7.3 to about 7.4. In some embodiments, the composition has a pH of 7.3 or 7.4.

In some embodiments, the composition has an osmolality ranging from 250 mOsm/kg to 350 mOsm/kg. For example, the composition has an osmolality ranging from 250 mOsm/kg to 360 mOsm/kg, 260 mOsm/kg to 340 mOsm/kg, 270 mOsm/kg to 330 mOsm/kg, 280 mOsm/kg to 320 mOsm/kg, 290 mOsm/kg to 310 mOsm/kg, or about 300 mOsm/kg. In some embodiments, the composition has an osmolarity of about 250 mOsm/kg, about 260 mOsm/kg, about 270 mOsm/kg, about 280 mOsm/kg, about 290 mOsm/kg, about 300 mOsm/kg, about 310 mOsm/kg, about 320 mOsm/kg, about 330 mOsm/kg, about 340 mOsm/kg, about 350 mOsm/kg, or about 360 mOsm/kg. In some embodiments, the composition has an osmolality ranging from about 275 mOsm/kg to about 330 mOsm/kg. In some embodiments, the composition has an osmolality of about 279 mOsm/kg, about 314 mOsm/kg, or about 329 mOsm/kg.

In some embodiments, the composition has a viscosity ranging from 0.5 cP to 5 cP. For example, the composition has a viscosity ranging from 0.5 cP to 5 cP, 0.75 cP to 4.5 cP, 1.0 cP to 4 cP, 1.2 cP to 3.5 cP, 1.3 cP to 3 cP, 1.4 cP to 2.5 cP, 1.5 cP to 2 cP, or 1.6 cP to 1.8 cP. In some embodiments, the composition has a viscosity of about 0.5 cP, 0.6 CP, 0.7 cP, 0.8 cP, 0.9 cP, 1.0 cP, 1.1 cP, 1.2 cP, 1.3 cP, 1.4 cP, 1.5 cP, 1.6 cP, 1.7 cP, 1.8 cP, 1.9 cP, or 2.0 cP. In some embodiments, the composition has a viscosity of about 1.4 cP or 1.6 cP.

The composition disclosed herein in any of its embodiments can be formulated for parenteral administration, such as, for example, in the form of aqueous or non-aqueous isotonic sterile injection solutions or suspensions. The term “parenteral”, as used herein, includes subcutaneous, intravenous, intraperitoneal, intramuscular, and intralesional, or infusion techniques.

The active ingredient(s) (e.g., the non-naturally occurring melanocortin analog) can be dissolved or suspended in the aforementioned carrier and/or excipient. Additional aqueous or non-aqueous carriers that may facilitate dissolution of the active ingredient include, but are not limited to, ethanol, benzyl alcohol, DMSO, polyethylene glycol, propylene glycol, corn oil, cottonseed oil, peanut oil, sesame oil, and/or various buffers.

In some embodiments, the composition formulated for parenteral administration (e.g., subcutaneous administration) comprises a non-naturally occurring melanocortin analog at a concentration at about 0.001 nmol, 0.005 nmol, 0.01 nmol, 0.02 nmol, 0.05 nmol, 0.1 nmol, 0.25 nmol, 0.5 nmol, 1 nmol, 2.5 nmol, 5 nmol, 10 nmol, 20 nmol, 25 nmol, 50 nmol, 100 nmol, 250 nmol, 500 nmol, or 1000 nmol, or even more, depending on the specific peptide selected, the desired therapeutic response, the route of administration, the formulation and other factors known to those of skill in the art.

In some embodiments, the composition formulated for parenteral administration (e.g., subcutaneous administration) comprises sodium acetate at a concentration at about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 105 mM, 110 mM, 120 mM, 130 mM, 140 mM, 150 mM, 160 mM, 170 mM, 180 mM, 190 mM, or 200 mM.

In some embodiments, the composition formulated for parenteral administration (e.g., subcutaneous administration) comprises Tris at a concentration at about 5 mM, 10 mM, 15 mM, 20 mM, 25 mM, 30 mM, 3 5 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, or 120 mM.

The compositions of the present technology may be formulated for intranasal delivery. In some embodiments, the intranasal composition comprises a melanocortin peptide or analog of the present technology and a carrier and/or excipient.

The carriers and/or excipients of the composition can generally include one or more of the following components: (i) one or more antioxidants, (ii) one or more preservatives, (iii) one or more buffers, (iv) one or more tonicity adjustors, (v) one or more surfactants, (vi) flavor, (vii) propellants, and/or (viii) a vehicle or solvent. In some embodiments, all components are compatible with the non-naturally occurring melanocortin analog (i.e., do not react or cause the non-naturally occurring melanocortin analog to react) and are homogeneously dispersed or dissolved uniformly in the composition.

In some embodiments, the carrier and/or excipient is isotonic to nasal fluids.

In order to achieve a desirable tonicity, the composition of the present technology can further include a salt such as sodium chloride, sodium succinate, sodium sulfate, potassium chloride, magnesium chloride, magnesium sulfate, and calcium chloride. In some embodiments, the salt is present in the composition in a concentration of 0.1 mg/mL to 50 mg/mL, 1 mg/mL to 25 mg/mL, or 5 mg/mL to 10 mg/mL, relative to a total volume of the composition. In some aspects, the salt is sodium chloride at a concentration of 0.9% wt.

The carriers and/or excipients of the composition also includes a pH buffered aqueous solution which comprises sodium acetate, Tris, and/or a phosphate buffer.

The water used herein can act as a diluent and include, without limitation, water for injection (WFI), sterile water, bacteriostatic water for injection (BWFI), distilled water, bidistilled water, deionized water, deionized distilled water, and reverse osmosis water. In some embodiments, the water present in the pH buffered aqueous solution is water for injection.

In some embodiments, the composition includes water (e.g., water for injection) in an amount of about 1 wt % to about 90 wt %, about 10 wt % to about 75 wt %, or about 25 wt % to about 50 wt %, relative to a total weight of the composition.

In some embodiments, sodium acetate is present in the composition in a concentration of 0.5 mg/mL to 50 mg/mL, relative to a total volume of the composition. For example, sodium acetate is present in the composition in a concentration of 0.5 mg/mL to 50 mg/mL, 1 mg/mL to 40 mg/mL, 2 mg/mL to 30 mg/mL, 4 mg/mL to 20 mg/mL, 5 mg/mL to 15 mg/mL, 6 mg/mL to 12 mg/mL, or 8 mg/mL to 10 mg/mL, relative to a total volume of the composition.

In some embodiments, sodium acetate is present in the composition in a molar concentration of about 80 mM to about 100 mM, relative to a total volume of the composition. For example, sodium acetate is present in the composition in a molar concentration of 80 mM, 85 mM, 87 mM, 90 mM, 95 mM, or 100 mM, relative to a total volume of the composition.

The term “Tris” represents tris (hydroxymethyl)aminomethane also known as Tris buffer, Tris base, TRIS, tromethamine, tromethamine buffer, Trizma®, Trisamine, Trometamol, Tromethane, Trisaminol, or THAM. In some embodiments, Tris is present in the composition in a concentration of 0.5 mg/mL to 50 mg/mL, relative to a total volume of the composition. For example, Tris is present in the composition in a concentration of 0.5 mg/mL to 50 mg/mL, 1 mg/mL to 40 mg/mL, 2 mg/mL to 30 mg/mL, 4 mg/mL to 20 mg/mL, 5 mg/mL to 15 mg/mL, 6 mg/mL to 12 mg/mL, or 8 mg/mL to 10 mg/mL, relative to a total volume of the composition.

In some embodiments, the pH buffered aqueous solution provides the composition with a pH equivalent or close to the physiological pH levels. This may reduce adverse injection site reactions and also provide the non-naturally occurring melanocortin analog with enhanced stability and resistance to aggregation and degradation.

In addition to sodium acetate and Tris, the composition can include other buffering agents. Non-limiting examples of additional buffering agents include saline, phosphate, phosphoric acid, citrate, succinate, gluconate, histidine, acetic acid, ascorbate, tartartic acid, maleic acid, glycine, lactate, lactic acid, ascorbic acid, imidazole, bicarbonate, carbonic acid, succinic acid, sodium benzoate, benzoic acid, gluconate, edetate, malate, imidazole, and mixtures thereof. In some embodiments, the composition comprises acetic acid as an additional buffering agent.

In some embodiments, sodium phosphate is present in the composition in a molar concentration of 5 mM to 700 mM, relative to a total volume of the composition. For example, sodium acetate is present in the composition in a molar concentration of 5 mM to 700 mM, 10 mM to 600 mM, 20 mM to 500 mM, 30 mM to 400 mM, 40 mM to 300 mM, 50 mM to 200 mM, 60 mM to 100 mM, or 70 mM to 80 mM, relative to a total volume of the composition.

The composition can also comprise a preservative agent. Exemplary preservative agents include, but are not limited to, ascorbic acid, cysteine hydrochloride, sodium bisulfate, sodium metabisulfite, sodium sulfite, ascorbyl palmitate, butylated hydroxyanisole (BHA), butylated hydroxytoluene (BHT), lecithin, propyl gallate, phenol, m-cresol, benzyl alcohol, benzalkonium chloride, alpha-tocopherol, citric acid, ethylenediamine tetraacetic acid (EDTA), sorbitol, tartaric acid, phosphoric acid, benzalkonium chloride, phenoxyethanol, and methyl paraben.

If present, the concentration of the preservative agent can range from 0.001 mg/mL to 50 mg/mL, 0.01 mg/mL to 25 mg/mL, 0.1 mg/mL to 10 mg/mL, or 1 mg/mL to 5 mg/mL, relative to a total volume of the composition.

In some embodiments, the composition is in the form of an aqueous solution or a suspension. In some embodiments, the composition is in the form of an emulsion. In some embodiments, the composition is in the form of an aqueous solution. In some embodiments, the composition is in the form of an aqueous solution which is clear, colorless, and/or free of visible foreign matter.

In some embodiments, the composition has a pH approximating the normal pH range of the nasal fluid. In some embodiments, the composition has a pH ranging from 5.5 to 6.5. In some embodiments, the composition has a pH of about 5.5, 5.6, 5.7, 5.8, 5.9, 6.0, 6.1, 6.2, 6.3, 6.4, or 6.5.

In some embodiments, the composition has an osmolality ranging from 250 mOsm/kg to 350 mOsm/kg. For example, the composition has an osmolality ranging from 250 mOsm/kg to 360 mOsm/kg, 260 mOsm/kg to 340 mOsm/kg, 270 mOsm/kg to 330 mOsm/kg, 280 mOsm/kg to 320 mOsm/kg, 290 mOsm/kg to 310 mOsm/kg, or about 300 mOsm/kg. In some embodiments, the composition has an osmolarity of about 250 mOsm/kg, about 260 mOsm/kg, about 270 mOsm/kg, about 280 mOsm/kg, about 290 mOsm/kg, about 300 mOsm/kg, about 310 mOsm/kg, about 320 mOsm/kg, about 330 mOsm/kg, about 340 mOsm/kg, about 350 mOsm/kg, or about 360 mOsm/kg. In some embodiments, the composition has an osmolality ranging from about 275 mOsm/kg to about 330 mOsm/kg. In some embodiments, the composition has an osmolality of about 279 mOsm/kg, about 314 mOsm/kg, or about 329 mOsm/kg.

In some embodiments, the composition has a viscosity ranging from 0.5 cP to 5 cP. For example, the composition has a viscosity ranging from 0.5 cP to 5 cP, 0.75 cP to 4.5 cP, 1.0 cP to 4 cP, 1.2 cP to 3.5 cP, 1.3 cP to 3 cP, 1.4 cP to 2.5 cP, 1.5 cP to 2 cP, or 1.6 cP to 1.8 cP. In some embodiments, the composition has a viscosity of about 0.5 cP, 0.6 CP, 0.7 cP, 0.8 cP, 0.9 cP, 1.0 cP, 1.1 cP, 1.2 cP, 1.3 cP, 1.4 cP, 1.5 cP, 1.6 cP, 1.7 cP, 1.8 cP, 1.9 cP, or 2.0 cP. In some embodiments, the composition has a viscosity of about 1.4 cP or 1.6 cP.

In some embodiments, the composition comprises one or more antioxidants. For example, the composition may comprise ascorbic acid, cysteine, sodium metabisulfite, propyl gallate, butylated hydroxytoluene, and/or butylated hydroxyanisole.

In some embodiments, the composition comprises a surfactant, such as a sorbitan ester.

In some embodiments, the composition comprises a flavoring or scent, such as an aromatic oil.

The active ingredient(s) (e.g., the non-naturally occurring melanocortin analog) can be dissolved or suspended in the aforementioned carrier and/or excipient. Additional aqueous or non-aqueous carriers that may facilitate dissolution of the active ingredient include, but are not limited to, ethanol, benzyl alcohol, DMSO, polyethylene glycol, propylene glycol, corn oil, cottonseed oil, peanut oil, sesame oil, and/or various buffers.

In some embodiments, the non-naturally occurring melanocortin analog is solubilized or suspended in a solvent or vehicle. In some aspects, the solvent or vehicle is purified water, ethyl alcohol, propylene glycol. The composition may comprise between 0.03% wt and 1% wt melanocortin solubilized or suspended in a solvent or vehicle. The composition may comprise about 0.03% wt, 0.05% wt, 0.1% wt, 0.15% wt, 0.2% wt, 0.25% wt, 0.3% wt, 0.35% wt, 0.4% wt, 0.45% wt, 0.5% wt, 0.55% wt, 0.6% wt, 0.65% wt 0.7% wt, 0.75% wt, 0.8% wt, 0.85% wt, 0.9% wt, 0.95% wt, or 1% wt melanocortin.

In some embodiments, the composition formulated for intranasal administration comprises a non-naturally occurring melanocortin analog at a concentration at about 0.001 nmol, 0.005 nmol, 0.01 nmol, 0.02 nmol, 0.05 nmol, 0.1 nmol, 0.25 nmol, 0.5 nmol, 1 nmol, 2.5 nmol, 5 nmol, 10 nmol, 20 nmol, 25 nmol, 50 nmol, 100 nmol, 250 nmol, 500 nmol, or 1000 nmol, or even more, depending on the specific peptide selected, the desired therapeutic response, the route of administration, the formulation and other factors known to those of skill in the art.

The composition may be formulated to be delivered by nose drop, spray device, or topical solution. In some embodiments, the pharamaceutical composition may be formulated as an aerosol, atomizer, inhalation, insufflation, metered-dose inhaler, nebulizer, or 108ydrobrom. In some embodiments, the composition includes a propellant, such as hydrofluoroalkane.

In some embodiments, the composition may be configured to be administered using a spray device or nasal inhaler. The spray device or nasal inhaler may be configured to deliver 1 ug to 100 μg per spray. In some embodiments, the spray device or nasal inhaler may be configured to deliver 1 ug to 100 μg, 5 ug to 90 ug, 10 ug to 80 ug, 15 ug to 70 μg, 20 ug to 60 μg, 25 ug, to 50 μg, or 30 ug to 40 ug per spray.

### Additional Embodiments of Non-Naturally Occurring Melanocortin Analogs

Additional aspects of non-naturally occurring melanocortin analogs that can be used in the methods of the present technology are described below.

Cyclized non-naturally occurring melanocortin analogs have shown improved efficacy and stability. See Balse-Srinivasan et al., J. Med. Chem. 46 (17): 3728-3733 (2003) and Bednarek et al., Biochem. Biophys. Res. Com. 286 (3): 641-645 (2001); Kavarana, et al., J. Med. Chem. 45 (12): 2644-2650 (2002). In one aspect, the non-naturally occurring melanocortin analog represented by Formula I is cyclized. The following represents a non-limiting list of examples of how the non-naturally occurring melanocortin analog represented by Formula I can be cyclized:
In Formula I, disulfide bond between R¹ or R² and R⁷ or Y¹, when R¹ or R² is cysteine and R⁷ or Y¹ is cysteine as described in Balse-Srinivasan et al., J. Med. Chem., 2003, 46 (23): 4965-4973. When Y¹ is cysteine, Y² is not absent, but is selected from the group consisting of D-threonine, L-threonine, D-proline, L-proline and a piperazin-2-one ring.

A lactam bridge between R¹ and R⁷, when R¹ is norleucine and R⁷ is glutamic acid, as described in Mayorov et al. J. Med. Chem. 49:1946-1952 (2006) and Bednarek et al., Biochem. Biophys. Res. Com. 286 (3): 641-645 (2001).

A side-chain lactam bridge between R² and R⁷, when R² is glutamic acid or aspartic acid and R⁷ is lysine, as described in Bednarek et al., Biochem. Biophys. Res. Com. 286 (3): 641-645 (2001).

A lactam bridge between R¹ and R⁷, when R¹ is succinic acid or o-pthalic acid and R⁷ is lysine, as described in Bednarek et al., Biochem. Biophys. Res. Com. 286 (3): 641-645 (2001) and Kavarana et al., J. Med. Chem. 45 (12): 2644-2650 (2002).

A lactam bridge between R² or R³ and R⁷, when R² or R³ is succinic acid and R⁷ is 2,3-diamino-propionic acid as described in Bednarek et al., Biochem. Biophys. Res. Com. 286 (3): 641-645 (2001).

A “backbone” cyclized peptide is formed by covalent bond formation between the C- and/or N-terminus of a linear peptide of interest. An example of this is described in the bonding of two amide nitrogens via a bridge consisting of alkyl groups and an amide, as described by Hess et al., J. Med. Chem. 50:6201-6211 (2007).

### Synthesis of Non-Naturally Occurring Melanocortin Analogs

The non-naturally occurring melanocortin analogs of the present technology may be readily synthesized by any known conventional procedure for the formation of a peptide linkage between amino acids. Such conventional procedures include, for example, any solution phase procedure permitting a condensation between the free alpha amino group of an amino acid or residue thereof having the carboxyl group or other reactive groups protected and the free primary carboxyl group of another amino acid or residue thereof having the amino group or other reactive groups protected. In an exemplary procedure, the peptides of the present technology may be synthesized by solid-phase synthesis and purified according to methods known in the art. Any of a number of well-known procedures utilizing a variety of resins and reagents may be used to prepare the peptides of the present technology.

The process for synthesizing the peptides may be carried out by a procedure whereby each amino acid in the desired sequence is added one at a time in succession to another amino acid or residue thereof or by a procedure whereby peptide fragments with the desired amino acid sequence are first synthesized conventionally and then condensed to provide the desired peptide. The resulting peptide is then cyclized to yield a cyclic peptide.

Solid phase peptide synthesis methods are well known and practiced in the art. In such methods, the synthesis of peptides can be carried out by sequentially incorporating the desired amino acid residues one at a time into the growing peptide chain according to the general principles of solid phase methods. These methods are disclosed in numerous references, including Merrifield, Angew Chem. 24:799-810 (1985) and Barany et al., The Peptides, Analysis, Synthesis and Biology, Vol. 2, Gross E. and Meienhofer J., Eds. Academic Press 1-284 (1980).

In chemical syntheses of peptides, reactive side chain groups of the various amino acid residues are protected with suitable protecting groups, which prevent a chemical reaction from occurring at that site until the protecting group is removed. Also common is the protection of the alpha amino group of an amino acid residue or fragment while that entity reacts at the carboxyl group, followed by the selective removal of the alpha amino protecting group to allow a subsequent reaction to take place at that site. Specific protecting for solid phase synthesis methods and solution phase synthesis methods groups are known to those having ordinary skill in the art.

Alpha amino groups may be protected by a suitable protecting group, including a urethane-type protecting group, such as benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz); aliphatic urethane-type protecting groups, such as t-butyloxycarbonyl (Boc), diisopropylmethoxycarbonyl, isopropoxycarbonyl, and allyloxycarbonyl. Fmoc is useful for alpha amino protection.

Guanidino groups may be protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf) and Boc. Pmc is a useful protecting group for Arg.

Solid phase synthesis is commenced from the C-terminal end of the peptide by coupling a protected alpha amino acid to a suitable resin. Such starting material is prepared by attaching an alpha amino-protected amino acid by an ester linkage to a p-benzyloxybenzyl alcohol (Wang) resin or a 2-chlorotrityl chloride resin, by an amide bond between an Fmoc-Linker, such as p-[(R,S)-α-[1-(9H-fluor-en-9-yl)-methoxyformamido]-2,4-dimethyloxybenzyl]-phenoxyacetic acid (Rink linker) to a benzhydrylamine (BHA) resin, or by other means well known in the art. Fmoc-Linker-BHA resin supports are commercially available and generally used when feasible. The resins are carried through repetitive cycles as necessary to add amino acids sequentially. The alpha amino Fmoc protecting groups are removed under basic conditions. Piperidine, piperazine, diethylamine, or morpholine (20-40% v/v) in N,N-dimethylformamide (DMF) may be used for this purpose.

Following removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. After the peptide is synthesized, if desired, the orthogonally protected side chain protecting groups may be removed using methods well known in the art for further derivatization of the peptide.

Reactive groups in a peptide can be selectively modified, either during solid phase synthesis or after removal from the resin. For example, peptides can be modified to obtain N-terminus modifications, such as acetylation, while on resin, or may be removed from the resin by use of a cleaving reagent and then modified. Methods for N-terminus modification, such as acetylation, and for C-terminus modification, such as amidation, are known in the art. Similarly, methods for modifying side chains of amino acids are well known to those skilled in the art of peptide synthesis. The choice of modifications made to reactive groups present on the peptide will be determined, in part, by the characteristics that are desired in the peptide.

The peptide can be cyclized prior to cleavage from the peptide resin. For cyclization through reactive side chain moieties, the desired side chains are deprotected, and the peptide suspended in a suitable solvent and a cyclic coupling agent added. Suitable solvents include, for example DMF, dichloromethane (DCM) or 1-methyl-2-pyrrolidone (NMP). Suitable cyclic coupling reagents include, for example, 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris (dimethylamino)phosphoniumhexafluorophosphate (BOP), benzotriazole-1-yl-oxy-tris (pyrrolidino)phosphoniumhexafluorophosphate (PyBOP), 2-(7-aza-1H-benzotriazol-1-yl)-1,1,3,3-tetramethyluronium tetrafluoroborate (TATU), 2-(2-oxo-1 (2H)-pyridyl)-1,1,3,3-tetramethyluronium tetrafluoroborate (TPTU) or N,N′-dicyclohexylcarbodiimide/1-hydroxybenzotriazole (DCCI/HOBt). Coupling is convention initiated by use of a suitable base, such as N,N-diispropylethylamine (DIPEA), sym-collidine or N-methylmorpholine (NMM).

Following cleavage of peptides from the solid phase following their synthesis, the peptide can be purified by any number of methods, such as reverse phase high performance liquid chromatography (RP-HPLC), using a suitable column, such as a C18 column. Other methods of separation or purification, such as methods based on the size or charge of the peptide, can also be employed. Once purified, the peptide can be characterized by any number of methods, such as high performance liquid chromatograph (HPLC), amino acid analysis, mass spectrometry, and the like.

### Salt Forms of Non-Naturally Occurring Melanocortin Analogs

The non-naturally occurring melanocortin analog peptides of the present technology may be in the form of any salt. The term “pharmaceutically acceptable salts” refers to salts prepared from non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Exemplary salts are the ammonium, calcium, lithium, magnesium, potassium, and sodium salts. Salts derived from organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the peptides of the present technology are basic, acid addition salts may be prepared from non-toxic acids, including inorganic and organic acids. Such acids include acetic, benzenesulfonic, benzoic, camphorsulfonic, carboxylic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Acid addition salts of the peptides of the present technology are prepared in a suitable solvent from the peptide and an excess of an acid, such as hydrochloric, hydrobromic, sulfuric, phosphoric, acetic, trifluoroacetic, citric, tartaric, maleic, succinic or methanesulfonic acid. The acetate salt form is especially useful. Where the peptides of the present technology include an acidic moiety, suitable salts may include alkali metal salts, such as sodium or potassium salts, or alkaline earth metal salts, such as calcium or magnesium salts.

### Use of Non-Naturally Occurring Melanocortin Analogs

The non-naturally occurring melanocortin analogs, pharmaceutically acceptable salts thereof, and pharmaceutical compositions including the non-naturally occurring melanocortin analogs of the present technology are useful to treat, reduce, or prevent conditions associated with antagonizing MC3R and/or MC4R. The non-naturally occurring melanocortin analogs, pharmaceutically acceptable salts thereof, and pharmaceutical compositions including the non-naturally occurring melanocortin analogs of the present technology are also useful to stimulate appetite, increase body weight and/or increase food consumption. The non-naturally occurring melanocortin analogs of the present technology may also lead to preventing or reducing risk of appetite loss or loss of appetite, reducing or preventing body weight loss, reducing or preventing loss of muscle mass, and/or reducing or preventing loss of fat mass of the subject. Examples of conditions associated with antagonizing MC3R and/or MC4R include, but are not limited to, psychological diseases and conditions, allergies, intolerances, gastrointestinal diseases and conditions, side-effects from a medication, substance abuse, viral infections, bacterial infections, food poisoning, dehydration, fatigue, hormonal imbalances, pain, cardiovascular diseases and conditions, anemia, autoimmune diseases and conditions, respiratory diseases and conditions, and inflammatory diseases and conditions.

### Methods for Appetite Stimulation

The present technology provides methods of stimulating appetite of a subject. The methods include administering to the subject a therapeutically effective amount of a non-naturally occurring melanocortin analog. The methods can promote appetite and food consumption, and increase or maintain body weight or BMI, muscle mass, and/or fat mass of the subject. The methods are also effective in treating nausea, emesis, and/or anorexia.

The subject applicable to the methods disclosed herein can be any subject who experiences involuntary loss of appetite, reduced appetite, decreased food consumption, and/or weight loss. For example, the subject can be a cancer patient, including those suffering from cancer cachexia, a metabolically unchallenged subject, or a healthy subject. The subject may also be at risk of experiencing involuntary loss of appetite, reduced appetite, decreased food consumption, and/or weight loss in the future. As used herein, being at risk of developing a condition “in the future” means that it is foreseeable that the subject will develop the condition within 1 week, 2 weeks, 3 weeks, 1 month, 2 months, 6 months, 1 year, or 2 years.

In general, the actual quantity of pharmaceutical compositions administered to a patient will vary between wide ranges depending upon the mode of administration, the formulation used, and the response desired. The dosage for treatment is administration, by any of the foregoing means or any other means known in the art, of an amount sufficient to bring about the desired therapeutic effect. Thus, a therapeutically effective amount includes an amount of a peptide, such as the melanocortin analogs (e.g., non-naturally occurring melanocortin analogs) of the present technology, or pharmaceutical composition that is sufficient to increase body weight or BMI and/or increase food consumption in a metabolically unchallenged subject. In some embodiments, therapeutically effective amount includes an amount of a peptide, such as the melanocortin analogs (e.g., non-naturally occurring melanocortin analogs) of the present technology, or pharmaceutical composition alleviates a feeding disorder in a patient, or to prevent or delay onset or recurrence of the feeding disorder, or for the management of the feeding disorder in patients with diseases or syndromes associated with cachexia, including secondary to immune disorders and cancer, or other diseases and conditions of the present technology.

Another aspect present technology is a method of increasing body weight of and/or increasing food consumption by a metabolically unchallenged subject, the method comprising administering to the metabolically unchallenged subject a therapeutically effective amount of a non-naturally occurring melanocortin analog comprising a sequence according to Formula (I),

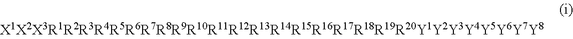

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, D-tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-blindol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), D-Nal(1′), 7-amino-7,8-dihydro4H-[1,2,3]triazolo[1,5-a][1,4]diazepin-6 (5H)-one (Aia), phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine;
the non-naturally occurring melanocortin analog is optionally cyclized through a moiety selected from the group consisting of:
a disulfide bond between R¹ or R² and R⁷ or X¹ when R¹ or R² is cysteine and R⁷ or X¹ is cysteine;
a disulfide bond between R² and any one of R⁵—R²⁰ when R² and the any one of R⁵—R²⁰ are selected from the group consisting of cysteine, Pen, and dPen;
a lactam bridge between R¹ and R⁷ when R¹ is norleucine and R⁷ is glutamic acid;
a side-chain lactam bridge between R² and R⁷ when R² is glutamic acid, aspartic acid, or CO-cis-CH—CH—CO, and R⁷ is lysine or ornithine;
a side-chain lactam bridge between R² and R⁸ when R² is glutamic acid or aspartic acid, R⁸ is lysine, and R⁷ is proline or tryptophan;
a lactam closure between R¹ and R⁷ when R¹ is succinic acid or o-phthalic acid and R⁷ is lysine; and
a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

In some embodiments, the non-naturally occurring melanocortin analog comprises any one of the sequences of SEQ ID Nos: 2-285. In some embodiments, the non-naturally occurring melanocortin analog comprises any one of the sequences of SEQ ID Nos: 2-187, 196-212, and 240-290.

In some embodiments, the non-naturally occurring melanocortin analog comprises a sequence of SEQ ID NO: 3.

In some embodiments, the non-naturally occurring melanocortin analog is present in a pharmaceutical composition.

In some embodiments, the pharmaceutical composition further comprises a pharmaceutical salt.

In some embodiments, the pharmaceutical composition further comprises a pharmaceutical carrier.

### Administration of Non-Naturally Occurring Melanocortin Analogs

Typically, the therapeutically effective amount of the non-naturally occurring melanocortin analog (e.g., to stimulate appetite of a subject, in terms of mg of the non-naturally occurring melanocortin analog per body weight of the subject (kg), can range from 0.001 mg/kg to 100 mg/kg, 0.01 mg/kg to 90 mg/kg, 0.1 mg/kg to 80 mg/kg, 0.5 mg/kg to 70 mg/kg, 1 mg/kg to 60 mg/kg, 1.2 mg/kg to 50 mg/kg, 1.4 mg/kg to 40 mg/kg, 1.6 mg/kg to 30 mg/kg, 1.8 mg/kg to 20 mg/kg, 2 mg/kg to 10 mg/kg, 2.2 mg/kg to 5 mg/kg, 2.4 mg/kg to 3 mg/kg, or about 2.5 mg/kg.

In some embodiments, the therapeutically effective amount of the non-naturally occurring melanocortin analog is from 0.001 mg/kg to 25 mg/kg per body weight of the subject. For example, the therapeutically effective amount of the non-naturally occurring melanocortin analog is from 0.001 mg/kg to 25 mg/kg, 0.005 mg/kg to 20 mg/kg, 0.01 mg/kg to 15 mg/kg, 0.05 mg/kg to 10 mg/kg, 0.1 mg/kg to 5 mg/kg, or 0.5 mg/kg to 1 mg/kg, per body weight of the subject.

In some embodiments, the therapeutically effective amount of the non-naturally occurring melanocortin analog is from 0.5 mg/kg to 10 mg/kg per body weight of the subject. For example, the therapeutically effective amount of the non-naturally occurring melanocortin analog is from 0.5 mg/kg to 10 mg/kg, 0.75 mg/kg to 9 mg/kg, 1 mg/kg to 8 mg/kg, 1.5 mg/kg to 7 mg/kg, 2 mg/kg to 6 mg/kg, or 3 mg/kg to 5 mg/kg, per body weight of the subject.

In some embodiments, the pharmaceutical composition is administered parenterally (e.g., subcutaneously) at a therapeutically effective dose of the non-naturally occurring melanocortin analog, which is at least about 0.001 nmol, 0.005 nmol, 0.01 nmol, 0.02 nmol, 0.03 nmol, 0.04 nmol, 0.05 nmol, 0.06 nmol, 0.07 nmol, 0.08 nmol, 0.09 nmol, 0.1 nmol, 0.2 nmol, 0.3 nmol, 0.4 nmol, 0.5 nmol, 0.6 nmol, 0.7 nmol, 0.8 nmol, 0.9 nmol, 1.0 nmol, 1.1 nmol, 1.2 nmol, 1.3 nmol, 1.4 nmol, 1.5 nmol, 1.6 nmol, 1.7 nmol, 1.8 nmol, 1.9 nmol, 2 nmol, 3 nmol, 4 nmol, 5 nmol, 6 nmol, 7 nmol, 8 nmol, 9 nmol, 10 nmol, 11 nmol, 12 nmol, 13 nmol, 14 nmol, 15 nmol, 16 nmol, 17 nmol, 18 nmol, 19 nmol, 20 nmol, 21 nmol, 22 nmol, 23 nmol, 24 nmol, 25 nmol, 26 nmol, 27 nmol, 28 nmol, 29 nmol, 30 nmol, 31 nmol, 32 nmol, 33 nmol, 34 nmol, 35 nmol, 36 nmol, 37 nmol, 38 nmol, 39 nmol, 40 nmol, 41 nmol, 42 nmol, 43 nmol, 44 nmol, 45 nmol, 46 nmol, 47 nmol, 48 nmol, 49 nmol, 50 nmol, 51 nmol, 52 nmol, 53 nmol, 54 nmol, 55 nmol, 56 nmol, 57 nmol, 58 nmol, 59 nmol, 60 nmol, 61 nmol, 62 nmol, 63 nmol, 64 nmol, 65 nmol, 66 nmol, 67 nmol, 68 nmol, 69 nmol, 70 nmol, 61 nmol, 72 nmol, 73 nmol, 74 nmol, 75 nmol, 76 nmol, 77 nmol, 78 nmol, 79 nmol, 80 nmol, 81 nmol, 82 nmol, 83 nmol, 84 nmol, 85 nmol, 86 nmol, 87 nmol, 88 nmol, 89 nmol, 90 nmol, 91 nmol, 92 nmol, 93 nmol, 94 nmol, 95 nmol, 96 nmol, 97 nmol, 98 nmol, 99 nmol, 100 nmol, 110 nmol, 120 nmol, 130 nmol, 140 nmol, 150 nmol, 160 nmol, 170 nmol, 180 nmol, 190 nmol, 200 nmol, 210 nmol, 220 nmol, 230 nmol, 240 nmol, 250 nmol, 260 nmol, 270 nmol, 280 nmol, 290 nmol, 300 nmol, 310 nmol, 320 nmol, 330 nmol, 340 nmol, 350 nmol, 360 nmol, 370 nmol, 380 nmol, 390 nmol, 400 nmol, 410 nmol, 420 nmol, 430 nmol, 440 nmol, 450 nmol, 460 nmol, 470 nmol, 480 nmol, 490 nmol, 500 nmol, 510 nmol, 520 nmol, 530 nmol, 540 nmol, 550 nmol, 660 nmol, 770 nmol, 880 nmol, 990 nmol, 600 nmol, 610 nmol, 620 nmol, 630 nmol, 640 nmol, 650 nmol, 660 nmol, 670 nmol, 680 nmol, 690 nmol, 700 nmol, 710 nmol, 720 nmol, 730 nmol, 740 nmol, 750 nmol, 760 nmol, 770 nmol, 780 nmol, 790 nmol, 800 nmol, 810 nmol, 820 nmol, 830 nmol, 840 nmol, 850 nmol, 860 nmol, 870 nmol, 880 nmol, 890 nmol, 900 nmol, 910 nmol, 920 nmol, 930 nmol, 940 nmol, 950 nmol, 960 nmol, 970 nmol, 980 nmol, 990 nmol, 1000 nmol, or even more.

In some embodiments, the pharmaceutical composition is administered parenterally (e.g., subcutaneously) at a therapeutically effective dose of the non-naturally occurring melanocortin analog, which is at least about 0.001 mg, 0.005 mg, 0.1 mg, 0.2 mg, 0.3 mg, 0.4 mg, 0.5 mg, 6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 61 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, 600 mg, 610 mg, 620 mg, 630 mg, 640 mg, 650 mg, 660 mg, 670 mg, 680 mg, 690 mg, 700 mg, 710 mg, 720 mg, 730 mg, 740 mg, 750 mg, 760 mg, 770 mg, 780 mg, 790 mg, 800 mg, 810 mg, 820 mg, 830 mg, 840 mg, 850 mg, 860 mg, 870 mg, 880 mg, 890 mg, 900 mg, 910 mg, 920 mg, 930 mg, 940 mg, 950 mg, 960 mg, 970 mg, 980 mg, 990 mg, 1000 mg, or even more.

In some embodiments of the methods of the present technology, at least one individual dose of the non-naturally occurring melanocortin analog is no more than about 1000 nmol, 990 nmol, 980 nmol, 970 nmol, 960 nmol, 950 nmol, 940 nmol, 930 nmol, 920 nmol, 910 nmol, 900 nmol, 890 nmol, 880 nmol, 870 nmol, 860 nmol, 850 nmol, 840 nmol, 830 nmol, 820 nmol, 810 nmol, 800 nmol, 790 nmol, 780 nmol, 770 nmol, 760 nmol, 750 nmol, 740 nmol, 730 nmol, 720 nmol, 710 nmol, 700 nmol, 690 nmol, 680 nmol, 670 nmol, 660 nmol, 650 nmol, 640 nmol, 630 nmol, 620 nmol, 610 nmol, 600 nmol, 590 nmol, 580 nmol, 570 nmol, 560 nmol, 550 nmol, 540 nmol, 530 nmol, 520 nmol, 510 nmol, 500 nmol, 490 nmol, 480 nmol, 470 nmol, 460 nmol, 450 nmol, 440 nmol, 430 nmol, 420 nmol, 410 nmol, 400 nmol, 390 nmol, 380 nmol, 370 nmol, 360 nmol, 350 nmol, 340 nmol, 330 nmol, 320 nmol, 310 nmol, 300 nmol, 290 nmol, 280 nmol, 270 nmol, 260 nmol, 250 nmol, 240 nmol, 230 nmol, 220 nmol, 210 nmol, 200 nmol, 190 nmol, 180 nmol, 170 nmol, 160 nmol, 150 nmol, 140 nmol, 130 nmol, 120 nmol, 110 nmol, 100 nmol, 99 nmol, 98 nmol, 97 nmol, 96 nmol, 95 nmol, 94 nmol, 93 nmol, 92 nmol, 91 nmol, 90 nmol, 89 nmol, 88 nmol, 87 nmol, 86 nmol, 85 nmol, 84 nmol, 83 nmol, 82 nmol, 81 nmol, 80 nmol, 79 nmol, 78 nmol, 77 nmol, 76 nmol, 75 nmol, 74 nmol, 73 nmol, 72 nmol, 71 nmol, 70 nmol, 69 nmol, 68 nmol, 67 nmol, 66 nmol, 65 nmol, 64 nmol, 63 nmol, 62 nmol, 61 nmol, 60 nmol, 59 nmol, 58 nmol, 57 nmol, 56 nmol, 95 nmol, 54 nmol, 53 nmol, 52 nmol, 51 nmol, 50 nmol, 49 nmol, 48 nmol, 47 nmol, 46 nmol, 45 nmol, 44 nmol, 43 nmol, 42 nmol, 41 nmol, 40 nmol, 39 nmol, 38 nmol, 37 nmol, 36 nmol, 35 nmol, 34 nmol, 33 nmol, 32 nmol, 31 nmol, 30 nmol, 29 nmol, 28 nmol, 27 nmol, 26 nmol, 25 nmol, 24 nmol, 23 nmol, 22 nmol, 21 nmol, 20 nmol, 19 nmol, 18 nmol, 17 nmol, 16 nmol, 15 nmol, 94 nmol, 13 nmol, 12 nmol, 11 nmol, 10 nmol, 9 nmol, 8 nmol, 7 nmol, 6, nmol, 5 nmol, 4 nmol, 3 nmol, 2 nmol, 1 nmol, 0.9 nmol, 0.8 nmol, 0.7 nmol, 0.6 nmol, 0.5 nmol, 0.4 nmol, 0.3 nmol, 0.2 nmol, 0.1 nmol, 0.05 nmol, 0.01 nmol, 0.005 nmol, 0.001 nmol, and even, in some aspects, less than about 0.001 nmol.

In some embodiments of the methods of the present technology, at least one individual dose of the non-naturally occurring melanocortin analog is no more than about 1000 mg, 990 mg, 980 mg, 970 mg, 960 mg, 950 mg, 940 mg, 930 mg, 920 mg, 910 mg, 900 mg, 890 mg, 880 mg, 870 mg, 860 mg, 850 mg, 840 mg, 830 mg, 820 mg, 810 mg, 800 mg, 790 mg, 780 mg, 770 mg, 760 mg, 750 mg, 740 mg, 730 mg, 720 mg, 710 mg, 700 mg, 690 mg, 680 mg, 670 mg, 660 mg, 650 mg, 640 mg, 630 mg, 620 mg, 610 mg, 600 mg, 590 mg, 580 mg, 570 mg, 560 mg, 550 mg, 540 mg, 530 mg, 520 mg, 510 mg, 500 mg, 490 mg, 480 mg, 470 mg, 460 mg, 450 mg, 440 mg, 430 mg, 420 mg, 410 mg, 400 mg, 390 mg, 380 mg, 370 mg, 360 mg, 350 mg, 340 mg, 330 mg, 320 mg, 310 mg, 300 mg, 290 mg, 280 mg, 270 mg, 260 mg, 250 mg, 240 mg, 230 mg, 220 mg, 210 mg, 200 mg, 190 mg, 180 mg, 170 mg, 160 mg, 150 mg, 140 mg, 130 mg, 120 mg, 110 mg, 100 mg, 99 mg, 98 mg, 97 mg, 96 mg, 95 mg, 94 mg, 93 mg, 92 mg, 91 mg, 90 mg, 89 mg, 88 mg, 87 mg, 86 mg, 85 mg, 84 mg, 83 mg, 82 mg, 81 mg, 80 mg, 79 mg, 78 mg, 77 mg, 76 mg, 75 mg, 74 mg, 73 mg, 72 mg, 71 mg, 70 mg, 69 mg, 68 mg, 67 mg, 66 mg, 65 mg, 64 mg, 63 mg, 62 mg, 61 mg, 60 mg, 59 mg, 58 mg, 57 mg, 56 mg, 95 mg, 54 mg, 53 mg, 52 mg, 51 mg, 50 mg, 49 mg, 48 mg, 47 mg, 46 mg, 45 mg, 44 mg, 43 mg, 42 mg, 41 mg, 40 mg, 39 mg, 38 mg, 37 mg, 36 mg, 35 mg, 34 mg, 33 mg, 32 mg, 31 mg, 30 mg, 29 mg, 28 mg, 27 mg, 26 mg, 25 mg, 24 mg, 23 mg, 22 mg, 21 mg, 20 mg, 19 mg, 18 mg, 17 mg, 16 mg, 15 mg, 94 mg, 13 mg, 12 mg, 11 mg, 10 mg, 9 mg, 8 mg, 7 mg, 6, mg, 5 mg, 4 mg, 3 mg, 2 mg, 1 mg, 0.9 mg, 0.8 mg, 0.7 mg, 0.6 mg, 0.5 mg, 0.4 mg, 0.3 mg, 0.2 mg, 0.1 mg, 0.05 mg, 0.01 mg, 0.005 mg, 0.001 mg, and in some aspects, even less.

In some embodiments of the methods of the present technology, a therapeutically effective dose of the non-naturally occurring melanocortin analog is at least about 0.001 mg/kg per body weight to about 10 mg/kg per body weight per day. In some aspects of the methods of the present technology, a therapeutically effective dose of the non-naturally occurring melanocortin analog is at least about 0.001 mg/kg, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 2.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10.0 mg/kg per body weight per day, or in some aspects, even more.

In some embodiments, the non-naturally occurring melanocortin analog is administered at least once daily in an amount ranging from 0.001 mg/kg to 25 mg/kg per body weight of the subject. For example, the non-naturally occurring melanocortin analog is administered to the subject at least once daily in an amount ranging from 0.001 mg/kg to 25 mg/kg, from 0.01 mg/kg to 20 mg/kg, from 0.1 mg/kg to 15 mg/kg, from 1 mg/kg to 10 mg/kg, or from 2 mg/kg to 5 mg/kg, per body weight of the subject.

In some embodiments, the non-naturally occurring melanocortin analog is administered at least once daily in an amount ranging from about 0.5 mg/kg to about 10 mg/kg per body weight of the subject. For example, the non-naturally occurring melanocortin analog is administered to the subject at least once daily in an amount ranging from about 0.5 mg/kg to about 10 mg/kg, from about 1 mg/kg to about 8 mg/kg, from about 2 mg/kg to about 6 mg/kg, or from about 4 mg/kg to about 5 mg/kg, per body weight of the subject.

In some embodiments, the non-naturally occurring melanocortin analog comprises a sequence of SEQ ID NO: 3, and the non-naturally occurring melanocortin analog is administered at least once daily in an amount ranging from 0.001 mg/kg to 25 mg/kg per body weight of the subject. For example, the non-naturally occurring melanocortin analog comprising a sequence of SEQ ID NO: 3 is administered to the subject at least once daily in an amount ranging from 0.001 mg/kg to 25 mg/kg, from 0.01 mg/kg to 20 mg/kg, from 0.1 mg/kg to 15 mg/kg, from 1 mg/kg to 10 mg/kg, or from 2 mg/kg to 5 mg/kg, per body weight of the subject.

In some embodiments, the non-naturally occurring melanocortin analog comprises a sequence of SEQ ID NO: 3, and the non-naturally occurring melanocortin analog is administered at least once daily in an amount ranging from 0.5 mg/kg to 10 mg/kg per body weight of the subject. For example, the non-naturally occurring melanocortin analog comprising a sequence of SEQ ID NO: 3 is administered to the subject at least once daily in an amount ranging from about 0.5 mg/kg to about 10 mg/kg, from about 1 mg/kg to about 8 mg/kg, from about 2 mg/kg to about 6 mg/kg, or from about 4 mg/kg to about 5 mg/kg, per body weight of the subject.

In some embodiments, the pharmaceutical composition is administered by a clinician. In other aspects, the pharmaceutical composition is self-administered. For example, the pharmaceutical composition may be administered in the morning, in the afternoon, or periodically throughout the day. The dose size may be adjusted to account for the frequency and timing of administration of the pharmaceutical composition, and that the daily dosage may, to some degree, be determined by the subject or a clinician based on estimated need, on the delivery system used, and on the presence or absence of other risk factors (e.g., hereditary risk factors or other environmental risk factors such as occupational risk factors and/or exposure to air pollution).

In some embodiments, it may be desirable to place an upper limit on single doses and/or daily dosage. Administration devices that limit or modulate self-administration of parenterally administered pharmaceuticals and other substances to prevent possible overdose by the subject are known in the art.

In some embodiments of the methods of the present technology, the pharmaceutical composition may be administered several times a month, several times a week, once each day, or even several times a day. Typically, a therapeutically effective dose is administered once each day. As a non-limiting example, an effective dose may be administered in one or more sessions, such as one portion of a dose is administered in the morning and the remaining portion of a dose is administered in the afternoon.

In some embodiments, the non-naturally occurring melanocortin analog or the pharmaceutical composition thereof is administered to the subject prior to or after a meal.

In some embodiments, the non-naturally occurring melanocortin analog is administered to the subject with a meal.

Dose frequency may be from once daily, twice daily, three times daily, or four times daily, to twice daily, four times daily, six times daily, eight times daily, ten times daily or more than ten times per day. In some embodiments, the dose frequency is from once daily to ten times daily, once daily to five times daily, twice daily, or once daily. Frequency of administration may be determined and adjusted over the course of care, and is generally, but not necessarily, based on symptoms and clinical findings.

In some embodiments, the administration pattern of the pharmaceutical composition of the present technology comprises administration of a single dose of the pharmaceutical composition three times every day, twice every day, once every day, once every 2 days, once every 3 days, once every 4 days, once every 5 days, once every 6 days, once every week, once every 2 weeks, once every 3 weeks, once every 4 weeks, once every 5 weeks, once every 6 weeks, once every 7 weeks, once every 8 weeks, once every 9 weeks, once every 10 weeks, once every 15 weeks, once every 20 weeks, or once every 24 weeks.

In some embodiments, the volume of a single dose of the pharmaceutical composition is up to about 10 mL, up to about 7.5 mL, up to about 5 mL, up to about 4 mL, up to about 3 mL, up to about 2 mL, up to about 1 mL, up to about 0.75 mL, up to about 0.5 mL, up to about 0.25 mL, up to about 0.1 mL, or up to about 0.01 mL.

In some embodiments, the volume of a single dose of the pharmaceutical composition is about 20 mL, about 19 mL, about 18 mL, about 17 mL, about 16 mL, about 15 mL, about 14 mL, about 13 mL, about 12 mL, about 11 mL, about 10 mL, about 9 mL, about 8 mL, about 7 mL, about 6 mL, about 5 mL, about 4 mL, about 3 mL, about 2 mL, about 1 mL, about 0.75 mL, about 0.5 mL, about 0.25 mL, about 0.1 mL, about 0.05 mL, or about 0.01 mL.

In some embodiments, the non-naturally occurring melanocortin analog or the pharmaceutical composition thereof is administered hourly (such as every hour, every 2 hours, every 4 hours, every 8 hours, etc.), once a day, twice a day, or three times a day. In some embodiments, the non-naturally occurring melanocortin analog or the pharmaceutical composition thereof is administered every morning, every evening, or every afternoon.

In some embodiments, the non-naturally occurring melanocortin analog or the pharmaceutical composition thereof can be administered as a dosing regimen comprising once, twice, or three times a day administration on a (i) weekly; (ii) every other week; (iii) one week of therapy followed by two, three or four weeks off; (iv) two weeks of therapy followed by one, two, three or four weeks off; (v) three weeks of therapy followed by one, two, three, four or five week off; (vi) four weeks of therapy followed by one, two, three, four or five week off; (vii) five weeks of therapy followed by one, two, three, four or five week off; or (viii) monthly schedule. The (i)-(viii) schedule can be repeated 2, 4, 6, 8, 10, 12 times, or more. In some embodiments, the non-naturally occurring melanocortin analog or the pharmaceutical composition thereof is administered at various dosages during the dosing regimen (e.g., a first dose with an effective amount of 10 mg/kg and a second dose with an effective amount of 5 mg/kg).

In some embodiments, the non-naturally occurring melanocortin analog or the pharmaceutical composition thereof can be administered once every 2 or 3 days, repeated for a total of at least 3 dosages, or twice per week for 4 to 6 weeks. The non-naturally occurring melanocortin analog or the pharmaceutical composition can be administered once every other week or even less frequently. Alternatively, the dosage regimen can be decreased to once every 2 or 3 weeks for 2-3 months. In some embodiments, the dosing regimen can be repeated at other intervals.

In some embodiments, the non-naturally occurring melanocortin analog or the pharmaceutical composition thereof is administered to the subject for at least 1 day, 1 week, 1 month, 3 months, 6 months, 1 year, 2 years, 3 years, 4 years, or 5 years.

In some embodiments, the non-naturally occurring melanocortin analog or the pharmaceutical composition thereof is administered to the subject for 1 day, 2 days, 3 days, 4 days, 5 days, 6 days, 7 days, 14 days, 21 days, 28 days, 35 days, 40 days, 45 days, 50 days, 60 days, 75 days, 90 days, 100 days, 110 days, or 120 days.

In some embodiments, the non-naturally occurring melanocortin analog crosses blood-brain-barrier (BBB) of the subject.

In some embodiments, the non-naturally occurring melanocortin analog is administered via intraperitoneal, intravenous, parenteral, subcutaneous, intramuscular, intracerebroventricular, or oral administration.

In some embodiments, the non-naturally occurring melanocortin analog is administered to the at least once daily in an amount ranging from 0.001 mg/kg to 25 mg/kg, from 0.01 mg/kg to 20 mg/kg, from 0.1 mg/kg to 15 mg/kg, from 1 mg/kg to 10 mg/kg, or from 2 mg/kg to 5 mg/kg body weight of the metabolically unchallenged subject.

In some embodiments, the non-naturally occurring melanocortin analog is administered at least once daily in an amount ranging from about 0.5 mg/kg to about 10 mg/kg, from about 1 mg/kg to about 8 mg/kg, from about 2 mg/kg to about 6 mg/kg, or from about 4 mg/kg to about 5 mg/kg, body weight of the metabolically unchallenged subject.

In some embodiments, the non-naturally occurring melanocortin analog is administered to the metabolically unchallenged subject for at least 1 day, 1 week, 1 month, 3 months, 6 months, 1 year, or 5 years.

In some embodiments, the non-naturally occurring melanocortin analog is administered to the metabolically unchallenged subject for 1 day, 7 days, 14 days, 21 days, 28 days, 35 days, 40 days, 45 days, 50 days, 60 days, 75 days, 90 days, 100 days, 110 days, or 120 days.

In some embodiments, the non-naturally occurring melanocortin analog or a pharmaceutical composition thereof can be administered hourly (such as every hour, every 2 hours, every 4 hours, every 8 hours, etc.), once a day, or twice a day. In some embodiments, the pharmaceutical composition may be administered every morning, every evening, or every afternoon. In some embodiments, the non-naturally occurring melanocortin analog or the pharmaceutical composition thereof may be administered before meal, after meal, or with meal.

In some embodiments, the non-naturally occurring melanocortin analog or the pharmaceutical composition thereof can be administered as a dosing regimen comprising once, twice, or three times a day administration on a (i) weekly; (ii) every other week; (iii) one week of therapy followed by two, three or four weeks off; (iv) two weeks of therapy followed by one, two, three or four weeks off; (v) three weeks of therapy followed by one, two, three, four or five week off; (vi) four weeks of therapy followed by one, two, three, four or five week off; (vii) five weeks of therapy followed by one, two, three, four or five week off; or (viii) monthly schedule. The (i)-(viii) schedule can be repeated 2, 4, 6, 8, 10, or 12 times or more. In some embodiments, the non-naturally occurring melanocortin analog or pharmaceutical composition thereof is administered at various dosages during the dosing regimen (e.g., a first dose with an effective amount of 10 mg/kg and a second dose with an effective amount of 5 mg/kg).

In some embodiments, the non-naturally occurring melanocortin analog or the pharmaceutical composition can be administered once every 2 or 3 days, repeated for a total of at least 3 dosages, or twice per week for 4-6 weeks. The non-naturally occurring melanocortin analog or the pharmaceutical composition can be administered once every other week or even less frequently. Alternatively, the dosage regimen can be decreased to once every 2 or 3 weeks for 2-3 months. In some embodiments, the dosing regimen can be repeated at other intervals.

### Subjects

In some embodiments, the subject is a mammal, including but not limited to a human, a non-human primate such as a chimpanzee, a domestic livestock or a farm animal such as a cow, a bison, sheep, a pig, a goat, a horse, a chicken, and a rooster, a domestic pet animal such as a dog, a cat, a rat, a mouse, and a rabbit, and a laboratory subject such as a rodent, including a rat, a mouse, and a guinea pig. In some embodiments, the subject is a human. In some embodiments, the subject is an animal such as a rat or a dog.

In some embodiments, the subject is a metabolically unchallenged subject.

In some embodiments, the subject has a body mass index (BMI) of 18.5 kg/m² to 25 kg/m². In some embodiments, the subject has a BMI of less than 20 kg/m². In some embodiments, the subject has a BMI of less than 18.5 kg/m².

In some embodiments, the subject is an underweight subject. Underweight subjects include those having a body weight about 3%, 5% or less, 10% or less, 20% or less, or 30% or less, than the lower end of “normal” BMI (e.g., 18.5 kg/m²).

In some embodiments, the subject is a patient who has experienced an involuntary weight loss prior to commencement of the administration. For example, the subject is a patient who has experienced a weight loss of 1% per month, 2% per month, 5% per month, or 10% per month for at least 2 months, at least 3 months, at least 6 months, at least 9 months, or at least 12 months, prior to commencement of the administration.

In some embodiments, the subject experiences loss of appetite, reduced appetite, decreased food consumption, and/or weight loss prior to the administration. In some embodiments, the subject experiences decrease in ease of eating prior to the administration. In certain embodiments, the loss of appetite, decreased food consumption, and/or weight loss is caused by cachexia.

In some embodiments, the subject has a cancer. In some embodiments, the loss of appetite, reduced appetite, decreased food consumption, and/or weight loss is caused by cancer cachexia.

In some embodiments, the cancer is at least one selected from the group consisting of bone cancer, testicular cancer, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer (e.g., small-cell lung cancer, non-small-cell lung cancer), ovarian cancer, breast cancer, prostate cancer, esophageal cancer, pancreatic cancer, glioma, leukemia, multiple myeloma, renal cell carcinoma, bladder cancer, cervical cancer, choriocarcinoma, colorectal cancer, oral cancer, skin cancer, and melanoma.

In some embodiments, the cancer is at least one selected from the group consisting of bone cancer, lung cancer (e.g., small-cell lung cancer, non-small-cell lung cancer), testicular cancer, breast cancer, ovarian cancer, cervical cancer, bladder cancer, and head and neck cancer.

In some embodiments, the cancer is non-small-cell lung cancer, colorectal cancer, stomach cancer, ovarian cancer, and/or pancreatic cancer.

In some embodiments, the subject with a cancer has previously undergone a treatment with the anticancer agent. In related embodiments, the subject has previously experienced one or more adverse side effects when treated with the anticancer agent. In some embodiments, such adverse side effects are selected from the group consisting of nausea, emesis, anorexia, weight loss, fatigue, fat mass loss, and muscle mass loss.

In some embodiments, the subject has a disease or condition in lieu of or in addition to cancer. The disease or condition can be selected from the group consisting of a psychological disease or condition, an allergy, an intolerance, a gastrointestinal disease or condition, a side-effect from a medication, substance use, a viral infection, a bacterial infection, food poisoning, dehydration, fatigue, hormonal imbalance, pain, a cardiovascular disease or condition, anemia, an autoimmune disease or condition, a respiratory disease or condition, and an inflammatory disease or condition.

In some embodiments, the psychological disease or condition is selected from the group consisting of depression, bipolar disorder, schizophrenia, anorexia nervosa, anxiety, grief, stress, bulimia, post-traumatic stress disorder, phobia, aversions such as smell, taste, sight, texture, social anxiety, shock, obsessive-compulsive disorder, eating disorders dementia, Alzheimer's, Parkinson's, multiple sclerosis.

In some embodiments, the allergy or intolerance is selected from the group consisting of gluten, dairy, soy, nut, and seed.

In some embodiments, the gastrointestinal disease or condition is selected from the group consisting of irritable bowel syndrome (IBS), celiac disease, and Crohn's disease. In some embodiments, the disease or condition is advanced age.

In some embodiments, the medication is selected from the group consisting of antibiotics, codeine, morphine, sleeping pills, blood pressure medications, diuretics, anabolic steroids, cardiovascular medications, including digoxin, fluoxetine, and hydralazine.

In some embodiments, the substance use is selected from the group consisting of cocaine, methamphetamines, heroin, and alcohol.

In some embodiments, the disease or condition is substance withdrawal.

In some embodiments, the cardiovascular disease or condition is chronic heart failure (CHF).

In some embodiments, the respiratory disease or condition is chronic obstructive pulmonary disease (COPD).

The subject can be any subject already with the disease or condition (e.g., cancer, depression, IBS). In some embodiments, the subject can be a subject which does not yet experience or exhibit symptoms of the disease or condition, or a subject predisposed to the disease or condition. In some embodiments, the subject is a person who is predisposed to cancer, e.g., a person with a family history of cancer.

In some embodiments, the subject has a disease or condition selected from the group consisting of a psychological disease or condition, an allergy, an intolerance, a gastrointestinal disease or condition, a side-effect from a medication, substance use, a viral infection, a bacterial infection, food poisoning, dehydration, fatigue, hormonal imbalance, pain, a cardiovascular disease or condition, anemia, an autoimmune disease or condition, a respiratory disease or condition, and an inflammatory disease or condition.

In some embodiments, the allergy or intolerance is selected from the group consisting of gluten, dairy, eggs, finfish, shellfish, soy, nuts and seeds.

In some embodiments, the substance use is selected from the group consisting of cocaine, methamphetamines, heroin, alcohol.

In some embodiments, the disease or condition is food poisoning.

In some embodiments, the disease or condition is dehydration.

In some embodiments, the viral infection, bacterial infection, or disease or condition caused by a viral infection or a bacterial infection is selected from the group consisting of upper respiratory infection, COVID-19, pneumonia, gastroenteritis, skin, meningitis, HIV, hepatitis, flu, common cold, urine infection.

In some embodiments, the disease or condition is selected from the group consisting of sleep deprivation, fatigue, chronic fatigue syndrome, nausea, loss of taste, smell, sight, and satiety.

In some embodiments, the disease or condition selected from the group consisting of pregnancy, hormonal therapy and menopause.

In some embodiments, the pain is caused by a condition or disease selected from the group consisting of fibromyalgia, migraines, nerve damage, post-surgical, oral pain, and dental pain.

In some embodiments, the cardiovascular disease or condition is selected from the group consisting of heart disease including congestive heart failure (CHF), postural orthostatic tachycardia syndrome, high blood pressure, and anemia.

In some embodiments, the inflammatory disease or condition is selected from the group consisting of lupus and rheumatoid arthritis.

In some embodiments, the chronic lung disease is chronic obstructive pulmonary disease (COPD).

In some embodiments, the metabolically unchallenged subject is a human.

In some embodiments, the metabolically unchallenged subject is an animal.

In some embodiments, the subject is a mammal, including but not limited to a human, a non-human primate such as a chimpanzee, a domestic livestock or a farm animal such as a cow, a bison, sheep, a pig, a goat, a horse, a chicken, and a rooster, a domestic pet animal such as a dog, a cat, a rat, a mouse, and a rabbit, and a laboratory subject such as a rodent, including a rat, a mouse, and a guinea pig.

In some embodiments, the subject is a human.

In some embodiments, the subject is a rat.

In some embodiments, the subject is a dog.

### Associated Outcomes of Administration

In some embodiments, the method of the present technology (i) stimulates appetite of the subject; (ii) increases food consumption of the subject; (iii) prevents or alleviates nausea, emesis, and/or anorexia in the subject; (iv) increases or maintains body weight or BMI of the subject; (v) prevents or reduces weight loss of the subject; (vi) increases or maintains muscle mass of the subject; (vii) prevents or reduces muscle mass loss of the subject; (viii) increases or maintains fat mass of the subject; and/or (ix) prevents or reduces fat mass loss of the subject.

In some embodiments, appetite of the subject is increased by at least 10% to 200% after the administration (e.g., administration of the therapeutically effective amount of the non-naturally occurring melanocortin analog or the pharmaceutical composition thereof). For example, appetite of the subject is increased by at least 10% to 200%, at least 20% to 175%, at least 30 to 150%, at least 40% to 125%, at least 50% to 100%, or at least 60% to 75%, after the administration.

In some embodiments, appetite of the subject is increased by at least 25% to 100% after the administration. For example, appetite of the subject is increased by at least 25% to 100%, at least 30% to 90%, at least 35 to 80%, at least 40% to 70%, at least 45% to 60%, or at least 50% to 55%, after the administration.

In some embodiments, appetite of the subject is increased, as measured by a decrease in the subject's scaling of satiety (e.g., easy or hard to eat) from 0 (extremely easy to eat) to 100 (extremely hard to eat). In some embodiments, the subject's scaling of satiety is decreased by 40% to 150% after administration. For example, the subject's scaling of satiety may be decreased by 40% to 150%, 45% to 125%, 50% to 100%, or 55% to 75% after administration. In some embodiments, the subject's scaling of satiety is decreased by 40% to 150%, 45% to 125%, 50% to 100%, or 55% to 75% within 8 days after administration

In some embodiments, appetite of the subject is increased, as measured by a decrease in the subject's scaling of satiety (e.g., easy or hard to eat) from 0 (extremely easy to eat) to 100 (extremely hard to eat). In some embodiments, the subject's scaling of satiety is decreased by at least 7, 10, 15, or 20 points after administration. For example, the subject's scaling of satiety may be decreased by at least 7 points after administration. In some embodiments, the subject's scaling of satiety is decreased by at least 7, 10, 15, or 20 points within 8 days after administration.

In some embodiments, appetite of the subject is increased, as measured by a decrease in the subject's scaling of satiety (e.g., easy or hard to eat) from 0 (extremely easy to eat) to 100 (extremely hard to eat). In some embodiments, the subject's scaling of satiety is decreased to a scaling below 15, 12, 10, 8, or 5 points after administration. In some embodiments, the subject's scaling of satiety is decreased to a scaling below 15, 12, 10, 8, or 5 points within 8 days after administration.

In some embodiments, appetite is increased for at least 1 day, 1 week, 1 month, 3 months, 6 months, 1 year, 2 years, 3 years, 4 years, or 5 years during the administration.

In some embodiments, appetite is increased for at least 1 day, 2 days, 3 days, 4 days, 5 days, 6 days, 7 days, 14 days, 21 days, 28 days, 35 days, 40 days, 45 days, 50 days, 60 days, 75 days, 90 days, 100 days, 110 days, or 120 days during the administration.

In some embodiments, the increased appetite is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

In some embodiments, the appetite is determined by a scale for measuring desire to eat, feeling of hunger, and/or level of satiety. For example, the appetite can be assessed through a daily questionnaire given at specified or random times of the day. In the questionnaire, subjects rate their hunger and/or desire to eat greater varieties of food using scales ranging from 0 (not at all) to 100 (extremely). In some embodiments, the appetite is assessed by the subject's rating of feeling of hunger using a scale ranging from 0 (not all all) to 100 (extremely). In some embodiments, the appetite is assessed by the subject's scaling of satiety (e.g., easy or hard to eat) from 0 (extremely easy to eat) to 100 (extremely hard to eat).

In some embodiments, the appetite is assessed between meals. In some embodiments, the appetite is assessed during a meal.

In some embodiments, the appetite is assessed after a meal, for example, 1 minute after a meal, 5 minutes, 10 minutes, 15 minutes, 30 minutes, 45 minutes, 1 hour, 2 hours, 3 hours, 4 hours, 5 hours, 6 hour, 7 hours, 8 hours, 9 hours, 10 hours, 11 hours, 12 hours, or 24 hours after a meal.

In some embodiments, the subject comprises two or more subjects, and the appetite is an average appetite (e.g., average rating of feeling of hunger, average scaling of satiety) of the two or more subjects. In some embodiments, the subject comprises at least 3 subjects, and the appetite is an average appetite of the at least 3 subjects. In some embodiments, the subject comprises at least 4 subjects, and the appetite is an average appetite of the at least 4 subjects. In some embodiments, the subject comprises at least 5 subjects, and the appetite is an average appetite of the at least 5 subjects. In some embodiments, the subject comprises at least 6 subjects, and the appetite is an average appetite of the at least 6 subjects. In some embodiments, the subject comprises at least 7 subjects, and the appetite is an average appetite of the at least 7 subjects. In some embodiments, the subject comprises at least 8 subjects, and the appetite is an average appetite of the at least 8 subjects. In some embodiments, the subject comprises at least 9 subjects, and the appetite is an average appetite of the at least 9 subjects. In some embodiments, the subject comprises at least 10 subjects, and the appetite is an average appetite of the at least 10 subjects. The subject can comprise e.g., at least 10, 15, 20, 30, 40, 50, 75, 100, 200, 300, 400, 500, 750, 1,000, 2,500, or 5,000 subjects, and the appetite is an average appetite of the at least 10, 15, 20, 30, 40, 50, 75, 100, 200, 300, 400, 500, 750, 1,000, 2,500, or 5,000 subjects.

In some embodiments, food consumption of the subject is increased by at least 25% to 2,000% after the administration (e.g., administration of the therapeutically effective amount of the non-naturally occurring melanocortin analog or the pharmaceutical composition thereof). For example, food consumption of the subject can be increased by at least at least 25% to 2,000%, at least 50% to 1,750%, at least 75% to 1,500%, at least 100% to 1,000%, at least 250% to 750%, or at least 400% to 500%, after the administration.

In some embodiments, food consumption of the subject is increased by at least 50% to 500% after the administration. For example, food consumption of the subject can be increased by at least at least 50% to 500%, at least 60% to 400%, at least 70% to 300%, at least 80% to 250%, at least 90% to 200%, at least 100% to 175%, or at least 125% to 150%, after the administration.

In some embodiments, food consumption is increased for at least 1 day, 1 week, 1 month, 3 months, 6 months, 1 year, or 5 years during the administration.

In some embodiments, food consumption is increased for at least 1 day, 2 days, 3 days, 4 days, 5 days, 6 days, 7 days, 14 days, 21 days, 28 days, 35 days, 40 days, 45 days, 50 days, 60 days, 75 days, 90 days, 100 days, 110 days, or 120 days during the administration.

In some embodiments, the increased food consumption is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

In some embodiments, the food consumption is determined by daily food consumption, which is total calories (kcal) consumed in 1 day or mass of food (grams or kilograms) consumed in 1 day. In some embodiments, the food consumption is determined by weekly food consumption, which is total calories (kcal) consumed in 1 week or mass of food (grams or kilograms) consumed in 1 week. In some embodiments, the daily food consumption is determined by the daily food consumed normalized to daily body weight of the subject.

In some embodiments, the subject comprises two or more subjects, and the food consumption is an average food consumption (e.g., average daily food consumption, average weekly food consumption) of the two or more subjects. In some embodiments, the subject comprises at least 3 subjects, and the food consumption is an average food consumption of the at least 3 subjects. In some embodiments, the subject comprises at least 4 subjects, and the food consumption is an average food consumption of the at least 4 subjects. In some embodiments, the subject comprises at least 5 subjects, and the food consumption is an average food consumption of the at least 5 subjects. In some embodiments, the subject comprises at least 6 subjects, and the food consumption is an average food consumption of the at least 6 subjects. In some embodiments, the subject comprises at least 7 subjects, and the food consumption is an average food consumption of the at least 7 subjects. In some embodiments, the subject comprises at least 8 subjects, and the food consumption is an average food consumption of the at least 8 subjects. In some embodiments, the subject comprises at least 9 subjects, and the food consumption is an average food consumption of the at least 9 subjects. In some embodiments, the subject comprises at least 10 subjects, and the food consumption is an average food consumption of the at least 10 subjects. The subject can comprise e.g., at least 10, 15, 20, 30, 40, 50, 75, 100, 200, 300, 400, 500, 750, 1,000, 2,500, or 5,000 subjects, and the food consumption is an average food consumption of the at least 10, 15, 20, 30, 40, 50, 75, 100, 200, 300, 400, 500, 750, 1,000, 2,500, or 5,000 subjects.

In some embodiments, the method prevents or alleviates anorexia in the subject.

In some embodiments, anorexia is determined by food consumption. In some embodiments, anorexia is determined by daily food consumption, and/or weekly food consumption. In some embodiments, anorexia is determined by cumulative food consumption for at least 2 days, at least 5 days, at least 7 days, at least 14 days, or at least 21 days, after the administration.

In some embodiments, the method prevents or alleviates nausea, emesis, or both, in the subject.

In some embodiments, body weight of the subject is increased by at least 5% to 200% after the administration (e.g., administration of the therapeutically effective amount of the non-naturally occurring melanocortin analog or the pharmaceutical composition thereof). For example, body weight of the subject is increased by at least 5% to 200%, at least 10% to 150%, at least 25% to 100%, or at least 50% to 75%, after the administration.

In some embodiments, body weight of the subject is increased by at least 5% to 25% after the administration. For example, body weight of the subject is increased by at least 5% to 25%, at least 10% to 20%, or at least 12% to 15%, after the administration.

In some embodiments, body weight of the subject is increased by at least 20% to 100% after the administration. For example, body weight of the subject is increased by at least 20% to 100%, at least 35% to 90%, at least 50% to 80%, or at least 60% to 70%, after the administration.

In some embodiments, body weight of the subject is increased by at least 2% to 10% between day 2 after the administration and day 7 after administration (e.g., administration of the therapeutically effective amount of the non-naturally occurring melanocortin analog or the pharmaceutical composition thereof). For example, body weight of the subject is increased by at least 2% to 10%, at least 2.5% to 7.5%, or at least 3% to 5% between day 2 after the administration and day 7 after the administration.

In some embodiments, body weight of the subject is increased by at least 1.5 kg, 1.75 kg, 2 kg, 2.25 kg, or 2.5 kg between day 2 after administration and day 7 after administration.

In some embodiments, the increased body weight is maintained for at least 1 week, 2 weeks, 3 weeks, 1 month, 3 months, 6 months, 1 year, or 5 years, after the administration is stopped.

In some embodiments, the method prevents or reduces weight loss (e.g., involuntary weight loss) in the subject.

In some embodiments, the body weight (or weight loss) is determined by monitoring daily body weight. In some embodiments, the body weight (or weight loss) is determined by monitoring daily body weight gain (or loss). In some embodiments, the body weight (or weight loss) is determined by monitoring cumulative body weight for at least 1 week, 2 weeks, 3 weeks, 4 weeks, 3 months, 6 months, 1 year, or 5 years.

In some embodiments, BMI of the subject is increased by at least 5% to 200% after the administration (e.g., administration of the therapeutically effective amount of the non-naturally occurring melanocortin analog or the pharmaceutical composition thereof). For example, BMI of the subject is increased by at least 5% to 200%, at least 10% to 150%, at least 25% to 100%, or at least 50% to 75%, after the administration.

In some embodiments, BMI of the subject is increased by at least 5% to 25% after the administration. For example, BMI of the subject is increased by at least 5% to 25%, at least 10% to 20%, or at least 12% to 15%, after the administration.

In some embodiments, body weight of the subject is increased by at least 20% to 100% after the administration. For example, BMI of the subject is increased by at least 20% to 100%, at least 35% to 90%, at least 50% to 80%, or at least 60% to 70%, after the administration.

In some embodiments, BMI of the subject is increased by at least 1% to 10% between day 2 after the administration and day 7 after administration (e.g., administration of the therapeutically effective amount of the non-naturally occurring melanocortin analog or the pharmaceutical composition thereof). For example, BMI of the subject is increased by at least 1% to 10%, at least 1.25% to 7.5%, or at least 1.5% to 5% between day 2 after the administration and day 7 after the administration.

In some embodiments, BMI of the subject is increased by at least 0.5, 0.55, 0.6, 0.65, or 0.7 between day 2 after administration and day 7 after administration.

In some embodiments, the increased BMI is maintained for at least 1 week, 2 weeks, 3 weeks, 1 month, 3 months, 6 months, 1 year, or 5 years, after the administration is stopped.

In some embodiments, the method prevents or reduces BMI reduction (e.g., involuntary weight loss) in the subject.

In some embodiments, the BMI is determined by monitoring daily BMI. In some embodiments, the BMI is determined by monitoring daily changes in BMI. In some embodiments, the BMI is determined by monitoring daily weight and calculating BMI therefrom. In some embodiments, the BMI is determined by monitoring daily changes in weight and calculating the change in BMI therefrom.

In some embodiments, muscle mass of the subject is increased by at least 1% to 100% after the administration (e.g., administration of the therapeutically effective amount of the non-naturally occurring melanocortin analog or the pharmaceutical composition thereof). For example, muscle mass of the subject is increased by at least 1% to 100%, at least 5% to 75%, at least 10% to 50%, or at least 20% to 35%, after the administration.

In some embodiments, muscle mass of the subject is increased by at least 5% to 15% after the administration. For example, muscle mass of the subject is increased by at least 5% to 15%, at least 6% to 14%, at least 7% to 12%, or at least 8% to 9%, after the administration.

In some embodiments, muscle mass of the subject is increased by at least 25% to 50% after the administration. For example, muscle mass of the subject is increased by at least 25% to 50%, at least 28% to 48%, at least 25% to 45%, or at least 30% to 40%, after the administration.

In some embodiments, the increased muscle mass is maintained for at least 1 week, 2 weeks, 3 weeks, 1 month, 3 months, 6 months, 1 year, or 5 years, after the administration is stopped.

In some embodiments, the method prevents or reduces muscle mass loss (e.g., involuntary muscle mass loss) in the subject.

In some embodiments, the muscle mass is cardiac muscle mass, skeletal muscle mass, or both. In some embodiments, the cardiac muscle mass is determined by measuring change in heart weight. In some embodiments, the skeletal muscle mass is determined by measuring change in gastrocnemius tissue weight. In related embodiments, the muscle mass change is cardiac muscle mass change, skeletal muscle mass change, or both. In some embodiments, cardiac muscle is determined by heart weight. In some embodiments, skeletal muscle mass is determined by gastrocnemius tissue weight. In some embodiments, the muscle mass changes can be assessed by using magnetic resonance imaging (MRI) and/or or nuclear magnetic resonance (NMR).

In some embodiments, fat mass of the subject is increased by at least 1% to 100% after the administration (e.g., administration of the therapeutically effective amount of the non-naturally occurring melanocortin analog or the pharmaceutical composition thereof). For example, fat mass of the subject is increased by at least 1% to 100%, at least 5% to 75%, at least 10% to 50%, or at least 20% to 35%, after the administration.

In some embodiments, fat mass of the subject is increased by at least 5% to 15% after the administration. For example, fat mass of the subject is increased by at least 5% to 15%, at least 6% to 14%, at least 7% to 12%, or at least 8% to 9%, after the administration.

In some embodiments, fat mass of the subject is increased by at least 25% to 50% after the administration. For example, fat mass of the subject is increased by at least 25% to 50%, at least 28% to 48%, at least 25% to 45%, or at least 30% to 40%, after the administration.

In some embodiments, the increased fat mass is maintained for at least 1 week, 2 weeks, 3 weeks, 1 month, 3 months, 6 months, 1 year, or 5 years, after the administration is stopped.

In some embodiments, the method prevents or reduces fat mass loss (e.g., involuntary fat mass loss) in the subject.

In some embodiments, the fat mass can be determined by the fat fold technique. In some embodiments, the fat mass changes can be assessed by using magnetic resonance imaging (MRI) and/or or nuclear magnetic resonance (NMR).

In some embodiments, the method of the present technology stimulates appetite in the subject compared to a control subject, wherein the control subject is not administered the non-naturally occurring melanocortin analog. In some embodiments, the control subject experiences loss of appetite, reduced appetite, decreased food consumption, and/or weight loss. In certain embodiments, the loss of appetite, reduced appetite, decreased food consumption, and/or weight loss in the control subject is caused by cachexia. In some embodiments, the control subject has a cancer. In some embodiments, the loss of appetite, reduced appetite, decreased food consumption, and/or weight loss in the control subject is caused by cancer cachexia. In some embodiments, the control subject has the aforementioned disease or condition of the subject in lieu of or in addition to the cancer.

In some embodiments, appetite of the subject is increased after the administration by at least 10% to 200%, relative to appetite of the control subject. For example, appetite of the subject is increased after the administration by at least 10% to 200%, at least 20% to 175%, at least 30 to 150%, at least 40% to 125%, at least 50% to 100%, or at least 60% to 75%, relative to appetite of the control subject.

In some embodiments, appetite of the subject is increased after the administration by at least 25% to 100%, relative to appetite of the control subject. For example, appetite of the subject is increased after the administration by at least 25% to 100%, at least 30% to 90%, at least 35 to 80%, at least 40% to 70%, at least 45% to 60%, or at least 50% to 55% after administration, relative to appetite of the control subject.

In some embodiments, appetite of the subject is increased, as measured by a decrease in the subject's scaling of satiety (e.g., easy or hard to eat) from 0 (extremely easy to eat) to 100 (extremely hard to eat) compared to the control subject. In some embodiments, the subject's scaling of satiety is decreased by 40% to 150% compared to the control subject. For example, the subject's scaling of satiety may be decreased by 40% to 150%, 45% to 125%, 55% to 100%, or 60% to 80% after administration compared to a control subject. In some embodiments, the subject's scaling of satiety is decreased by 40% to 150%, 45% to 125%, 55% to 100%, or 60% to 80% within 8 days after administration relative to the appetite of a control subject.

In some embodiments, appetite of the subject is increased, as measured by a decrease in the subject's scaling of satiety (e.g., easy or hard to eat) from 0 (extremely easy to eat) to 100 (extremely hard to eat). In some embodiments, the subject's scaling of satiety is decreased by at least 10, 12, 14, 16, 18, or 20 points after administration, relative to that of a control subject. For example, the subject's scaling of satiety may be decreased by 14 points after administration relative to that of a control subject.

In some embodiments, appetite is increased for at least 1 day, 1 week, 1 month, 3 months, 6 months, 1 year, 2 years, 3 years, 4 years, or 5 years during the administration.

In some embodiments, appetite is increased for at least 1 day, 2 days, 3 days, 4 days, 5 days, 6 days, 7 days, 14 days, 21 days, 28 days, 35 days, 40 days, 45 days, 50 days, 60 days, 75 days, 90 days, 100 days, 110 days, or 120 days during the administration.

In some embodiments, the increased appetite is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

In some embodiments, the appetite is determined by a scale for measuring desire to eat, feeling of hunger, and/or level of satiety. For example, the appetite can be assessed through a daily questionnaire given at specified or random times of the day. In the questionnaire, subjects rate their hunger and/or desire to eat greater varieties of food using scales ranging from 0 (not at all) to 100 (extremely). In some embodiments, the appetite is assessed by the subject's or the control subject's rating of feeling of hunger using a scale ranging from 0 (not all all) to 100 (extremely). In some embodiments, the appetite is assessed by the subject's or the control subject's scaling of satiety (e.g., easy or hard to eat) from 0 (extremely easy to eat) to 100 (extremely hard to eat).

In some embodiments, the appetite is assessed between meals. In some embodiments, the appetite is assessed during a meal.

In some embodiments, the appetite is assessed after a meal, for example, 1 minute after a meal, 5 minutes, 10 minutes, 15 minutes, 30 minutes, 45 minutes, 1 hour, 2 hours, 3 hours, 4 hours, 5 hours, 6 hour, 7 hours, 8 hours, 9 hours, 10 hours, 11 hours, 12 hours, or 24 hours after a meal.

In some embodiments, the subject comprises two or more subjects, and the appetite is an average appetite (e.g., average rating of feeling of hunger, average scaling of satiety) of the two or more subjects. In some embodiments, the subject comprises at least 3 subjects, and the appetite is an average appetite of the at least 3 subjects. In some embodiments, the subject comprises at least 4 subjects, and the appetite is an average appetite of the at least 4 subjects. In some embodiments, the subject comprises at least 5 subjects, and the appetite is an average appetite of the at least 5 subjects. In some embodiments, the subject comprises at least 6 subjects, and the appetite is an average appetite of the at least 6 subjects. In some embodiments, the subject comprises at least 7 subjects, and the appetite is an average appetite of the at least 7 subjects. In some embodiments, the subject comprises at least 8 subjects, and the appetite is an average appetite of the at least 8 subjects. In some embodiments, the subject comprises at least 9 subjects, and the appetite is an average appetite of the at least 9 subjects. In some embodiments, the subject comprises at least 10 subjects, and the appetite is an average appetite of the at least 10 subjects. The subject can comprise e.g., at least 10, 15, 20, 30, 40, 50, 75, 100, 200, 300, 400, 500, 750, 1,000, 2,500, or 5,000 subjects, and the appetite is an average appetite of the at least 10, 15, 20, 30, 40, 50, 75, 100, 200, 300, 400, 500, 750, 1,000, 2,500, or 5,000 subjects.

In some embodiments, the control subject comprises two or more control subjects, and the appetite is an average appetite (e.g., average rating of feeling of hunger, average scaling of satiety) of the two or more control subjects. In some embodiments, the control subject comprises at least 3 control subjects, and the appetite is an average appetite of the at least 3 control subjects. In some embodiments, the control subject comprises at least 4 control subjects, and the appetite is an average appetite of the at least 4 control subjects. In some embodiments, the control subject comprises at least 5 control subjects, and the appetite is an average appetite of the at least 5 control subjects. In some embodiments, the control subject comprises at least 6 control subjects, and the appetite is an average appetite of the at least 6 control subjects. In some embodiments, the subject comprises at least 7 control subjects, and the appetite is an average appetite of the at least 7 control subjects. In some embodiments, the control subject comprises at least 8 control subjects, and the appetite is an average appetite of the at least 8 control subjects. In some embodiments, the control subject comprises at least 9 control subjects, and the appetite is an average appetite of the at least 9 control subjects. In some embodiments, the control subject comprises at least 10 control subjects, and the appetite is an average appetite of the at least 10 control subjects. The control subject can comprise e.g., at least 10, 15, 20, 30, 40, 50, 75, 100, 200, 300, 400, 500, 750, 1,000, 2,500, or 5,000 control subjects, and the appetite is an average appetite of the at least 10, 15, 20, 30, 40, 50, 75, 100, 200, 300, 400, 500, 750, 1,000, 2,500, or 5,000 control subjects.

In some embodiments, food consumption of the subject is increased after the administration by at least 25% to 2,000%, relative to food consumption of the control subject. For example, food consumption of the subject can be increased after the administration by at least at least 25% to 2,000%, at least 50% to 1,750%, at least 75% to 1,500%, at least 100% to 1,000%, at least 250% to 750%, or at least 400% to 500%, relative to food consumption of the control subject.

In some embodiments, food consumption of the subject is increased after the administration by at least 50% to 500%, relative to food consumption of the control subject. For example, food consumption of the subject can be increased after the administration by at least at least 50% to 500%, at least 60% to 400%, at least 70% to 300%, at least 80% to 250%, at least 90% to 200%, at least 100% to 175%, or at least 125% to 150%, relative to food consumption of the control subject.

In some embodiments, food consumption is increased for at least 1 day, 1 week, 1 month, 3 months, 6 months, 1 year, 2 years, 3 years, 4 years, or 5 years during the administration.

In some embodiments, food consumption is increased for at least 1 day, 2 days, 3 days, 4 days, 5 days, 6 days, 7 days, 14 days, 21 days, 28 days, 35 days, 40 days, 45 days, 50 days, 60 days, 75 days, 90 days, 100 days, 110 days, or 120 days during the administration.

In some embodiments, the increased food consumption is maintained for at least 1 day, 2 days, 3 days, 4 days, 5 days, 6 days, 1 week, 2 weeks, 3 weeks, 1 month, 3 months, 6 months, 1 year, 2 years, 3 years, 4 years, or 5 years, after the administration is stopped.

In some embodiments, the food consumption is determined by total calories (kcal) consumed in 1 day or mass of food (grams or kilograms) consumed in 1 day. In some embodiments, the food consumption is determined by daily food consumption, which is total calories (kcal) consumed in 1 day or mass of food (grams or kilograms) consumed in 1 day. In some embodiments, the food consumption is determined by weekly food consumption, which is total calories (kcal) consumed in 1 week or mass of food (grams or kilograms) consumed in 1 week. In some embodiments, the daily food consumption is determined by the daily food consumed normalized to daily body weight.

In some embodiments, the subject comprises two or more subjects, and the food consumption is an average food consumption (e.g., average daily food consumption, average weekly food consumption) of the two or more subjects. In some embodiments, the subject comprises at least 3 subjects, and the food consumption is an average food consumption of the at least 3 subjects. In some embodiments, the subject comprises at least 4 subjects, and the food consumption is an average food consumption of the at least 4 subjects. In some embodiments, the subject comprises at least 5 subjects, and the food consumption is an average food consumption of the at least 5 subjects. In some embodiments, the subject comprises at least 6 subjects, and the food consumption is an average food consumption of the at least 6 subjects. In some embodiments, the subject comprises at least 7 subjects, and the food consumption is an average food consumption of the at least 7 subjects. In some embodiments, the subject comprises at least 8 subjects, and the food consumption is an average food consumption of the at least 8 subjects. In some embodiments, the subject comprises at least 9 subjects, and the food consumption is an average food consumption of the at least 9 subjects. In some embodiments, the subject comprises at least 10 subjects, and the food consumption is an average food consumption of the at least 10 subjects. The subject can comprise e.g., at least 10, 15, 20, 30, 40, 50, 75, 100, 200, 300, 400, 500, 750, 1,000, 2,500, or 5,000 subjects, and the food consumption is an average food consumption of the at least 10, 15, 20, 30, 40, 50, 75, 100, 200, 300, 400, 500, 750, 1,000, 2,500, or 5,000 subjects.

In some embodiments, the control subject comprises two or more control subjects, and the food consumption is an average food consumption (e.g., average daily food consumption, average weekly food consumption) of the two or more control subjects. In some embodiments, the control subject comprises at least 3 control subjects, and the food consumption is an average food consumption of the at least 3 control subjects. In some embodiments, the control subject comprises at least 4 control subjects, and the food consumption is an average food consumption of the at least 4 control subjects. In some embodiments, the control subject comprises at least 5 control subjects, and the food consumption is an average food consumption of the at least 5 control subjects. In some embodiments, the control subject comprises at least 6 control subjects, and the food consumption is an average food consumption of the at least 6 control subjects. In some embodiments, the control subject comprises at least 7 control subjects, and the food consumption is an average food consumption of the at least 7 control subjects. In some embodiments, the control subject comprises at least 8 control subjects, and the food consumption is an average food consumption of the at least 8 control subjects. In some embodiments, the control subject comprises at least 9 control subjects, and the food consumption is an average food consumption of the at least 9 control subjects. In some embodiments, the control subject comprises at least 10 control subjects, and the food consumption is an average food consumption of the at least 10 control subjects. The control subject can comprise e.g., at least 10, 15, 20, 30, 40, 50, 75, 100, 200, 300, 400, 500, 750, 1,000, 2,500, or 5,000 control subjects, and the food consumption is an average food consumption of the at least 10, 15, 20, 30, 40, 50, 75, 100, 200, 300, 400, 500, 750, 1,000, 2,500, or 5,000 control subjects.

In some embodiments, body weight of the subject is increased after the administration, and weight gain of the subject after the administration is at least 5% to 200% greater than that of the control subject. For example, weight gain of the subject after the administration is at least 5% to 200%, at least 10% to 150%, at least 25% to 100%, or at least 50% to 75% greater than that of the control subject.

In some embodiments, body weight of the subject is increased after the administration, and weight gain of the subject after the administration is at least 5% to 25% greater than that of the control subject. For example, weight gain of the subject after the administration is at least 5% to 25%, at least 10% to 20%, or at least 12% to 15% greater than that of the control subject.

In some embodiments, body weight of the subject is increased after the administration, and weight gain of the subject after the administration is at least 20% to 100% greater than that of the control subject. For example, weight gain of the subject after the administration is at least 20% to 100%, at least 35% to 90%, at least 50% to 80%, or at least 60% to 70% greater than that of the control subject.

In some embodiments, body weight of the subject is increased between day 2 after the administration and day 7 of the administration, and weight gain of the subject between days 2 and 7 after administration is at least 20% to 150% greater than that of the control subject. For example, weight gain of the subject between day 2 and day 7 after administration is at least 20% to 150%, 40% to 125%, 60% to 110%, or 80% to 100% greater than that of the control subject.

In some embodiments, body weight of the subject is increased between day 2 after the administration and day 7 of the administration, and weight gain of the subject between days 2 and 7 after administration is at least 0.5 kg, 0.75 kg, 1 kg, 1.25 kg, 1.5 kg, 1.75 kg, or 2 kg greater than that of the control subject.

In some embodiments, the increased body weight is maintained for at least 1 week, 2 weeks, 3 weeks, 1 month, 3 months, 6 months, 1 year, 2 years, 3 years, 4 years, or 5 years, after the administration is stopped.

In some embodiments, the body weight (or weight gain) is determined by monitoring daily body weight. In some embodiments, the body weight (or weight gain) is determined by monitoring daily body weight gain (or loss). In some embodiments, the body weight (or weight gain) is determined by monitoring cumulative body weight for at least 1 week, 2 weeks, 3 weeks, 4 weeks, 3 months, 6 months, 1 year, 2 years, 3 years, 4 years, or 5 years.

In some embodiments, BMI of the subject is increased after the administration, and BMI of the subject after the administration is at least 5% to 200% greater than that of the control subject. For example, BMI of the subject after the administration is at least 5% to 200%, at least 10% to 150%, at least 25% to 100%, or at least 50% to 75% greater than that of the control subject.

In some embodiments, BMI of the subject is increased after the administration, and BMI of the subject after the administration is at least 5% to 25% greater than that of the control subject. For example, BMI of the subject after the administration is at least 5% to 25%, at least 10% to 20%, or at least 12% to 15% greater than that of the control subject.

In some embodiments, BMI of the subject is increased after the administration, and BMI of the subject after the administration is at least 20% to 100% greater than that of the control subject. For example, BMI of the subject after the administration is at least 20% to 100%, at least 35% to 90%, at least 50% to 80%, or at least 60% to 70% greater than that of the control subject.

In some embodiments, BMI of the subject is increased between day 2 after the administration and day 7 of the administration, and BMI increase of the subject between days 2 and 7 after administration is at least 20% to 100% greater than that of the control subject. For example, increase in the subject's BMI between day 2 and day 7 after administration is at least 20% to 100%, at least 35% to 90%, at least 50% to 85%, or at least 60% to 80% % greater than that of the control subject.

In some embodiments, BMI of the subject is increased between day 2 after the administration and day 7 of the administration, and increase in the subject's BMI between days 2 and 7 after administration is at least 0.25, 0.3, 0.35, or 0.4 greater than that of the control subject.

In some embodiments, the increased BMI is maintained for at least 1 week, 2 weeks, 3 weeks, 1 month, 3 months, 6 months, 1 year, 2 years, 3 years, 4 years, or 5 years, after the administration is stopped.

In some embodiments, the BMI is determined by monitoring daily BMI. In some embodiments, the BMI is determined by monitoring daily changes in BMI. In some embodiments, the BMI is determined by monitoring daily weight and calculating BMI therefrom. In some embodiments, the BMI is determined by monitoring daily changes in weight and calculating the change in BMI therefrom.

In some embodiments, muscle mass of the subject is increased after the administration, and muscle mass gain of the subject after the administration is at least 1% to 100% greater than that of the control subject. For example, muscle mass gain of the subject after the administration is at least 1% to 100%, at least 5% to 75%, at least 10% to 50%, or at least 20% to 35% greater than that of the control subject.

In some embodiments, muscle mass of the subject is increased after the administration, and muscle mass gain of the subject after the administration is at least 5% to 15% greater than that of the control subject. For example, muscle mass gain of the subject after the administration is at least 5% to 15%, at least 6% to 14%, at least 7% to 12%, or at least 8% to 9% greater than that of the control subject.

In some embodiments, muscle mass of the subject is increased after the administration, and muscle mass gain of the subject after the administration is at least 25% to 50% greater than that of the control subject. For example, muscle mass gain of the subject after the administration is at least 25% to 50%, at least 28% to 48%, at least 25% to 45%, or at least 30% to 40% greater than that of the control subject.

In some embodiments, the increased muscle mass is maintained for at least 1 week, 2 weeks, 3 weeks, 1 month, 3 months, 6 months, 1 year, 2 years, 3 years, 4 years, or 5 years, after the administration is stopped.

In some embodiments, the muscle mass is cardiac muscle mass, skeletal muscle mass, or both. In some embodiments, the cardiac muscle mass is determined by measuring change in heart weight. In some embodiments, the skeletal muscle mass is determined by measuring change in gastrocnemius tissue weight. In related embodiments, the muscle mass change is cardiac muscle mass change, skeletal muscle mass change, or both. In some embodiments, cardiac muscle is determined by heart weight. In some embodiments, skeletal muscle mass is determined by gastrocnemius tissue weight. In some embodiments, the muscle mass changes can be assessed by using magnetic resonance imaging (MRI) and/or or nuclear magnetic resonance (NMR).

In some embodiments, fat mass of the subject is increased after the administration, and fat mass gain of the subject after the administration is at least 1% to 100% greater than that of the control subject. For example, fat mass gain of the subject after the administration is at least 1% to 100%, at least 5% to 75%, at least 10% to 50%, or at least 20% to 35% greater than that of the control subject.

In some embodiments, fat mass of the subject is increased after the administration, and fat mass gain of the subject after the administration is at least 5% to 15% greater than that of the control subject. For example, fat mass gain of the subject after the administration is at least 5% to 15%, at least 6% to 14%, at least 7% to 12%, or at least 8% to 9% greater than that of the control subject.

In some embodiments, fat mass of the subject is increased after the administration, and fat mass gain of the subject after the administration is at least 25% to 50% greater than that of the control subject. For example, fat mass gain of the subject after the administration is at least 25% to 50%, at least 28% to 48%, at least 25% to 45%, or at least 30% to 40% greater than that of the control subject.

In some embodiments, the increased fat mass is maintained for at least 1 week, 2 weeks, 3 weeks, 1 month, 3 months, 6 months, 1 year, 2 years, 3 years, 4 years, or 5 years, after the administration is stopped.

In some embodiments, the fat mass can be determined by the fat fold technique. In some embodiments, the fat mass changes can be assessed by using magnetic resonance imaging (MRI) and/or or nuclear magnetic resonance (NMR).

In some embodiments, the metabolically unchallenged subject experiences loss of appetite, reduced appetite, a loss of food consumption, and/or weight loss prior to the administration.

In some embodiments, body weight of the metabolically unchallenged subject is increased after the administration by at least 5% to 200%, at least 10% to 150%, at least 25% to 100%, or at least 50% to 75%, relative to body weight of the control subject before the administration, wherein the control subject is a metabolically unchallenged subject.

In some embodiments, the metabolically unchallenged subject and the control subject are the same subject.

In some embodiments, the metabolically unchallenged subject and the control subject are different subjects.

In some embodiments, body weight of the metabolically unchallenged subject is increased after the administration by at least about 5% to about 25%, at least about 10% to about 20%, or at least about 12% to about 15%, relative to body weight of the control subject before the administration, wherein the control subject is a metabolically unchallenged subject.

In some embodiments, body weight of the metabolically unchallenged subject is increased after the administration by at least about 20% to about 100%, at least about 35% to about 90%, at least about 50% to about 80%, or at least about 60% to about 70%, relative to body weight of the control subject before the administration, wherein the control subject is a metabolically unchallenged subject.

In some embodiments, body weight of the metabolically unchallenged subject is increased between day 2 after the administration and day 7 of the administration, and weight gain of the subject between days 2 and 7 after administration is at least 20% to 150%, 40% to 125%, 60% to 110%, or 80% to 100% relative to that of the control subject, wherein the control subject is a metabolically unchallenged subject.

In some embodiments, body weight of the metabolically unchallenged subject is increased between day 2 after the administration and day 7 of the administration, and weight gain of the subject between days 2 and 7 after administration is at least 0.5 kg, 0.75 kg, 1 kg, 1.25 kg, 1.5 kg, 1.75 kg, or 2 kg greater than that of the control subject, wherein the control subject is a metabolically unchallenged subject.

In some embodiments, body weight of the metabolically unchallenged subject is increased after the administration by at least 5% to 200%, at least 10% to 150%, at least 25% to 100%, or at least 50% to 75%, relative to body weight of the control subject, wherein the control subject is a metabolically unchallenged subject who experiences loss of appetite, reduced appetite, and/or weight loss.

In some embodiments, body weight of the metabolically unchallenged subject is increased after the administration by at least about 5% to about 25%, at least about 10% to about 20%, or at least about 12% to about 15%, relative to body weight of the control subject, wherein the control subject is a metabolically unchallenged subject who experiences loss of appetite, reduced appetite, and/or weight loss.

In some embodiments, body weight of the metabolically unchallenged subject is increased after the administration by at least about 20% to about 100%, at least about 35% to about 90%, at least about 50% to about 80%, or at least about 60% to about 70%, relative to body weight of the control subject, wherein the control subject is a metabolically unchallenged subject who experiences loss of appetite, reduced appetite, and/or weight loss.

In some embodiments, body weight of the metabolically unchallenged subject is increased between day 2 after the administration and day 7 of the administration, and weight gain of the subject between days 2 and 7 after administration is at least 20% to 150%, 40% to 125%, 60% to 110%, or 80% to 100% relative to that of the control subject, wherein the control subject is a metabolically unchallenged subject who experiences loss of appetite, reduced appetite, and/or weight loss.

In some embodiments, body weight of the metabolically unchallenged subject is increased between day 2 after the administration and day 7 of the administration, and weight gain of the subject between days 2 and 7 after administration is at least 0.5 kg, 0.75 kg, 1 kg, 1.25 kg, 1.5 kg, 1.75 kg, or 2 kg greater than that of the control subject, wherein the control subject is a metabolically unchallenged subject who experiences loss of appetite, reduced appetite, and/or weight loss.

In some embodiments, the increased body weight of the metabolically unchallenged subject is maintained for at least 1 week, 2 weeks, 3 weeks, 1 month, 3 months, 6 months, 1 year, or 5 years, after the administration is stopped.

In some embodiments, BMI of the metabolically unchallenged subject is increased after the administration by at least 5% to 200%, at least 10% to 150%, at least 25% to 100%, or at least 50% to 75%, relative to BMI of the control subject before the administration, wherein the control subject is a metabolically unchallenged subject.

In some embodiments, BMI of the metabolically unchallenged subject is increased after the administration by at least about 5% to about 25%, at least about 10% to about 20%, or at least about 12% to about 15%, relative to BMI of the control subject before the administration, wherein the control subject is a metabolically unchallenged subject.

In some embodiments, BMI of the metabolically unchallenged subject is increased after the administration by at least about 20% to about 100%, at least about 35% to about 90%, at least about 50% to about 80%, or at least about 60% to about 70%, relative to BMI of the control subject before the administration, wherein the control subject is a metabolically unchallenged subject.

In some embodiments, BMI of the metabolically unchallenged subject is increased between day 2 after the administration and day 7 of the administration, and BMI increase of the subject between days 2 and 7 after administration is at least 20% to 100%, at least 35% to 90%, at least 50% to 85%, or at least 60% to 80% greater than that of the control subject, wherein the control subject is a metabolically unchallenged subject.

In some embodiments, BMI of the metabolically unchallenged subject is increased between day 2 after the administration and day 7 of the administration, and increase in the subject's BMI between days 2 and 7 after administration is at least 0.25, 0.3, 0.35, or 0.4 greater than that of the control subject, wherein the control subject is a metabolically unchallenged subject.

In some embodiments, the increased BMI of the metabolically unchallenged subject is maintained for at least 1 week, 2 weeks, 3 weeks, 1 month, 3 months, 6 months, 1 year, or 5 years, after the administration is stopped.

In some embodiments, BMI of the metabolically unchallenged subject is increased after the administration by at least 5% to 200%, at least 10% to 150%, at least 25% to 100%, or at least 50% to 75%, relative to BMI of the control subject, wherein the control subject is a metabolically unchallenged subject who experiences loss of appetite, reduced appetite, and/or weight loss.

In some embodiments, BMI of the metabolically unchallenged subject is increased after the administration by at least about 5% to about 25%, at least about 10% to about 20%, or at least about 12% to about 15%, relative to BMI of the control subject, wherein the control subject is a metabolically unchallenged subject who experiences loss of appetite, reduced appetite, and/or weight loss.

In some embodiments, BMI of the metabolically unchallenged subject is increased after the administration by at least about 20% to about 100%, at least about 35% to about 90%, at least about 50% to about 80%, or at least about 60% to about 70%, relative to BMI of the control subject, wherein the control subject is a metabolically unchallenged subject who experiences loss of appetite, reduced appetite, and/or weight loss.

In some embodiments, BMI of the metabolically unchallenged subject is increased between day 2 after the administration and day 7 of the administration, and BMI increase of the subject between days 2 and 7 after administration is at least 20% to 100%, at least 35% to 90%, at least 50% to 85%, or at least 60% to 80% greater than that of the control subject, wherein the control subject is a metabolically unchallenged subject, wherein the control subject is a metabolically unchallenged subject who experiences loss of appetite, reduced appetite, and/or weight loss.

In some embodiments, BMI of the metabolically unchallenged subject is increased between day 2 after the administration and day 7 of the administration, and increase in the subject's BMI between days 2 and 7 after administration is at least 0.25, 0.3, 0.35, or 0.4 greater than that of the control subject, wherein the control subject is a metabolically unchallenged subject who experiences loss of appetite, reduced appetite, and/or weight loss.

In some embodiments, the increased BMI of the metabolically unchallenged subject is maintained for at least 1 week, 2 weeks, 3 weeks, 1 month, 3 months, 6 months, 1 year, or 5 years, after the administration is stopped.

In some embodiments, food consumption of the metabolically unchallenged subject is increased after the administration by at least 50% to 2,000%, at least 75% to 1,500%, at least 100% to 1,000%, at least 250% to 750%, or at least 400% to 500%, relative to food consumption of the metabolically unchallenged subject before the administration.

In some embodiments, food consumption of the metabolically unchallenged subject is increased after the administration by at least about 100%, relative to food consumption of the metabolically unchallenged subject before the administration.

In some embodiments, food consumption of the metabolically unchallenged subject is increased after the administration by at least about 1,000%, relative to food consumption of the metabolically unchallenged subject before the administration.

In some embodiments, food consumption of the metabolically unchallenged subject is increased after the administration by at least 50% to 2,000%, at least 75% to 1,500%, at least 100% to 1,000%, at least 250% to 750%, or at least 400% to 500%, relative to food consumption of the control subject, wherein the control subject is a metabolically unchallenged subject who experiences loss of appetite, reduced appetite, and/or weight loss.

In some embodiments, food consumption is increased for at least 1 day, 1 week, 1 month, 3 months, 6 months, 1 year, or 5 years during the administration.

In some embodiments, food consumption is increased for at least 1 day, 7 days, 14 days, 21 days, 28 days, 35 days, 40 days, 45 days, 50 days, 60 days, 75 days, 90 days, 100 days, 110 days, or 120 days during the administration.

In some embodiments, the increased food consumption is maintained for at least 1 week, 2 weeks, 3 weeks, 1 month, 3 months, 6 months, 1 year, or 5 years, after the administration is stopped.

In some embodiments, food consumption is determined by total calories (kcal) consumed in 1 day or mass of food (grams or kilograms) consumed in 1 day.

In some embodiments, one or more side effects exhibited by the metabolically unchallenged subject after administration of the non-naturally occurring melanocortin analog are reduced compared to one or more side effects exhibited by a control subject that was administered a natural melanocortin.

In some embodiments, the control subject is a metabolically unchallenged subject.

In some embodiments, the muscle mass is skeletal muscle mass, cardiac muscle mass, or both.

In some embodiments, the increased body weight or BMI comprises increased muscle mass, increased fat mass, or both.

### Combination Therapies

In some embodiments, the subject has a cancer (e.g., as above specified), and the method of the present technology further comprises administering an anticancer agent to the subject. In some embodiments, the anticancer agent is at least one chemotherapeutic agent.

In some embodiments, the at least one chemotherapeutic agent comprises one or more chemotherapeutic agents selected from the group consisting of a platinum-coordination complex, an antimetabolite, a tubulin binding agent, an alkylating antineoplastic agent, and a cytotoxic antibiotic. In some embodiments, the chemotherapy agent comprises a platinum-coordination complex. In some embodiments, the chemotherapy agent comprises an antimetabolite. In some embodiments, the chemotherapy agent comprises a tubulin binding agent. In some embodiments, the chemotherapy agent comprises an alkylating antineoplastic agent. In some embodiments, the chemotherapy agent comprises a cytotoxic antibiotic.

In some embodiments, the platinum-coordination complex is cisplatin.

In some embodiments, the antimetabolite is 5-fluorouracil (5-FU).

In some embodiments, the tubulin binding agent is vincristine.

In some embodiments, the alkylating antineoplastic agent is cyclophosphamide. In some embodiments, the cytotoxic antibiotic is doxorubicin.

In some embodiments, the administration in the method disclosed herein reduces or prevents one or more adverse side effects, such as deleterious, undesirable, or unwanted side effects associated with cancer or anticancer agent. Such side effects include, but are not limited to, cancer cachexia, chemotherapy-induced cachexia, anorexia (e.g., decreasing food intake, decreasing appetite), nausea, emesis, weight loss (e.g., decreasing body weight or BMI, increasing weight loss), fatigue, fat mass loss (e.g., deceasing fat mass, increasing loss of fat mass), and muscle mass loss (e.g., decreasing muscle mass, increasing loss of muscle mass).

In some embodiments, the anticancer agent (e.g., chemotherapeutic agent) and the non-naturally occurring melanocortin analog are administered simultaneously as a single composition. In some embodiments, the anticancer agent and the non-naturally occurring melanocortin analog are present in different pharmaceutical compositions and are administered sequentially. In some embodiments, the anticancer agent and the non-naturally occurring melanocortin analog are present in different pharmaceutical compositions and are administered simultaneously (i.e., concurrently) but separately.

In some embodiments, the method of the present technology further comprises administering an appetite-regulating agent to the subject. Non-limiting examples of appetite-regulating agent include neuropeptide Y (NPY), agouti-related protein (AgRP), Ghrelin, growth hormone GH (e.g., human growth hormone (hGH)), insulin-like growth factor 1 (IGF-1), insulin-like growth factor (IGF) binding protein-3 (IGFBP-3), and alkaline phosphatase (ALP).

In some embodiments, the method of the present technology prevents or reduces fatigue in the subject. In some embodiments, fatigue is determined by monitoring locomotor activity. The method of the present technology can further facilitate maintenance of physical functioning and/or facilitate recovery of physical function in e.g., subjects recovering from major surgeries including, but not limited to, open heart surgery, insertion of a shoulder prosthesis, and amputations.

### Examples

The following examples are intended to illustrate various embodiments of the present technology. As such, the specific embodiments discussed are not to be construed as limitations on the scope of the present technology. It will be apparent to one skilled in the art that various equivalents, changes, and modifications may be made without departing from the scope of present technology, and it is understood that such equivalent embodiments, are to be included herein. Further, all references cited herein are hereby incorporated by reference in their entirety, as if fully set forth herein.

### Example 1: Peptide Synthesis-Generic

The non-naturally occurring melanocortin analogs of the present technology were synthesized by conventional procedures (e.g., solution-phase procedure, solid-phase synthesis) for the formation of a peptide linkage between amino acids. The solution-phase procedure involved a condensation between the free alpha amino group of an amino acid or derivative thereof having the carboxyl group or other reactive groups protected and the free primary carboxyl group of another amino acid or derivative thereof having the amino group or other reactive groups protected. The solid-phase synthesis utilized a variety of resins and reagents and may involve additional purification steps.

The process for synthesizing the non-naturally occurring melanocortin analogs was generally performed by a procedure as follows. Each amino acid in the desired sequence of the non-naturally occurring melanocortin analogs was added one at a time in succession to another amino acid or derivative thereof or by a procedure whereby peptide fragments with the desired amino acid sequence were first synthesized conventionally and then condensed to provide the desired peptide. In most cases, the resulting peptide was then cyclized to yield a cyclic peptide.

Solid-phase peptide synthesis was carried out by sequentially incorporating the desired amino acid residues one at a time into the growing peptide chain coupled to a solid-phase support according to the general principles of solid phase methods (see Merrifield, Angew Chem. 24:799-810 (1985) and Barany et al., The Peptides, Analysis, Synthesis and Biology, Vol. 2, Gross E. and Meienhofer J., Eds. Academic Press 1-284 (1980)). An exemplary solid-phase synthesis of non-naturally occurring melanocortin analogs is provided below.

Initially, the C-terminal amino acid residue of the non-naturally occurring melanocortin analog was coupled to a solid-phase support, e.g., a solid-phase resin. Coupling of the C-terminal amino acid residue and the solid-phase support may be carried out according to any method know in the art. Depending on the coupling method, the alpha-amine of the C-terminal amino acid residue may or may not be protected with an amine protecting group, as described below. Likewise, the carboxyl group of the amino acid residue may or may not be activated prior to coupling to the solid-phase support in order to increase its electrophilicity. Some methods of coupling rely on the formation of an ester bond between the carboxyl group of the amino acid and a reactive handle on the solid-phase resin. For example, an amino acid residue may be coupled to a p-benzyloxybenzyl alcohol resin (Wang) or a 2-chlorotrityl chloride resin via an ester linkage. Some methods of coupling rely on the formation of an aminde bond between the carboxyl group of the amino acid and a reactive handle on the solid-phase resin For example, an amino acid residue may be coupled to a benzhydrylamine (BHA) resin through an Fmoc-linker such as, for example, p-[(R,S)-α-[1-(9H-fluor-en-9-yl)-methoxyformamido]-2,4-dimethyloxybenzyl]-phenoxyacetic acid (Rink linker) via an amide linkage.

The non-naturally occurring melanocortin analog was then synthesized by sequential amino acid addition or combination of peptide fragments. Subsequently, the peptide was cleaved from the solid-phase support and purified by methods known in the art, such as, for example, reverse phase high performance liquid chromatography (RP-HPLC) using a suitable column, such as a C18 column. Additionally, or alternatively, other methods of separation or purification were employed, including, but not limited to, methods based on the size or charge of the peptide. Once purified, the peptide was characterized by methods such as high-performance liquid chromatograph (HPLC), amino acid analysis, mass spectrometry, and the like.

### Example 2: Peptide Synthesis-Protecting Groups

During synthesis of the non-naturally occurring melanocortin analogs, reactive side chain groups of the various amino acid residues were protected with suitable protecting groups, which prevented undesirable chemical reaction from occurring at that site until the protecting group was removed.

Additionally, protection of the alpha amino group of an amino acid residue or fragment was performed while that entity reacting with the carboxyl group, followed by the selective removal of the alpha amino protecting group to allow a subsequent reaction to take place at that site. Specific protecting groups for solid phase synthesis methods and solution phase synthesis methods are known to those having ordinary skill in the art. Alpha amino groups were protected by a suitable protecting group, including a urethane-type protecting group, such as benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz); aliphatic urethane-type protecting groups, such as t-butyloxycarbonyl (Boc), diisopropylmethoxycarbonyl, isopropoxycarbonyl, and allyloxycarbonyl. Fmoc was also used for alpha amino protection. Guanidino groups, if present, were protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf) and Boc. Pmc was used as a protecting group for Arg.

Alpha aminoprotecting groups may be removed under basic conditions, such as, for example, using a solution of piperidine, piperazine, diethylamine, or morpholine (20-40% v/v) in N,N-dimethylformamide (DMF). In synthesis methods in which alpha amino protecting groups were used, protecting groups were removed after synthesis of the peptide and before or after cleavage of the solid-phase support.

### Example 3: Peptide Synthesis-Additional Modifications

If necessary, the peptides were further modified to obtain N-terminus modifications, such as acetylation, while on resin, or were removed from the resin by use of a cleaving reagent and then modified. Likewise, C-terminus modification (e.g., amidation), was performed if needed.

Additionally, the cyclized peptide structures were obtained prior to cleavage from the peptide resin. For cyclization through reactive side chain moieties, the desired side chains were deprotected, and the peptide suspended in a suitable solvent and a cyclic coupling agent added. Suitable solvents, for example DMF, dichloromethane (DCM) or 1-methyl-2-pyrrolidone (NMP), were used for the cyclization. Suitable cyclic coupling reagents (e.g., 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris (dimethylamino)phosphoniumhexafluorophosphate (BOP), benzotriazole-1-yl-oxy-tris (pyrrolidino)phosphoniumhexafluorophosphate (PyBOP), 2-(7-aza-1H-benzotriazol-1-yl)-1,1,3,3-tetramethyluronium tetrafluoroborate (TATU), 2-(2-oxo-1 (2H)-pyridyl)-1,1,3,3-tetramethyluronium tetrafluoroborate (TPTU), N,N′-dicyclohexylcarbodiimide/1-hydroxybenzotriazole (DCCI/HOBt)) were also used for the cyclization. Coupling was initiated by a suitable base, such as N,N-diispropylethylamine (DIPEA), sym-collidine or N-methylmorpholine (NMM).

### Example 4: Biological Data

The antagonist activity of exemplary non-naturally occurring melanocortin analogs (SEQ ID NOs: 3, 4, 6, 9-11, 14, 15, and 291-309) at the melanocortin 3 receptor (MC3R) and melanocortin 4 (MC4R) receptor was measured via cAMP accumulation assay, according to the following procedure.

Recombinant cell lines shown in Table 1 were stimulated with melanocortin receptor antagonists and evaluated for cyclic adenosine monophosphate (cAMP) levels as a measurement of receptor activation. cAMP levels were quantified using Homogeneous Time-Resolved Fluorescence (HTRF). The stimulation and CAMP measurement were performed sequentially. First, the compound stimulated the G-protein coupled receptor (GPCR) which then induced downstream signaling, which was measured soon after. Cell lines and stimulated receptors used are detailed in Table 1.

TABLE 1
Cell lines

| Cell Line | Receptor |
|---|---|
| HEK293/CRE-luc/MC3R | MC3R |
| HEK293/MC4R/CRE-GFP | MC4R |

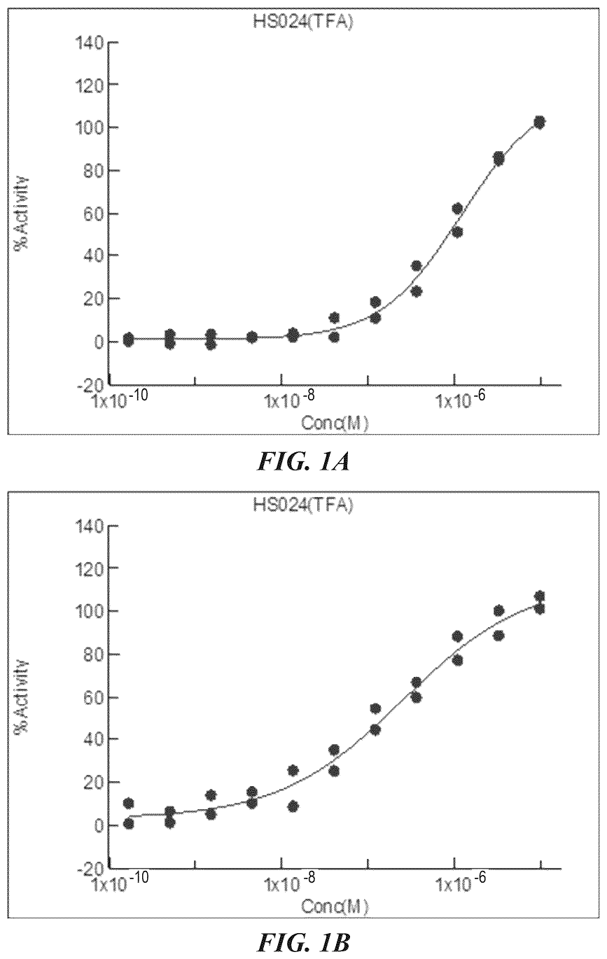

Twenty-seven exemplary synthetic peptides were tested for antagonist activity at the human MC3R and MC4R at concentrations ranging from 0.1 nM to 1,000 nM. After stimulation, the level of cyclic adenosine monophosphate (CAMP) in each well was quantified via Homogenous Time-Resolved Fluorescence (HTRF) as a measurement of receptor activity. Dose-response curves and estimated IC₅₀ values were then obtained for each antagonist peptide against the MC3R and MC4R. HS024 (TFA) and was used as a positive control for the antagonist activity of MC3R (FIG. 1A) and MC4R (FIG. 1B).

### Synthetic Peptide Grouping

Synthetic peptides were grouped based on structural similarities. Group A included synthetic peptides A1 and A2, which are linear peptides comprising the motif Pro-Dnal(2′)-Arg-Trp. Group A peptides are provided in Table 2.

TABLE 2
Group A synthetic peptides

| SEQ ID | Synthetic | |
|---|---|---|
| NO | Peptide | Formula |
| 14 | A1 | Ac-Nle-Asp-Pro-Dnal(2′)-Arg-Trp-Lys-Dval- |
| | | Dpro-NH₂ |
| 15 | A2 | Ac-Nle-Pro-Dnal(2′)-Arg-Trp-Dval-Dpro-NH₂ |

Group B included synthetic peptides B1 to B9, which are cyclic and linear peptides comprising the motif Pro-Phe-Phe-Arg-Trp-Lys (SEQ ID NO: 393). In particular, the cyclic peptides, B1-B3, comprise the cyclic motif c(Asp-Pro-Phe-Phe-Arg-Trp-Lys) (SEQ ID NO: 394). Linear peptides B4-B9 differ from each other by N-terminal and/or C-terminal groups. Group B peptides are provided in Table 3.

TABLE 3
Group B synthetic peptides

| SEQ ID | Synthetic | |
|---|---|---|
| NO | Peptide | Formula |
| 291 | B1 | Ac-Nle-c(Asp-Pro-Phe-Phe-Arg-Trp-Lys)-Dval- |
| | | Dpro-NH₂ |
| 292 | B2 | Ac-Leu-c(Asp-Pro-Phe-Phe-Arg-Trp-Lys)-Val- |
| | | Pro-NH₂ |
| 293 | B3 | Leu-c(Asp-Pro-Phe-Phe-Arg-Trp-Lys)-Val-Pro |
| 294 | B4 | Leu-Pro-Phe-Phe-Arg-Trp-Val-Pro |
| 295 | B5 | Ile-Pro-Phe-Phe-Arg-Trp-Val-Pro |
| 296 | B6 | Val-Pro-Phe-Phe-Arg-Trp-Val-Pro |
| 297 | B7 | Leu-Asp-Pro-Phe-Phe-Arg-Trp-Lys-Val-Pro |
| 298 | B8 | Ile-Asp-Pro-Phe-Phe-Arg-Trp-Lys-Val-Pro |
| 299 | B9 | Val-Asp-Pro-Phe-Phe-Arg-Trp-Lys-Val-Pro |

Group C included synthetic peptides C1 to C9, which are cyclic and linear peptides comprising the motif Pro-Trp-Arg-Trp-Lys (SEQ ID NO: 395) or a derivative thereof. In particular, the cyclic peptides, C1-C3, comprise the cyclic motif c(Asp-Pro-Trp-Arg-Trp-Lys) (SEQ ID NO: 396). Linear peptides C4-C9 comprise the motif Pro-Trp-Arg-Trp-Lys (SEQ ID NO: 395) or Pro-Trp-Arg-Trp-Val (SEQ ID NO: 397). Group C peptides are provided in Table 4.

TABLE 4
Group C synthetic peptides

| SEQ ID | Synthetic | |
|---|---|---|
| NO | Peptide | Formula |
| 300 | C1 | Ac-Nle-c(Asp-Pro-Trp-Arg-Trp-Lys)-Dval- |
| | | Dpro-NH₂ |
| 301 | C2 | Ac-Leu-c(Asp-Pro-Trp-Arg-Trp-Lys)-Val- |
| | | Pro-NH₂ |
| 302 | C3 | Leu-c(Asp-Pro-Trp-Arg-Trp-Lys)-Val-Pro |
| 303 | C4 | Leu-Pro-Trp-Arg-Trp-Val-Pro |
| 304 | C5 | Ile-Pro-Trp-Arg-Trp-Val-Pro |
| 305 | C6 | Val-Pro-Trp-Arg-Trp-Val-Pro |
| 306 | C7 | Leu-Asp-Pro-Trp-Arg-Trp-Lys-Val-Pro |
| 307 | C8 | Ile-Asp-Pro-Trp-Arg-Trp-Lys-Val-Pro |
| 308 | C9 | Val-Asp-Pro-Trp-Arg-Trp-Lys-Val-Pro |

Group D included synthetic peptides D1 to D5, and known MC3R/MC4R antagonists B02 and B07, all of which are cyclic peptides comprising the motif c(Asp-Pro-His-Dnal(2′)-Arg-Trp-Lys) (SEQ ID NO: 398) or a derivative thereof. The synthetic peptides in Group D are derived from known MC3R/MC4R antagonist B07. Group D synthetic peptides are provided in Table 5.

TABLE 5
Group D synthetic peptides

| SEQ ID | Synthetic | |
|---|---|---|
| NO | Peptide | Formula |
| 6 | D1 | Ac-Nle-_{c}(Asp-Pro-His-Dnal(2′)-Arg-Trp-Lys)-Dval-Dpro-NH₂ |
| 9 | D2 | Ac-Nle-_{c}(Asp-His-Dnal(2′)-Arg-Trp-Pro-Lys)-Dval-Dpro-NH₂ |
| 4 | D3 | Ac-Nle-_{c}(Asp-Hyp-Dnal(2′)-Arg-Trp-Lys)-Dval-Dpro-NH₂ |
| 10 | D4 | Ac-Nle-_{c}(Asp-Hyp-Dnal(2′)-Arg-Trp-Pro-Lys)-Dval-Dpro-NH₂ |
| 11 | D5 | Ac-Nle-_{c}(Asp-Pro-His-Dnal(2′)-Arg-Trp-Lys)-Dpro-Dval-NH₂ |
| 309 | TCMCB02 | Ac-Nle-c[Asp-Pro-dNal(2′)-Arg-Trp-Lys]-dPro-dVal-NH₂ |
| 3 | TCMCB07 | Ac-Nle-c[Asp-Pro-dNal(2′)-Arg-Trp-Lys]-dVal-dPro-NH₂ |

### Antagonist Activity of Non-Naturally Occurring Melanocortin Analogs on MC3R

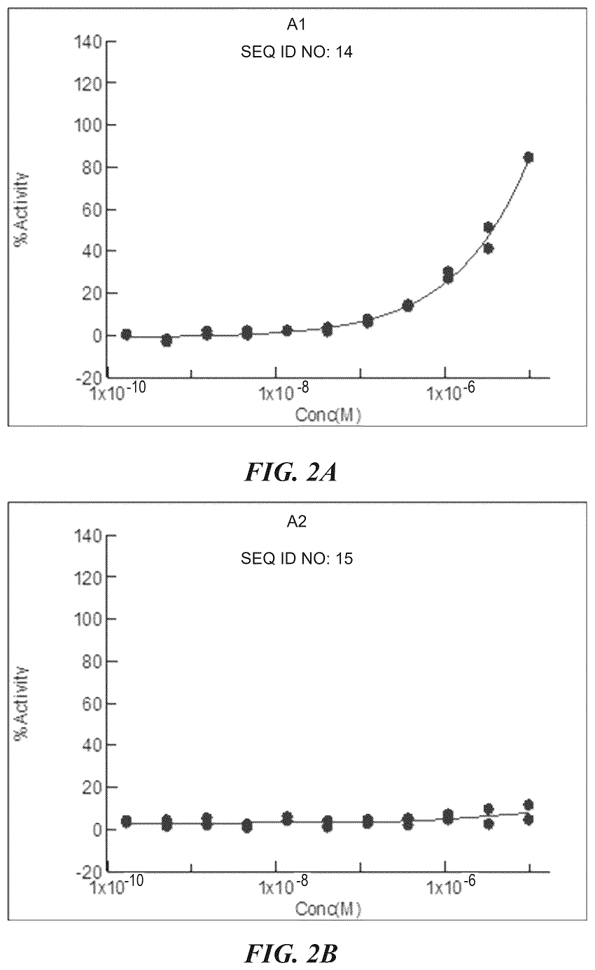

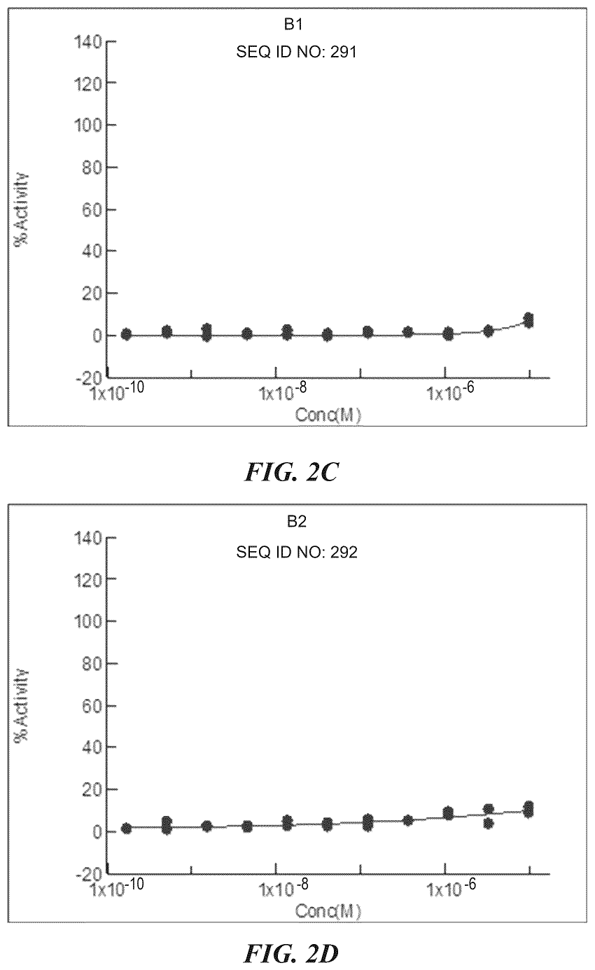

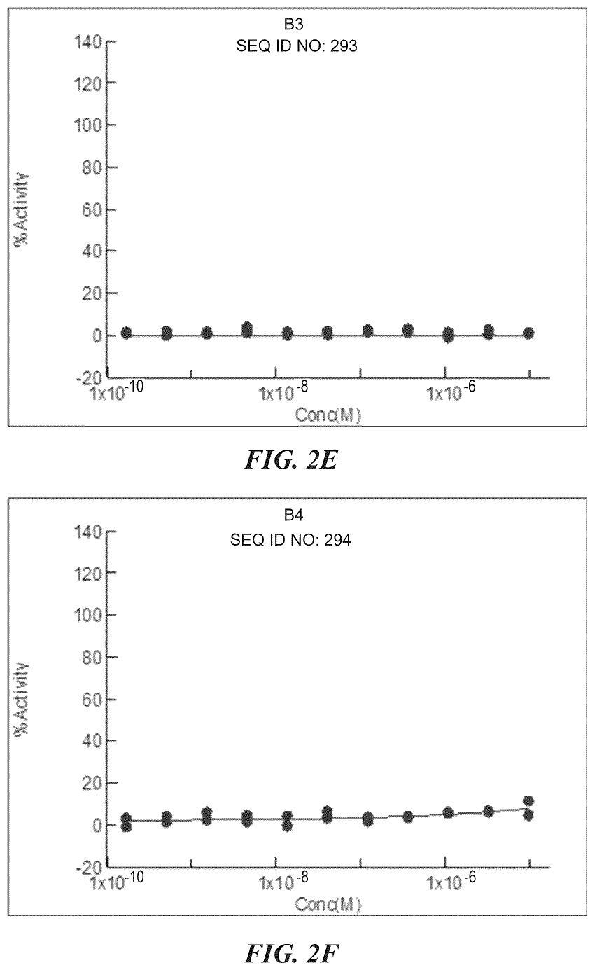

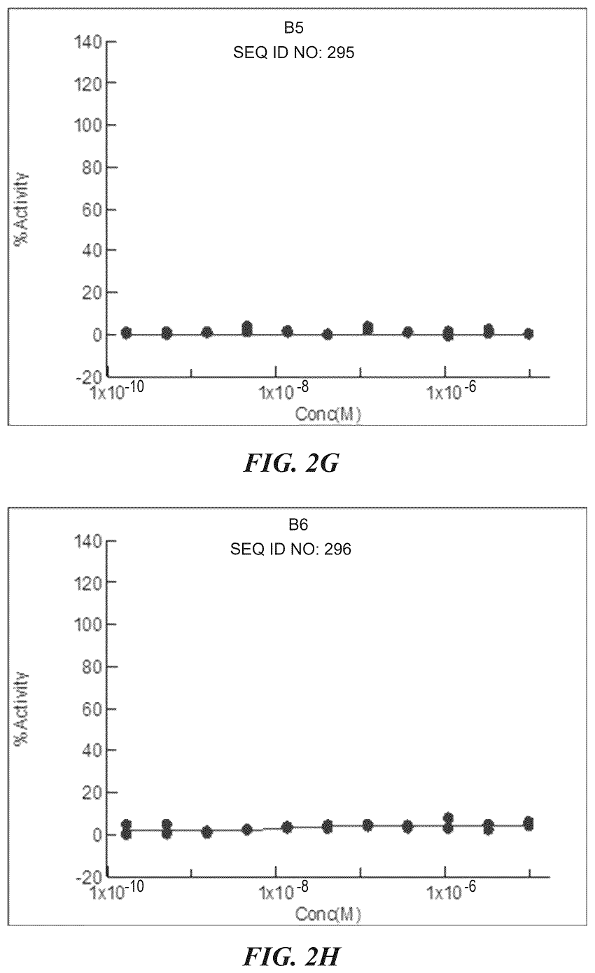

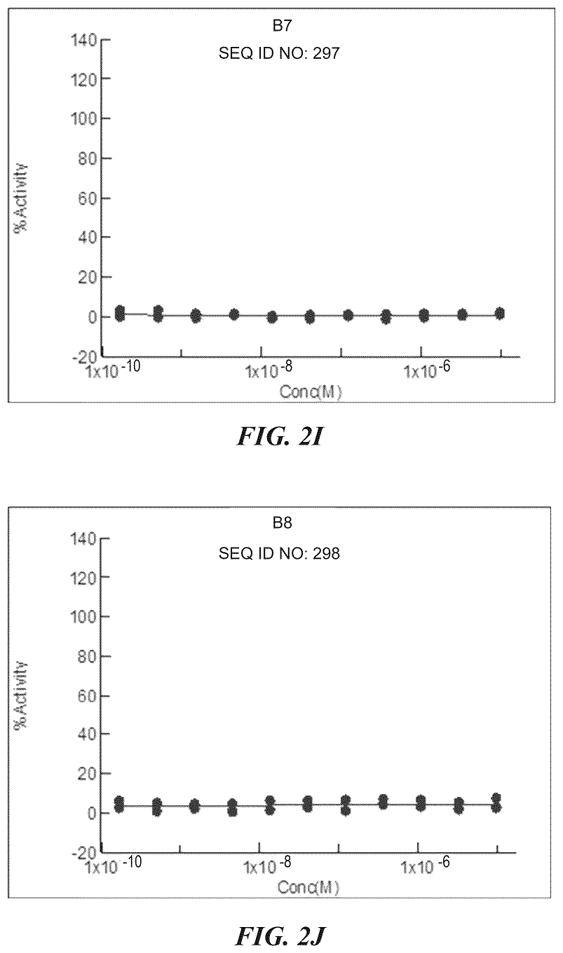

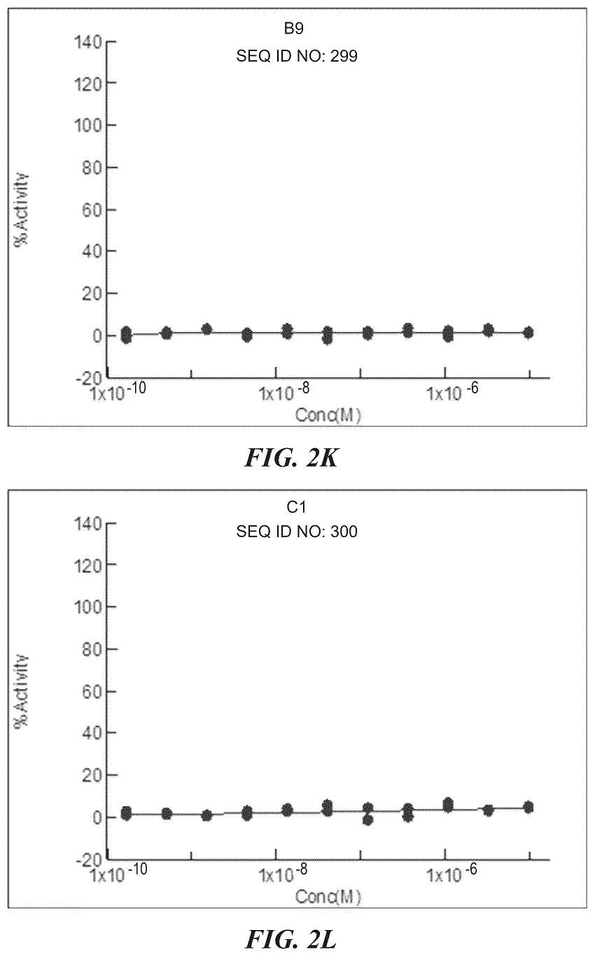

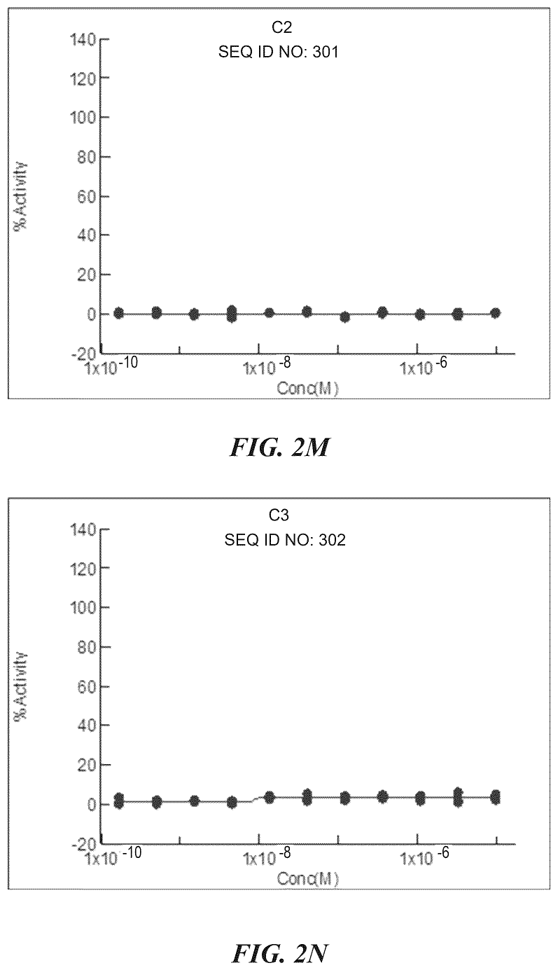

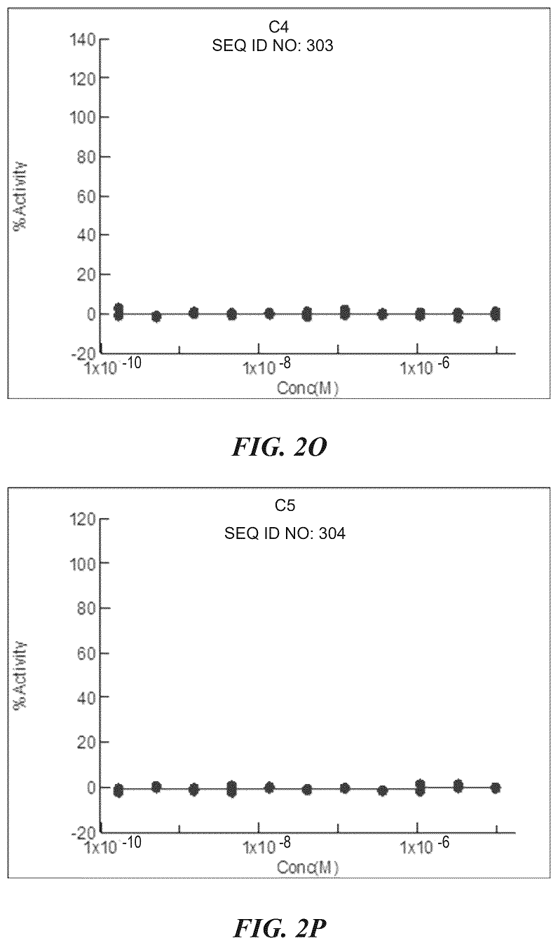

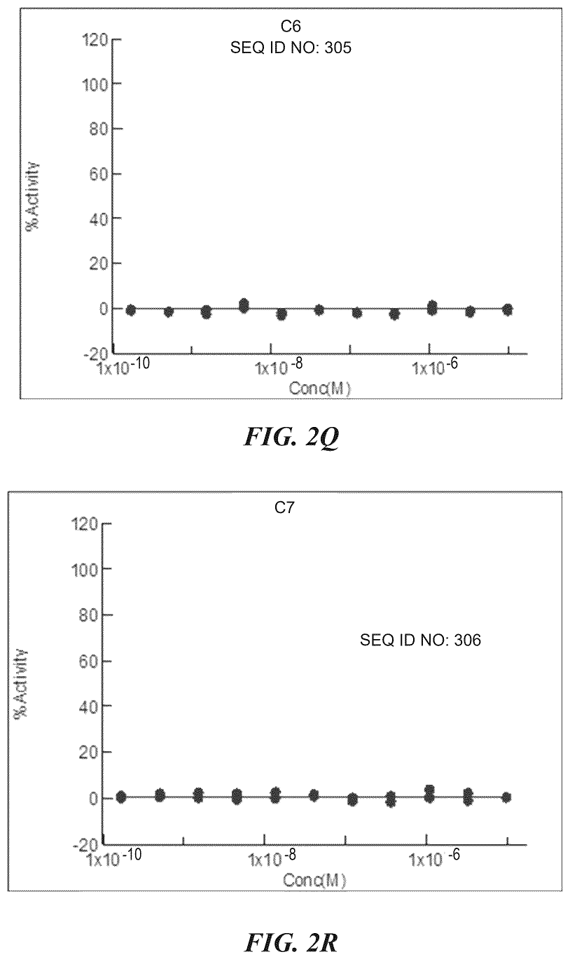

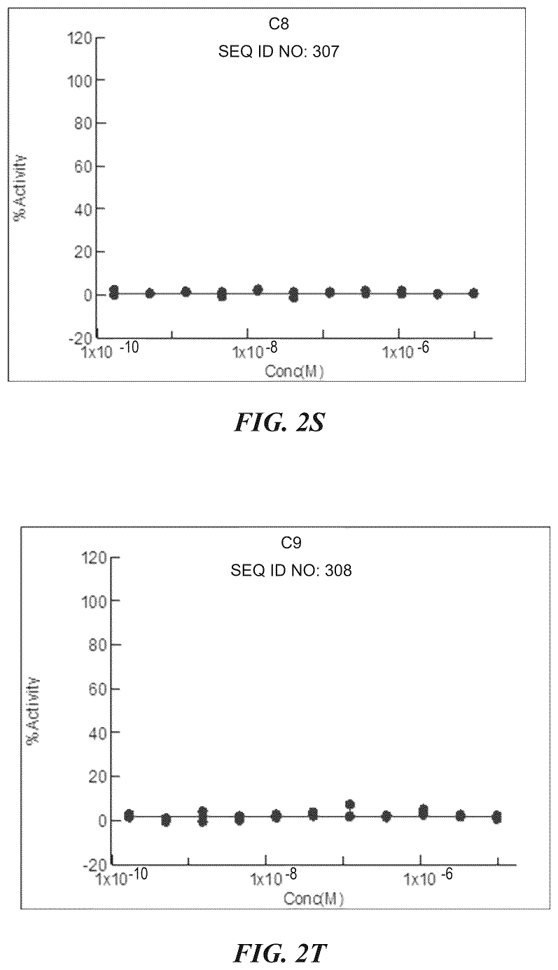

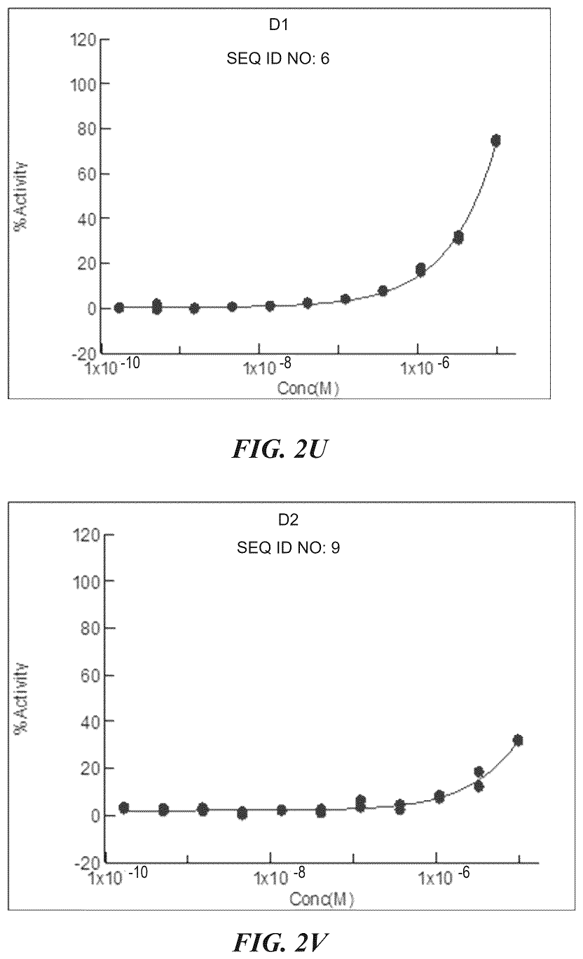

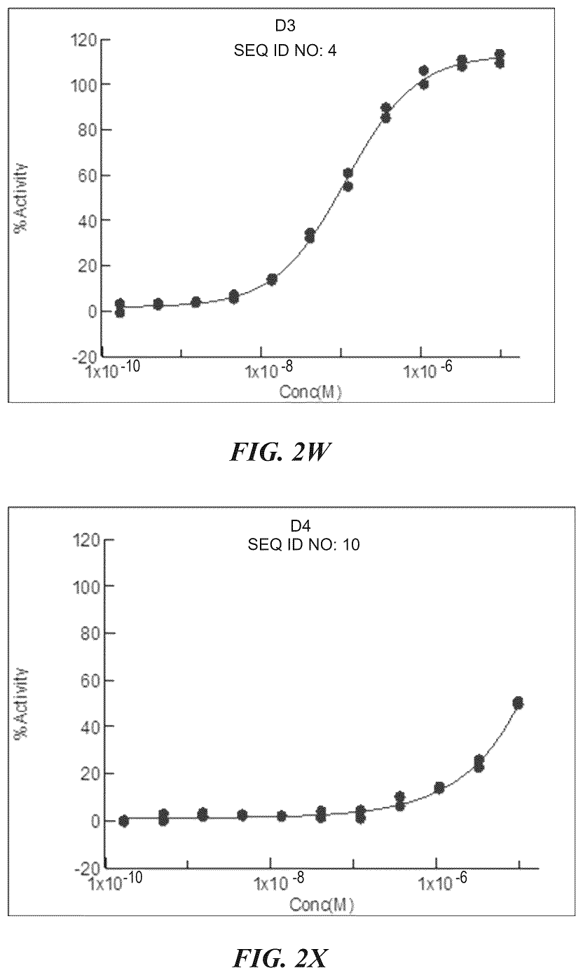

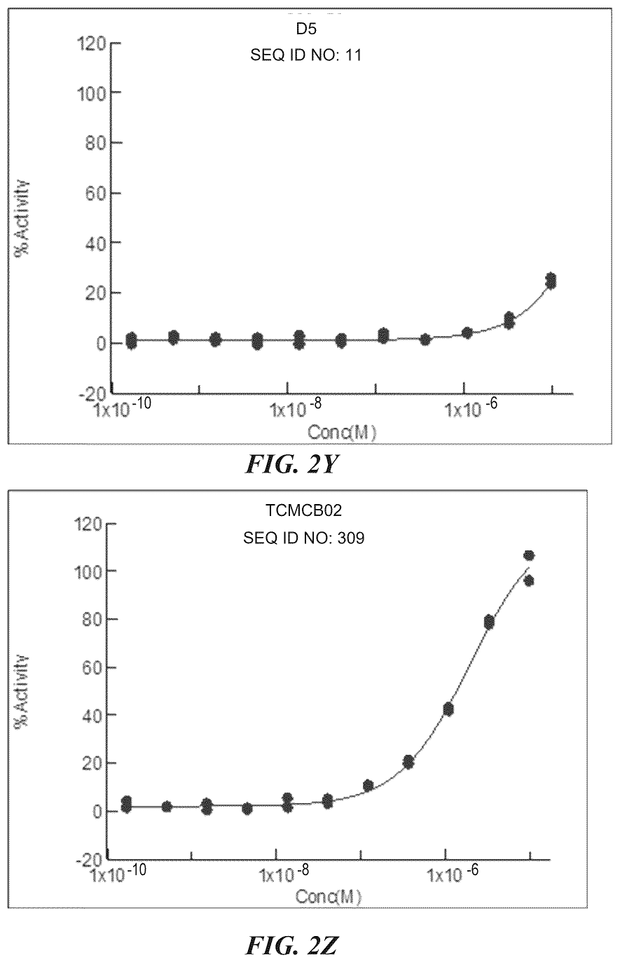

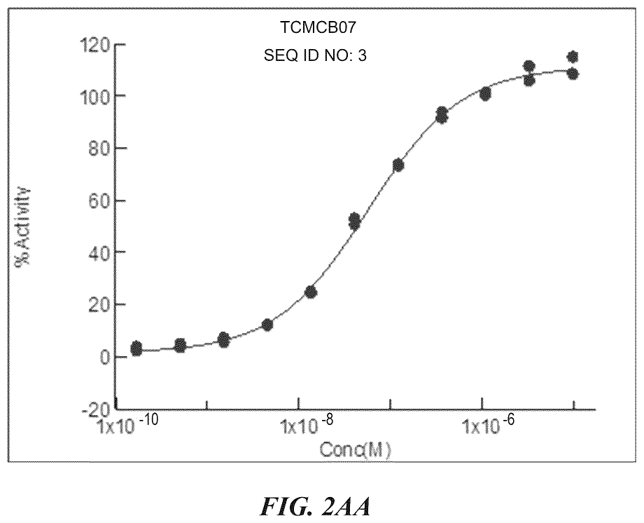

Administration of some non-naturally occurring melanocortin analogs inhibited MC3R activity, as measured by cAMP levels. Among the non-naturally occurring melanocortin analogs (FIG. 2A-AA), A1 (B07L), D1, D3, TCMCB02, and TCMCB07 all had IC₅₀ values under 10000 nM, with TCMCB07 and D3 having the lowest IC₅₀ at 43.42 nM and 87.00 nM, respectively (Table 6).

TABLE 6
Dose-response results of melanocortin
analogs and control against MC3R

| Synthetic | Max | | Hill | |
|---|---|---|---|---|
| Peptide | Activation % | IC₅₀ (nM)* | Coefficient | Top (%) |
| HS024(TFA) | 102.29 | 867.28 | 0.93 | 117.07 |
| A1 | 84.42 | 3659.81 | 0.55 | 1002.64 |
| A2 | 7.92 | nd | 0.82 | 9.73 |
| B1 | 6.86 | nd | 1.05 | — |
| B2 | 10.28 | nd | 0.34 | 20.30 |
| B3 | 0.83 | nd | 1.00 | 100.00 |
| B4 | 7.81 | nd | 0.36 | 29.62 |
| B5 | 0.14 | nd | −0.76 | 736.17 |
| B6 | 5.00 | nd | 1.62 | 4.27 |
| B7 | 1.43 | nd | −0.84 | 7754.77 |
| B8 | 4.86 | nd | 12.04 | 4.40 |
| B9 | 1.08 | nd | 2.26 | 1.22 |
| C1 | 4.55 | nd | 0.23 | 6.49 |
| C2 | 0.27 | nd | −0.48 | 0.42 |
| C3 | 3.32 | nd | 49.34 | 3.18 |
| C4 | −0.32 | nd | −5.34 | — |
| C5 | −0.49 | nd | 13.82 | −0.10 |
| C6 | −0.80 | nd | 1.00 | 100.00 |
| C7 | 0.16 | nd | −0.13 | 1.86 |
| C8 | 0.47 | nd | −57.42 | 0.80 |
| C9 | 1.24 | nd | 2.95 | −64.92 |
| D1 | 74.48 | 5805.09 | 0.73 | 4952.72 |
| D2 | 31.76 | nd | 0.84 | 177.39 |
| D3 | 111.24 | 87.00 | 0.98 | 113.20 |
| D4 | 50.10 | >10000 | 0.65 | 2741.58 |
| D5 | 24.60 | >10000 | 1.04 | 472.61 |
| TCMCB02 | 101.16 | 1355.79 | 1.03 | 120.45 |
| TCMCB07 | 111.61 | 43.42 | 0.85 | 111.40 |

*nd = not determined

### Antagonist Activity of Non-Naturally Occurring Melanocortin Analogs on MC4R

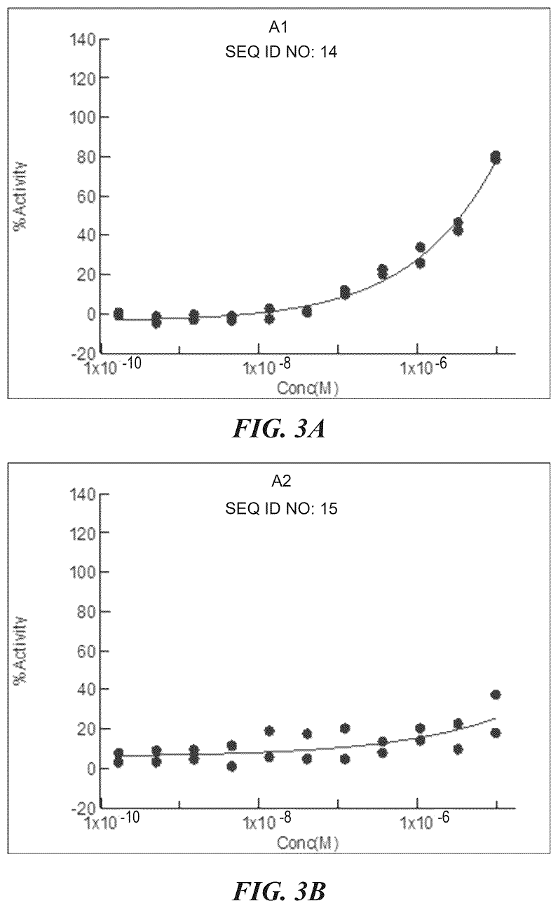

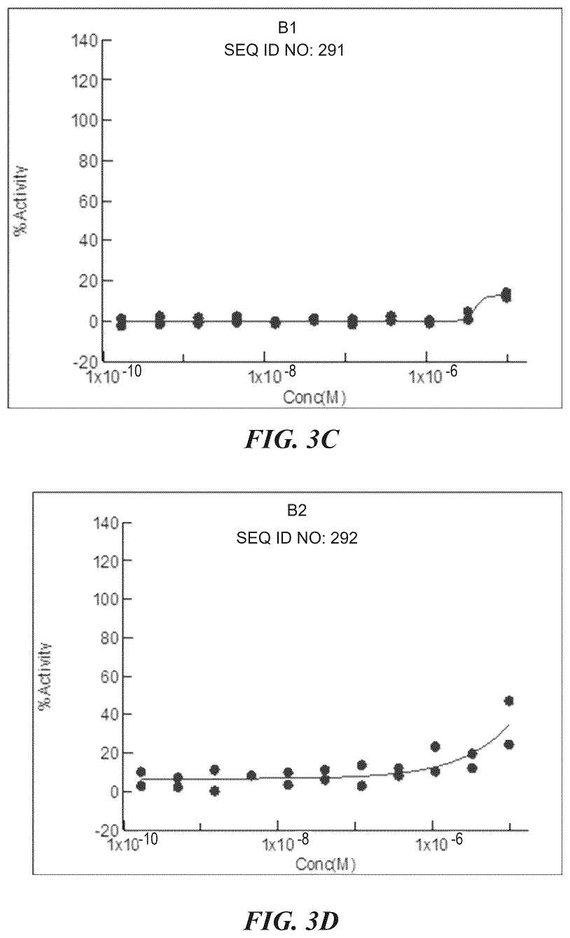

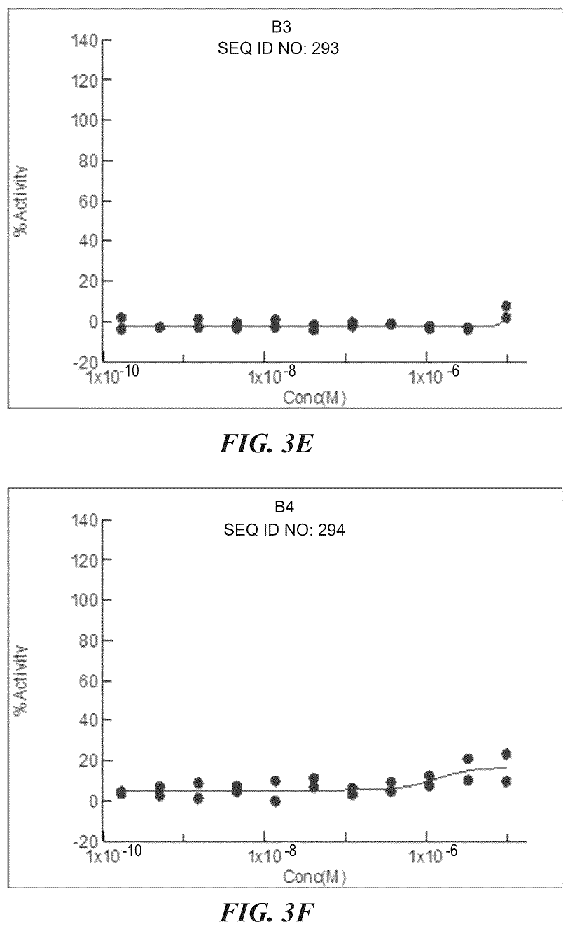

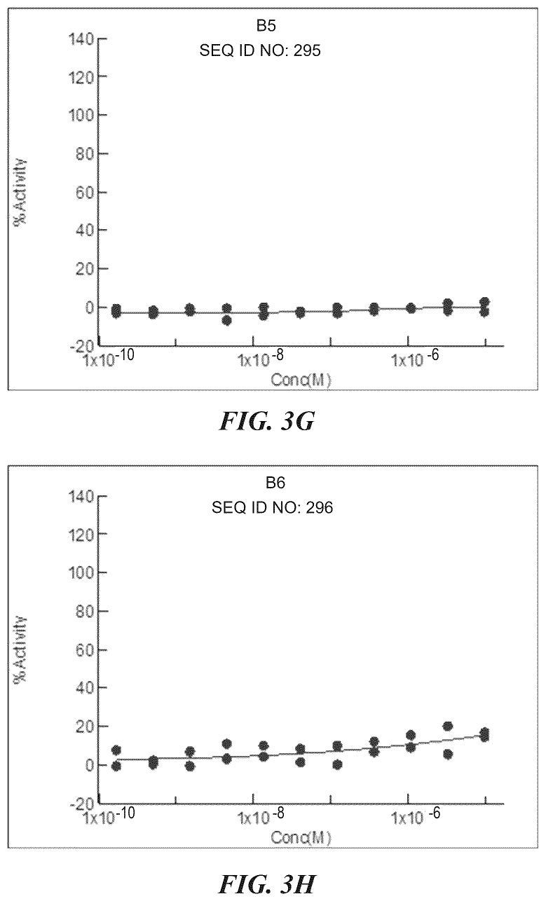

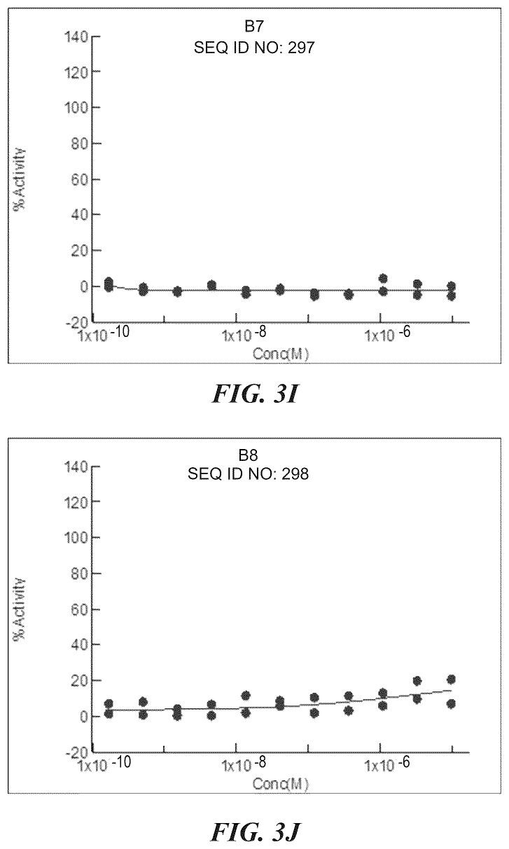

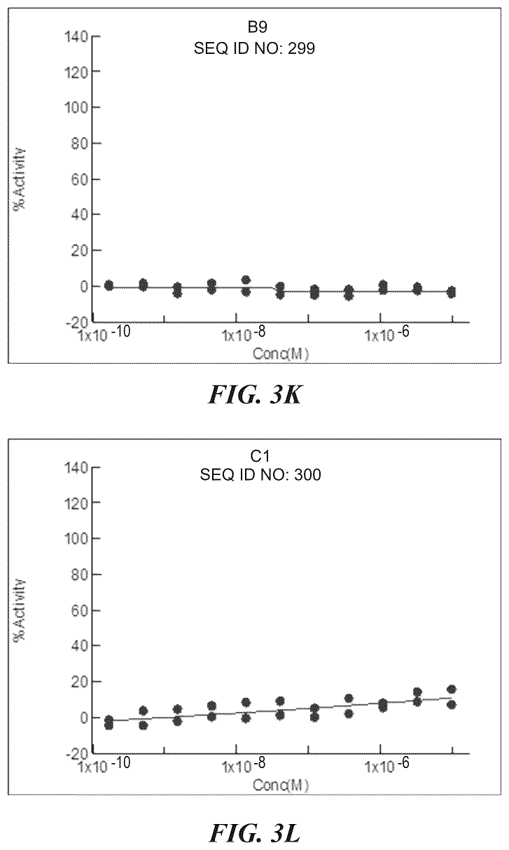

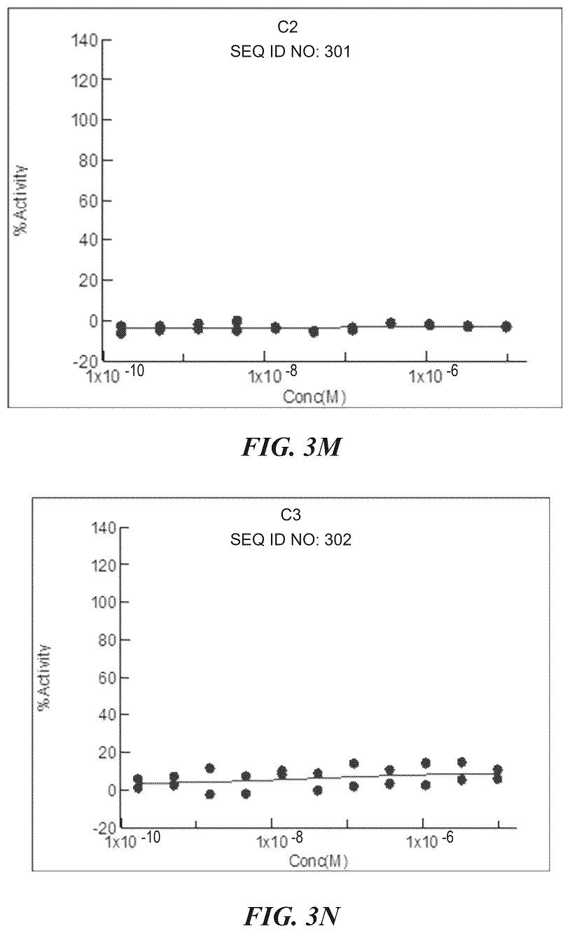

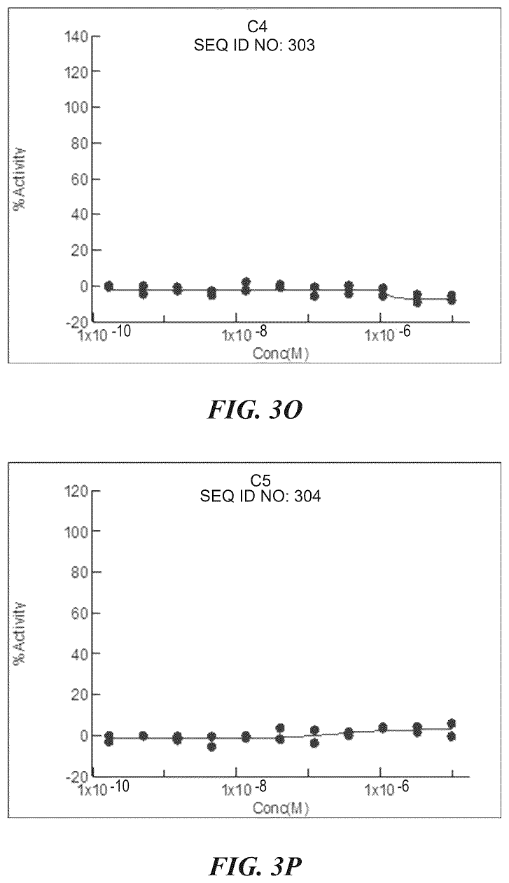

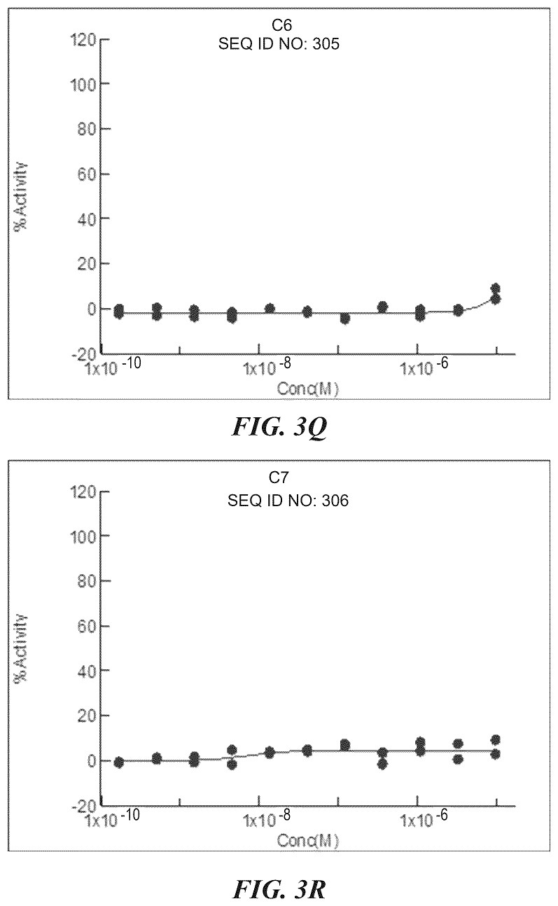

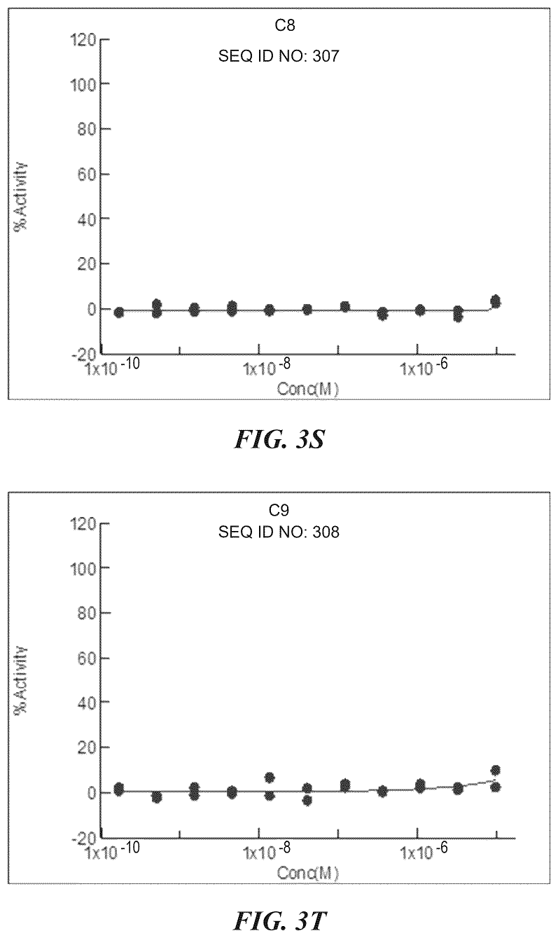

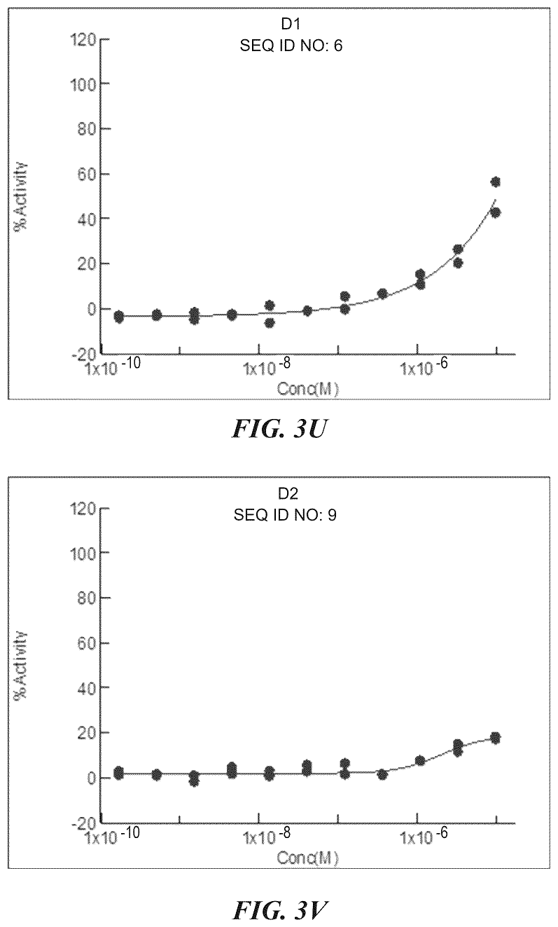

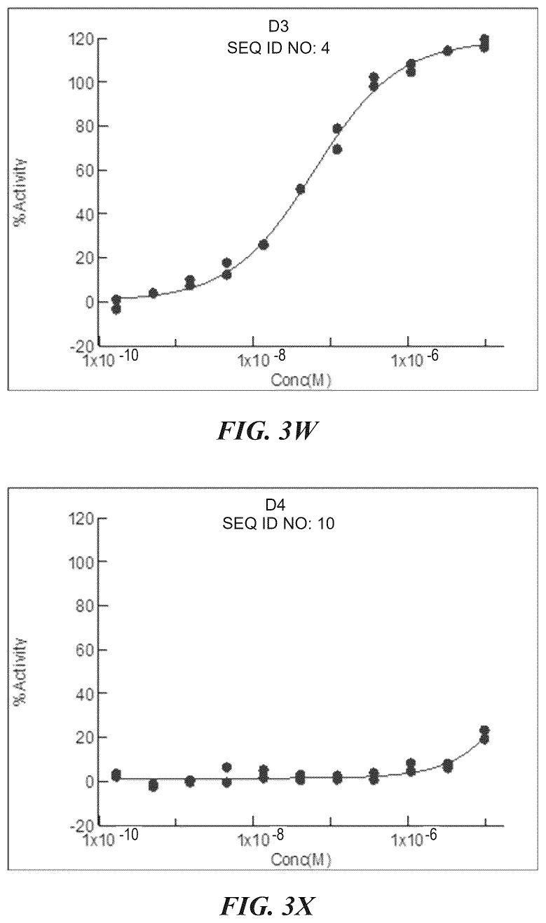

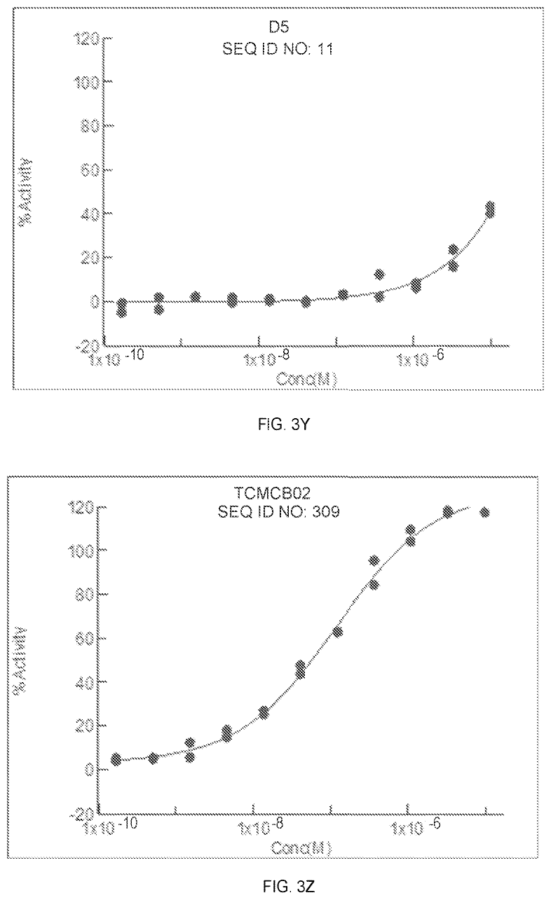

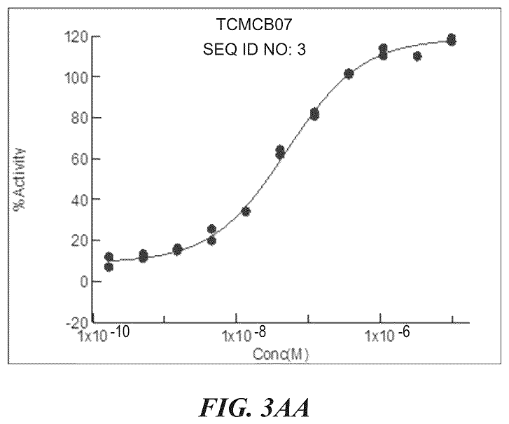

Administration of some non-naturally occurring melanocortin analogs inhibited MC4R activity, as measured by cAMP levels. Among the melanocortin analogs (FIG. 3A-AA), A1, D3, TCMCB02, and TCMCB07 all had IC₅₀ values under 10000 nM, with TCMCB07 and D3 having the lowest IC₅₀ values at 26.89 nM and 40.62 nM, respectively (Table 7).

TABLE 7
Dose-response results of non-naturally occurring
melanocortin analogs and control against MC4R

| Synthetic | Max | | Hill | |
|---|---|---|---|---|
| Peptide | Activation % | IC₅₀ (nM)* | Coefficient | Top (%) |
| HS024(TFA) | 103.83 | 153.04 | 0.61 | 113.93 |
| A1 | 79.28 | 3607.26 | 0.43 | 1195.94 |
| A1 | 27.41 | nd | 0.32 | 652.03 |
| B1 | 12.65 | nd | 7.57 | 12.66 |
| B2 | 35.52 | nd | 0.69 | 729.66 |
| B3 | 4.41 | nd | 9.04 | — |
| B4 | 16.14 | nd | 1.90 | 16.60 |
| B5 | −0.16 | nd | 1.22 | −0.16 |
| B6 | 15.38 | nd | 0.28 | 37.93 |
| B7 | −2.96 | nd | −1.76 | 516.09 |
| B8 | 13.55 | nd | 0.49 | 20.03 |
| B9 | −3.71 | nd | −19.71 | −0.55 |
| C1 | 11.12 | nd | 0.08 | 51.32 |
| C2 | −3.30 | nd | 48.53 | −2.85 |
| C3 | 8.07 | nd | 0.44 | 9.36 |
| C4 | −6.88 | nd | −7.31 | −1.97 |
| C5 | 2.47 | nd | 1.13 | 3.02 |
| C6 | 6.41 | nd | 2.07 | 321.26 |
| C7 | 5.86 | nd | 1.62 | 4.61 |
| C8 | 3.02 | nd | 10.57 | — |
| C9 | 5.89 | nd | 0.64 | 219.95 |
| D1 | 49.37 | >10000 | 0.55 | 2739.03 |
| D2 | 17.57 | nd | 1.55 | 18.76 |
| D3 | 117.49 | 40.62 | 0.80 | 119.03 |
| D4 | 21.03 | nd | 0.92 | 9354.39 |
| D5 | 41.47 | nd | 0.67 | 2724.46 |
| B02 | 120.24 | 57.10 | 0.69 | 127.47 |
| B07 | 117.90 | 26.89 | 0.83 | 118.91 |

*nd = not determined

### Example 5. Cardiovascular Activity of Non-Naturally Occurring Melanocortin Analogs

These studies were performed in urethane-anesthetized rats, since anesthesia is required for six-lead ECG monitoring. Urethane does not appear to affect the melanocortin/RFamide cardiovascular effects that were first reported in conscious rats. Gruber et al., Hypertension 6 (4): 468-74 (1984); Gruber et al., Am. J. Physiol. 257 (4 Pt 2): R681-694 (1989). Similar cardiovascular effects were found in experimental and clinical studies of melanocortin drugs. Nordheim et al., Peptides 27 (2): 438-443 (2006); Greenfield et al., N. Engl. J. Med. 360 (1): 44-52 (2009). Based on relative dose-effects for melanocortin cardiovascular effects, humans may be significantly more sensitive than rats. Initial selection of doses was guided by FDA exploratory IND studies with a recommendation for at least a 100-fold excess of the human dose.

The non-naturally occurring melanocortin analogs tested contained various peptides, such as RFamide-like motifs near the C-terminus (e.g., Arg-Trp-Lys; MT-II, PT-141, SHU9119); a multi-residue (2-4 residues)C-terminal amino acid extension that places the Arg-Trp motif deeper within the peptide chain (e.g., MT-I, MT-II, or PT-141); or are peptide mimetics modeled after His-Phe-Arg-Trp(SEQ ID NO: 1) (i.e., SHU9119) (see Table 8). Mutulis et al., J. Med. Chem. 47 (18): 4613-4626 (2004).

Peptides, agonists, or antagonists, or a peptide mimetic with an Arg-Trp(like) sequence near the C-terminus (“unprotected” melanocortin peptide) have significant and prolonged pressor activity (Table 9). A striking aspect of unprotected melanocortin peptides is their ability to produce cardiac arrhythmias, both tachycardia and bradycardia. See FIGS. 4A-4E. Cardiac arrhythmias were reported in the Phase 2 Clinical Trial of a melanocortin drug candidate. See, e.g., Gupta, Palatin, King Pharma Delay Late-Stage Trial Plans for Drug. Reuters. Aug. 30, 2007; King Pharmaceuticals and Palatin Technologies Delay Immediate Plans for Phase 3 Clinical Program with Bremelanotide for Erectile Dysfunction. Medical News Today. Aug. 31, 2007; Mishra, Palatin says King Pharma ends drug agreement on FDA concern. Reuters. Monday Sep. 10, 2007.

TABLE 8
Structures and Actions of Non-naturally occurring melanocortin analogs

| | | | SEQ ID |
|---|---|---|---|
| Peptide | Structure | Action | NO: |
| MT-I | Ac-Ser-Tyr-Nle-Glu-His-DPhe-Arg-Trp-Gly- | Agonist | 310 |
| | Lys-Pro-Val-NH2 | | |
| MT-II | Ac-Nle-c[Asp-His-DPhe-Arg-Trp-Lys]-NH2 | Agonist | 311 |
| PT-141 | Ac-Nle-c[Asp-His-DPhe-Arg-Trp-Lys]-COOH | Agonist | 312 |
| SHU9119 | Ac-Nle-c[Asp-His-DNal(2′)-Arg-Trp-Lys]-NH2 | Antagonist | 313 |
| PH932 | Ac-NLe-c[Asp-Pro-DNal(2′)-Arg-Trp-Lys]-Pro- | Antagonist | 314 |
| | Val-NH2 | | |
| THIQ | N-[(3R)-1,2,3,4-tetrahydroisoquinolinium-3- | Agonist | — |
| | ylcarbonyl]-(1R)-1-(4-chlorobenzyl)-2-[4- | | |
| | cyclohexyl-4-(1H-1,2,4-triazol-1- | | |
| | ylmethyl)piperidin-1-yl]-2-oxoethylamine | | |

Metabolically Stabilized Non-naturally occurring melanocortin analogs

| | | | |
|---|---|---|---|
| TCMCB01 | Ac-Nle-c[Asp-Pro-DNal(2′)-Arg-Trp-Lys]-DThr- | Antagonist | 315 |
| | DPro-DThr | | |
| TCMCB02 | Ac-Nle-c[Asp-Pro-DNal(2′)-Arg-Trp-Lys]-DPro- | Antagonist | 309 |
| | DVal-Nh2 | | |
| TCMCB03 | Ac-Nle-c[Asp-His-DNal(2′)-Arg-Trp-Lys]-DPro- | Antagonist | 316 |
| | DVal-Nh2 | | |
| TCMCB04 | Ac-Nle-c[D-Asp-Pro-DNal(2′)-Arg-Trp-DLys]- | Antagonist | 317 |
| | DPro-DVal-Nh2 | | |
| TCMCB05 | Ac-Nle-c[Asp-Pro-DNal(2′)-Arg-Trp-Lys]- | Antagonist | 318 |
| | β³Pro-β²Val-Nh2 | | |
| TCMCB06 | Ac-Nle-c[Asp-Pro-DNal(2′)-Arg-Trp-Lys]- | Antagonist | 319 |
| | β³Pro-β³Val-Nh2 | | |
| TCMCB07 | Ac-Nle-c[Asp-Pro-DNal(2′)-Arg-Trp-Lys]-DVal- | Antagonist | 3 |
| | DPro-Nh2 | | |

TABLE 9
Cardiovascular Effects of Non-naturally occurring melanocortin analogs
Cardiovascular Effects of Melanocortin Peptides

| | Peak MAP | |
|---|---|---|
| | Response | |
| | (Δ mm Hg)/ | |
| | Peak Heart Rate | Duration |
| Melanocortin Peptide, Dose, n | Response (BPM) | (min) |
| MT-I, 250 nmol, n = 5 | 0/0 | 0 |
| MT-II, 250 nmol, n = 5 | 22 ± 5/0 | 42 ± 11 |
| Bremelanotide (PT-141), | 32 ± 6/44 ± 6 | 140 ± 53 |
| 250 nmol, n = 5 | | |
| SHU-919, 250 nmol, n = 5 | 16 ± 5/3 ± 14 | 29 ± 11 |
| PG-932, 200 nmol, n = 5 | 5 ± 3/13 ± 09 | 4 ± 3 |
| THIQ, 300 nmol, n = 5 | 22 ± 11/81 ± 09 | 19 ± 4 |
| TCMCB01, 600 nmol, n = 5 | 0/0 | 0 |

### Example 6. Anti-Cachexia Activity of Metabolically Stabilized Non-Naturally Occurring Melanocortin Analogs

The anti-cachectic effects of the first-generation non-naturally occurring melanocortin analogs (TCMCB01; SEQ ID NO: 315) were examined in the Lewis sarcoma rat model, and the 1st-7th-generation non-naturally occurring melanocortin analogs in the LPS model of cachexia. The non-naturally occurring melanocortin analogs possessed enhanced anti-cachectic activity compared to the parent compound, PG932, and no cardiovascular or dose-limiting behavioral side effects typically found in some other non-naturally occurring melanocortin analogs.

The Lewis model is a methylcholanthrene-induced sarcoma that does not metastasize. This sarcoma is an aggressive form of cancer that produces significant cachexia. Popp et al., Cancer 49 (6): 1212-1220 (1982). In the protocol, “treatment” began 8-days after tumor implantation, with a significant decrease in food intake from baseline. Body composition was determined by NMR prior to tumor implantation, and on the final day of the experiment after tumor resection, by ECHO MRI (NMR). Tumor implantation produces cachexia on or about the sixth day. The non-naturally occurring melanocortin analog or saline was administered after day 8 and continued through day 12.

The LPS model uses rats were maintained on a 12 h light/dark schedule with ad libitum access to food and water. Animals were handled daily for a minimum of three consecutive days to decrease non-specific handling stress. On the day of the experiment, individually housed animals received intraperitoneal injections of LPS dissolved in 0.5% low-endotoxin BSA, 0.9% saline or 0.5% BSA in 0.9% saline alone, and were returned to their home cage. The drug or control was administered (ICV, IP, or PO, depending on the type of study) 1-hour after LPS injection.

### First-Generation Metabolically Stabilized Non-Naturally Occurring Melanocortin Analogs: TCMCB01

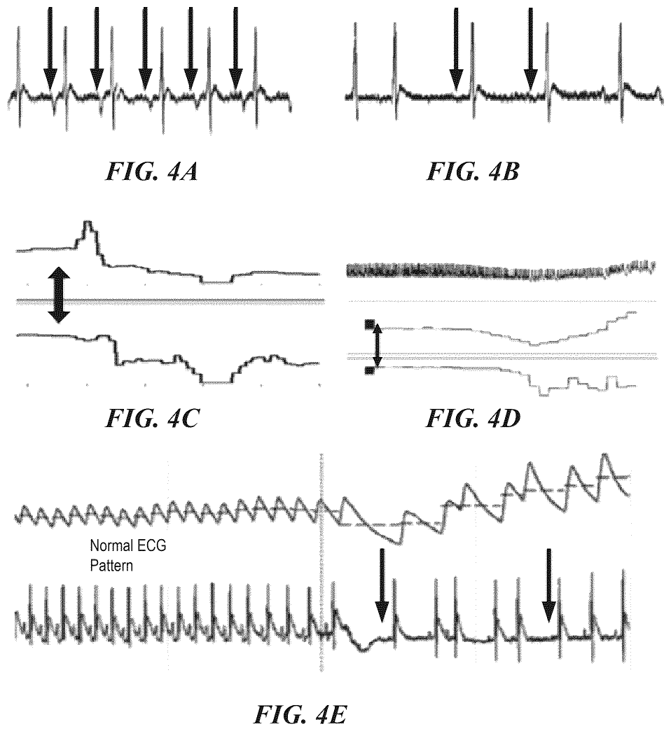

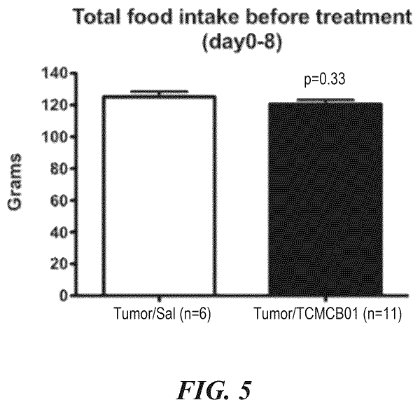

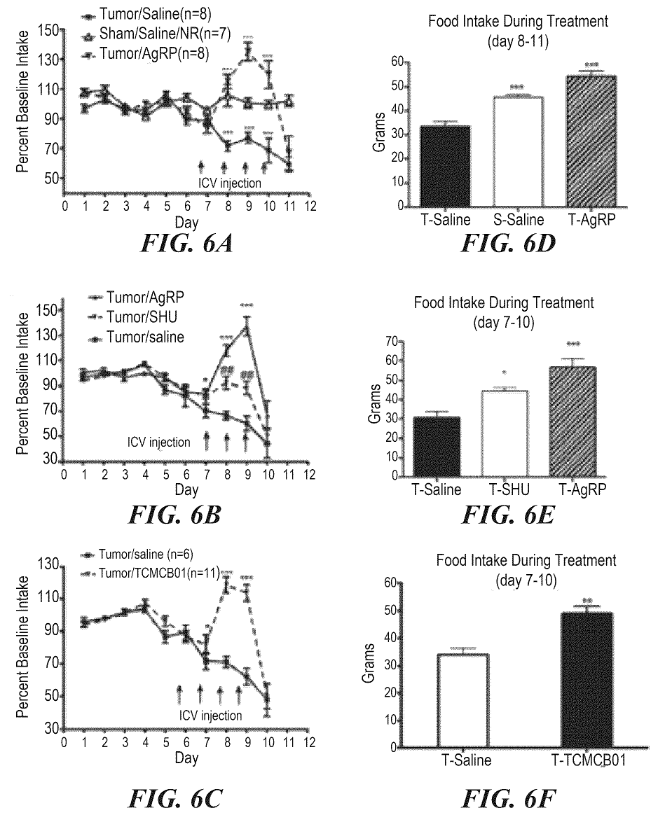

Using the Lewis model, treatment with TCMCB01 (see Table 8), one time per day at a dose of 2 nmol, reversed the cachexia-anorexia of the tumor burden, ICV saline had no effect. Prior to tumor implantation, both groups (drug or saline) had similar food intake (14 g/day). See FIG. 5. After tumor implantation, in saline-treated/tumor bearing controls, food intake decreased 35% to 9 g/day by day 8. FIGS. 6A and 6D. However, treatment with TCMCB01 reversed anorexia to a food intake similar to the control state. See FIGS. 6C and 6F. TCMCB01 was as potent as the natural melanocortin system inhibitor Agouti Related Protein (AGRP), and significantly more potent than SHU9119.

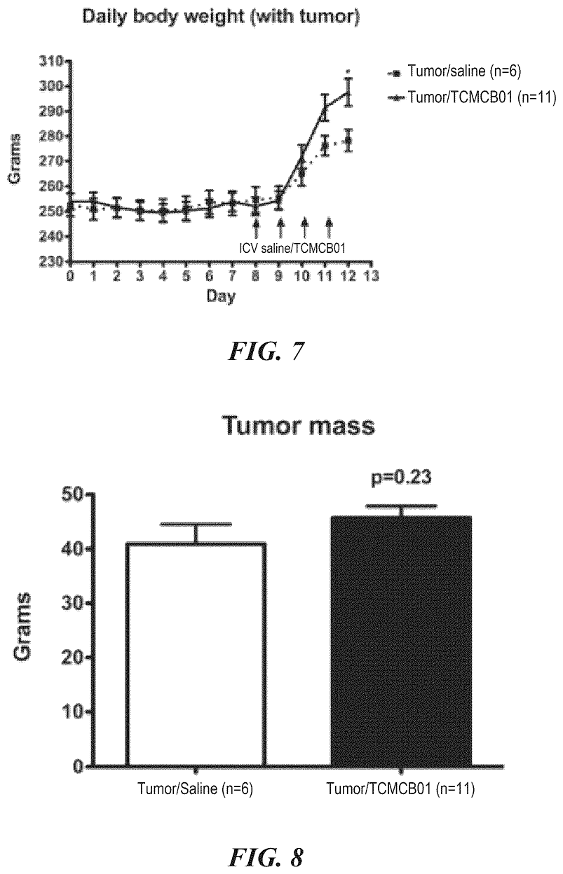

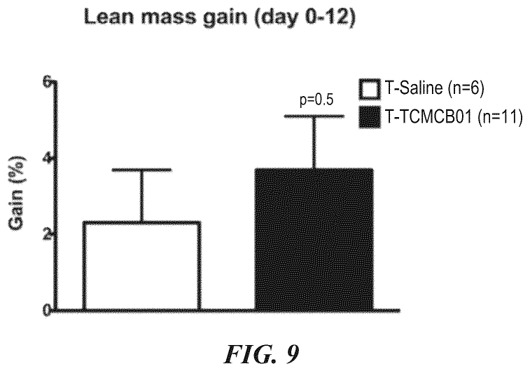

Both groups increased their body weight after tumor implantation, but the drug treated group was significantly greater than saline controls (FIG. 7). In the saline-treated rats, this weight gain was due to the tumor mass. Tumor weight (40 g) was equivalent in saline-treated and drug-treated group(p=0.2) indicating a food-intake related body weight gain. FIG. 8. While NMR analysis (after tumor excision) showed TCMCB01 treatment increased lean body mass, statistical significance was only p=0.05 (FIG. 9). However, this may be due to the number of NMR observations made (i.e., one pre-treatment and one post-treatment). Food intake and body weight were determined daily. Additional NMR observations or an increased number of observations (i.e., “n”) may have resolved this dichotomy.

An important aspect to these results was that previously published results with other synthetic non-naturally occurring melanocortin analogs only produced a partial reversal of feeding in cachexia models. The typical result was <50% reversal. The naturally occurring melanocortin antagonist protein, AGRP, produced a 100% reversal of cachexia-anorexia syndrome. Marks et al., Cancer Res. 61 (4): 1432-1438 (2001). In a related study (not shown), TCMCB01's anti-cachectic effect in the Lewis model was 20% greater than AGRP, essentially producing hyperphagia. This feeding effect was produced at a dose that was one tenth of that used with previous synthetic non-naturally occurring melanocortin analogs. These results suggest that C-terminal “protection” significantly enhanced melanocortin antagonist actions. However, TCMCB01 did not have peripheral activity (assessed in the LPS model). Thus, there was insufficient blood brain barrier-transport for use as a therapeutic agent. The second-generation non-naturally occurring melanocortin analog, TCMCB02, was then investigated.

### Second-Generation Stabilized Non-Naturally Occurring Melanocortin Analog: TCMCB02

The second-generation stabilized non-naturally occurring melanocortin analog, TCMCB02 (SEQ ID NO: 309) has the same sequence and cyclization of TCMCB01, (see Table 8), i.e., with the same melanocortin pharmacophore. However, the C-terminal peptide sequence extension is D-Pro-D-Val-NH₂. This molecule was designed to combine the blood brain barrier penetrating activity of PG932 with an enzymatically resistant C-terminus. Sutton et al., Peptides 29 (1): 104-111 (2008); Marks et al., unpublished data.

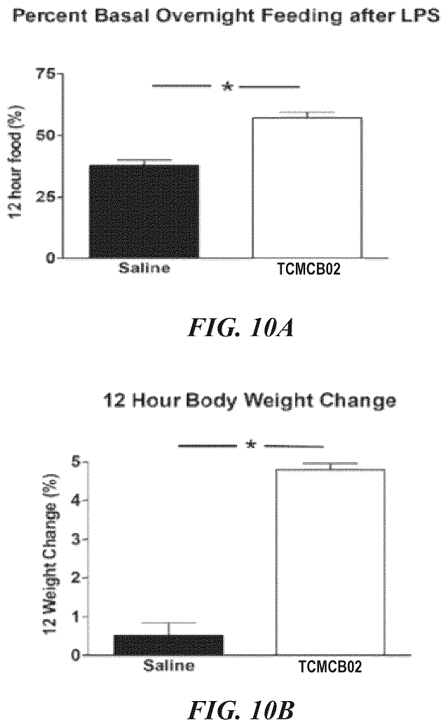

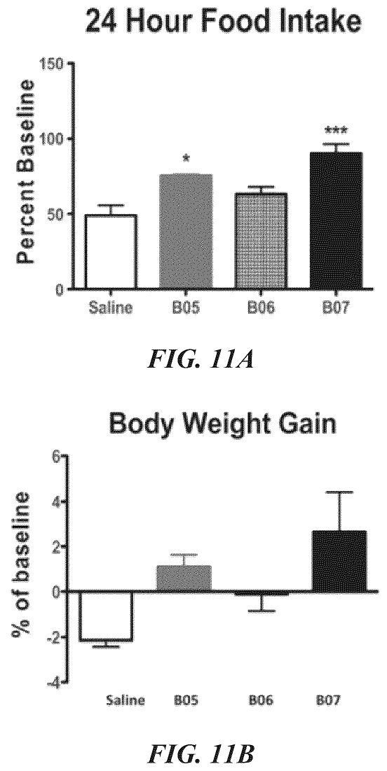

The anti-anorectic activity of TCMCB02 was examined in a two-step process. Using the rat LPS-induced cachexia model, it was confirmed that ICV TCMCB02 maintained direct anti-cachectic activity equivalent to TCMCB01. A peripheral administration study was performed where IP administration of TCMCB02 (2 mg/kg) was given one hour after LPS-administration. The subsequent 12-hour feeding data is shown in FIGS. 10A-10B, and the effects on body weight in FIG. 11A-11B. Rats primarily eat at night; they lose weight during the day and gain it back at night when they eat food, as reflected in FIGS. 10A-10B. These data indicate that TCMCB02 has peripheral activity, significantly reversing LPS-induced cachexia.

Data from several other cyclic melanocortin peptides suggests that blood brain barrier transport and oral activity appear to co-exist. See, e.g., Hess et al., J. Med. Chem. 50 (24): 6201-6211 (2007); Hess et al., J. Med. Chem. 51 (4): 1026-1034 (2008). Because TCMCB02 has 10-fold greater anti-cachectic activity than the “parent” compound (TCMCB01), it appears that the D-amino acid C-terminal di-peptide provides chronic protection. This enhances therapeutic efficacy and prevents expression of any over-lapping pharmacophore side effects. Thus, two non-naturally occurring melanocortin analogs with D-amino acids and di- or tri-peptide C-termini showed enhanced efficacy, and no cardiovascular or behavioral side effects. However, as is discussed below, TCMCB02 did not have oral activity; i.e., a lack of trans-gastro-intestinal epithelial transport.

### Basis for Trans-Epithelial Transport of Peptide Drugs

Active transport carriers for di- and tri-peptides have been described, and these transported peptides can serve as carriers for small cyclic peptides or non-peptide drugs. Brandsch, Amino Acids 31 (2): 119-136 (2006); Brandsch et al., J. Pharm. Pharmacol. 60 (5): 543-58 (2008). For example, the peptide transporters PEPT1 and PEPT2 are known to mediate di- and tri-peptide (oligopeptide) transport respectively through the gastrointestinal tract and the blood-brain-barrier (ependymal cells and choroid plexus). However, there has never been a detailed description of the requirements that allow an oligopeptide transport system to carry attached drugs across the gastrointestinal barrier or the blood brain barrier, or an approach that allows the transport of drugs greater than 500-600 Daltons. A detailed examination of the structural requirements of di- and tri-peptides that can be linked to molecules for active transport, and whether small changes in peptide structure are important for overall molecule transport had not been conducted previously. Furthermore, different molecules may function better with regards transport having specific peptides added to their C- or N-terminus, and a peptide sequence that works well as a transporter at one terminus may not work at the other.

Third-Generation Non-Naturally Occurring Melanocortin Analog: TCMCB03 (i.e., His6 PG932)

Initial comparisons of the two best-characterized non-naturally occurring melanocortin analogs, SHU9119 and PG932, could conclude that PG932 was active peripherally, while SHU9119 was not (see Table 8). Sutton et al., Peptides 29 (1): 104-111 (2008). This question of whether a His6 (SHU9119) or Pro6 (PG932) in the pharmacophore produces a superior anti-cachectic effect went unanswered. TCMCB03 (SEQ ID NO: 316) has a His6 (see Table 8). A comparison of TCMCB02 (Pro6) and TCMCB03 (His6) showed that the His-for-Pro substitution enhanced anti-cachexia when given ICV in the LPS experimental model. However, in an IP administration study, TCMCB03 failed to cross the blood brain barrier.

Using Pro, an imidic acid, rather than an amino acid as one of the residues in a cyclic peptide reduces free rotation: a secondary amine forms one of the peptide bonds of the Pro residue. Reducing free rotation is an important factor in cyclic peptide active transport through epithelial barriers. Hess et al., J. Med. Chem. 50 (24): 6201-6211 (2007). Results herein confirm this hypothesis. In addition, the sequence of the di-peptide extension of the cyclic structure is also important (see the fifth- and seventh-generation polypeptides, below). Collectively, these data are consistent with a oligopeptide transport system functioning as the drug/cyclic peptide carrier, as previously described. Vabeno et al., Bioorg. Med. Chem. 13 (6): 1977-1988 (2005); Kikuchi et al., J. Pharm. Sci. 98 (5): 1775-1787 (2009); Wang et al., J. Biomed. Sci. 17:71 (2010).

Fourth-Generation Non-Naturally Occurring Melanocortin Analog: TCMCB04 (i.e., D-Asp⁵ D-Lys¹⁰ TCMCB02)

The historical basis for cyclizing MCs. was that a reverse turn conformation was required for receptor binding. Sawyer et al., Proc. Nat. Acad. Sci. USA 79 (6): 1751-1755 (1982); Sawyer et al., J. Med. Chem. 25 (9): 1022-1027 (1982). Initially, a D-amino acid substitution in the middle of the melanocortin pharmacophore enhanced biological activity: presumably because a D-residue will reduce the tendency of an L-amino acid sequence to form an α-helix, but stabilize a β-turn. Sawyer et al., Proc. Nat. Acad. Sci. USA 77 (10): 5754-5758 (1980).

Cyclized melanocortins were the next historical step; first di-sulfide bridging was utilized, then lactam bridges were used. It was thought that using D-amino acid residues to form the lactam bridge in cyclic melanocortins would further stabilize the cyclic structure, producing superior biological activity. TCMCB04 (SEQ ID NO: 317), the D-Asp⁵, D-Lys¹⁰ derivative of TCMCB02, a drug candidate with blood brain barrier transport properties was synthesized (see Table 8). Given ICV, this polypeptide had superior anti-cachectic activity compared to the parent compound in the LPS assay. However, the D-Asp⁵, D-Lys¹⁰ modification eliminated blood brain barrier transport, demonstrating that both blood brain barrier (this polypeptide) and GI transport (TCMCB02) depend on the stereospecificity of the derivative.

The role of polypeptide structure in peptide active transport by the di- or tri-peptide transport systems has not been thoroughly investigated. Results herein show that slight chemical and/or stereochemical changes in polypeptide structure has significant effects on the transport properties of the drug-peptide transporter complex. Several important factors were identified that promote melanocortin-di-peptide achieve transport.

Role of Dipeptide Structure in Drug-Dipeptide Complex Transport; Fifth- and Sixth-Generation Non-naturally occurring melanocortin analogs: TCMCB05 and TCMCB06

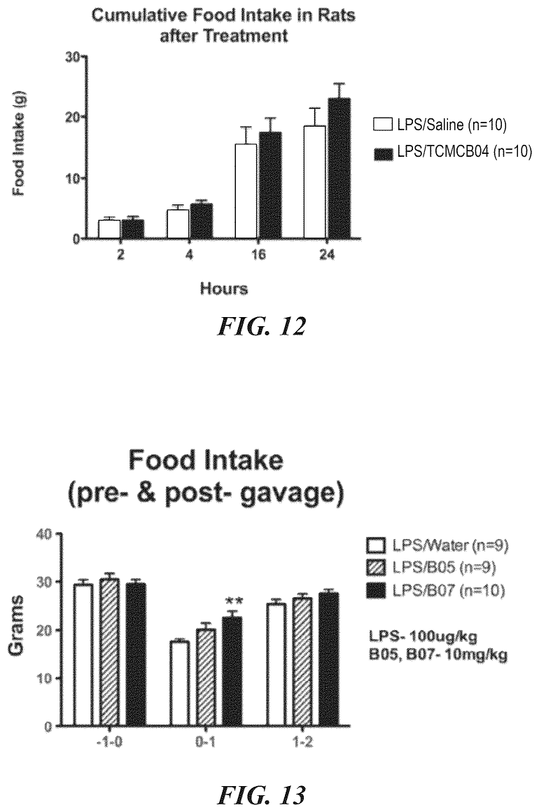

The TCMCB05 (i.e., β³-Pro¹¹, β²-Val¹²; SEQ ID NO: 318) and TCMCB06 (i.e., β³-Pro¹¹, β³-Val¹²; SEQ ID NO: 319) polypeptides were designed to combine the stereochemistry of L-amino acids with the resistance to enzymatic degradation of D-residues (see Table 8). Amino or imidic acids with their D-amino and/or carboxyl groups displaced by a β-carbon maintain their side-chain stereochemistry and are more stable to enzymatic degradation. Lew et al., FASEB J. 15 (9): 1664-1666 (2001); Nachman et al., Peptides 30 (3): 608-615 (2009). Both TCMCB05 and TCMCB06 had 10-fold greater molar activity than TCMCB02 in LPS-induced cachexia following ICV-administration. However, only TCMCB05 (33-Pro-β²-Val) had IP anti-cachexia activity as measured by enhancing food intake and weight gain following LPS-administration. TCMCB05 (Ac-Nle-c[Asp-Pro-D-Nal(2′-Arg-Trp-Lys]-β³-Pro-β²-Val-NH₂ (SEQ ID NO: 318)), differs from TCMB07 by having a β-Pro-β-Val C-terminal extension, whereas TCMB07 has the reversed C-terminal extension with D-amino acids (Ac-Nle-c[Asp-Pro-D-Nal(2′)-Arg-Trp-Lys]-D-Val-D-Pro-NH₂ (SEQ ID NO: 3)). As shown in FIG. 13, only TCMCB07 produced a significant increase in feeding. As reflected in FIGS. 12 and 13, changing two residues in the “load” from L-to D-amino acids blocked blood-brain barrier transport; transport is stereospecific, effectively eliminating paracellular movement through the blood-brain barrier as the TCMCB02 mechanism of action. The stereospecificity is inconsistent with a movement governed by the “Rule of Five” (Lipinski et al., Adv. Drug Deliv. Rev. 46 (1-3): 3-26 (2001).

Seventh-Generation Non-naturally occurring melanocortin analogs: TCMCB07 (i.e., D-Val¹¹, D-Pro¹²TCMCB02)

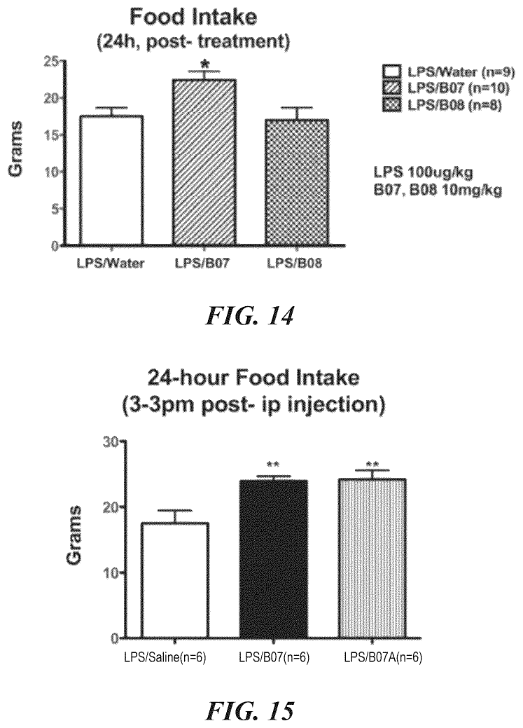

TCMCB07 (SEQ ID NO: 3) is based on TCMCB02 with the C-terminal di-peptide sequence of TCMCB02 reversed see Table 8). This analog was designed to test the sequence specificity for transport. This polypeptide maintained ICV activity. However, it had 10-fold more peripheral (IP) LPS anti-cachectic activity than TCMCB02, suggesting that the C-terminal sequence affected specific blood brain barrier transport (FIGS. 14-15). These data demonstrate that a C-terminal Val-Pro sequence (Ac-Nle-c[Asp-Pro-D-Nal(2′)-Arg-Trp-Lys]-D-Val-D-Pro-NH₂ (SEQ ID NO: 3); TCMCB07) composed of D-residues imparts oral activity to the drug molecule and causes a significant enhancement of food intake. The same di-peptide sequence composed of β-residues does not (e.g., Ac-Nle-c[Asp-Pro-D-Nal(2′)-Arg-Trp-Lys]-32-Val-3-Pro-NH₂ (SEQ ID NO: 390); TCMCB08). Two conclusions can be drawn about the structure required for oral activity. First, a TCMCB02 vs. TCMCB07 comparison shows that oral activity is di-peptide sequence specific: Pro-Val only has blood-brain barrier transport; Val-Pro has both gastrointestinal and blood-brain barrier transport. Second, TCMCB08 has peripheral activity (data not shown) but no gastrointestinal transport. Thus, oral activity is dependent on the movement of the α-carbon amino and/or carboxyl groups (of the di-peptide) to the β-carbon. The blood-brain barrier oligopeptide transporter accepts a particular β-residue di-peptide, while the gastrointestinal oligopeptide transporter does not. TCMCB07 and TCMCB07A have the same primary peptide sequence except that TCMCB07 has a C-terminal amide moiety and TCMCB07A does not, i.e., Ac-Nle-c[Asp-Pro-D-Nal(2′)-Arg-Trp-Lys]-D-Val-D-Pro-NH₂ (SEQ ID NO: 3) (TCMCB07) vs. Ac-Nle-c[Asp-Pro-D-Nal(2′)-Arg-Trp-Lys]-D-Val-D-Pro(SEQ ID NO: 2) (TCMCB07A). Peripheral administration of TCMCB07 or TCMCB07A produces equivalent stimulation of feeding in LPS-induced cachexia. Thus, the C-terminal amide moiety has no effect on transport but may stabilize the peptide against degradation.

This result providing additional evidence for peptide transport independent of the “Rule of Five” effect, and supported the possibility of a specific transporter.

### Summary of TCMCB05-TCMCB07 Antagonists

An important aspect of C- or N-terminal stabilization is in the residue selection process. Variables include the stereochemistry of the residue (e.g., D- or L-conformation); the chemical moieties of the side chain; and which carbon atom (α-, β-, γ-, etc.) the amino- or carboxyl-groups are attached to. For example, the non-naturally occurring melanocortin analog PG932 has a C-terminal di-peptide sequence of Pro-Val-amide. This C-terminal extension is not part of the cyclized melanocortin pharmacophore of the molecule. Thus, the extension does not play a role in receptor binding or activation. Changing these two residues (Pro-Val) to the D-configuration increases in vivo stability and/or apparent efficacy by 10-fold, as assessed by the dose required to alleviate bacterial toxin-induced cachexia in rats following intracerebroventricular (ICV) administration. This effect is not mediated by an apparent effect in receptor interaction, but rather stability against enzymatic degradation. The TCMCB01 sequence with a C-terminal D-configuration dipeptide has blood-brain barrier transport. An additional 10-fold reduction in the (ICV) dose required for bacterial toxin anti-cachexia is produced by changing the D-configuration C-terminal di-peptide to β-Pro-β-Val-amide (both residues in the L-configuration). Thus, L-amino acid residues of the β-type are more stabilizing to the C-terminus of a peptide than their equivalent (side-chain group) α-types in the D-configuration.

### Example 7. Reversal of Cachexia-Induced Lethargy

In animals and humans, cachexia produces “lethargy,” i.e., the aversion to activity. Grossberg et al., J. Neurosci. 31 (31): 11376-11386. Once-daily peripheral dosing with TCMCB02, TCMCB05, or TCMCB07 (see Table 8) for 48 hours (i.e., 2 IP doses) restored “normal” behavior to rats, irrespective of tumor load. This therapeutic effect is dependent on a stabilized non-naturally occurring melanocortin analog C-terminal extension. Video recordings characterized control animal behavior as neo-exploratory movement about the cage, i.e., repeated “sniffing” the air to presumably sample odors (e.g., neighboring animals), grooming, and intermittent bouts of eating and drinking. Caldecott-Hazard et al., J. Neurosci. 8 (6): 1951-1961 (1988).

In contrast, a Lewis sarcoma rat exhibits no neo-exploratory behavior. The animal typically remains in one position for an extended period of time (>1 minute). Occasionally it will change position, assuming the new position for an extended period. The rats show no grooming behavior, evident in the appearance of their coat and eyes. Food intake is greatly reduced.

The restoration of normal behavior following treatment with the polypeptides is independent of food intake. Lethargy is not produced by the reduction in caloric intake, nor reversed solely by increased food intake. A recent study describes the non-melanocortin basis for cachexia-induced lethargy. Grossberg et al., J. Neurosci. 31 (31): 11376-11386. Based on this, TCMCB01-TCMCB07 are thought to activate the orexin system. However, it should be appreciated that merely having an automated recording of movement does not reveal the qualitative aspects of what is occurring physiologically. As an illustrative example, restoring appetite (melanocortin drug therapy) to a cachectic animal will cause it to seek food. When the animal is provided food, it will sit near the food hopper and eat (reduced movement). If an animal is not provided food, it may move around the cage seeking food (increased movement).

### Example 8. Non-Naturally Occurring Melanocortin 3- and 4-Receptor Antagonists

Non-naturally occurring melanocortin analogs can function as MC3R and MC4R antagonists. Such antagonists are useful for treating disorders of impaired appetite and lean body mass wasting. See Table 10. This includes cachexia-anorexia syndrome, anorexia nervosa, dermatomyositis, polymyositis, muscular dystrophy, sarcopenia, and other muscle mass wasting syndromes.

Non-naturally occurring melanocortin analogs are also useful as regulators of the delta (δ) opioid system. They have direct agonist at the delta opioid receptor, indirect opioid agonist actions through inhibition of the central melanocortin system. These effects enhance the exogenous opioid actions and prevent development of exogenous opioid tolerance. Thus, non-naturally occurring melanocortin analogs may be useful alone or in combination with opioid analogs.

In addition, non-naturally occurring melanocortin analogs can function as melanocortin 5 receptor agonists. Thus, they are potentially useful for treating syndromes of decreased/impaired exocrine gland secretion; for example dry and/or dry mouth. These latter disorders are characterized by a relative lack of lacrimal gland (eye) and/or salivary gland secretions. Table 10 contains a list of melanocortin 3- and 4-receptor antagonists and melanocortin 5 receptor agonists useful with the present technology.

TABLE 10
Melanocortin 3- and 4-Receptor Antagonists and Melanocortin 5-Receptor Agonists

| | | SEQ ID |
|---|---|---|
| Peptide | Structure | NO: |
| TCMCB345 1 | Ac-Nle-c[Asp-Pro-DNal(2′)-Arg-Trp-Lys]-DThr-DPro-DThr | 320 |
| TCMCB345 2 | Ac-Nle-c[Asp-His-DNal(2′)-Arg-Trp-Lys]-DThr-DPro-DThr | 321 |
| TCMCB345 3 | Ac-Nle-c[DAsp-Pro-DNal(2′)-Arg-Trp-DLys]-DThr-DPro-DThr | 322 |
| TCMCB345 4 | Ac-Nle-c[DAsp-His-DNal(2′)-Arg-Trp-DLys]-DThr-DPro-DThr | 323 |
| TCMCB345 5 | Ac-Nle-c[Asp-Pro-DNal(2′)-Arg-Trp-Lys]-DPro-DVal-NH2 | 324 |
| TCMCB345 6 | Ac-Nle-c[Asp-His-DNal(2′)-Arg-Trp-Lys]-DPro-DVal-NH2 | 325 |
| TCMCB345 7 | Ac-Nle-c[DAsp-Pro-DNal(2′)-Arg-Trp-DLys]-DPro-DVal-NH2 | 326 |

### Melanocortin 3- and 4-Receptor Agonists

Melanocortin receptor agonists are potent inhibitors of feeding/appetite and stimulate metabolism. See Table 11. These actions have been shown to be useful in diseases or conditions producing excess body weight, since melanocortin agonist treatment contributes to weight loss. Table 11 contains a list of MC3R and MC4R agonists useful with the present technology.

TABLE 11
Melanocortin 3- and 4- Receptor Agonists

| | | SEQ ID |
|---|---|---|
| Peptide | Structure | NO: |
| TCMCA34 1 | Ac-Nle-c[Asp-Pro-DPhe-Arg-Trp-Lys]-DThr-DPro-DThr | 327 |
| TCMCA34 2 | Ac-Nle-c[Asp-His-DPhe-Arg-Trp-Lys]-DThr-DPro-DThr | 328 |
| TCMCA34 3 | Ac-Nle-c[DAsp-Pro-DPhe-Arg-Trp-DLys]-DThr-DPro-DThr | 329 |
| TCMCA34 4 | Ac-Nle-c[DAsp-His-DPhe-Arg-Trp-DLys]-DThr-DPro-DThr | 330 |
| TCMCA34 5 | Ac-Nle-c[Asp-Pro-DPhe-Arg-Trp-Lys]-DPro-DVal-Nh2 | 331 |
| TCMCA34 6 | Ac-Nle-c[DAsp-His-DPhe-Arg-Trp-DLys]-DPro-DVal-Nh2 | 332 |
| TCMCA34 7 | Ac-Nle-c[Asp-Pro-DNal(2′)-Arg-Trp-Lys]-DPro-DVal-Nh2 | 333 |
| TCMCA34 8 | Ac-Nle-c[Asp-His-DNal(2′)-Arg-Trp-Lys]-DPro-DVal-Nh2 | 334 |
| TCMCA34 9 | Ac-Nle-c[Asp-Pro-DPhe-Arg-Trp-Lys]-DVal-DPro-NH2 | 335 |
| TCMCA34 10 | Ac-Nle-c[Asp-His-DPhe-Arg-Trp-Lys]-DVal-DPro-NH2 | 336 |
| TCMCA34 11 | Ac-Nle-c[DAsp-Pro-DPhe-Arg-Trp-DLys]-DVal-DPro-NH2 | 337 |
| TCMCA34 12 | Ac-Nle-c[DAsp-His-DNal(2′)-Arg-Trp-DLys]-DVal-DPro-NH2 | 338 |
| TCMCA34 13 | Ac-Nle-c[Asp-Pro-DPhe-Arg-Trp-Lys]-βPro-βVal-NH2 | 339 |
| TCMCA34 14 | Ac-Nle-c[Asp-His-DPhe-Arg-Trp-Lys]-βPro-βVal-NH2 | 340 |
| TCMCA34 15 | Ac-Nle-c[DAsp-Pro-DPhe-Arg-Trp-DLys]-βPro-βVal-NH2 | 341 |
| TCMCA34 16 | Ac-Nle-c[DAsp-His-DPhe-Arg-Trp-DLys]-βPro-βVal-NH2 | 342 |
| TCMCA34 17 | Ac-Nle-c[Asp-Pro-DPhe-Arg-Trp-Lys]-βVal-βPro-NH2 | 343 |
| TCMCA34 18 | Ac-Nle-c[Asp-His-DPhe-Arg-Trp-Lys]-βVal-βPro-NH2 | 344 |
| TCMCA34 19 | Ac-Nle-c[DAsp-Pro-DPhe-Arg-Trp-DLys]-βVal-βPro-NH2 | 345 |
| TCMCA34 20 | Ac-Nle-c[DAsp-His-DNal(2′)-Arg-Trp-DLys]-βVal-βPro-NH2 | 346 |
| TCMCA34 21 | Ac-Nle-c[Asp-Pro-DPhe-Arg-Trp-Lys]-βPro-βPro-NH2 | 347 |
| TCMCA34 22 | Ac-Nle-c[Asp-His-DPhe-Arg-Trp-Lys]-βPro-βPro-NH2 | 348 |
| TCMCA34 23 | Ac-Nle-c[DAsp-Pro-DNal(2′)-Arg-Trp-DLys]-βPro-βPro-NH2 | 349 |
| TCMCA34 24 | Ac-Nle-c[DAsp-His-DNal(2′)-Arg-Trp-DLys]-βPro-βPro-NH2 | 350 |
| TCMCA34 25 | Ac-Nle-c[Asp-Pro-DPhe-Arg-Trp-Lys]-βVal-βVal-NH2 | 351 |
| TCMCA34 26 | Ac-Nle-c[Asp-His-DPhe-Arg-Trp-Lys]-βVal-βVal-NH2 | 352 |
| TCMCA34 27 | Ac-Nle-c[DAsp-Pro-Phe-Arg-Trp-DLys]-βVal-βVal-NH2 | 353 |
| TCMCA34 28 | Ac-Nle-c[DAsp-His-DPhe-Arg-Trp-DLys]-βVal-βVal-NH2 | 354 |
| TCMCA34 29 | Ac-Nle-c[Asp-Pro-DPhe-Arg-Trp-Lys]-(3-methyl)-βVal-βVal-NH2 | 355 |
| TCMCA34 30 | Ac-Nle-c[Asp-His-DPhe-Arg-Trp-Lys]-(3-methyl)-βVal-βVal-NH2 | 356 |
| TCMCA34 31 | Ac-Nle-c[DAsp-Pro-DPhe-Arg-Trp-DLys]-(3-methyl)-βVal-βVal-NH2 | 357 |
| TCMCA34 32 | Ac-Nle-c[DAsp-His-DPhe-Arg-Trp-DLys]-(3-methyl)-βVal-βVal-NH2 | 358 |
| TCMCA34 33 | Ac-Nle-c[Asp-Pro-DPhe-Arg-Trp-Lys]-(3-methyl)-βVal-βPro-NH2 | 359 |
| TCMCA34 34 | Ac-Nle-c[Asp-His-DPhe-Arg-Trp-Lys]-(3-methyl)-βVal-βPro-NH2 | 360 |
| TCMCA34 35 | Ac-Nle-c[DAsp-Pro-DPhe-Arg-Trp-DLys]-(3-methyl)-βVal-βPro-NH2 | 361 |
| TCMCA34 36 | Ac-Nle-c[DAsp-His-DPhe-Arg-Trp-DLys]-(3-methyl)-βVal-βPro-NH2 | 362 |
| TCMCA34 37 | Ac-Nle-c[Asp-Pro-DPhe-Arg-Trp-Lys]-βThr-βPro-βThr | 363 |
| TCMCA34 38 | Ac-Nle-c[Asp-His-DPhe-Arg-Trp-Lys]-βThr-βPro-βThr | 364 |
| TCMCA34 39 | Ac-Nle-c[DAsp-Pro-DPhe-Arg-Trp-DLys]-βThr-βPro-βThr | 365 |
| TCMCA34 40 | Ac-Nle-c[DAsp-His-DPhe-Arg-Trp-DLys]-βThr-βPro-βThr | 366 |
| TCMCA34 41 | Ac-Nle-c[Asp-Pro-DPhe-Arg-Trp-Lys]-DPro-DAla-NH2 | 367 |
| TCMCA34 42 | Ac-Nle-c[Asp-His-DPhe-Arg-Trp-Lys]-DPro-DAla-NH2 | 368 |
| TCMCA34 43 | Ac-Nle-c[DAsp-Pro-DPhe-Arg-Trp-DLys]-DPro-DAla-NH2 | 369 |
| TCMCA34 44 | Ac-Nle-c[DAsp-His-DPhe-Arg-Trp-DLys]-DPro-DAla-NH2 | 370 |
| TCMCA34 45 | Ac-Nle-c[Asp-Pro-DPhe-Arg-Trp-Lys]-DAla-DPro-NH2 | 371 |
| TCMCA34 46 | Ac-Nle-c[Asp-His-DPhe-Arg-Trp-Lys]-DAla-DPro-NH2 | 372 |
| TCMCA34 47 | Ac-Nle-c[DAsp-Pro-DPhe-Arg-Trp-DLys]-DAla-DPro-NH2 | 373 |
| TCMCA34 48 | Ac-Nle-c[DAsp-His-DPhe-Arg-Trp-DLys]-DAla-DPro-NH2 | 374 |
| TCMCA34 49 | Ac-Nle-c[Asp-Pro-DPhe-Arg-Trp-Lys]-βPro-βAla-NH2 | 375 |
| TCMCA34 50 | Ac-Nle-c[Asp-His-DPhe-Arg-Trp-Lys]-βPro-βAla-NH2 | 376 |
| TCMCA34 51 | Ac-Nle-c[DAsp-Pro-DPhe-Arg-Trp-DLys]-βPro-βAla-NH2 | 377 |
| TCMCA34 52 | Ac-Nle-c[DAsp-His-DPhe-Arg-Trp-DLys]-βPro-βAla-NH2 | 378 |
| TCMCA34 53 | Ac-Nle-c[Asp-Pro-DPhe-Arg-Trp-Lys]-βAla-βPro-NH2 | 379 |
| TCMCA34 54 | Ac-Nle-c[Asp-His-DPhe-Arg-Trp-Lys]-βAla-βPro-NH2 | 380 |
| TCMCA34 55 | Ac-Nle-c[DAsp-Pro-DPhe-Arg-Trp-DLys]-βAla-βPro-NH2 | 381 |
| TCMCA34 56 | Ac-Nle-c[DAsp-His-DPhe-Arg-Trp-DLys]-βAla-βPro-NH2 | 382 |
| TCMCA34 57 | Ac-Nle-c[Asp-Pro-DPhe-Arg-Trp-Lys]-βVal-βAla-NH2 | 383 |
| TCMCA34 58 | Ac-Nle-c[Asp-His-DPhe-Arg-Trp-Lys]-βVal-βAla-NH2 | 384 |
| TCMCA34 59 | Ac-Nle-c[DAsp-Pro-DPhe-Arg-Trp-DLys]-βVal-βAla-NH2 | 385 |
| TCMCA34 60 | Ac-Nle-c[DAsp-His-DPhe-Arg-Trp-DLys]-βVal-βAla-NH2 | 386 |
| TCMCA34 61 | Ac-Nle-c[Asp-Pro-DPhe-Arg-Trp-Lys]-(3-methyl)-βVal-βAla-NH2 | 387 |
| TCMCA34 62 | Ac-Nle-c[Asp-His-DPhe-Arg-Trp-Lys]-(3-methyl)-βVal-βAla-NH2 | 388 |
| TCMCA34 63 | Ac-Nle-c[DAsp-Pro-DPhe-Arg-Trp-DLys]-(3-methyl)-βVal-βAla-NH2 | 389 |

### Lipopolysaccharide Cachexia Assay

In the experiments performed in accordance with the present technology, a series of melanocortin receptor antagonist peptides were examined in a bacterial endotoxin inducing cachexia assay. For this assay, a rat is challenged with a single peripheral injection of lipopolysaccharide (LPS). LPS is a heat-stable lipo-polysaccharide, the major component of the outer membrane (capsule) of gram negative bacteria. In non-capsulated strains, it appears as a cell surface protein. LPS produces a strong immune response, including secretion of the proinflammatory cytokines Tumor Necrosis Factor α (TNFα) and interleukin-1 (IL-1). Proinflammatory cytokines are known factors that induce cachexia.

Rats are maintained on a 12-hour light/dark cycle. Animals are handled daily for a minimum of 5 consecutive days to acclimate them to the handling required for the experiment. This is critical for feeding experiments, because stressed rats do not eat. On the day of the experiment, individually housed animals receive intraperitoneal (IP) injections of LPS dissolved in 0.5% low-endotoxin BSA, 0.9% saline or 0.5% BSA in 0.9% saline alone, and returned in their home cage. Melanocortin receptor antagonist peptides were administered (IP or by gavage, depending on the type of study) one hour after the LPS injection. The extent of cachexia is measured by the reduction in food intake, measured every two hours. The cachexia typically lasts only a single day. However, rats lose up to 5% of their body weight during a 12-hour light cycle, making up this loss plus food needed for normal growth during the 12-hour dark cycle. Thus, failure to eat appropriately during a single dark cycle is determinable from the animal's weight and any changes thereof.

### SUMMARY

The number of new therapeutic agents has declined steadily over the past 30 years, despite the increasing use of combinatorial chemistry and high throughput screening by pharmaceutical company. Horrobin, J. Roy. Soc. Med. 93:341-345 (2000); Scannell et al., Nature Rev. Drug Discov. 11:191-200 (2012). These techniques can generate literally thousands of compounds with enhanced receptor affinity. However, major reasons for drug candidate failure are not addressed by merely focusing an enhanced receptor affinity. Huggins et al., J. Med. Chem. 55:1424-1444 (2012).

Small molecules (often peptide mimetics) were supposed to correct the pharmacokinetic defects inherent in therapeutic peptides. Small molecules are typically produced by combinatorial chemistry, combined with high throughput assay screening of target receptor binding. Increasingly, however, merely using enhanced binding affinity as a metric of success in a drug development strategy does not result in enhanced specificity or reduced toxicity. Horrobin, J. Roy. Soc. Med. 93:341-345 (2000). The enhanced specificity and reduced toxicity are characteristics of biological peptides. Cirillo et al., Curr. Med. Chem. 18:2854-2866 (2011); Sun, Modern Chem. Applications 1:1-2 (2013). However, peptides used as pharmaceuticals can lack gastrointestinal (GI) transport and organ uptake to reach the target receptor. Herrera-Ruiz and Knipp, J. Pharm. Sci. 92:691-714 (2003); Huggins et al., J. Med. Chem. 55:1424-1444 (2012).

Over the past 30 years, there have been examples of peptides, both synthetic and natural, with blood brain barrier penetrating-ability due to unknown mechanisms. Rodriguez et al., Ann. NY Acad. Sci. 689:537-539 (1993); Sefler et al., J. Med. Chem. 38:249-257 (1995); Sutton et al., Peptides 29:104-111 (2008). It was hypothesized that there might be a common factor or mechanism mediating this transport, and that it could be possible to pre-hoc produce epithelial barrier penetrating peptides: e.g., peptides with oral and blood brain barrier penetrating ability.

Lipinski's “Rule of 5” is a series of metrics defining physical characteristics of molecules with GI tract or blood brain barrier paracellular (between cell) transport (e.g., molecular weights of no greater than 500 g/mol). Lipinski et al., Adv. Drug Deliv. Rev. 46:3-26 (2001). These “rules” suggest that peptides (all with molecular weights greater than 1000 Da) were moving through epithelial barriers by a different, probably transcellular, mechanism. Movement by an oligopeptide transport system is a probable explanation.

Small (oligo) peptide transport systems have been shown to be present on many epithelial barriers, including the GI tract and the blood brain barrier. Herrera-Ruiz et al., AAPS Pharm Sci 3: E9 (2001); Smith et al., Adv. Drug Deliv. Rev. 56:1765-1791 (2004). There have been successful attempts to use these transporters to carry poorly absorbed therapeutic agents, including peptides, across the GI tract. Yang et al., Pharm. Res. 16:1331-1343 (1999); Kikuchi et al., J. Pharm. Sci. 98:1775-1787 (2009). However, these drugs were of rather low molecular weight (500 Da). A major benefit in using peptide (active) transporters to carry therapeutic agents across epithelial barriers (i.e., the intestine for oral activity) is that there is a good degree of trans-species similarity, compared to paracellular (passive) transport. Cao et al., Pharm. Res. 23:1675-1686 (2006). Peptide transporters are the mechanism through which many of the breakdown products of proteins are absorbed and distributed throughout the body. Because this is a fundamental property of living organisms, it is highly conserved.

By testing a series of non-naturally occurring melanocortin analogs with progressive derivatizations, oral and blood-brain barrier transport activity in peptides of greater than 1000 molecular weight were obtained. These effects were amino acid specific and stereospecific, and were independent of the total sum of charged and lipophilic residues. These effects are in stark variance with Lipinski's Rule of 5. However, these results are consistent with a ligand-receptor interaction mediating the transcellular movement of these peptides.

A series of melanocortin receptor antagonist peptides were examined in a bacterial toxin inducing cachexia assay. In this assay, a rat was challenged with a single peripheral injection of lipopolysaccharide (LPS), a bacterial toxin that rapidly induces acute cachexia. The extent of cachexia was measured as a reduction in food intake, measured every two hours. The cachexia typically lasts only a single day.

Non-naturally occurring melanocortin analogs have been shown to be potent anti-cachexia agents, with the ability to reverse cachexia-induced anorexia and restore lean body mass. Marks et al., Cancer Res. 61:1432-1438 (2001); DeBoer, Curr. Opin. Clin. Nutr. Metab. Care 10:457-462 (2007). Bacterial toxin cachexia lends itself to a rapid assay procedure for evaluating the activity of anti-cachexia drugs given by various routes of administration. A critical aspect to melanocortin antagonist anti-cachexia activity is access to the central nervous system (blood-brain barrier transport). Many peptide and small molecule non-naturally occurring melanocortin analogs that have excellent anti-cachexia activity when administered intraventricular (IVt) are unable to affect feeding when given peripherally. U.S. Pat. No. 7,342,089.

Previous studies demonstrated the importance of metabolically stable multi-residue C-terminal extensions to suppress the undesirable cardiovascular side effects of non-naturally occurring melanocortin analogs. Nordheim et al., Peptides 27:438-443 (2006); Greenfield et al., N. Engl. J. Med. 360:44-52 (2009); and U.S. Patent Application Publication No. US 2012/0220525, which is hereby incorporated by reference. One of the molecules produced using this strategy was Ac-Nle-c[Asp-Pro-D-Nal(2′)-Arg-Trp-Lys]-D-Thr-D-Pro-D-Thr (SEQ ID NO: 315) (TCMCB01). While showing little cardiovascular activity, it only produced an anti-cachexia effect when given by IVt administration; there was no blood-brain barrier transport. A series of other metabolically stable oligopeptide extensions in the TCMCB01 molecule were examined.

The initial molecule in the synthesis strategy was: Ac-Nle-c[Asp-Pro-D-Nal(2′)-Arg-Trp-Lys]-D-Pro-D-Val-NH₂ (SEQ ID NO: 309) (TCMCB02) and contains a metabolically stable C-terminal di-peptide extension (D-Pro-D-Val-amide). While this molecule produced anti-cachexia following peripheral administration (i.e., blood-brain barrier transport activity), it had no oral activity. Therefore, the role of other regions of the molecule, i.e. away from the C-terminal di-peptide, were examined for mediating epithelial transport.

For example, the importance of the Pro substitution for His within the cyclized pharmacophore of non-naturally occurring melanocortin analogs has been queried. Sutton et al., Peptides 29:104-111 (2008). The TCMCB02 candidate was compared to its His-substituted derivative: Ac-Nle-c[Asp-His-D-Nal(2′)-Arg-Trp-Lys]-D-Pro-D-Val-amide (SEQ ID NO: 316) (TCMCB03). This molecule had superior anti-cachexia activity following ICV administration, indicating that it was a better antagonist at the central melanocortin system. However, TCMCB03 did not possess as good peripheral (intraperitoneal/IP administration) anti-cachexia activity; it had less blood-brain barrier transport activity. This demonstrated that the structure of the peptide being transported (the “load”) by the C-terminal di-peptide is as important as the “vehicle.” A potential explanation for this effect is that substituting Pro for His decreases intramolecular free rotation, since Pro is an imidic acid: its α-carbon has a secondary amino group forming a peptide bond. These data are consistent with suggestions that a reduction in free rotation enhances the trans-epithelial movement of peptides. Vabeno et al., Bioorg. Med. Chem. 13:1977-1988 (2005); Hess et al., J. Med. Chem. 51:1026-1034 (2008). Cyclization is another approach that can decrease the degree of free rotation typically inherent in a small peptide, and can enhance active peptide transport. Vabeno et al., Bioorg. Med. Chem. 13:1977-1988 (2005).

The basis for cyclizing melanocortins was that a reverse turn conformation was required for receptor binding. Sawyer et al., Proc. Nat. Acad. Sci. USA 79:1751-1755 (1982); Sawyer et al., J. Med. Chem. 25:1022-1027 (1982). Initially, a D-amino acid substitution in the middle of the melanocortin pharmacophore enhanced biological activity; presumably because a D-residue will reduce the tendency of an L-amino acid sequence to form an α-helix, but stabilize a β-turn. Sawyer et al., Proc. Nat. Acad. Sci. USA 77:5754-5758 (1980). Cyclized melanocortins were the next step; first di-sulfide bridging, then a lactam bridge. It was hypothesized that using D-amino acid residues to form the lactam bridge in cyclic MCs would further stabilize the cyclic structure, producing superior biological activity. The D-Asp⁵, D-Lys¹⁰ derivative of TCMCB02; Ac-Nle-c[D-Asp-Pro-D-Nal(2′)-Arg-Trp-D-Lys]-D-Pro-D-Val-NH₂ (SEQ ID NO: 317) (TCMCB04) was synthesized and tested.

Given ICV, this derivative had superior ICV anti-cachectic activity (stimulation of feeding in the LPS-induced cachexia model) compared to TCMCB02. However, the derivatization eliminated blood-brain barrier transport; IP administration of the peptide produced no anti-cachexia activity.

The TCMCB04 data demonstrated the stereospecificity of the mechanism responsible for blood-brain barrier transport; stereospecificity is further evidence against paracellular transport. Finally, when taken with the TCMCB03 data (His⁶ vs. Pro⁶), it was apparent that relatively minor alterations in the structure of the molecule being transported produce profound changes in transport properties.

A series of derivatives were designed, seeking to combine the steric properties of L-amino acids with the resistance to enzymatic degradation of D-residues. Amino or imidic acid peptide residues with their α-amino and/or carboxyl groups displaced to the β-carbon maintain their side-chain steric structure, but are stable to enzymatic degradation. Lew et al., FASEB J. 15:1664-1666 (2001); Nachman et al., Peptides 30:608-615 (2009).

Derivatives of TCMCB02 were synthesized, replacing the C-terminal di-peptide with its B amino acid equivalents: Ac-Nle-c[Asp-Pro-D-Nal(2′)-Arg-Trp-Lys]-β³-Pro-2-Val-NH₂ (SEQ ID NO: 318) (TCMCB05) or Ac-Nle-c[Asp-Pro-D-Nal(2′)-Arg-Trp-Lys]-β³-Pro-β³-Val-NH₂ (SEQ ID NO: 319) (TCMCB06). Following ICV administration, both TCMCB05 and TCMCB06 had molar activity equivalent to TCMCB02 in LPS-induced cachexia. However, only TCMCB05 had peripheral anti-cachexia activity following LPS administration and provided evidence for β-residue subtype specific blood-brain barrier transport properties in the di-peptide extension (amino group on β carbon preferred over the carboxyl on the β carbon). The ability to differentiate between β amino acid subtypes is a characteristic of an oligopeptide transport mechanism. Brodin et al., Pharmacol. Toxicol. 90:285-296 (2002). TCMCB05, however, was not orally active as an anti-cachexia agent.

In order to increase transport activity in the peptides, the di-peptide sequence specificity for transport properties was investigated. The sequence of the C-terminal di-peptide in TCMCB02, was reversed, producing the derivative Ac-Nle-c[Asp-Pro-D-Nal(2′)-Arg-Trp-Lys]-D-Val-D-Pro-NH₂ (SEQ ID NO: 3) (TCMCB07). The TCMCB07 peptide had about 10-times more activity following peripheral administration than TCMCB02. In addition, TCMCB02 was orally active as an anti-cachexia agent.

TCMCB07 and TCMCB07A are the same peptide except that TCMCB07 has a C-terminal amide moiety and TCMCB07A does not. Peripheral administration of TCMCB07 or TCMCB07A produces equivalent stimulation of feeding in LPS-induced cachexia. Thus, the C-terminal amide moiety has no effect on transport, but may stabilize the peptide from degradation.

Reversing the C-terminal di-peptide sequence (e.g., Pro-Val to Val-Pro) improved transport in the D-residue di-peptide derivatives. Accordingly, β-residue extensions with a reversed sequence (i.e., -Pro-β-Val changed to β-Val-β-Pro) were investigated. The reversed di-peptide sequence of TCMCB05, i.e., Ac-Nle-c[Asp-Pro-D-Nal(2′)-Arg-Trp-Lys]-32-Val-β³-Pro-NH₂ (SEQ ID NO: 390) (TCMCB08); had good blood-brain barrier transport, but not as much oral activity as TCMCB07. The reversed sequence of TCMCB06, Ac-Nle-c[Asp-Pro-D-Nal(2′)-Arg-Trp-Lys]-β³-Val-β³-Pro-NH₂ (SEQ ID NO: 391) (TCMCB09), was inactive peripherally.

The rapid appearance in blood of orally administered proline-rich peptides was some of the first evidence for active transport of peptides through the GI tract, and an Xaa-Pro dipeptide is a substrate for the PEPY1. Given these data, the C-terminal di-Pro derivative of TCMCB02 was synthesized and tested: Ac-Nle-c[Asp-Pro-D-Nal(2′)-Arg-Trp-Lys]-β³-Pro-β³-Pro-NH₂ (SEQ ID NO: 392) (TCMCB10). While maintaining direct inhibitory effects (ICV administration in LPS cachexia) on the central melanocortin system, TCMCB10 showed no peripheral activity/a failure to cross the blood-brain barrier.

TABLE 12
Metabolically Stabilized Non-naturally occurring melanocortin analogs

| | | | SEQ ID |
|---|---|---|---|
| Peptide | Structure | Action | NO: |
| TCMCB01 | Ac-Nle-c[Asp-Pro-DNal(2′)-Arg-Trp-Lys]- | Antagonist | 315 |
| | DThr-DPro-DThr | | |
| TCMCB02 | Ac-Nle-c[Asp-Pro-DNal(2′)-Arg-Trp-Lys]- | Antagonist | 309 |
| | DPro-DVal-NH2 | | |
| TCMCB03 | Ac-Nle-c[Asp-His-DNal(2′)-Arg-Trp-Lys]- | Antagonist | 316 |
| | DPro-DVal-NH2 | | |
| TCMCB04 | Ac-Nle-c[D-Asp-Pro-DNal(2′)-Arg-Trp-DLys]- | Antagonist | 317 |
| | DPro-DVal-NH2 | | |
| TCMCB05 | Ac-Nle-c[Asp-Pro-DNal(2′)-Arg-Trp-Lys]- | Antagonist | 318 |
| | β³Pro-β²Val-NH2 | | |
| TCMCB06 | Ac-Nle-c[Asp-Pro-DNal(2′)-Arg-Trp-Lys]- | Antagonist | 319 |
| | β³Pro-β³Val-NH2 | | |
| TCMCB07 | Ac-Nle-c[Asp-Pro-DNal(2′)-Arg-Trp-Lys]- | Antagonist | 3 |
| | DVal-DPro-NH2 | | |
| TCMCB07A | Ac-Nle-c[Asp-Pro-DNal(2′)-Arg-Trp-Lys]- | Antagonist | 2 |
| | DVal-DPro | | |
| TCMCB08 | Ac-Nle-c[Asp-Pro-DNal(2′)-Arg-Trp-Lys]- | Antagonist | 390 |
| | β²Val-β³Pro-NH2 | | |
| TCMCB09 | Ac-Nle-c[Asp-Pro-DNal(2′)-Arg-Trp-Lys]- | Antagonist | 391 |
| | β³Val-β³Pro-NH2 | | |
| TCMCB10 | Ac-Nle-c[Asp-Pro-DNal(2′)-Arg-Trp-Lys]- | Antagonist | 392 |
| | β³Pro-β³Pro-NH2 | | |

TABLE 13
Summary of Activities of Metabolically Stabilized
Non-naturally occurring melanocortin analogs

| | | | | | Anti- |
|---|---|---|---|---|---|
| | | SEQ | Oral | Periph. | cachectic |
| Cmpd | Melanocortin Antagonist | ID NO: | Activity | Activity | Activity |
| TCMCB01 | Ac-Nle-c[Asp-Pro-D-Nal(2′)-Arg-Trp- | 315 | − | − | + |
| | Lys]-D-Thr-D-Pro-D-Thr | | | | |
| TCMCB02 | Ac-Nle-c[Asp-Pro-D-Nal(2′)-Arg-Trp- | 309 | − | + | + |
| | Lys]-D-Pro-D-Val-NH₂ | | | | |
| TCMCB03 | Ac-Nle-c[Asp-His-D-Nal(2′)-Arg-Trp- | 316 | − | − | ++ |
| | Lys]-D-Pro-D-Val-NH₂ | | | | |
| TCMCB04 | Ac-Nle-c[D-Asp-Pro-D-Nal(2′)-Arg- | 317 | − | − | ++ |
| | Trp-D-Lys]-D-Pro-D-Val-NH₂ | | | | |
| TCMCB05 | Ac-Nle-c[Asp-Pro-D-Nal(2′)-Arg-Trp- | 318 | − | + | ++ |
| | Lys]-β³-Pro-β²-Val-NH₂ | | | | |
| TCMCB06 | Ac-Nle-c[Asp-Pro-D-Nal(2′)-Arg-Trp- | 319 | − | − | ++ |
| | Lys]-β³-Pro-β³-Val-NH₂ | | | | |
| TCMCB07 | Ac-Nle-c[Asp-Pro-D-Nal(2′)-Arg-Trp- | 3 | + | ++ | +++ |
| | Lys]-D-Val-D-Pro-NH₂ | | | | |
| TCMCB07A | Ac-Nle-c[Asp-Pro-D-Nal(2′)-Arg-Trp- | 2 | + | ++ | +++ |
| | Lys]-D-Val-D-Pro | | | | |
| TCMCB08 | Ac-Nle-c[Asp-Pro-D-Nal(2′)-Arg-Trp- | 390 | −+ | ++ | +++ |
| | Lys]-β²-Val-β³-Pro-NH₂ | | | | |
| TCMCB09 | Ac-Nle-c[Asp-Pro-D-Nal(2′)-Arg-Trp- | 391 | − | − | +++ |
| | Lys]-β³-Val-β³-Pro-NH₂ | | | | |
| TCMCB10 | Ac-Nle-c[Asp-Pro-D-Nal(2′)-Arg-Trp- | 392 | − | − | +++ |
| | Lys]-β³-Pro-β³-Pro-NH₂ | | | | |

### Example 9. Physicochemical Evidence for Transport of Non-Naturally Occurring Melanocortin Analogs through the Rat Blood Brain Barrier

About 60 years ago, the prevailing view that dietary protein absorption occurred after these molecules were broken down to their constituent amino acids was challenged by the evidence for rapid intestinal absorption of di and tri-peptides. Prockop et al., Lancet 2:527-528 (1962); Brandsch et al., J. Pharm. Pharmacol. 60:543-585 (2008). In subsequent decades, the presence of several peptide transport systems for di- and tri-peptides was well documented. Most of this work has focused on the PEPT1 and PEPT2 transport systems, since PEPT1 appears to be the predominant system mediating intestinal absorption of protein-derived peptides. Brodin et al., Pharmacol. Toxicol. 90:285-296 (2002); Brandsch et al., J. Pharm. Pharmacol. 60:543-585 (2008).

The discovery that peptide mimetic antibiotics undergo intestinal absorption via PEPT1 stimulated work on the use of the PEPT1 transport system to enhance the intestinal absorption of drugs with poor oral bioavailability. Sugawara et al., J. Pharm. Sci. 89:781-789 (2000); Terada et al., Pflug. Arch. Eur. J. Physiol. 440:679-684 (2000); Vabeno et al., Bioorg. Med. Chem. 13:1977-1988 (2005); Brandsch et al., J. Pharm. Pharmacol. 60:543-585 (2008); Kikuchi et al., J. Pharm. Sci. 98:1775-1787 (2009). However, the evidence suggested that only molecular complexes of a relatively small size, on the order of di or tri-peptides, could utilize this system. The subsequent discovery of other oligopeptide transport systems did not produce the intense scrutiny of structural transport requirements to which the PEPT family was subjected. Herrera-Ruiz and Knipp, J. Pharm. Sci. 92:691-714 (2003); Smith et al., Adv. Drug Deliv. Rev. 56:1765-1791 (2004).

The stereospecific, di-peptide sequence specific, and β-residue subtype specific nature of oral activity in the non-naturally occurring melanocortin analogs are requirements that effectively eliminate physical chemical properties as factors mediating movement through epithelial barriers. These structural requirements, however, point to a trans-cellular small peptide transporter. The transporter is similar, but different, in the blood brain barrier (a PEPT2-containing organ) versus the gastrointestinal tract.

There are parallels between the results present technology and what is known about the intestinal epithelial peptide transport system, PEPT1. The peptides present technology and PEPT1 show stereospecificity, sequence specificity, and β-amino acid subtype specificity. Addison et al., Clin. Sci. Mol. Med. 49:313-322 (1975); Adibi, Am. J. Physiol. 272: E723-736 (1997); Brandsch, Amino Acids 31:119-136 (2006). Further, there is much greater activity for a di-peptide sequence where there is a proline at the C-terminus and an aliphatic non-polar residue (e.g., Gly, Val, Leu) at the penultimate position, i.e., Val-Pro would have about 10-times more transport activity than Pro-Val. Brandsch, Amino Acids 31:119-136 (2006). This is consistent with comparisons of non-naturally occurring melanocortin analogs present technology with D-Pro-Val vs. D-Val-D-Pro C-terminal extensions. However, there are also differences, because many molecules present technology comprise blocked amino and carboxyl termini, as well as a D-amino acid residues in the di-peptide transport sequence. These structural factors are reported to be unfavored for PEPT1 transport. Terada et al., Pflug. Arch. Eur. J. Physiol. 440:679-684 (2000).

Structural requirements are present technology that permit oligopeptide transport for much larger molecular weights than have previously reported (e.g., greater than 1200 Da). These studies with melanocortins could facilitate the development of orally active synthetic peptide analogs of natural peptides with molecular weights in excess of 2000 Da. Minor changes in the transported drug's structure produce major changes in transport properties, even though the di-peptide is largely unchanged.

### TCMCB07 and the Blood-Brain Barrier

Urethane-anesthetized rats had polyethylene (PE)-tubing cannulas placed in their femoral artery and vein. Each rat was then placed in a small animal stereotaxic apparatus, their head fixed in the lateral plane with ear bars, and their head placed in a 45-50° downward angle by placing their nose below an adjustable tooth bar. This position placed the animal's cistern magna at the highest vertical position of its body. The dural covering of the dorsal cistern magna was exposed.

A stainless steel cannula, with one end attached to PE-tubing, was placed in a stereotaxic tower cannula holder (open end down). The free end of the PE-tubing was attached to a 100 UL gas-tight Hamilton syringe with a 28-gauge needle. The cannula was adjusted to a position that approximated the middle of the exposed cistern magna, and the cannula lowered until it touched the dura mater. A vertical stereotaxic reading was recorded, and the cannula slowly lowered until it pierced the dural membrane. The dura has a degree of flexibility such that the cannula can depress it before it pierces it. After it was pierced, the dural rises to its original position, placing the cannula tip within the cistern magna space. The position of the cannula tip was again noted on the vertical axis of the tower. Typically, the cannula tip was 1.6-1.8 mm below the original dura-touch position. The cannula tip was then adjusted, if needed, to 1.8 mm below the dura, and the cannula sealed to the dura using skin glue. Samples of cisternal cerebrospinal (CSF) fluid were removed through cannula and PE-tubing by slowly drawing on the Hamilton syringe.

For the experimental procedure, baseline samples of blood and CSF were taken (“O” time points), followed by the IV administration of 750 nmol/kg TCMCB07. Samples of CSF (50 μL) and blood (1 mL) were collected at the following time points: 0, 5, 15, 30, and 90 minutes. CSF samples were diluted with acetonitrile-HCL to a concentration of 20% acetonitrile, and chromatographed on a microparticulate C-18 reversed-phase high performance liquid chromatography (HPLC) column with a 20-40% acetonitrile-aqueous HCL gradient over 20 minutes. Blood was collected with heparin from the arterial line, and the plasma was isolated by centrifugation. Both CSF and plasma were stored at −80° C. until analyzed by (HPLC) with spectrofluoremetric monitoring at 230 nm excitation and 237 nm emission (the fluorescence maximum for the Nal residue). The spectrofluorometer effluent was collected every 30 seconds, and samples representing fluorometer peaks potentially positive for the Nal residue were subjected to mass spectrometry molecular mass analysis.

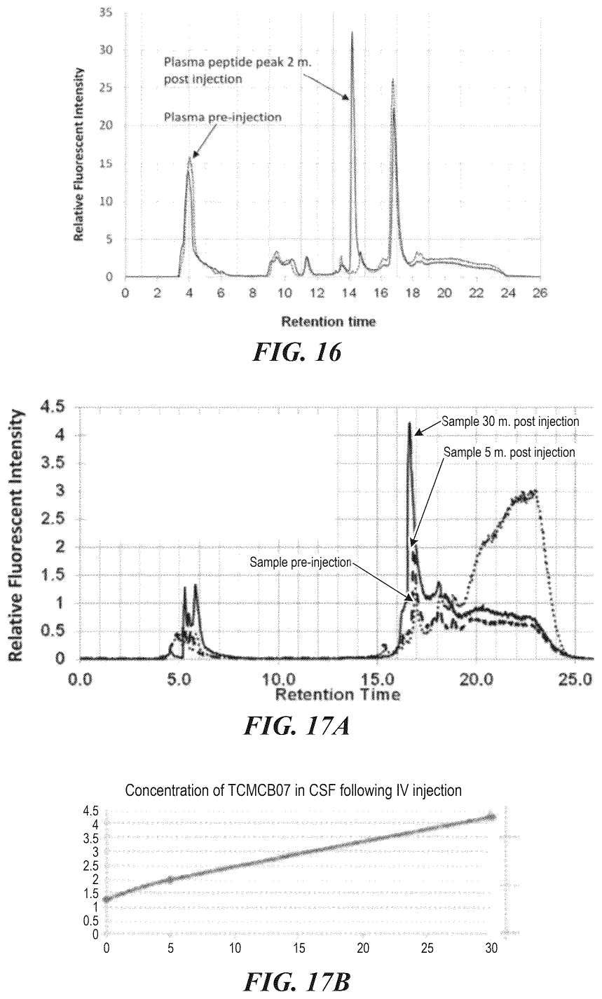

FIGS. 16 and 17A-17B show high performance liquid chromatograms of plasma (FIG. 16) and CSF (FIG. 17), pre- and post-IV administration of TCMCB07 (750 nmol/kg). A naphthylalanine (Nal residue)-positive fluorescence peak is seen in plasma at the exact retention time of TCMCB07 (FIG. 16). The CSF had a Nal-positive fluorescence peak with a slightly earlier retention time than TCMCB07 (FIG. 17A). The peak height increases over time after IV drug administration, suggesting it is a TCMCB07 metabolite (FIG. 17B).

Subsequent analysis of the collected HPLC-spectrofluorometer effluent by mass spectrometry confirmed the presence of TCMCB07 in CSF at the predicted HPLC retention time, even though an obvious Nal-fluorescence positive peak could not be detected. However, mass spectrometry is about 10.000-fold more sensitive than fluorescence.

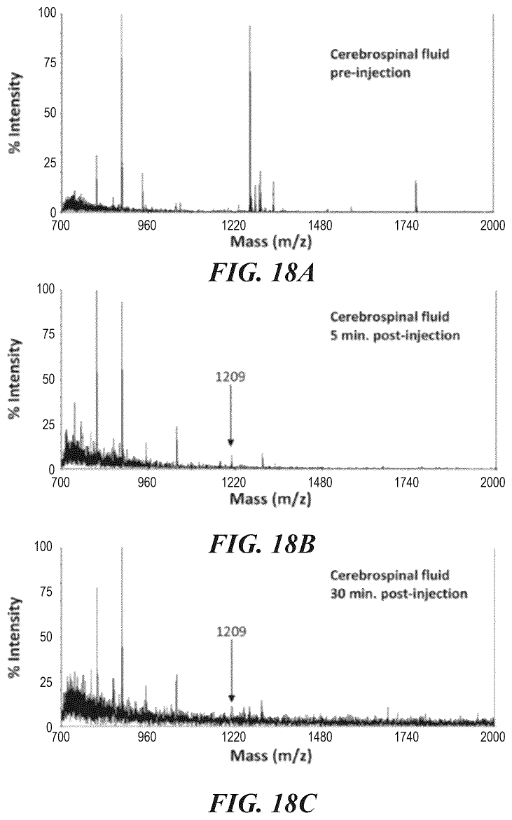

The presumed TCMCB07 metabolite at 12.9 minutes retention time showed a molecular mass of 1209.0 Daltons, 22 Daltons less than TCMCB07. This peptide was only present in CSF after IV administration of TCMCB07; i.e., not present in “0” time CSF samples (FIG. 18A-18C). This suggests that the Nal-positive peak at 12.9 minutes retention time is a TCMCB07 breakdown product.

These results provide direct physical evidence of TCMCB07 transport through the blood-brain-barrier that is consistent with the biological evidence of TCMCB07 therapeutic effects observed in cachexia.

### Example 10. Studies of Non-Naturally Occurring Melanocortin Analogs on Body Weight and Food Consumption

The impact of the non-naturally occurring melanocortin analog (TCMCB07; SEQ ID NO: 3) on body weight and food consumption was examined in rats. Specifically, a cohort of male rats and a cohort of female rats were each separated into four groups and administered 0.5 mg/kg/day TCMCB07 (Group 2), 3 mg/kg/day TCMCB07 (Group 3), or 10 mg/kg/day TCMCB07 (Group 4), respectively, for 28 days. Group 1 served as a control and received the vehicle (0 mg/kg/day TCMCB07). The body weights and food consumption of the rats were monitored from 3 days prior to the first dose until 56 days after the first dose.

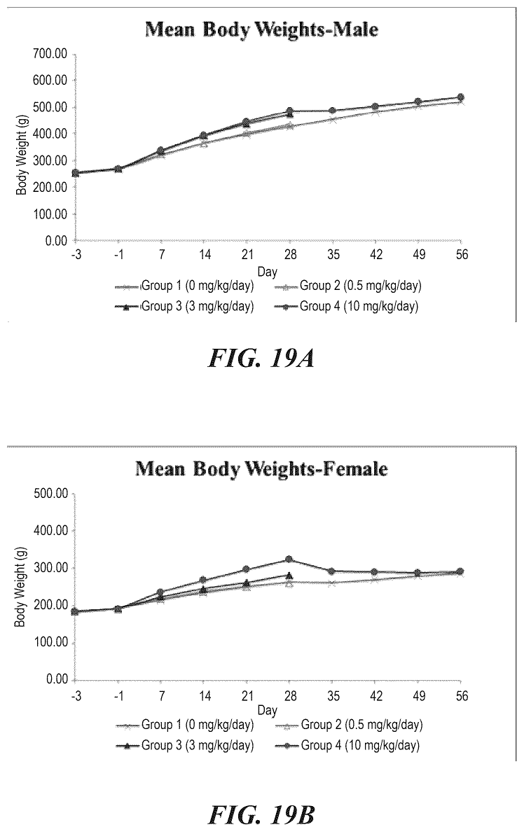

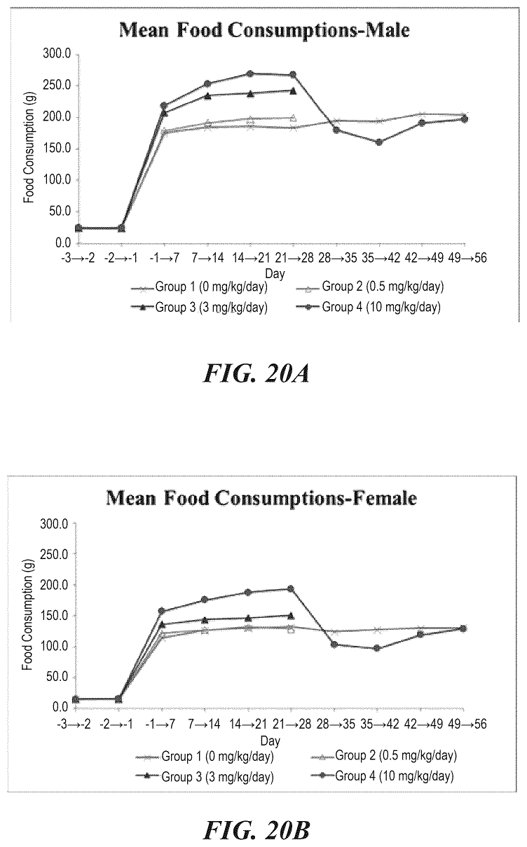

As shown in FIGS. 19A-19B and 20A-20B, drug (TCMCB07) treated groups (Groups 2-4) exhibited significant body weight gain and increased food intake.

The impact of the non-naturally occurring melanocortin analog (TCMCB07; SEQ ID NO: 3) on body weight and food consumption was examined in dogs. Specifically, a cohort of male dogs and a cohort of female dogs were each separated into four groups and administered 0.5 mg/kg/day TCMCB07 (Group 2), 3 mg/kg/day TCMCB07 (Group 3), or 10 mg/kg/day TCMCB07 (Group 4), respectively, for 28 days. Group 1 served as a control and received the vehicle (0 mg/kg/day TCMCB07). The body weights and food consumption of the dogs were monitored from 3 days prior to the first dose until 56 days after the first dose.

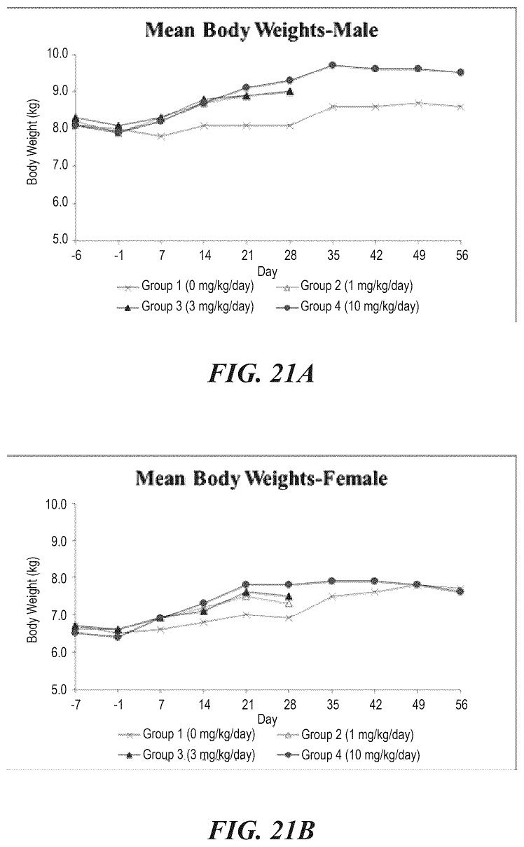

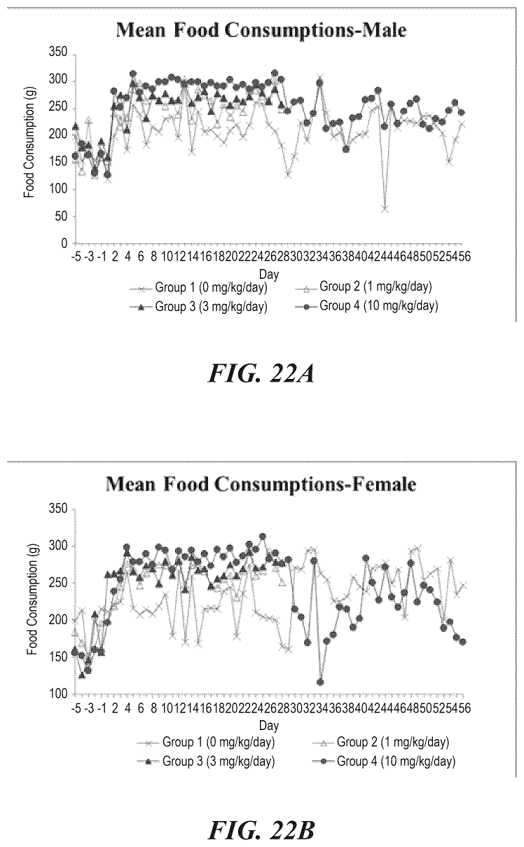

As shown in FIGS. 21A-21B and 22A-22B, drug (TCMCB07) treated groups exhibited significant body weight gain and increased food intake.

### Body Composition and Lean Mass Restoration Studies

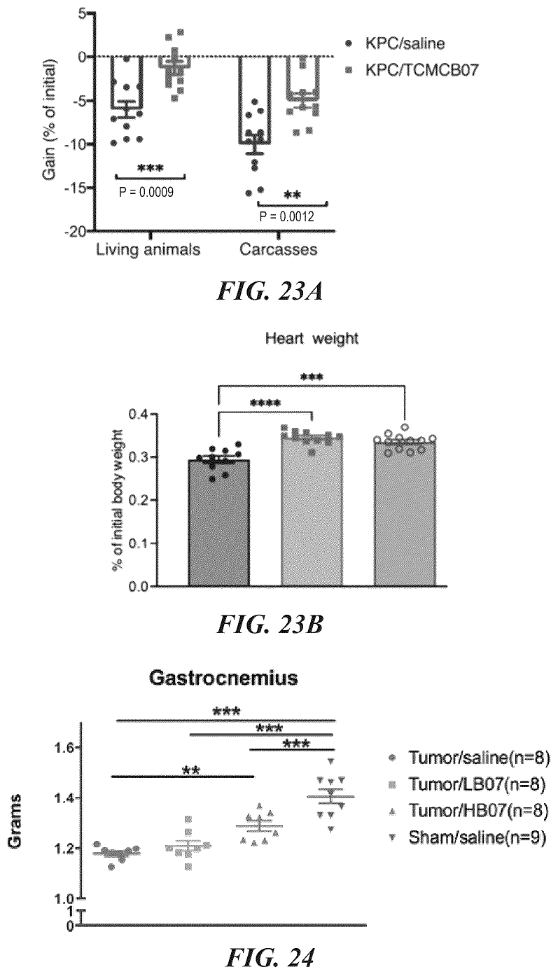

As shown in FIG. 23A, KPC model in mice shows substantial difference in overall lean mass increase between control (KPC/saline) and experimental groups (KPC/TCMCB07) as measured by living weight and carcass weight (% gain over initial weight). FIG. 23B shows preservation of cardiac mass during experiment in rats. The preservation of cardiac mass can be a substantial driver of survival increase and a substantial reduction in key comorbidities. The study also shows that the increase in cardiac mass is proportional to lean body mass, demonstrating a non-specific lean muscle mass increase effect. FIG. 24 shows preservation of gastrocnemius mass with low and high dose β⁰⁷ administration in Lewis Sarcoma model in rats.

### Example 11. Phase 1 Clinical Trial with TCMCB07

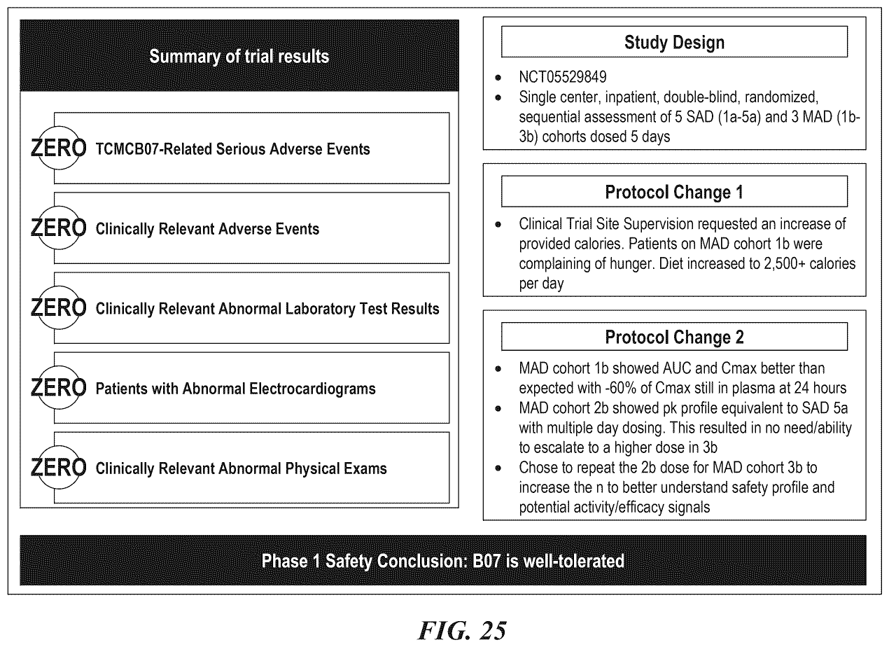

Safety results of the single center, inpatient, double-blind, randomized, sequential assessment of 5 SAD (1a-5a) and 3 MAD (1b-3) cohorts dosed over 5 days TCMCB07 Phase 1 Clinical Trial (NCT05529849) have been summarized in FIG. 25. The entirety of the TCMCB07 Phase 1 Clinical Trial (NCT05529849) is incorporated herein by reference.

As shown in FIG. 25, there are zero drug-related SAEs, zero CAEs, zero clinically relevant abnormal laboratory test results, zero patients having abnormal ECGs, and zero clinically relevant abnormal physical exams.

### Patients Receiving TCMCB07 Show Increased BMI, Weight, and Appetite.

To determine whether TCMCB07 increases BMI, weight, and appetite, the results from TCMCB07 Phase 1 Clinical Trial (NCT05529849) were unblinded and used to compare BMI, weight, and appetite between patients receiving TCMCB07 and placebo.

To determine whether patients treated with TCMCB07 had significantly increased BMI or weight compared to the placebo group over time, cohorts 2 and 3 from the clinical trial were combined and linear mixed effects models were used for all the following analyses to take account of the correlation among observations from the same subject.

BMI was calculated using the weight measured as each day and the height obtained on the screening day: BMI=weight (kg)/(height (m))₂.

A linear mixed effects model was used to take account of the correlation among observations from the same subject. The model included treatment effect, day (of measurement from day0 to day8), day by treatment interaction, and batch. The results showed there was no significant difference between TCMCB07 and placebo group averaged across the testing time (P=0.5934), no significantly different batch effect (P=0.9821), and no significantly different treatment interaction (P=0.5934). However, the day effect showed significant correlation with BMI (P<. 0001), which indicates that BMI changed over time.

The BMI was also compared between the two groups for each day and the results showed there was no significant difference between TCMCB07 and placebo group on each day (p-value>0.48 for all comparisons).

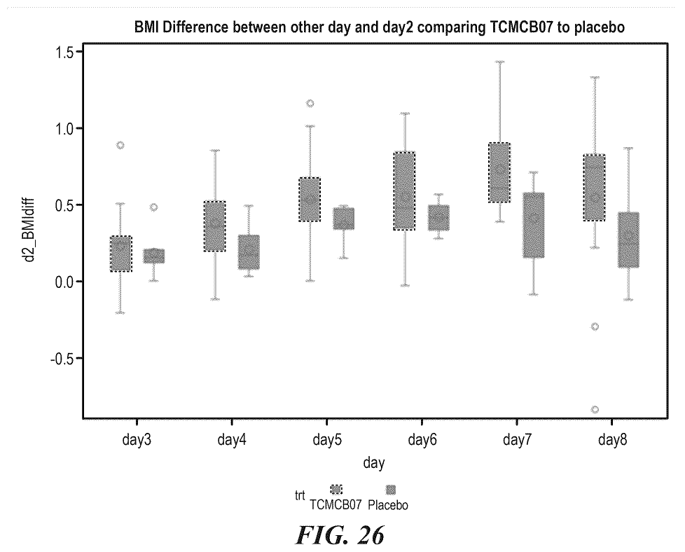

As shown in FIG. 26, the BMI change from each day after day2 was compared to day2 comparing TCMCB07 to placebo as listed in Table 14. The results showed that BMI change between day2 and day7 for the TCMCB07 group is significantly higher than that of the placebo group without adjustment for multiple comparisons, but the difference was not significant comparing other days to day2 between TCMCB07 and placebo group. In general, there is a higher BMI difference (comparing other days to day2) for the TCMCB07 group comparing to the placebo group for all the testing days (Table 15). TCMCB07 group had higher BMI change (mean=0.4934) comparing to day2 than the placebo group (mean=0.3128) averaged across from day3 to 8 but didn't reach significance (P=0.1243).

TABLE 14
BMI difference between TCMCB07 and placebo
group of other days vs day 2 comparison

| | | | Diff1 − | Standard | | | |
|---|---|---|---|---|---|---|---|
| Label | TCMCB07 | placebo | day 2 diff | Error | p-values | 95% CI | |
| Day 8 vs | 0.5437 | 0.2947 | 0.2489 | 0.2256 | 0.2822 | −0.2201 | 0.7180 |
| day 2 | | | | | | | |
| day 7 vs | 0.7294 | 0.4089 | 0.3206 | 0.1440 | 0.0366 | 0.02187 | 0.6192 |
| day 2 | | | | | | | |
| day 6 vs | 0.5501 | 0.4167 | 0.1334 | 0.1439 | 0.364 | −0.1650 | 0.4317 |
| day 2 | | | | | | | |
| day 5 vs | 0.5330 | 0.3651 | 0.1679 | 0.1160 | 0.1618 | −0.07264 | 0.4085 |
| day 2 | | | | | | | |
| day 4 vs | 0.3752 | 0.2059 | 0.1692 | 0.1118 | 0.1444 | −0.06267 | 0.4011 |
| day 2 | | | | | | | |
| day 3 vs | 0.2289 | 0.1853 | 0.04360 | 0.1038 | 0.6785 | −0.1716 | 0.2588 |
| day 2 | | | | | | | |

As shown in FIG. 25 and Table 15, the BMI of patients treated with TCMCB07 was generally greater than the BMI of patients treated with vehicle control.

TABLE 15
Summary of BMI over time for combined
Cohort2 and 3-unblinded data

| | | N | | | Std | | |
|---|---|---|---|---|---|---|---|
| trt | day | Obs | N | Mean | Dev | Minimum | Maximum |
| TCMCB07 | day 0 | 18 | 18 | 27.077 | 2.252 | 20.988 | 29.758 |
| | day 1 | 18 | 18 | 27.066 | 2.067 | 21.358 | 29.861 |
| | day 2 | 18 | 18 | 26.966 | 2.218 | 21.080 | 29.861 |
| | day 3 | 18 | 18 | 27.195 | 2.194 | 21.265 | 29.652 |
| | day 4 | 18 | 18 | 27.341 | 2.164 | 21.512 | 29.890 |
| | day 5 | 18 | 18 | 27.499 | 2.154 | 21.636 | 29.861 |
| | day 6 | 18 | 18 | 27.516 | 2.101 | 21.698 | 29.831 |
| | day 7 | 18 | 18 | 27.695 | 2.208 | 21.667 | 30.249 |
| | day 8 | 17 | 17 | 27.441 | 2.225 | 21.605 | 29.582 |
| Placebo | day 0 | 6 | 6 | 26.663 | 1.970 | 24.539 | 29.024 |
| | day 1 | 6 | 6 | 26.613 | 2.051 | 24.465 | 29.103 |
| | day 2 | 6 | 6 | 26.554 | 1.795 | 24.613 | 28.965 |
| | day 3 | 6 | 6 | 26.739 | 1.702 | 25.097 | 28.965 |
| | day 4 | 6 | 6 | 26.760 | 1.744 | 24.911 | 28.995 |
| | day 5 | 6 | 6 | 26.919 | 1.724 | 24.985 | 29.114 |
| | day 6 | 6 | 6 | 26.970 | 1.863 | 24.948 | 29.532 |
| | day 7 | 6 | 6 | 26.963 | 1.664 | 25.171 | 29.012 |
| | day 8 | 6 | 6 | 26.848 | 1.688 | 25.059 | 29.054 |

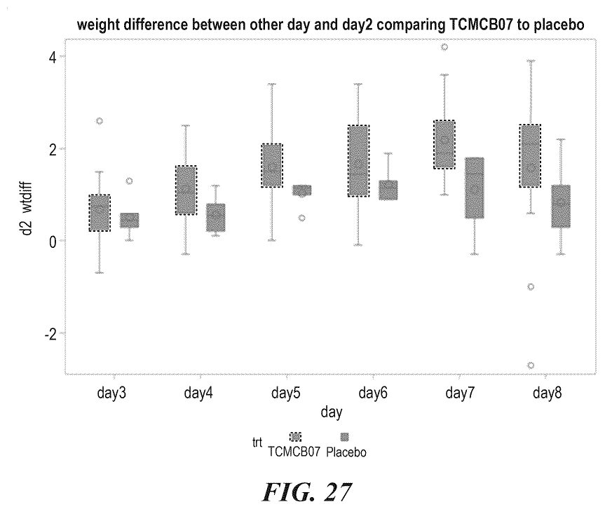

As shown in FIG. 27, the body weight change from each day after day2 was compared to day2 comparing TCMCB07 to placebo as listed in Table 16. Similar to the BMI results, weight change between day2 and day7 for the TCMCB07 group is significantly higher than that of the placebo group without adjustment for multiple comparisons, but the difference was not significant comparing other days to day2 between TCMCB07 and placebo group, which could be due to the small sample size.

TABLE 16
Weight difference between TCMCB07 and placebo
group of other days vs day 2 comparison

| | | | Diff1 − | Standard | | | |
|---|---|---|---|---|---|---|---|
| Label | TCMCB07 | placebo | day 2 diff | Error | p-values | 95% CI | |
| Day 8 vs | 1.5731 | 0.8333 | 0.7398 | 0.6752 | 0.2856 | −0.6643 | 2.1439 |
| day 2 | | | | | | | |
| day 7 vs | 2.1833 | 1.1167 | 1.0667 | 0.4232 | 0.0195 | 0.1889 | 1.9444 |
| day 2 | | | | | | | |
| day 6 vs | 1.6611 | 1.2167 | 0.4444 | 0.4428 | 0.3264 | −0.4739 | 1.3628 |
| day 2 | | | | | | | |
| day 5 vs | 1.600 | 1.0333 | 0.5667 | 0.3518 | 0.1215 | −0.1629 | 1.2963 |
| day 2 | | | | | | | |
| day 4 vs | 1.1333 | 0.5667 | 0.5667 | 0.3262 | 0.0963 | −0.1098 | 1.2431 |
| day 2 | | | | | | | |
| day 3 vs | 0.6889 | 0.5167 | 0.1857 | 0.1722 | 0.5792 | −0.4623 | 0.8067 |
| day 2 | | | | | | | |

In general, there is a higher weight difference (comparing other days to day2) for the TCMCB07 group comparing to the placebo group for all the testing days (Table 17, and FIG. 26). This weight difference is close to be significant averaged across time from day3 to day8 comparing TCMCB07 to placebo group(P=0.0894).

TABLE 17
weight summary over time-unblinded data

| trt | day | N Obs | N | Mean | Std Dev | Minimum | Maximum |
|---|---|---|---|---|---|---|---|
| Drug | day 0 | 18 | 18 | 81.111 | 9.253 | 66.700 | 98.100 |
| | day 1 | 18 | 18 | 81.100 | 9.089 | 67.200 | 100.000 |
| | day 2 | 18 | 18 | 80.783 | 9.274 | 66.900 | 100.000 |
| | day 3 | 18 | 18 | 81.472 | 9.248 | 66.900 | 99.300 |
| | day 4 | 18 | 18 | 81.917 | 9.238 | 66.600 | 100.100 |
| | day 5 | 18 | 18 | 82.383 | 9.202 | 67.600 | 100.000 |
| | day 6 | 18 | 18 | 82.444 | 9.158 | 67.200 | 99.900 |
| | day 7 | 18 | 18 | 82.967 | 9.277 | 67.900 | 101.300 |
| | day 8 | 17 | 17 | 81.388 | 8.032 | 67.900 | 99.000 |
| Placebo | day 0 | 6 | 6 | 78.400 | 15.517 | 63.200 | 97.200 |
| | day 1 | 6 | 6 | 78.317 | 15.963 | 62.800 | 97.200 |
| | day 2 | 6 | 6 | 78.083 | 15.286 | 63.800 | 97.000 |
| | day 3 | 6 | 6 | 78.600 | 15.095 | 64.100 | 97.000 |
| | day 4 | 6 | 6 | 78.650 | 15.082 | 64.000 | 97.100 |
| | day 5 | 6 | 6 | 79.117 | 15.139 | 65.000 | 97.500 |
| | day 6 | 6 | 6 | 79.300 | 15.530 | 64.900 | 98.900 |
| | day 7 | 6 | 6 | 79.200 | 14.822 | 65.600 | 96.700 |
| | day 8 | 6 | 6 | 78.917 | 15.118 | 64.300 | 97.300 |

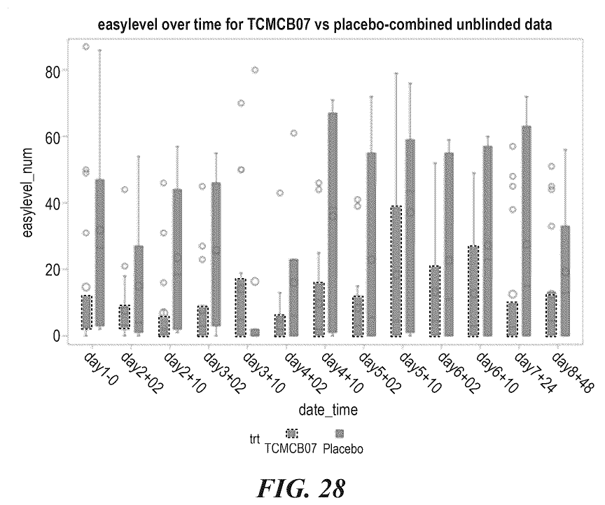

As shown in Table 18 and FIG. 28, appetite generally increased in patients treated with TCMCB07 compared to patients treated with placebo. The appetite was assessed by the patient's scaling of satiety (e.g., easy or hard to eat) from 0 (extremely easy to eat) to 100 (extremely hard to eat). The results showed that there was a significant difference satiety at time-2 (P=0.0892) on days 3-6, and on time+10 on days 2-6 (P=0.0497), as well as including both time-2 and +10 (P=0.0655) from day3 to day6 between subjects on TCMCB07 and those on placebo averaged across time after controlling for batch effect, where subjects on TCMCB07 found it easier to eat compared to those on placebo.

The difference was not significant on days 7 and 8 (P=0.2833), and it was close to be significant including all the time points from days 2 to 8 (P=0.0789). The TCMCB07 group has general lower score over time, meaning patients receiving TCMCB07 generally felt it was easier to eat.

TABLE 18
ease of eating comparison between TCMCB07 and placebo at each time point

| | | | Drug − | | | |
|---|---|---|---|---|---|---|
| date_time | TCMCB07 | Placebo | placebo | p-values | 95% CI | |
| day 1 − 0 | 14.6667 | 31.8333 | −17.1667 | 0.1834 | −43.1532 | 8.8199 |
| day 2 − 02 | 7.6111 | 15 | −7.3889 | 0.242 | −20.1726 | 5.3949 |
| day 2 + 10 | 6.8889 | 23.5 | −16.6111 | 0.0391 | −32.3031 | −0.9192 |
| day 3 − 02 | 7.8333 | 25.6667 | −17.8333 | 0.0025 | −28.5932 | −7.0734 |
| day 3 + 10 | 14.1111 | 20.3333 | −6.2222 | 0.5934 | −30.2003 | 17.7558 |
| day 4 − 02 | 5.3889 | 16 | −10.6111 | 0.0825 | −22.7203 | 1.498 |
| day 4 + 10 | 9.6667 | 36 | −26.3333 | 0.0058 | −44.1093 | −8.5573 |
| day 5 − 02 | 8.3333 | 23 | −14.6667 | 0.0492 | −29.2783 | −0.05499 |
| day 5 + 10 | 18.3333 | 37.1667 | −18.8333 | 0.1163 | −42.7642 | 5.0976 |
| day 6 − 02 | 13.2222 | 22.6667 | −9.4444 | 0.2711 | −26.85 | 7.9611 |
| day 6 + 10 | 12.7222 | 27.1667 | −14.4444 | 0.0738 | −30.415 | 1.5261 |
| day 7 + 24 | 12.5556 | 27.5 | −14.944 | 0.1182 | −34.041 | 4.1521 |
| day 8 + 48 | 12.5 | 19.1667 | −6.6667 | 0.4124 | −23.2786 | 9.9452 |

### Enumerated Embodiments

Clause 1. A method of stimulating appetite of a subject in need thereof, the method comprising: administering to the subject a therapeutically effective amount of a non-naturally occurring melanocortin analog comprising a sequence according to Formula (I), administering to the subject a therapeutically effective amount of a non-naturally occurring melanocortin analog comprising a sequence according to Formula (I),

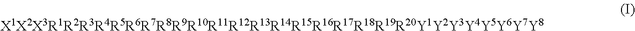

wherein:
R1 is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R2 is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R3 is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-α][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);;
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
   wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, -alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, 3-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine; and
wherein the non-naturally occurring melanocortin analog comprises one or more of the following features: (i) X² or X³ is present; (ii) R¹ is absent or is selected from the group consisting of dArg, dMet, dIIe, dLeu, dVal, dAla, Ala, Tle, dTle, DNle, Nva, Gly, dPro, dCys, dPhe, dTyr, dGIn, dAsn, transPro(guan), cisPro(guan), dTyr, Tyr, and Dmt; (iii) R² is absent; (iv) R³ is selected from the group consisting of dPro, Pro-Gly, and dAla; (v) R⁴ is selected from the group consisting of His, Trp, and Phe; (vi) R⁵ is Phe; (vii) R⁷ is absent; (viii) R⁸ is present; (ix) R⁹ and/or R¹⁰ is present; (x) one or more of R¹¹—R²⁰ are present; and (xi) Y⁴ is present; and
the non-naturally occurring melanocortin analog is cyclized through a moiety selected from the group consisting of:
   a disulfide bond between R¹ or R² and R⁷ or X¹ when R¹ or R² is cysteine and R⁷ or X¹ is cysteine;
   a disulfide bond between R² and any one of R⁵—R²⁰ when R² and the any one of R⁵—R²⁰ are selected from the group consisting of cysteine, Pen, and dPen;
   a lactam bridge between R¹ and R⁷ when R¹ is norleucine and R⁷ is glutamic acid;
   a side-chain lactam bridge between R² and R⁷ when R² is glutamic acid, aspartic acid, or CO-cis-CH—CH—CO, and R⁷ is lysine or ornithine;
   a side-chain lactam bridge between R² and R⁸ when R² is glutamic acid or aspartic acid, R⁸ is lysine, and R⁷ is proline or tryptophan;
   a lactam closure between R¹ and R⁷ when R¹ is succinic acid or o-phthalic acid and R⁷ is lysine; and
   a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

Clause 2. The method of clause 1, wherein the N-terminus, if present, is modified by a functional group selected from the group consisting of an acyl group, an imine group, an amide group, a urea group, a carbamate group, a sulfonamide group, and an alkylamine group.

Clause 3. The method of clause 2, wherein the N-terminus, if present, is modified by an acyl group.

Clause 4. The method of clause 3, wherein the acyl group is acetyl group.

Clause 5. The method of clause 3, wherein the acyl group is formyl group.

Clause 6. The method of clause 2, wherein the N-terminus, if present, is modified by an imine group.

Clause 7. The method of clause 2, wherein the N-terminus, if present, is modified by an amide group.

Clause 8. The method of clause 7, wherein the amide group is a pyroglutamyl (pGlu) group.

Clause 9. The method of clause 7, wherein the amide group is derived from a fatty acid.

Clause 10. The method of clause 1, wherein the N-terminus, if present, is not modified.

Clause 11. The method of clause 1, wherein the C-terminus is modified by a functional group selected from the group consisting of an amide group, an ester group, and an aldehyde group.

Clause 12. The method of clause 11, wherein the C-terminus is modified by an amide group.

Clause 13. The method of clause 12, wherein the amide group is an —NHalkyl amide group or an —NHaryl amide group.

Clause 14. The method of clause 13, wherein the—NHaryl amide group is p-nitroanilide group or 7-amino-4-methylcoumarin.

Clause 15. The method of clause 11, wherein the C-terminus is modified by an ester group.

Clause 16. The method of clause 1, wherein the C-terminus is not modified.

Clause 17. The method of clause 1, wherein R¹ is absent, and R² is D-aspartic acid.

Clause 18. The method of clause 17, wherein X¹, X², and X³ are absent.

Clause 19. The method of clause 1, wherein R⁴ is D-Nal(2′).

Clause 20. The method of clause 1, wherein Y³-Y⁸ are absent.

Clause 21. The method of clause 20, wherein: Y¹ is D-valine and Y² is D-proline;
or Y¹ is D-proline and Y² is D-valine.

Clause 22. The method of clause 1, wherein Y³ is present and Y⁴-Y⁸ are absent.

Clause 23. The method of clause 22, wherein: Y¹ is D-valine or D-proline; Y² is D-valine or D-proline; and/or Y³ is D-valine or D-proline.

Clause 24. The method of clause 22, wherein: Y¹ is D-valine, Y² is D-valine, and Y³ is D-proline; Y¹ is D-proline, Y² is D-valine, and Y³ is D-valine; Y¹ is D-valine, Y² is D-proline, and Y³ is D-valine; or Y¹ is D-proline, Y² is D-valine, and Y³ is D-proline.

Clause 25. The method of clause 1, wherein Y³ and Y⁴ are present, and Y⁵-Y⁸ are absent.

Clause 26. The method of clause 25, wherein: Y¹ is D-valine or D-proline; Y² is D-valine or D-proline; Y³ is D-valine or D-proline; and/or Y⁴ is D-valine or D-proline.

Clause 27. The method of clause 25, wherein: Y¹ is D-valine, Y² is D-valine, Y³ is D-valine, and Y⁴ is D-proline; Y¹ is D-proline, Y² is D-valine, Y³ is D-valine, and Y⁴ is D-valine; Y¹ is D-valine, Y² is D-proline, Y³ is D-valine, and Y⁴ is D-valine; Y¹ is D-valine, Y² is D-valine, Y³ is D-proline, and Y⁴ is D-valine; or Y¹ is D-valine, Y² is D-proline, Y³ is D-valine, and Y⁴ is D-proline.

Clause 28. The method of clause 1, wherein R¹, R², and R⁷ are present and

R⁸—R²⁰ are absent, and the sequence of Formula (I) is cyclized through R² and R⁷ via a lactam bond.

Clause 29. The method of clause 28, wherein: R¹ is acetylated norleucine; R² is aspartic acid; R³ is selected from the group consisting of proline, hydroxyproline, and D-hydroxyproline; R⁴ is dNal(2′); R⁵ is arginine; R⁶ is D-tryptophan or L-tryptophan; R⁷ is lysine; Y¹ is D-valine; and/or Y² is D-proline.

Clause 30. The method of clause 28, wherein the sequence of Formula (I) is: Ac-Nle-c(Asp-Pro-dNal2′-Arg-Trp-Lys)-dVal-dPro-NH₂ (SEQ ID NO: 3); or Ac-Nle-c(Asp-Hyp-dNal2′-Arg-Trp-Lys)-dVal-dPro-NH₂ (SEQ ID NO: 4), wherein c represents cyclization through R² and R⁷ via a lactam bond.

Clause 31. The method of clause 1, wherein R¹, R², R⁷, and R⁸ are present and R⁹—R²⁰ are absent, and the sequence of Formula (I) is cyclized through R² and R⁸ via a lactam bond.

Clause 32. The method of clause 31, wherein: R¹ is acetylated norleucine; R² is aspartic acid; R³ is selected from the group consisting of proline, hydroxyproline, D-hydroxyproline, phenylalanine, and histidine; R⁴ is histidine or dNal(2′); R⁵ is dNal(2′) or arginine; R⁶ is selected from the group consisting of arginine, D-tryptophan, and L-tryptophan; R⁷ is tryptophan or proline; R⁸ is lysine; Y¹ is selected from the group consisting of D-valine, D-leucine, and D-isoleucine; and/or Y² is D-proline.

Clause 33. The method of clause 31, wherein the sequence of Formula (I) is selected from the group consisting of: Ac-Nle-c(Asp-Pro-His-dNal(2′)-Arg-Trp-Lys)-dVal-dPro-NH₂ (SEQ ID NO: 6); Ac-Nle-c(Asp-Phe-His-dNal(2′)-Arg-Trp-Lys)-dLeu-dPro-NH₂ (SEQ ID NO: 7); Ac-Nle-c(Asp-Phe-His-dNal(2′)-Arg-Trp-Lys)-dIIe-dPro-NH₂ (SEQ ID NO: 8); Ac-Nle-c(Asp-His-dNal(2′)-Arg-Trp-Pro-Lys)-dVal-dPro-NH₂ (SEQ ID NO: 9); Ac-Nle-c(Asp-Hyp-dNal(2′)-Arg-Trp-Pro-Lys)-dVal-dPro-NH₂ (SEQ ID NO: 10); and Ac-Nle-c(Asp-Pro-His-dNal(2′)-Arg-Trp-Lys)-dPro-dVal-NH₂ (SEQ ID NO: 11; D5), wherein c represents cyclization through R² and R⁸ via a lactam bond.

Clause 34. The method of clause 1, wherein R¹—R² and R⁷—R¹⁰ are present and R¹¹—R²⁰ are absent, and the sequence of Formula (I) is cyclized through R² and R¹° via a lactam bond.

Clause 35. The method of clause 34, wherein the sequence of Formula (I) is Ac-Nle-c(Asp-Phe-Phe-Pro-His-dNal(2′)-Arg-Trp-Lys)-dVal-dPro-NH₂ (SEQ ID NO: 12), wherein c represents cyclization through R² and R¹⁰ via a lactam bond.

Clause 36. The method of clause 1, wherein R¹—R² and R⁷—R¹⁰ are present and R¹¹—R²⁰ are absent, and the sequence of Formula (I) is cyclized through R⁴ and R¹⁰ via a lactam bond.

Clause 37. The method of clause 36, wherein the sequence of Formula (I) is Ac-Nle-Phe-Phe-c(Asp-Phe-His-dNal(2′)-Arg-Trp-Lys)-dVal-Dpro-NH₂ (SEQ ID NO: 13), wherein c represents cyclization through R⁴ and R¹⁰ via a lactam bond.

Clause 38. The method of clause 16, wherein the sequence of Formula (I) is Ac-Nle-c(Asp-Pro-dNal(2′)-Arg-Trp-Lys)-dVal-dPro(SEQ ID NO: 2).

Clause 39. The method of clause 1, wherein the sequence of Formula (I) is linear.

Clause 40. The method of clause 39, wherein the sequence of Formula (I) is Ac-Nle-Asp-Pro-dNal(2′)-Arg-Trp-Lys-dVal-dPro-NH₂ (SEQ ID NO: 14) or Ac-Nle-Pro-dNal(2′)-Arg-Trp-dVal-dPro-NH₂ (SEQ ID NO: 15).

Clause 41. The method of clause 1, wherein the sequence of Formula (I) is selected from the group consisting of: Ac-dArg-c(Asp-Pro-dNal(2′)-Arg-Trp-Lys)-dVal-dPro-NH₂ (SEQ ID NO: 16); Ac-dMet-c(Asp-Pro-dNal(2′)-Arg-Trp-Lys)-dVal-dPro-NH₂ (SEQ ID NO: 17); Ac-dIIe-c(Asp-Pro-dNal(2′)-Arg-Trp-Lys)-dVal-dPro-NH₂ (SEQ ID NO: 18); Ac-dLeu-c(Asp-Pro-dNal(2′)-Arg-Trp-Lys)-dVal-dPro-NH₂ (SEQ ID NO: 19); Ac-dVal-c(Asp-Pro-dNal(2′)-Arg-Trp-Lys)-dVal-dPro-NH₂ (SEQ ID NO: 20); Ac-dAla-c(Asp-Pro-dNal(2′)-Arg-Trp-Lys)-dVal-dPro-NH₂ (SEQ ID NO: 21); Ac-Ala-c(Asp-Pro-dNal(2′)-Arg-Trp-Lys)-dVal-dPro-NH₂ (SEQ ID NO: 22); Ac-Tle-c(Asp-Pro-dNal(2′)-Arg-Trp-Lys)-dVal-dPro-NH₂ (SEQ ID NO: 23); Ac-dTle-c(Asp-Pro-dNal(2′)-Arg-Trp-Lys)-dVal-dPro-NH₂ (SEQ ID NO: 24); Ac-dNle-c(Asp-Pro-dNal(2′)-Arg-Trp-Lys)-dVal-dPro-NH₂ (SEQ ID NO: 25); Ac-Nva-c(Asp-Pro-dNal(2′)-Arg-Trp-Lys)-dVal-dPro-NH₂ (SEQ ID NO: 26); Ac-Gly-c(Asp-Pro-dNal(2′)-Arg-Trp-Lys)-dVal-dPro-NH₂ (SEQ ID NO: 27); Ac-dPro-c(Asp-Pro-dNal(2′)-Arg-Trp-Lys)-dVal-dPro-NH₂ (SEQ ID NO: 28); Ac-dCys-c(Asp-Pro-dNal(2′)-Arg-Trp-Lys)-dVal-dPro-NH₂ (SEQ ID NO: 29); Ac-dPhe-c(Asp-Pro-dNal(2′)-Arg-Trp-Lys)-dVal-dPro-NH₂ (SEQ ID NO: 30); Ac-dTyr-c(Asp-Pro-dNal(2′)-Arg-Trp-Lys)-dVal-dPro-NH₂ (SEQ ID NO: 31); Ac-dGln-c(Asp-Pro-dNal(2′)-Arg-Trp-Lys)-dVal-dPro-NH₂ (SEQ ID NO: 32); Ac-dAsn-c(Asp-Pro-dNal(2′)-Arg-Trp-Lys)-dVal-dPro-NH₂ (SEQ ID NO: 33); Ac-transPro(guan)-c(Asp-Pro-dNal(2′)-Arg-Trp-Lys)-dVal-dPro-NH₂ (SEQ ID NO: 34); Ac-cisPro(guan)-c(Asp-Pro-dNal(2′)-Arg-Trp-Lys)-dVal-dPro-NH₂ (SEQ ID NO: 35); dTyr-c(Asp-Pro-dNal(2′)-Arg-Trp-Lys)-dVal-dPro-NH₂ (SEQ ID NO: 36); Tyr-c(Asp-Pro-dNal(2′)-Arg-Trp-Lys)-dVal-dPro-NH₂ (SEQ ID NO: 37); and Dmt-c(Asp-Pro-dNal(2′)-Arg-Trp-Lys)-dVal-dPro-NH₂ (SEQ ID NO: 38), wherein c represents cyclization through R² and R⁷ via a lactam bond.

Clause 42. The method of clause 1, wherein the sequence of Formula (I) is selected from the group consisting of: Ac-Nle-c(dAsp-Pro-dNal(2′)-Arg-Trp-Lys)-dVal-dPro-NH₂ (SEQ ID NO: 39); Ac-Nle-c(Asp-Pro-dNal(2′)-Arg-Trp-dLys)-dVal-dPro-NH₂ (SEQ ID NO: 40); Ac-Nle-c(Cys-Pro-dNal(2′)-Arg-Trp-Cys)-dVal-dPro-NH₂ (SEQ ID NO: 41); Ac-Nle-c (dCys-Pro-dNal(2′)-Arg-Trp-Cys)-dVal-dPro-NH₂ (SEQ ID NO: 42); Ac-Nle-c(Cys-Pro-dNal(2′)-Arg-Trp-dCys)-dVal-dPro-NH₂ (SEQ ID NO: 43); Ac-Nle-c(dCys-Pro-dNal(2′)-Arg-Trp-dCys)-dVal-dPro-NH₂ (SEQ ID NO: 44); Ac-Nle-c(Cys-dNal(2′)-Arg-Trp-Cys)-dVal-dPro-NH₂ (SEQ ID NO: 45); Ac-Nle-c(dCys-dNal(2′)-Arg-Trp-Cys)-dVal-dPro-NH₂ (SEQ ID NO: 46); Ac-Nle-c(Cys-dNal(2′)-Arg-Trp-dCys)-dVal-dPro-NH₂ (SEQ ID NO: 47); Ac-Nle-c (dCys-dNal(2′)-Arg-Trp-dCys)-dVal-dPro-NH₂ (SEQ ID NO: 48); Ac-Nle-c(Cys-Pro-dNal(2′)-Arg-Trp-Pen)-dVal-dPro-NH₂ (SEQ ID NO: 49); Ac-Nle-c(dCys-Pro-dNal(2′)-Arg-Trp-Pen)-dVal-dPro-NH₂ (SEQ ID NO: 50); Ac-Nle-c(Pen-Pro-dNal(2′)-Arg-Trp-Cys)-dVal-dPro-NH₂ (SEQ ID NO: 51); Ac-Nle-c(Pen-Pro-dNal(2′)-Arg-Trp-dCys)-dVal-dPro-NH₂ (SEQ ID NO: 52); Ac-Nle-c(Pen-Pro-dNal(2′)-Arg-Trp-Pen)-dVal-dPro-NH₂ (SEQ ID NO: 53); Ac-Nle-c(dPen-Pro-dNal(2′)-Arg-Trp-Pen)-dVal-dPro-NH₂ (SEQ ID NO: 54); Ac-Nle-c(dPen-Pro-dNal(2′)-Arg-Trp-dPen)-dVal-dPro-NH₂ (SEQ ID NO: 55); Ac-Nle-c(Pen-Pro-dNal(2′)-Arg-Trp-dPen)-dVal-dPro-NH₂ (SEQ ID NO: 56); Ac-Nle-c(Cys-dNal(2′)-Arg-Trp-Pen)-dVal-dPro-NH₂ (SEQ ID NO: 57); Ac-Nle-c(dCys-dNal(2′)-Arg-Trp-Pen)-dVal-dPro-NH₂ (SEQ ID NO: 58); Ac-Nle-c(Pen-dNal(2′)-Arg-Trp-Cys)-dVal-dPro-NH₂ (SEQ ID NO: 59); Ac-Nle-c(Pen-dNal(2′)-Arg-Trp-dCys)-dVal-dPro-NH₂ (SEQ ID NO: 60); Ac-Nle-c(Pen-dNal(2′)-Arg-Trp-Pen)-dVal-dPro-NH₂ (SEQ ID NO: 61); Ac-Nle-c(dPen-dNal(2′)-Arg-Trp-Pen)-dVal-dPro-NH₂ (SEQ ID NO: 62); Ac-Nle-c(dPen-dNal(2′)-Arg-Trp-dPen)-dVal-dPro-NH₂ (SEQ ID NO: 63); Ac-Nle-c(Pen-dNal(2′)-Arg-Trp-dPen)-dVal-dPro-NH₂ (SEQ ID NO: 64); Ac-Nle-c(Asp-Pro-dNal(2′)-Arg-Trp-Orn)-dVal-dPro-NH₂ (SEQ ID NO: 65); Ac-Nle-c(Asp-Pro-dNal(2′)-Arg-Trp-dOrn)-dVal-dPro-NH₂ (SEQ ID NO: 66); Ac-Nle-c(Glu-Pro-dNal(2′)-Arg-Trp-Orn)-dVal-dPro-NH₂ (SEQ ID NO: 67); and Ac-Nle-c(Glu-Pro-dNal(2′)-Arg-Trp-dOrn)-dVal-dPro-NH₂ (SEQ ID NO: 68), wherein c represents cyclization through R² and R⁶ or R⁷ via a lactam bond or a disulfide bond.

Clause 43. The method of clause 1, wherein the sequence of Formula (I) is selected from the group consisting of: Ac-Nle-c[Asp-dNal(2′)-Arg-Trp-Lys]-dVal-dPro-NH₂ (SEQ ID NO: 69); Ac-Nle-c[Asp-Ala-dNal(2′)-Arg-Trp-Lys]-dVal-dPro-NH₂ (SEQ ID NO: 70); Ac-Nle-c(Asp-dPro-dNal(2′)-Arg-Trp-Lys)-dVal-dPro-NH₂ (SEQ ID NO: 71); Ac-Nle-c(Asp-dAla-dNal(2′)-Arg-Trp-Lys)-dVal-dPro-NH₂ (SEQ ID NO: 72); Ac-Nle-c(Asp-dMet-dNal(2′)-Arg-Trp-Lys)-dVal-dPro-NH₂ (SEQ ID NO: 73); Ac-Nle-c(Asp-Pro-Gly-dNal(2′)-Arg-Trp-Lys)-dVal-dPro-NH₂ (SEQ ID NO: 74); Ac-Nle-c(Asp-Gly-Gly-dNal(2′)-Arg-Trp-Lys)-dVal-dPro-NH₂ (SEQ ID NO: 75); Ac-Nle-c[Asp-Gly-Dnal(2′)-Arg-Trp-Lys]-dVal-dPro-NH₂ (SEQ ID NO: 76); Ac-Nle-c[Asp-Leu-Dnal(2′)-Arg-Trp-Lys]-dVal-dPro-NH₂ (SEQ ID NO: 77); Ac-Nle-c[Asp-Ile-Dnal(2′)-Arg-Trp-Lys]-dVal-dPro-NH₂ (SEQ ID NO: 78); Ac-Nle-c[Asp-Val-Dnal(2′)-Arg-Trp-Lys]-dVal-dPro-NH₂ (SEQ ID NO: 79); Ac-Nle-c[Asp-dLeu-dNal(2′)-Arg-Trp-Lys]-dVal-dPro-NH₂ (SEQ ID NO: 80); Ac-Nle-c[Asp-dIIe-dNal(2′)-Arg-Trp-Lys]-dVal-dPro-NH₂ (SEQ ID NO: 81); Ac-Nle-c[Asp-dVal-dNal(2′)-Arg-Trp-Lys]-dVal-dPro-NH₂ (SEQ ID NO: 82); Ac-Nle-c[Asp-Trp-dNal(2′)-Arg-Trp-Lys]-dVal-dPro-NH₂ (SEQ ID NO: 83); Ac-Nle-c[Asp-dTrp-dNal(2′)-Arg-Trp-Lys]-dVal-dPro-NH₂ (SEQ ID NO: 84); Ac-Nle-c[Asp-transPro(guan)-dNal(2′)-Arg-Trp-Lys]-dVal-dPro-NH₂ (SEQ ID NO: 85); and Ac-Nle-c[Asp-cisPro(guan)-dNal(2′)-Arg-Trp-Lys]-dVal-dPro-NH₂ (SEQ ID NO: 86), wherein c represents cyclization through R² and R⁷ via a lactam bond.

Clause 44. The method of clause 1, wherein the sequence of Formula (I) is selected from the group consisting of: Ac-Nle-c(Asp-Pro-dNal(2′)-Trp-Lys)-dVal-dPro-NH₂ (SEQ ID NO: 87); Ac-Nle-c(Asp-Pro-dNal(2′)-Lys-Trp-Lys)-dVal-dPro-NH₂ (SEQ ID NO: 88); Ac-Nle-c(Asp-Pro-dNal(2′)-dLys-Trp-Lys)-dVal-dPro-NH₂ (SEQ ID NO: 89); Ac-Nle-c(Asp-Pro-dNal(2′)-dArg-Trp-Lys)-dVal-dPro-NH₂ (SEQ ID NO: 90); Ac-Nle-c(Asp-Pro-dNal(2′)-Orn-Trp-Lys)-dVal-dPro-NH₂ (SEQ ID NO: 91); Ac-Nle-c(Asp-Pro-dNal(2′)-dOrn-Trp-Lys)-dVal-dPro-NH₂ (SEQ ID NO: 92); Ac-Nle-c(Asp-Pro-dNal(2′)-His-Trp-Lys)-dVal-dPro-NH₂ (SEQ ID NO: 93); Ac-Nle-c(Asp-Pro-dNal(2′)-Ala-Trp-Lys)-dVal-dPro-NH₂ (SEQ ID NO: 94); Ac-Nle-c(Asp-Pro-dNal(2′)-Gly-Trp-Lys)-dVal-dPro-NH₂ (SEQ ID NO: 95); Ac-Nle-c(Asp-Pro-dNal(2′)-Asp-Trp-Lys)-dVal-dPro-NH₂ (SEQ ID NO: 96); Ac-Nle-c(Asp-Pro-dNal(2′)-Glu-Trp-Lys)-dVal-dPro-NH₂ (SEQ ID NO: 97); Ac-Nle-c(Asp-Pro-dNal(2′)-dHis-Trp-Lys)-dVal-dPro-NH₂ (SEQ ID NO: 98); Ac-Nle-c(Asp-Pro-dNal(2′)-dAla-Trp-Lys)-dVal-dPro-NH₂ (SEQ ID NO: 99); Ac-Nle-c(Asp-Pro-dNal(2′)-dAsp-Trp-Lys)-dVal-dPro-NH₂ (SEQ ID NO: 100); and Ac-Nle-c(Asp-Pro-dNal(2′)-dGlu-Trp-Lys)-dVal-dPro-NH₂ (SEQ ID NO: 101), wherein c represents cyclization through R² and R⁷ via a lactam bond.

Clause 45. The method of clause 1, wherein the sequence of Formula (I) is selected from the group consisting of: Ac-Nle-c(Asp-Pro-dNal(2′)-Arg-Lys)-dVal-dPro-NH₂ (SEQ ID NO: 102); Ac-Nle-c(Asp-Pro-dNal(2′)-Arg-Nal(1′)-Lys)-dVal-dPro-NH₂ (SEQ ID NO: 103); Ac-Nle-c(Asp-Pro-dNal(2′)-Arg-Aia-Lys)-dVal-dPro-NH₂ (SEQ ID NO: 104); Ac-Nle-c(Asp-Pro-dNal(2′)-Arg-Phe-Lys)-dVal-dPro-NH₂ (SEQ ID NO: 105); Ac-Nle-c(Asp-Pro-dNal(2′)-Arg-Tyr-Lys)-dVal-dPro-NH₂ (SEQ ID NO: 106); Ac-Nle-c(Asp-Pro-dNal(2′)-Arg-His-Lys)-dVal-dPro-NH₂ (SEQ ID NO: 107); Ac-Nle-c(Asp-Pro-dNal(2′)-Arg-Ala-Lys)-dVal-dPro-NH₂ (SEQ ID NO: 108); Ac-Nle-c(Asp-Pro-dNal(2′)-Arg-dNal(1′)-Lys)-dVal-dPro-NH₂ (SEQ ID NO: 109); Ac-Nle-c(Asp-Pro-dNal(2′)-Arg-dPhe-Lys)-dVal-dPro-NH₂ (SEQ ID NO: 110); Ac-Nle-c(Asp-Pro-dNal(2′)-Arg-dNal(2′)-Lys)-dVal-dPro-NH₂ (SEQ ID NO: 111); Ac-Nle-c(Asp-Pro-dNal(2′)-Arg-dTyr-Lys)-dVal-dPro-NH₂ (SEQ ID NO: 112); Ac-Nle-c(Asp-Pro-dNal(2′)-Arg-dHis-Lys)-dVal-dPro-NH₂ (SEQ ID NO: 113); and Ac-Nle-c(Asp-Pro-dNal(2′)-Arg-dAla-Lys)-dVal-dPro-NH₂ (SEQ ID NO: 114), wherein c represents cyclization through R² and R⁷ via a lactam bond.

Clause 46. The method of clause 1, wherein the sequence of Formula (I) is Ac-Nle-c(Asp-Pro-Bip-Arg-Trp-Lys)-dVal-dPro-NH₂ (SEQ ID NO: 115), wherein c represents cyclization through R² and R⁷ via a lactam bond.

Clause 47. The method of clause 1, wherein the sequence of Formula (I) is selected from the group consisting of: Ac-Nle-c[Asp-Pro-dNal(2′)-Arg-Trp-Lys]-Val-Pro-NH₂ (SEQ ID NO: 116); Ac-Nle-c[Asp-Pro-dNal(2′)-Arg-Trp-Lys]-dVal-dPro-OH (SEQ ID NO: 117); Ac-Nle-c[Asp-Pro-dNal(2′)-Arg-Trp-Lys]-dPro-dVal-OH (SEQ ID NO: 118); Ac-Nle-c[Asp-Pro-dNal(2′)-Arg-Trp-Lys]-dVal-Hyp-NH₂ (SEQ ID NO: 119); Ac-Nle-c[Asp-Pro-dNal(2′)-Arg-Trp-Lys]-dVal-dHyp-NH₂ (SEQ ID NO: 120); Ac-Nle-c[Asp-Pro-dNal(2′)-Arg-Trp-Lys]-Val-Hyp-NH₂ (SEQ ID NO: 121); Ac-Nle-c[Asp-Pro-dNal(2′)-Arg-Trp-Lys]-Val-dHyp-NH₂ (SEQ ID NO: 122); Ac-Nle-c[Asp-Pro-dNal(2′)-Arg-Trp-Lys]-Hyp-dVal-NH₂ (SEQ ID NO: 123); Ac-Nle-c[Asp-Pro-dNal(2′)-Arg-Trp-Lys]-dHyp-dVal-NH₂ (SEQ ID NO: 124); Ac-Nle-c[Asp-Pro-dNal(2′)-Arg-Trp-Lys]-Hyp-Val-NH₂ (SEQ ID NO: 125); Ac-Nle-c[Asp-Pro-dNal(2′)-Arg-Trp-Lys]-dHyp-Val-NH₂ (SEQ ID NO: 126); Ac-Nle-c[Asp-Pro-dNal(2′)-Arg-Trp-Lys]-dVal-dVal-NH₂ (SEQ ID NO: 127); Ac-Nle-c[Asp-Pro-dNal(2′)-Arg-Trp-Lys]-dPro-dPro-NH₂ (SEQ ID NO: 128); Ac-Nle-c[Asp-Pro-dNal(2′)-Arg-Trp-Lys]-dVal-NH₂ (SEQ ID NO: 129); Ac-Nle-c[Asp-Pro-dNal(2′)-Arg-Trp-Lys]-dPro-NH₂ (SEQ ID NO: 130); Ac-Nle-c[Asp-Pro-dNal(2′)-Arg-Trp-Lys]-Val-NH₂ (SEQ ID NO: 131); Ac-Nle-c[Asp-Pro-dNal(2′)-Arg-Trp-Lys]-Pro-NH₂ (SEQ ID NO: 132); Ac-Nle-c[Asp-Pro-dNal(2′)-Arg-Trp-Lys]-Ala-NH₂ (SEQ ID NO: 133); Ac-Nle-c[Asp-Pro-dNal(2′)-Arg-Trp-Lys]-dAla-NH₂ (SEQ ID NO: 134); Ac-Nle-c[Asp-Pro-dNal(2′)-Arg-Trp-Lys]-dHyp-NH₂ (SEQ ID NO: 135); Ac-Nle-c[Asp-Pro-dNal(2′)-Arg-Trp-Lys]-Hyp-NH₂ (SEQ ID NO: 136); Ac-Nle-c[Asp-Pro-dNal(2′)-Arg-Trp-Lys]-dAla-dAla-NH₂ (SEQ ID NO: 137); Ac-Nle-c[Asp-Pro-dNal(2′)-Arg-Trp-Lys]-Ala-Ala-NH₂ (SEQ ID NO: 138); Ac-Nle-c[Asp-Pro-dNal(2′)-Arg-Trp-Lys]-Gly-Gly-NH₂ (SEQ ID NO: 139); Ac-Nle-c[Asp-Pro-dNal(2′)-Arg-Trp-Lys]-dVal-Asp-NH₂ (SEQ ID NO: 140); Ac-Nle-c[Asp-Pro-dNal(2′)-Arg-Trp-Lys]-dVal-Arg-NH₂ (SEQ ID NO: 141); Ac-Nle-c[Asp-Pro-dNal(2′)-Arg-Trp-Lys]-dVal-Asn-NH₂ (SEQ ID NO: 142); Ac-Nle-c[Asp-Pro-dNal(2′)-Arg-Trp-Lys]-dVal-dAsp-NH₂ (SEQ ID NO: 143); Ac-Nle-c[Asp-Pro-dNal(2′)-Arg-Trp-Lys]-dVal-dArg-NH₂ (SEQ ID NO: 144); Ac-Nle-c[Asp-Pro-dNal(2′)-Arg-Trp-Lys]-dVal-dAsn-NH₂ (SEQ ID NO: 145); Ac-Nle-c[Asp-Pro-dNal(2′)-Arg-Trp-Lys]-Asp-dPro-NH₂ (SEQ ID NO: 146); Ac-Nle-c[Asp-Pro-dNal(2′)-Arg-Trp-Lys]-Arg-dPro-NH₂ (SEQ ID NO: 147); Ac-Nle-c[Asp-Pro-dNal(2′)-Arg-Trp-Lys]-Asn-dPro-NH₂ (SEQ ID NO: 148); Ac-Nle-c[Asp-Pro-dNal(2′)-Arg-Trp-Lys]-dAsp-dPro-NH₂ (SEQ ID NO: 149); Ac-Nle-c[Asp-Pro-dNal(2′)-Arg-Trp-Lys]-dArg-dPro-NH₂ (SEQ ID NO: 150); Ac-Nle-c[Asp-Pro-dNal(2′)-Arg-Trp-Lys]-dAsn-dPro-NH₂ (SEQ ID NO: 151); Ac-Nle-c[Asp-Pro-dNal(2′)-Arg-Trp-Lys]-Asp-NH₂ (SEQ ID NO: 152); Ac-Nle-c[Asp-Pro-dNal(2′)-Arg-Trp-Lys]-Arg-NH₂ (SEQ ID NO: 153); Ac-Nle-c[Asp-Pro-dNal(2′)-Arg-Trp-Lys]-Asn-NH₂ (SEQ ID NO: 154); Ac-Nle-c[Asp-Pro-dNal(2′)-Arg-Trp-Lys]-dAsp-NH₂ (SEQ ID NO: 155); Ac-Nle-c[Asp-Pro-dNal(2′)-Arg-Trp-Lys]-dArg-NH₂ (SEQ ID NO: 156); Ac-Nle-c[Asp-Pro-dNal(2′)-Arg-Trp-Lys]-dAsn-NH₂ (SEQ ID NO: 157); Ac-Nle-c[Asp-Pro-dNal(2′)-Arg-Trp-Lys]-Lys-Pro-Val-NH₂ (SEQ ID NO: 158); Ac-Nle-c[Asp-Pro-dNal(2′)-Arg-Trp-Lys]-Lys-dPro-dVal-NH₂ (SEQ ID NO: 159); Ac-Nle-c[Asp-Pro-dNal(2′)-Arg-Trp-Lys]-dLys-dPro-dVal-NH₂ (SEQ ID NO: 160); Ac-Nle-c[Asp-Pro-dNal(2′)-Arg-Trp-Lys]-Lys-dPro-NH₂ (SEQ ID NO: 161); Ac-Nle-c[Asp-Pro-dNal(2′)-Arg-Trp-Lys]-dLys-dPro-NH₂ (SEQ ID NO: 162); Ac-Nle-c[Asp-Pro-dNal(2′)-Arg-Trp-Lys]-Lys-Val-Pro-NH₂ (SEQ ID NO: 163); Ac-Nle-c[Asp-Pro-dNal(2′)-Arg-Trp-Lys]-Lys-dVal-dPro-NH₂ (SEQ ID NO: 164); Ac-Nle-c[Asp-Pro-dNal(2′)-Arg-Trp-Lys]-dLys-dVal-dPro-NH₂ (SEQ ID NO: 165); Ac-Nle-c[Asp-Pro-dNal(2′)-Arg-Trp-Lys]-Arg-Pro-Val-NH₂ (SEQ ID NO: 166); Ac-Nle-c[Asp-Pro-dNal(2′)-Arg-Trp-Lys]-Arg-dPro-dVal-NH₂ (SEQ ID NO: 167); Ac-Nle-c[Asp-Pro-dNal(2′)-Arg-Trp-Lys]-dArg-dPro-dVal-NH₂ (SEQ ID NO: 168); Ac-Nle-c[Asp-Pro-dNal(2′)-Arg-Trp-Lys]-Arg-Val-Pro-NH₂ (SEQ ID NO: 169); Ac-Nle-c[Asp-Pro-dNal(2′)-Arg-Trp-Lys]-Arg-dVal-dPro-NH₂ (SEQ ID NO: 170); Ac-Nle-c[Asp-Pro-dNal(2′)-Arg-Trp-Lys]-dArg-dVal-dPro-NH₂ (SEQ ID NO: 171); Ac-Nle-c[Asp-Pro-dNal(2′)-Arg-Trp-Lys]-dTle-dVal-dPro-NH₂ (SEQ ID NO: 172); Ac-Nle-c[Asp-Pro-dNal(2′)-Arg-Trp-Lys]-dTle-dPro-NH₂ (SEQ ID NO: 173); Ac-Nle-c[Asp-Pro-dNal(2′)-Arg-Trp-Lys]-dTle-dVal-NH₂ (SEQ ID NO: 174); Ac-Nle-c[Asp-Pro-dNal(2′)-Arg-Trp-Lys]-dTle-dTle-NH₂ (SEQ ID NO: 175); Ac-Nle-c[Asp-Pro-dNal(2′)-Arg-Trp-Lys]-dTle-dTle-dPro-NH₂ (SEQ ID NO: 176); Ac-Nle-c[Asp-Pro-dNal(2′)-Arg-Trp-Lys]-dTle-dTle-dVal-NH₂ (SEQ ID NO: 177); Ac-Nle-c[Asp-Pro-dNal(2′)-Arg-Trp-Lys]-dPro-dTle-NH₂ (SEQ ID NO: 178); and Ac-Nle-c[Asp-Pro-dNal(2′)-Arg-Trp-Lys]-dVal-dTle-NH₂ (SEQ ID NO: 179), wherein c represents cyclization through R² and R⁷ via a lactam bond.

Clause 48. The method of clause 1, wherein the sequence of Formula (I) is selected from the group consisting of: Ac-Nle-c(Asp-Pro-dNal(2′)-Arg-Trp-Lys)-dVal-dVal-dPro-NH₂ (SEQ ID NO: 180); Ac-Nle-c(Asp-Pro-dNal(2′)-Arg-Trp-Lys)-dPro-dVal-dPro-NH2 (SEQ ID NO: 181); Ac-Nle-c(Asp-Pro-dNal(2′)-Arg-Trp-Lys)-dVal-dVal-dVal-dPro-NH2 (SEQ ID NO: 182); Ac-Nle-c(Asp-Pro-dNal(2′)-Arg-Trp-Lys)-dVal-dPro-dVal-dPro-NH2 (SEQ ID NO: 183); and Ac-Nle-c(Asp-Pro-dNal(2′)-Arg-Trp-Lys)-dVal-dVal-dVal-dVal-dVal-dVal-dPro-NH2 (SEQ ID NO: 184), wherein c represents cyclization through R² and R⁷ via a lactam bond.

Clause 49. The method of clause 1, wherein X¹ is present and is acetylated norleucine, and R¹ is present and is norleucine.

Clause 50. The method of clause 49, wherein X² is present and is norleucine.

Clause 51. The method of clause 49, wherein X³ is present and is norleucine.

Clause 52. The method of claim49, wherein the sequence of Formula (I) is selected from the group consisting of: Ac-Nle-Nle-c(Asp-Pro-dNal(2′)-Arg-Trp-Lys)-dVal-dPro-NH2 (SEQ ID NO: 185); Ac-Nle-Nle-Nle-c(Asp-Pro-dNal(2′)-Arg-Trp-Lys)-dVal-dPro-NH2 (SEQ ID NO: 186); and Ac-Nle-Nle-Nle-Nle-c(Asp-Pro-dNal(2′)-Arg-Trp-Lys)-dVal-dPro-NH2 (SEQ ID NO: 187), wherein c represents cyclization through R² and R⁷ via a lactam bond.

Clause 53. The method of clause 1, wherein the sequence of Formula (I) is selected from the group consisting of: Ac-Nle-c[Asp-Pro-Pro-dNal(2′)-Arg-Trp-Lys]-dVal-dPro-NH2 (SEQ ID NO: 195); Ac-Nle-c[Asp-Trp-Pro-dNal(2′)-Arg-Trp-Lys]-dVal-dPro-NH2 (SEQ ID NO: 196); Ac-Nle-c[Asp-Pro-dNal(2′)-Arg-dTrp-Lys]-dVal-dPro-NH2 (SEQ ID NO: 197); c[CO-cis-CH═CH—CO-Pro-D-Nal(2′)-Arg-Trp-Lys]-dVal-dPro-NH2 (SEQ ID NO: 198); Ac-Nle-c[Asp-Aba-D-Phe-Arg-Trp-Lys]-dVal-dPro-NH2 (SEQ ID NO: 199); Ac-Nle-c[Asp-β-Ala-D-Nal(2′)-Arg-Trp-Lys]-dVal-dPro-NH2 (SEQ ID NO: 200); Ac-Nle-c[Asp-Mamb-D-Nal(2′)-Arg-Trp-Lys]-dVal-dPro-NH2 (SEQ ID NO: 201); Ac-Nle-c[Asp-Acpc-D-Nal(2′)-Arg-Trp-Lys]-dVal-dPro-NH2 (SEQ ID NO: 202); Ac-c[Cys-Arg-D-Phe-Cys]-Trp-dVal-dPro-NH2 (SEQ ID NO: 203); Ac-Nle-c[Asp-Pro-dNal(2′)-Arg-Trp-Lys]-Trp-NH2 (SEQ ID NO: 204); Ac-Nle-c(Asp-Aba-dNal(2′)-Arg-Trp-Lys)-dVal-dPro-NH2 (SEQ ID NO: 205); Ac-Nle-c(Asp-Aia-dNal(2′)-Arg-Trp-Lys)-dVal-dPro-NH2 (SEQ ID NO: 206); Ac-Nle-c(Asp-Ata-dNal(2′)-Arg-Trp-Lys)-dVal-dPro-NH2 (SEQ ID NO: 207); Ac-Nle-c(Asp-Aia-dPhe-Arg-Trp-Lys)-dVal-dPro-NH2 (SEQ ID NO: 208); Ac-Nle-c(Asp-Ata-dPhe-Arg-Trp-Lys)-dVal-dPro-NH2 (SEQ ID NO: 209); Ac-Nle-c(Asp-Aba-p(CI)dPhe-Arg-Trp-Lys)-dVal-dPro-NH2 (SEQ ID NO: 210); Ac-Nle-c(Asp-Aia-p(CI)dPhe-Arg-Trp-Lys)-dVal-dPro-NH2 (SEQ ID NO: 211); and Ac-Nle-c(Asp-Ata-p(CI)dPhe-Arg-Trp-Lys)-dVal-dPro-NH2 (SEQ ID NO: 212), wherein c represents cyclization through R² and R⁷ or R⁸ via a lactam bond or through R² and R⁵ via a disulfide bond.

Clause 54. The method of clause 1, wherein the sequence of Formula (I) is selected from the group consisting of: Ac-Nle-c[Asp-Aic-dNal(2′)-Arg-Trp-Lys]-dVal-dPro-NH2 (SEQ ID NO: 240); Ac-Nle-c[Asp-Cpe-dNal(2′)-Arg-Trp-Lys]-dVal-dPro-NH2 (SEQ ID NO: 241); Ac-Nle-c[Asp-Che-dNal(2′)-Arg-Trp-Lys]-dVal-dPro-NH2 (SEQ ID NO: 242); Ac-Nle-c[Asp-Oic-dNal(2′)-Arg-Trp-Lys]-dVal-dPro-NH2 (SEQ ID NO: 243); Ac-Nle-c[Asp-loc-dNal(2′)-Arg-Trp-Lys]-dVal-dPro-NH2 (SEQ ID NO: 244); Ac-Nle-c[Asp-Tic-dNal(2′)-Arg-Trp-Lys]-dVal-dPro-NH2 (SEQ ID NO: 245); Ac-Nle-c[Asp-Pro-dNal(2′)-Arg-Trp-Pro-Lys]-dVal-dPro-NH2 (SEQ ID NO: 246); Ac-Nle-c[Asp-His-dNal(2′)-Pro-Trp-Lys]-dVal-dPro-NH2 (SEQ ID NO: 247); Ac-Nle-c[Asp-His-dNal(2′)-transPro(guan)-Trp-Lys]-dVal-dPro-NH2 (SEQ ID NO: 248); Ac-Nle-c[Asp-His-dNal(2′)-cisPro(guan)-Trp-Lys]-dVal-dPro-NH2 (SEQ ID NO: 249); Ac-Nle-c[Asp-Pro-dNal(2′)-Pro-Trp-Lys]-dVal-dPro-NH2 (SEQ ID NO: 250); Ac-Nle-c[Asp-Pro-dNal(2′)-transPro(guan)-Trp-Lys]-dVal-dPro-NH2 (SEQ ID NO: 251); Ac-Nle-c[Asp-Pro-dNal(2′)-cisPro(guan)-Trp-Lys]-dVal-dPro-NH2 (SEQ ID NO: 252); Ac-Nle-c(Asp-Pro-dNal(2′)-Arg-Aia-Lys)-dVal-dPro-NH2 (SEQ ID NO: 253); Ac-Nle-c(Asp-Pro-dNal(2′)-Arg-Aba-Lys)-dVal-dPro-NH2 (SEQ ID NO: 254); Ac-Nle-c(Asp-Pro-dNal(2′)-Arg-Ata-Lys)-dVal-dPro-NH2 (SEQ ID NO: 255); Ac-Nle-c[Asp-Glu-dNal(2′)-Arg-Trp-Lys]-dVal-dPro-NH2 (SEQ ID NO: 256); Ac-Nle-c[Asp-Glu-dNal(2′)-Arg-Trp-Gly-Lys]-dVal-dPro-NH2 (SEQ ID NO: 257); Ac-Nle-c[Asp-Pro-Glu-dNal(2′)-Arg-Trp-Lys]-dVal-dPro-NH2 (SEQ ID NO: 258); Ac-Nle-c[Asp-Pro-Glu-dNal(2′)-Arg-Trp-Gly-Lys]-dVal-dPro-NH2 (SEQ ID NO: 259); Ac-Nle-c[Asp-Pro-dNal(2′)-Arg-Trp-Gly-Lys]-dVal-dPro-NH2 (SEQ ID NO: 260); Ac-Nle-c[Asp-Pro-dNal(2′)-Arg-Trp-Gly-Lys]-dPro-dPro-Lys-Asp-NH2 (SEQ ID NO: 261); Ac-Nle-c[Asp-Pro-dNal(2′)-Arg-Trp-Gly-Lys]-dPro-dPro-dLys-dAsp-NH2 (SEQ ID NO: 262); Ac-Glu-c[Asp-Pro-dNal(2′)-Arg-Trp-Gly-Lys]-dPro-dPro-Lys-Asp-NH2 (SEQ ID NO: 263); and Ac-Nle-c[Asp-Pro-dNal(2′)-Arg-Trp-Lys]-dPro-dPro-dLys-dAsp-NH2 (SEQ ID NO: 264), wherein c represents cyclization through R² and R⁷ via a lactam bond.

Clause 55. The method of clause 1, wherein the sequence of Formula (I) is selected from the group consisting of: Ac-Nle-c[Asp-Glu-His-dNal(2′)-Arg-Trp-Lys]-dVal-dPro-NH2 (SEQ ID NO: 265); Ac-Nle-c[Asp-Glu-His-dNal(2′)-Arg-Trp-Gly-Lys]-dVal-dPro-NH2 (SEQ ID NO: 266); Ac-Nle-c(Asp-dAla-His-dNal(2′)-Arg-Trp-Lys)-dVal-dPro-NH2 (SEQ ID NO: 267); Ac-Arg-c(Asp-dAla-His-dNal(2′)-Arg-Trp-Lys)-dVal-dPro-NH2 (SEQ ID NO: 268); Ac-Arg-c(Asp-dAla-His-dPhe-Arg-Trp-Lys)-dVal-dPro-NH2 (SEQ ID NO: 269); Ac-Arg-c (Cys-dAla-His-dNal(2′)-Arg-Trp-Cys)-dVal-dPro-NH2 (SEQ ID NO: 270); Ac-dArg-c(Asp-dAla-His-dNal(2′)-Arg-Trp-Lys)-dVal-dPro-NH2 (SEQ ID NO: 271); Ac-Arg-c(Asp-dAla-dNal(2′)-Arg-Trp-Lys)-dVal-dPro-NH2 (SEQ ID NO: 272); Ac-dArg-c(Asp-dAla-dNal(2′)-Arg-Trp-Lys)-dVal-dPro-NH2 (SEQ ID NO: 273); Ac-Nle-c(Asp-Ala-His-dNal(2′)-Arg-Trp-Lys)-dVal-dPro-NH2 (SEQ ID NO: 274); Ac-Arg-c(Asp-Ala-His-dNal(2′)-Arg-Trp-Lys)-dVal-dPro-NH2 (SEQ ID NO: 275); Ac-dArg-c(Asp-Ala-His-dNal(2′)-Arg-Trp-Lys)-dVal-dPro-NH2 (SEQ ID NO: 276); Ac-Nle-c(Asp-Trp-Arg-dNal(2′)-Pro-Lys)-dVal-dPro-NH2 (SEQ ID NO: 277); Ac-Nle-c(Asp-Pro-Trp-dNal(2′)-Lys)-dVal-dPro-NH2 (SEQ ID NO: 278); Ac-Nle-c(Asp-Pro-Trp-Arg-dNal(2′)-Pro-Lys)-dVal-dPro-NH2 (SEQ ID NO: 279); Ac-Nle-c(Asp-Pro-Trp-Arg-dNal(2′)-Lys)-dVal-dPro-NH2 (SEQ ID NO: 280); Ac-Nle-c(Lys-Trp-Arg-dNal(2′)-Pro-Asp)-dVal-dPro-NH2 (SEQ ID NO: 281) Ac-Arg-c(Asp-dAla-His-dPhe-Arg-Trp-Lys)-NH2 (SEQ ID NO: 282); Ac-Arg-c (Cys-dAla-His-dNal(2′)-Arg-Trp-Cys)-NH2 (SEQ ID NO: 283); and Ac-Arg-c(Asp-dAla-His-dNal(2′)-Arg-Trp-Lys)-NH2 (SEQ ID NO: 284), wherein c represents cyclization through R² and any of R⁵—R⁸ via a lactam bond or a disulfide bond.

Clause 56. The method of clause 1, wherein the sequence of Formula (I) is: Ac-dVal-dPro-c(Asp-Trp-Arg-dNal(2′)-Pro-Lys)-Nle-NH2 (SEQ ID NO: 285); Ac-dVal-dPro-c (Lys-Trp-Arg-dNal(2′)-Pro-Asp)-Nle-NH2 (SEQ ID NO: 286); Ac-dVal-dPro-c(Asp-Trp-Arg-dNal(2′)-Pro-Lys)-Nle-Nle-NH2 (SEQ ID NO: 287); Ac-dVal-dPro-c (Lys-Trp-Arg-dNal(2′)-Pro-Asp)-Nle-Nle-NH2 (SEQ ID NO: 288); Ac-dVal-dPro-c(Asp-Trp-Arg-dNal(2′)-Pro-Lys)-dVal-dPro-NH2 (SEQ ID NO: 289); Ac-dVal-dPro-c (Lys-Trp-Arg-dNal(2′)-Pro-Asp)-dVal-dPro-NH2 (SEQ ID NO: 290), wherein c represents cyclization through R¹ and any one of R⁶ via a lactam bond.

Clause 57. The method of clause 1, wherein the non-naturally occurring melanocortin analog comprises any one of the sequences of SEQ ID NOs: 2-290.

Clause 58. The method of clause 1, wherein the non-naturally occurring melanocortin analog comprises any one of the sequences of SEQ ID NOs: 2-187, 196-212, and 240-290.

Clause 59. The method of clause 1, wherein the non-naturally occurring melanocortin analog comprises a sequence of SEQ ID NO: 3.

Clause 60. The method of any one of clauses 1-59, wherein the non-naturally occurring melanocortin analog is present in a composition.

Clause 61. The method of clause 60, wherein the composition further comprises a pharmaceutical salt.

Clause 62. The method of clause 60, wherein the composition further comprises a pharmaceutical carrier.

Clause 63. The method of clause 60, wherein the non-naturally occurring melanocortin analog is present in the composition in a concentration of 0.1 mg/mL to 500 mg/mL, relative to a total volume of the composition.

Clause 64. The method of clause 63, wherein the non-naturally occurring melanocortin analog comprises a sequence of SEQ ID NO: 3, and wherein the non-naturally occurring melanocortin analog is present in the composition in a concentration of 5 mg/ml to 100 mg/mL, relative to a total volume of the composition.

Clause 65. The method of clause 64, wherein the non-naturally occurring melanocortin analog is present in the composition in a concentration of about 50 mg/mL, relative to a total volume of the composition.

Clause 66. The method of any one of clauses 1-65, wherein the non-naturally occurring melanocortin analog is administered via intraperitoneal, intravenous, parenteral, subcutaneous, intramuscular, intracerebroventricular, intranasal, or oral administration.

Clause 67. The method of any one of clauses 60-66, wherein the composition comprising the non-naturally occurring melanocortin analog is administered to the subject parenterally.

Clause 68. The method of any one of clauses 60-67, wherein the composition comprising the non-naturally occurring melanocortin analog is administered to the subject subcutaneously.

Clause 69. The method of any one of clauses 1-68, wherein the non-naturally occurring melanocortin analog crosses the blood-brain-barrier of the subject.

Clause 70. The method of any one of clauses 1-69, wherein the therapeutically effective amount of the non-naturally occurring melanocortin analog is from 0.001 mg/kg to 25 mg/kg per body weight of the subject.

Clause 71. The method of any one of clauses 1-70, wherein the therapeutically effective amount of the non-naturally occurring melanocortin analog is from 0.5 mg/kg to 10 mg/kg per body weight of the subject.

Clause 72. The method of any one of clauses 1-71, wherein the non-naturally occurring melanocortin analog is administered at least once daily in an amount ranging from 0.001 mg/kg to 25 mg/kg per body weight of the subject.

Clause 73. The method of any one of clauses 1-72, wherein the non-naturally occurring melanocortin analog is administered at least once daily in an amount ranging from about 0.5 mg/kg to about 10 mg/kg per body weight of the subject.

Clause 74. The method of clause 72, wherein the non-naturally occurring melanocortin analog comprises a sequence of SEQ ID NO: 3, and the non-naturally occurring melanocortin analog is administered at least once daily in an amount ranging from 0.001 mg/kg to 25 mg/kg per body weight of the subject.

Clause 75. The method of clause 73, wherein the non-naturally occurring melanocortin analog comprises a sequence of SEQ ID NO: 3, and the non-naturally occurring melanocortin analog is administered at least once daily in an amount ranging from 0.5 mg/kg to 10 mg/kg per body weight of the subject.

Clause 76. The method of any one of clauses 1-75, wherein the non-naturally occurring melanocortin analog is administered to the subject for at least 1 day, 1 week, 1 month, 3 months, 6 months, 1 year, or 5 years.

Clause 77. The method of any one of clauses 1-75, wherein the non-naturally occurring melanocortin analog is administered to the subject for 1 day, 5 days, 7 days, 14 days, 21 days, 28 days, 35 days, 40 days, 45 days, 50 days, 60 days, 75 days, 90 days, 100 days, 110 days, or 120 days.

Clause 78. The method of clause 62, wherein the pharmaceutical carrier comprises water.

Clause 79. The method of any one of clauses 1-78, wherein the subject experiences loss of appetite, reduced appetite, decreased food consumption, and/or weight loss prior to the administration.

Clause 80. The method of clause 79, wherein the loss of appetite, reduced appetite, decreased food consumption, and/or weight loss is caused by cachexia.

Clause 81. The method of any one of clauses 1-80, wherein the method (i) stimulates appetite of the subject; (ii) increases food consumption by the subject; (iii) prevents or alleviates nausea, emesis, and/or anorexia in the subject; (iv) increases or maintains body weight or BMI of the subject; (v) prevents or reduces weight loss of the subject; (vi) increases or maintains muscle mass of the subject; (vii) prevents or reduces muscle mass loss of the subject; (viii) increases or maintains fat mass of the subject; and/or (ix) prevents or reduces fat mass loss of the subject.

Clause 82. The method of any one of clauses 1-81, wherein the method increases body weight or BMI of and/or increases food consumption by the subject.

Clause 83. The method of clause 81, wherein appetite of the subject is increased by at least 10% to 200% after the administration.

Clause 84. The method of clause 81, wherein appetite of the subject is increased by at least 25% to 100% after the administration.

Clause 85. The method of clause 83 or 84, wherein appetite is increased for at least 1 day, 1 week, 1 month, 3 months, 6 months, 1 year, or 5 years during the administration.

Clause 86. The method of clause 83 or 84, wherein appetite is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration.

Clause 87. The method of any one of clauses 83-86, wherein the increased appetite is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

Clause 88. The method of any one of clauses 81 and 83-87, wherein the appetite is assessed by a scale for measuring desire to eat, feeling of hunger, and/or satiety.

Clause 89. The method of clause 88, wherein the appetite is assessed between meals.

Clause 90. The method of any one of clauses 81 and 83-89, wherein the subject comprises two or more subjects, and wherein the appetite is an average appetite of the two or more subjects.

Clause 91. The method of clause 81 or 82, wherein food consumption of the subject is increased by at least 25% to 2,000% after the administration.

Clause 92. The method of clause 81 or 82, wherein food consumption of the subject is increased by at least 50% to 500% after the administration.

Clause 93. The method of clause 91 or 92, wherein food consumption is increased for at least 1 day, 1 week, 1 month, 3 months, 6 months, 1 year, or 5 years during the administration.

Clause 94. The method of clause 91 or 92, wherein food consumption is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration.

Clause 95. The method of any one of clauses 91-94, wherein the increased food consumption is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

Clause 96. The method of any one of clauses 81-82 and 91-95, wherein the food consumption is determined by total calories (kcal) consumed in 1 day or mass of food (grams or kilograms) consumed in 1 day.

Clause 97. The method of clause 81, wherein anorexia is determined by food consumption.

Clause 98. The method of clause 81 or 82, wherein body weight of the subject is increased by at least 5% to 200% after the administration.

Clause 99. The method of clause 81 or 82, wherein body weight of the subject is increased by at least 5% to 25% after the administration.

Clause 100. The method of clause 81 or 82, wherein body weight of the subject is increased by at least 20% to 100% after the administration.

Clause 101. The method of any one of clauses 98-100, wherein the increased body weight is maintained for at least 1 week, 2 weeks, 3 weeks, 1 month, 3 months, 6 months, 1 year, or 5 years, after the administration is stopped.

Clause 102. The method of clause 81, wherein muscle mass of the subject is increased by at least 1% to 100% after the administration.

Clause 103. The method of clause 81, wherein muscle mass of the subject is increased by at least 5% to 15% after the administration.

Clause 104. The method of clause 81, wherein muscle mass of the subject is increased by at least 25% to 50% after the administration.

Clause 105. The method of any one of clauses 102-104, wherein the increased muscle mass is maintained for at least 1 week, 2 weeks, 3 weeks, 1 month, 3 months, 6 months, 1 year, or 5 years, after the administration is stopped.

Clause 106. The method of any one of clauses 81 and 102-105, wherein the muscle mass is cardiac muscle mass, skeletal muscle mass, or both.

Clause 107. The method of clause 81, wherein fat mass of the subject is increased by at least 1% to 100% after the administration.

Clause 108. The method of clause 81, wherein fat mass of the subject is increased by at least 5% to 15% after the administration.

Clause 109. The method of clause 81, wherein fat mass of the subject is increased by at least 25% to 50% after the administration.

Clause 110. The method of any one of clauses 107-109, wherein the increased fat mass is maintained for at least 1 week, 2 weeks, 3 weeks, 1 month, 3 months, 6 months, 1 year, or 5 years, after the administration is stopped.

Clause 111. The method of any one of clauses 1-80, wherein the method stimulates appetite in the subject compared to a control subject, wherein the control subject is not administered the non-naturally occurring melanocortin analog.

Clause 112. The method of clause 111, wherein the control subject experiences loss of appetite, reduced appetite, decreased food consumption, and/or weight loss.

Clause 113. The method of clause 112, wherein the loss of appetite, reduced appetite, decreased food consumption, and/or weight loss is caused by cachexia.

Clause 114. The method of any one of clauses 111-113, wherein appetite of the subject is increased after the administration by at least 10% to 200%, relative to appetite of the control subject.

Clause 115. The method of any one of clauses 111-113, wherein appetite of the subject is increased after the administration by at least 25% to 100%, relative to appetite of the control subject.

Clause 116. The method of clause 114 or 115, wherein appetite is increased for at least 1 day, 1 week, 1 month, 3 months, 6 months, 1 year, or 5 years during the administration.

Clause 117. The method of clause 59 or 60, wherein appetite is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration.

Clause 118. The method of any one of clauses 114-117, wherein the increased appetite is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

Clause 119. The method of any one of clauses 114-118, wherein the appetite is assessed by a scale for measuring desire to eat, feeling of hunger, and/or level of satiety.

Clause 120. The method of clause 119, wherein the appetite is assessed between meals.

Clause 121. The method of any one of clauses 111-120, wherein the subject comprises two or more subjects, and wherein the appetite of the subject is an average appetite of the two or more subjects.

Clause 122. The method of any one of clauses 111-121, wherein the control subject comprises two or more control subjects, and wherein the appetite of the control subject is an average appetite of the two or more control subjects.

Clause 123. The method of any one of clauses 111-113, wherein food consumption of the subject is increased after the administration by at least 25% to 2,000%, relative to food consumption of the control subject.

Clause 124. The method of any one of clauses 111-113, wherein food consumption of the subject is increased after the administration by at least 50% to 500%, relative to food consumption of the control subject.

Clause 125. The method of clause 123 or 124, wherein food consumption is increased for at least 1 day, 1 week, 1 month, 3 months, 6 months, 1 year, or 5 years during the administration.

Clause 126. The method of clause 123 or 124, wherein food consumption is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration.

Clause 127. The method of any one of clauses 123-126, wherein the increased food consumption is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

Clause 128. The method of any one of clauses 123-127, wherein the food consumption is determined by total calories (kcal) consumed in 1 day or mass of food (grams or kilograms) consumed in 1 day.

Clause 129. The method of any one of clauses 111-113, wherein body weight of the subject is increased after the administration, and wherein weight gain of the subject after the administration is at least 5% to 200% greater than that of the control subject.

Clause 130. The method of any one of clauses 111-113, wherein body weight of the subject is increased after the administration, and wherein weight gain of the subject after the administration is at least 5% to 25% greater than that of the control subject.

Clause 131. The method of any one of clauses 111-113, wherein body weight of the subject is increased after the administration, and wherein weight gain of the subject after the administration is at least 20% to 100% greater than that of the control subject.

Clause 132. The method of any one of clauses 129-131, wherein the increased body weight is maintained for at least 1 week, 2 weeks, 3 weeks, 1 month, 3 months, 6 months, 1 year, or 5 years, after the administration is stopped.

Clause 133. The method of any one of clauses 111-113, wherein muscle mass of the subject is increased after the administration, and wherein muscle mass gain of the subject after the administration is at least 1% to 100% greater than that of the control subject.

Clause 134. The method of any one of clauses 111-113, wherein muscle mass of the subject is increased after the administration, and wherein muscle mass gain of the subject after the administration is at least 5% to 15% greater than that of the control subject.

Clause 135. The method of any one of clauses 111-113, wherein muscle mass of the subject is increased after the administration, and wherein muscle mass gain of the subject after the administration is at least 25% to 50% greater than that of the control subject.

Clause 136. The method of any one of clauses 133-135, wherein the increased muscle mass is maintained for at least 1 week, 2 weeks, 3 weeks, 1 month, 3 months, 6 months, 1 year, or 5 years, after the administration is stopped.

Clause 137. The method of any one of clauses 111-113, wherein fat mass of the subject is increased after the administration, and wherein fat mass gain of the subject after the administration is at least 1% to 100% greater than that of the control subject.

Clause 138. The method of any one of clauses 111-113, wherein fat mass of the subject is increased after the administration, and wherein fat mass gain of the subject after the administration is at least 5% to 15% greater than that of the control subject.

Clause 139. The method of any one of clauses 111-113, wherein fat mass of the subject is increased after the administration, and wherein fat mass gain of the subject after the administration is at least 25% to 50% greater than that of the control subject.

Clause 140. The method of any one of clauses 137-139, wherein the increased fat mass is maintained for at least 1 week, 2 weeks, 3 weeks, 1 month, 3 months, 6 months, 1 year, or 5 years, after the administration is stopped.

Clause 141. The method of any one of clauses 1-140, wherein the subject is a human.

Clause 142. The method of any one of clauses 1-140, wherein the subject is an animal.

Clause 143. The method of clause 141, wherein the subject has a body mass index (BMI) of 18.5 kg/m² to 25 kg/m².

Clause 144. The method of clause 141, wherein the subject has a BMI of less than 20 kg/m².

Clause 145. The method of clause 141, wherein the subject has a BMI of less than 18.5 kg/m².

Clause 146. The method of any one of clauses 1-80, wherein the subject is a metabolically unchallenged subject, and wherein the method increases body weight of and/or increases food consumption by the metabolically unchallenged subject compared to a control subject.

Clause 147. The method of clause 146, wherein the metabolically unchallenged subject and the control subject are the same subject.

Clause 148. The method of clause 146, wherein the metabolically unchallenged subject and the control subject are different subjects.

Clause 149. The method of any one of clauses 146-148, wherein the metabolically unchallenged subject experiences loss of appetite, reduced appetite, a loss of food consumption, and/or weight loss prior to the administration.

Clause 150. The method of any one of clauses 146-149, wherein the non-naturally occurring melanocortin analog is administered to the at least once daily in an amount ranging from 0.001 mg/kg to 25 mg/kg per body weight of the metabolically unchallenged subject.

Clause 151. The method of any one of clauses 146-150, wherein the non-naturally occurring melanocortin analog is administered at least once daily in an amount ranging from about 0.5 mg/kg to about 10 mg/kg per body weight of the metabolically unchallenged subject.

Clause 152. The method of any one of clauses 146-151, wherein the non-naturally occurring melanocortin analog is administered to the metabolically unchallenged subject for at least 1 day, 1 week, 1 month, 3 months, 6 months, 1 year, or 5 years.

Clause 153. The method of any one of clauses 146-151, wherein the non-naturally occurring melanocortin analog is administered to the metabolically unchallenged subject for 1 day, 7 days, 14 days, 21 days, 28 days, 35 days, 40 days, 45 days, 50 days, 60 days, 75 days, 90 days, 100 days, 110 days, or 120 days.

Clause 154. The method of any one of clauses 146-153, wherein body weight of the metabolically unchallenged subject is increased after the administration by at least 5% to 200%, relative to body weight of the control subject before the administration, wherein the control subject is a metabolically unchallenged subject.

Clause 155. The method of any one of clauses 146-153, wherein body weight of the metabolically unchallenged subject is increased after the administration by at least about 5% to about 25%, relative to body weight of the control subject before the administration, wherein the control subject is a metabolically unchallenged subject.

Clause 156. The method of any one of clauses 146-153, wherein body weight of the metabolically unchallenged subjects increased after the administration by at least about 20% to about 100%, relative to body weight of the control subject before the administration, wherein the control subject is a metabolically unchallenged subject.

Clause 157. The method of any one of clauses 146-153, wherein body weight of the metabolically unchallenged subject is increased after the administration by at least 5% to 200%, relative to body weight of the control subject, wherein the control subject is a metabolically unchallenged subject who experiences loss of appetite, reduced appetite, and/or weight loss.

Clause 158. The method of any one of clauses 146-153, wherein body weight of the subject is increased after the administration by at least about 5% to about 25%, relative to body weight of the control subject, wherein the control subject is a metabolically unchallenged subject who experiences loss of appetite, reduced appetite, and/or weight loss.

Clause 159. The method of any one of clauses 146-153, wherein body weight of the subject is increased after the administration by at least about 20% to about 100%, relative to body weight of the control subject, wherein the control subject is a metabolically unchallenged subject who experiences loss of appetite, reduced appetite, and/or weight loss.

Clause 160. The method of any one of clauses 154-159, wherein the increased body weight is maintained for at least 1 week, 2 weeks, 3 weeks, 1 month, 3 months, 6 months, 1 year, or 5 years, after the administration is stopped.

Clause 161. The method of any one of clauses 146-160, wherein food consumption of the metabolically unchallenged subject is increased after the administration by at least 50% to 2,000%, relative to food consumption of the metabolically unchallenged subject before the administration.

Clause 162. The method of any one of clauses 146-160, wherein food consumption of the metabolically unchallenged subject is increased after the administration by at least about 100%, relative to food consumption of the metabolically unchallenged subject before the administration.

Clause 163. The method of any one of clauses 146-160, wherein food consumption of the metabolically unchallenged subject is increased after the administration by at least about 1,000%, relative to food consumption of the metabolically unchallenged subject before the administration.

Clause 164. The method of any one of clauses 146-160, wherein food consumption of the metabolically unchallenged subject is increased after the administration by at least 50% to 2,000%, relative to food consumption of the control subject, wherein the control subject is a metabolically unchallenged subject who experiences loss of appetite, reduced appetite, and/or weight loss.

Clause 165. The method of any one of clauses 146-164, wherein food consumption is increased for at least 1 day, 1 week, 1 month, 3 months, 6 months, 1 year, or 5 years during the administration.

Clause 166. The method of any one of clauses 146-164, wherein food consumption is increased for at least 1 day, 7 days, 14 days, 21 days, 28 days, 35 days, 40 days, 45 days, 50 days, 60 days, 75 days, 90 days, 100 days, 110 days, or 120 days during the administration.

Clause 167. The method of any one of clauses 161-166, wherein the increased food consumption is maintained for at least 1 week, 2 weeks, 3 weeks, 1 month, 3 months, 6 months, 1 year, or 5 years, after the administration is stopped.

Clause 168. The method of any one of clauses 146-167, wherein food consumption is determined by total calories (kcal) consumed in 1 day or mass of food (grams or kilograms) consumed in 1 day.

Clause 169. The method of any one of clauses 146-167, wherein one or more side effects exhibited by the metabolically unchallenged subject after administration of the non-naturally occurring melanocortin analog are reduced compared to one or more side effects exhibited by a control subject that was administered a natural melanocortin.

Clause 170. The method of any one of clauses 146-169, the control subject is a metabolically unchallenged subject.

Clause 171. The method of any one of clauses 1-86, wherein the metabolically unchallenged subject has a disease or condition selected from the group consisting of a psychological disease or condition, an allergy, an intolerance, a gastrointestinal disease or condition, a side-effect from a medication, substance use, a viral infection, a bacterial infection, food poisoning, dehydration, fatigue, hormonal imbalance, pain, a cardiovascular disease or condition, anemia, an autoimmune disease or condition, a respiratory disease or condition, and an inflammatory disease or condition.

Clause 172. The method of clause 171, wherein the psychological disease or condition is selected from the group consisting of depression, bipolar disorder, schizophrenia, anorexia nervosa, anxiety, grief, stress, bulimia, post-traumatic stress disorder, phobia, aversions such as smell, taste, sight, texture, social anxiety, shock, obsessive-compulsive disorder, eating disorders dementia, Alzheimer's, Parkinson's, multiple sclerosis.

Clause 173. The method of clause 171, wherein the allergy or intolerance is selected from the group consisting of gluten, dairy, soy, nut and seed.

Clause 174. The method of clause 171, wherein the gastrointestinal disease or condition is selected from the group consisting of irritable bowel syndrome (IBS), celiac disease, and Crohn's disease.

Clause 175. The method of clause 171, wherein the disease or condition is advanced age.

Clause 176. The method of clause 171, wherein the medication is selected from the group consisting of antibiotics, codeine, morphine, sleeping pills, blood pressure medications, diuretics, anabolic steroids, cardiovascular medications, including digoxin, fluoxetine and hydralazine.

Clause 177. The method of clause 171, wherein the substance use is selected from the group consisting of cocaine, methamphetamines, heroin, alcohol.

Clause 178. The method of clause 171, wherein the disease or condition is substance withdrawal.

Clause 179. The method of clause 171, wherein the disease or condition is food poisoning.

Clause 180. The method of clause 171, wherein the disease or condition is dehydration.

Clause 181. The method of clause 171, wherein the viral infection, bacterial infection, or disease or condition caused by a viral infection or a bacterial infection is selected from the group consisting of upper respiratory infection, COVID-19, pneumonia, gastroenteritis, skin, meningitis, HIV, hepatitis, flu, common cold, urine infection.

Clause 182. The method of clause 171, wherein the disease or condition is selected from the group consisting of sleep deprivation, fatigue, chronic fatigue syndrome, nausea, loss of taste, smell, sight, and satiety.

Clause 183. The method of clause 171, wherein the disease or condition is pregnancy or hormonal therapy.

Clause 184. The method of clause 171, wherein the pain is caused by a condition or disease selected from the group consisting of fibromyalgia, migraines, nerve damage, postural orthostatic tachycardia syndrome, post-surgical, oral pain, and dental pain.

Clause 185. The method of clause 171, wherein the disease or condition is selected from the group consisting of heart disease, high blood pressure, anemia, lupus, rheumatoid arthritis, chronic lung disease, inflammatory conditions, and menopause.

Clause 186. The method of any one of clauses 146-185, wherein the metabolically unchallenged subject is a human.

Clause 187. The method of any one of clauses 146-185, wherein the metabolically unchallenged subject is an animal.

Clause 188. The method of any one of clauses 146-187, wherein the increased body weight or BMI comprises increased muscle mass, increased fat mass, or both.

Clause 189. The method of clause 188, wherein the muscle mass is skeletal muscle mass, cardiac muscle mass, or both.

Clause 190. The method of any one of clauses 146-187, wherein the increased body weight or BMI comprises increased lean mass.

Clause 191. The method of any one of clauses 1-145, wherein the subject has a cancer.

Clause 192. The method of clause 191, wherein the cancer is at least one selected from the group consisting of bone cancer, testicular cancer, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colorectal cancer, oral cancer, skin cancer, and melanoma.

Clause 193. The method of clause 191 or 192, wherein the cancer is at least one selected from the group consisting of bone cancer, lung cancer, testicular cancer, breast cancer, ovarian cancer, cervical cancer, bladder cancer, and head and neck cancer.

Clause 194. The method of clause 191 or 192, wherein the cancer is at least one selected from the group consisting of non-small-cell lung cancer, colorectal cancer, stomach cancer, ovarian cancer, and pancreatic cancer.

Clause 195. The method of claim any one of clauses 191-194, further comprising administering an anticancer agent to the subject.

Clause 196. The method of clause 195, wherein the anticancer agent is at least one chemotherapeutic agent.

Clause 197. The method of clause 196, wherein the at least one chemotherapeutic agent comprises one or more chemotherapeutic agents selected from the group consisting of a platinum-coordination complex, an antimetabolite, a tubulin binding agent, an alkylating antineoplastic agent, and a cytotoxic antibiotic.

Clause 198. The method of any one of clauses 1-197, further comprising administering an appetite-regulating agent to the subject.

Clause 199. The method of any one of clauses 1-198, wherein the subject does not experience an adverse event following administration of the non-naturally occurring melanocortin analog.

Clause 200. The method of any one of clauses 1-199, wherein the subject does not experience an adverse event attributable to the non-naturally occurring melanocortin analog following administration.

Clause 201. The method of clause 81, wherein appetite of the subject is assessed by a scale for measuring satiety.

Clause 202. The method of clauses 88 or 201, wherein satiety decreased by at least 40% to 150% within 8 days after the administration.

Clause 203. The method of clauses 88 or 201, wherein satiety decreased by at least 7 points within 8 days after the administration.

Clause 204. The method of clauses 88 or 201, wherein satiety decreased below 15 within 8 days after the administration.

Clause 205. The method of clause 81 or 82, wherein body weight of the subject is increased by at least 2% to 10% between day 2 after the administration and day 7 after the administration.

Clause 206. The method of clause 81 or 82, wherein body weight of the subject is increased by at least 1.5 kg between day 2 after the administration and day 7 after the administration.

Clause 207. The method of clause 81 or 82, wherein BMI of the subject is increased by at least 1% to 10% between day 2 after the administration and day 7 after the administration.

Clause 208. The method of clause 81 or 82, wherein BMI of the subject is increased by at least 0.5 between day 2 after administration and day 7 after administration.

Clause 209. The method of any one of clauses 114-119, wherein appetite of the subject is assessed by a scale for measuring satiety.

Clause 210. The method of any one of clauses 119 or 209, wherein satiety of the subject is decreased after the administration by at least 40% to 150%, relative to satiety of the control subject.

Clause 211. The method of any one of clauses 119 or 209, wherein satiety of the subject is decreased after the administration by at least 14 points relative to satiety of the control subject.

Clause 212. The method of any one of clauses 111-113, wherein body weight of the subject is increased between day 2 after the administration and day 7 after the administration, and wherein weight gain of the subject between day 2 after the administration and day 7 after the administration is at least 20% to 150% greater than that of the control subject.

Clause 213. The method of any one of clauses 111-113, wherein body weight of the subject is increased between day 2 after the administration and day 7 after the administration, and wherein weight gain of the subject between day 2 after the administration and day 7 after administration is at least 0.5 kg greater than that of the control subject.

Clause 214. The method of any one of clauses 111-113, wherein BMI of the subject is increased between day 2 after the administration and day 7 after the administration, and wherein BMI increase of the subject between day 2 after the administration and day 7 after administration is at least 20% to 100% greater than that of the control subject.

Clause 215. The method of any one of clauses 111-113, wherein BMI of the subject is increased between day 2 after the administration and day 7 after the administration, and wherein BMI increase of the subject between day 2 after the administration and day 7 after administration is at least 0.25 greater than that of the control subject.

Clause 216. Use of a non-naturally occurring melanocortin analog for stimulating appetite of a subject in need thereof, the method comprising: administering to the subject a therapeutically effective amount of the non-naturally occurring melanocortin analog, wherein the non-naturally occurring melanocortin analog comprises a sequence according to Formula (I), administering to the subject a therapeutically effective amount of a non-naturally occurring melanocortin analog comprising a sequence according to Formula (I),

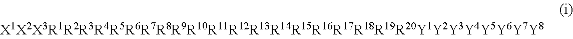

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-α][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);;
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
   wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine; and
wherein the non-naturally occurring melanocortin analog comprises one or more of the following features: (i) X² or X³ is present; (ii) R¹ is absent or is selected from the group consisting of dArg, dMet, dlle, dLeu, dVal, dAla, Ala, Tle, dTle, DNle, Nva, Gly, dPro, dCys, dPhe, dTyr, dGln, dAsn, transPro(guan), cisPro(guan), dTyr, Tyr, and Dmt; (iii) R² is absent; (iv) R³ is selected from the group consisting of dPro, Pro-Gly, and dAla; (v) R⁴ is selected from the group consisting of His, Trp, and Phe; (vi) R⁵ is Phe; (vii) R⁷ is absent; (viii) R⁸ is present; (ix) R⁹ and/or R¹⁰ is present; (x) one or more of R¹¹—R²⁰ are present; and (xi) Y⁴ is present; and
the non-naturally occurring melanocortin analog is cyclized through a moiety selected from the group consisting of:
   a disulfide bond between R¹ or R² and R⁷ or X¹ when R¹ or R² is cysteine and R⁷ or X¹ is cysteine;
   a disulfide bond between R² and any one of R⁵—R²⁰ when R² and the any one of R⁵—R²⁰ are selected from the group consisting of cysteine, Pen, and dPen;
   a lactam bridge between R¹ and R⁷ when R¹ is norleucine and R⁷ is glutamic acid;
   a side-chain lactam bridge between R² and R⁷ when R² is glutamic acid, aspartic acid, or CO-cis-CH═CH—CO, and R⁷ is lysine or ornithine;
   a side-chain lactam bridge between R² and R⁸ when R² is glutamic acid or aspartic acid, R⁸ is lysine, and R⁷ is proline or tryptophan;
   a lactam closure between R¹ and R⁷ when R¹ is succinic acid or o-phthalic acid and R⁷ is lysine; and
   a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

Clause 217. The use of clause 216, wherein the N-terminus, if present, is modified by a functional group selected from the group consisting of an acyl group, an imine group, an amide group, a urea group, a carbamate group, a sulfonamide group, and an alkylamine group.

Clause 218. The use of clause 217, wherein the N-terminus, if present, is modified by an acyl group.

Clause 219. The use of clause 218, wherein the acyl group is acetyl group.

Clause 220. The use of clause 219, wherein the acyl group is formyl group.

Clause 221. The use of clause 217, wherein the N-terminus, if present, is modified by an imine group.

Clause 222. The use of clause 217, wherein the N-terminus, if present, is modified by an amide group.

Clause 223. The use of clause 222, wherein the amide group is a pyroglutamyl (pGlu) group.

Clause 224. The use of clause 222, wherein the amide group is derived from a fatty acid.

Clause 225. The use of clause 216, wherein the N-terminus, if present, is not modified.

Clause 226. The use of clause 216, wherein the C-terminus is modified by a functional group selected from the group consisting of an amide group, an ester group, and an aldehyde group.

Clause 227. The use of clause 226, wherein the C-terminus is modified by an amide group.

Clause 228. The use of clause 227, wherein the amide group is an —NHalkyl amide group or an —NHaryl amide group.

Clause 229. The use of clause 228, wherein the—NHaryl amide group is p-nitroanilide group or 7-amino-4-methylcoumarin.

Clause 230. The use of clause 226, wherein the C-terminus is modified by an ester group.

Clause 231. The use of clause 216, wherein the C-terminus is not modified.

Clause 232. The use of clause 216, wherein R¹ is absent, and R² is D-aspartic acid.

Clause 233. The use of clause 232, wherein X¹, X², and X³ are absent.

Clause 234. The use of clause 216, wherein R⁴ is D-Nal(2′).

Clause 235. The use of clause 216, wherein Y³-Y⁸ are absent.

Clause 236. The use of clause 235, wherein: Y¹ is D-valine and Y² is D-proline; or Y¹ is D-proline and Y² is D-valine.

Clause 237. The use of clause 216, wherein Y³ is present and Y⁴-Y⁸ are absent.

Clause 238. The use of clause 237, wherein: Y¹ is D-valine or D-proline; Y² is D-valine or D-proline; and/or Y³ is D-valine or D-proline.

Clause 239. The use of clause 237, wherein: Y¹ is D-valine, Y² is D-valine, and Y³ is D-proline; Y¹ is D-proline, Y² is D-valine, and Y³ is D-valine; Y¹ is D-valine, Y² is D-proline, and Y³ is D-valine; or Y¹ is D-proline, Y² is D-valine, and Y³ is D-proline.

Clause 240. The use of clause 216, wherein Y³ and Y⁴ are present, and Y⁵-Y⁸ are absent.

Clause 241. The use of clause 240, wherein: Y¹ is D-valine or D-proline; Y² is D-valine or D-proline; Y³ is D-valine or D-proline; and/or Y⁴ is D-valine or D-proline.

Clause 242. The use of clause 241, wherein: Y¹ is D-valine, Y² is D-valine, Y³ is D-valine, and Y⁴ is D-proline; Y¹ is D-proline, Y² is D-valine, Y³ is D-valine, and Y⁴ is D-valine; Y¹ is D-valine, Y² is D-proline, Y³ is D-valine, and Y⁴ is D-valine; Y¹ is D-valine, Y² is D-valine, Y³ is D-proline, and Y⁴ is D-valine; or Y¹ is D-valine, Y² is D-proline, Y³ is D-valine, and Y⁴ is D-proline.

Clause 243. The use of clause 216, wherein R¹, R², and R⁷ are present and R⁸-R²⁰ are absent, and the sequence of Formula (I) is cyclized through R² and R⁷ via a lactam bond.

Clause 244. The use of clause 243, wherein: R¹ is acetylated norleucine; R² is aspartic acid; R³ is selected from the group consisting of proline, hydroxyproline, and D-hydroxyproline; R⁴ is dNal(2′); R⁵ is arginine; R⁶ is D-tryptophan or L-tryptophan; R⁷ is lysine; Y¹ is D-valine; and/or Y² is D-proline.

Clause 245. The use of clause 245, wherein the sequence of Formula (I) is: Ac-Nle-c(Asp-Pro-dNal2′-Arg-Trp-Lys)-dVal-dPro-NH2 (SEQ ID NO: 3); or Ac-Nle-c(Asp-Hyp-dNal2′-Arg-Trp-Lys)-dVal-dPro-NH2 (SEQ ID NO: 4), wherein c represents cyclization through R² and R⁷ via a lactam bond.

Clause 246. The use of clause 216, wherein R¹, R², R⁷, and R⁸ are present and R⁹—R²⁰ are absent, and the sequence of Formula (I) is cyclized through R² and R⁸ via a lactam bond.

Clause 247. The use of clause 246, wherein: R¹ is acetylated norleucine; R² is aspartic acid; R³ is selected from the group consisting of proline, hydroxyproline, D-hydroxyproline, phenylalanine, and histidine; R⁴ is histidine or dNal(2′); R⁵ is dNal(2′) or arginine; R⁶ is selected from the group consisting of arginine, D-tryptophan, and L-tryptophan; R⁷ is tryptophan or proline; R⁸ is lysine; Y¹ is selected from the group consisting of D-valine, D-leucine, and D-isoleucine; and/or Y² is D-proline.

Clause 248. The use of clause 247, wherein the sequence of Formula (I) is selected from the group consisting of: Ac-Nle-c(Asp-Pro-His-dNal(2′)-Arg-Trp-Lys)-dVal-dPro-NH2 (SEQ ID NO: 6); Ac-Nle-c(Asp-Phe-His-dNal(2′)-Arg-Trp-Lys)-dLeu-dPro-NH2 (SEQ ID NO: 7); Ac-Nle-c(Asp-Phe-His-dNal(2′)-Arg-Trp-Lys)-dIIe-dPro-NH2 (SEQ ID NO: 8); Ac-Nle-c(Asp-His-dNal(2′)-Arg-Trp-Pro-Lys)-dVal-dPro-NH2 (SEQ ID NO: 9); Ac-Nle-c(Asp-Hyp-dNal(2′)-Arg-Trp-Pro-Lys)-dVal-dPro-NH2 (SEQ ID NO: 10); and Ac-Nle-c(Asp-Pro-His-dNal(2′)-Arg-Trp-Lys)-dPro-dVal-NH2 (SEQ ID NO: 11; D5), wherein c represents cyclization through R² and R⁸ via a lactam bond.

Clause 249. The use of clause 216, wherein R¹—R² and R⁷—R¹⁰ are present and R¹¹—R²⁰ are absent, and the sequence of Formula (I) is cyclized through R² and R¹⁰ via a lactam bond.

Clause 250. The use of clause 249, wherein the sequence of Formula (I) is Ac-Nle-c(Asp-Phe-Phe-Pro-His-dNal(2′)-Arg-Trp-Lys)-dVal-dPro-NH2 (SEQ ID NO: 12), wherein c represents cyclization through R² and R¹⁰ via a lactam bond.

Clause 251. The use of clause 216, wherein R¹—R² and R⁷—R¹⁰ are present and R¹¹—R²⁰ are absent, and the sequence of Formula (I) is cyclized through R⁴ and R¹° via a lactam bond.

Clause 252. The use of clause 251, wherein the sequence of Formula (I) is Ac-Nle-Phe-Phe-c(Asp-Phe-His-dNal(2′)-Arg-Trp-Lys)-dVal-Dpro-NH2 (SEQ ID NO: 13), wherein c represents cyclization through R⁴ and R¹⁰ via a lactam bond.

Clause 253. The use of clause 232, wherein the sequence of Formula (I) is Ac-Nle-c(Asp-Pro-dNal(2′)-Arg-Trp-Lys)-dVal-dPro(SEQ ID NO: 2).

Clause 254. The use of clause 216, wherein the sequence of Formula (I) is linear.

Clause 255. The use of clause 254, wherein the sequence of Formula (I) is Ac-Nle-Asp-Pro-dNal(2′)-Arg-Trp-Lys-dVal-dPro-NH2 (SEQ ID NO: 14) or Ac-Nle-Pro-dNal(2′)-Arg-Trp-dVal-dPro-NH2 (SEQ ID NO: 15).

Clause 256. The use of clause 216, wherein the sequence of Formula (I) is selected from the group consisting of: Ac-dArg-c(Asp-Pro-dNal(2′)-Arg-Trp-Lys)-dVal-dPro-NH2 (SEQ ID NO: 16); Ac-dMet-c(Asp-Pro-dNal(2′)-Arg-Trp-Lys)-dVal-dPro-NH2 (SEQ ID NO: 17); Ac-dIIe-c(Asp-Pro-dNal(2′)-Arg-Trp-Lys)-dVal-dPro-NH2 (SEQ ID NO: 18); Ac-dLeu-c(Asp-Pro-dNal(2′)-Arg-Trp-Lys)-dVal-dPro-NH2 (SEQ ID NO: 19); Ac-dVal-c(Asp-Pro-dNal(2′)-Arg-Trp-Lys)-dVal-dPro-NH2 (SEQ ID NO: 20); Ac-dAla-c(Asp-Pro-dNal(2′)-Arg-Trp-Lys)-dVal-dPro-NH2 (SEQ ID NO: 21); Ac-Ala-c(Asp-Pro-dNal(2′)-Arg-Trp-Lys)-dVal-dPro-NH2 (SEQ ID NO: 22); Ac-Tle-c(Asp-Pro-dNal(2′)-Arg-Trp-Lys)-dVal-dPro-NH2 (SEQ ID NO: 23); Ac-dTle-c(Asp-Pro-dNal(2′)-Arg-Trp-Lys)-dVal-dPro-NH2 (SEQ ID NO: 24); Ac-dNle-c(Asp-Pro-dNal(2′)-Arg-Trp-Lys)-dVal-dPro-NH2 (SEQ ID NO: 25); Ac-Nva-c(Asp-Pro-dNal(2′)-Arg-Trp-Lys)-dVal-dPro-NH2 (SEQ ID NO: 26); Ac-Gly-c(Asp-Pro-dNal(2′)-Arg-Trp-Lys)-dVal-dPro-NH2 (SEQ ID NO: 27); Ac-dPro-c(Asp-Pro-dNal(2′)-Arg-Trp-Lys)-dVal-dPro-NH2 (SEQ ID NO: 28); Ac-dCys-c(Asp-Pro-dNal(2′)-Arg-Trp-Lys)-dVal-dPro-NH2 (SEQ ID NO: 29); Ac-dPhe-c(Asp-Pro-dNal(2′)-Arg-Trp-Lys)-dVal-dPro-NH2 (SEQ ID NO: 30); Ac-dTyr-c(Asp-Pro-dNal(2′)-Arg-Trp-Lys)-dVal-dPro-NH2 (SEQ ID NO: 31); Ac-dGIn-c(Asp-Pro-dNal(2′)-Arg-Trp-Lys)-dVal-dPro-NH2 (SEQ ID NO: 32); Ac-dAsn-c(Asp-Pro-dNal(2′)-Arg-Trp-Lys)-dVal-dPro-NH2 (SEQ ID NO: 33); Ac-transPro(guan)-c(Asp-Pro-dNal(2′)-Arg-Trp-Lys)-dVal-dPro-NH2 (SEQ ID NO: 34); Ac-cisPro(guan)-c(Asp-Pro-dNal(2′)-Arg-Trp-Lys)-dVal-dPro-NH2 (SEQ ID NO: 35); dTyr-c(Asp-Pro-dNal(2′)-Arg-Trp-Lys)-dVal-dPro-NH2 (SEQ ID NO: 36); Tyr-c(Asp-Pro-dNal(2′)-Arg-Trp-Lys)-dVal-dPro-NH2 (SEQ ID NO: 37); and Dmt-c(Asp-Pro-dNal(2′)-Arg-Trp-Lys)-dVal-dPro-NH2 (SEQ ID NO: 38), wherein c represents cyclization through R² and R⁷ via a lactam bond.

Clause 257. The use of clause 216, wherein the sequence of Formula (I) is selected from the group consisting of: Ac-Nle-c(dAsp-Pro-dNal(2′)-Arg-Trp-Lys)-dVal-dPro-NH2 (SEQ ID NO: 39); Ac-Nle-c(Asp-Pro-dNal(2′)-Arg-Trp-dLys)-dVal-dPro-NH2 (SEQ ID NO: 40); Ac-Nle-c(Cys-Pro-dNal(2′)-Arg-Trp-Cys)-dVal-dPro-NH2 (SEQ ID NO: 41); Ac-Nle-c (dCys-Pro-dNal(2′)-Arg-Trp-Cys)-dVal-dPro-NH2 (SEQ ID NO: 42); Ac-Nle-c(Cys-Pro-dNal(2′)-Arg-Trp-dCys)-dVal-dPro-NH2 (SEQ ID NO: 43); Ac-Nle-c(dCys-Pro-dNal(2′)-Arg-Trp-dCys)-dVal-dPro-NH2 (SEQ ID NO: 44); Ac-Nle-c(Cys-dNal(2′)-Arg-Trp-Cys)-dVal-dPro-NH2 (SEQ ID NO: 45); Ac-Nle-c(dCys-dNal(2′)-Arg-Trp-Cys)-dVal-dPro-NH2 (SEQ ID NO: 46); Ac-Nle-c(Cys-dNal(2′)-Arg-Trp-dCys)-dVal-dPro-NH2 (SEQ ID NO: 47); Ac-Nle-c (dCys-dNal(2′)-Arg-Trp-dCys)-dVal-dPro-NH2 (SEQ ID NO: 48); Ac-Nle-c(Cys-Pro-dNal(2′)-Arg-Trp-Pen)-dVal-dPro-NH2 (SEQ ID NO: 49); Ac-Nle-c(dCys-Pro-dNal(2′)-Arg-Trp-Pen)-dVal-dPro-NH2 (SEQ ID NO: 50); Ac-Nle-c(Pen-Pro-dNal(2′)-Arg-Trp-Cys)-dVal-dPro-NH2 (SEQ ID NO: 51); Ac-Nle-c(Pen-Pro-dNal(2′)-Arg-Trp-dCys)-dVal-dPro-NH2 (SEQ ID NO: 52); Ac-Nle-c(Pen-Pro-dNal(2′)-Arg-Trp-Pen)-dVal-dPro-NH2 (SEQ ID NO: 53); Ac-Nle-c(dPen-Pro-dNal(2′)-Arg-Trp-Pen)-dVal-dPro-NH2 (SEQ ID NO: 54); Ac-Nle-c(dPen-Pro-dNal(2′)-Arg-Trp-dPen)-dVal-dPro-NH2 (SEQ ID NO: 55); Ac-Nle-c(Pen-Pro-dNal(2′)-Arg-Trp-dPen)-dVal-dPro-NH2 (SEQ ID NO: 56); Ac-Nle-c(Cys-dNal(2′)-Arg-Trp-Pen)-dVal-dPro-NH2 (SEQ ID NO: 57); Ac-Nle-c(dCys-dNal(2′)-Arg-Trp-Pen)-dVal-dPro-NH2 (SEQ ID NO: 58); Ac-Nle-c(Pen-dNal(2′)-Arg-Trp-Cys)-dVal-dPro-NH2 (SEQ ID NO: 59); Ac-Nle-c(Pen-dNal(2′)-Arg-Trp-dCys)-dVal-dPro-NH2 (SEQ ID NO: 60); Ac-Nle-c(Pen-dNal(2′)-Arg-Trp-Pen)-dVal-dPro-NH2 (SEQ ID NO: 61); Ac-Nle-c(dPen-dNal(2′)-Arg-Trp-Pen)-dVal-dPro-NH2 (SEQ ID NO: 62); Ac-Nle-c(dPen-dNal(2′)-Arg-Trp-dPen)-dVal-dPro-NH2 (SEQ ID NO: 63); Ac-Nle-c(Pen-dNal(2′)-Arg-Trp-dPen)-dVal-dPro-NH2 (SEQ ID NO: 64); Ac-Nle-c(Asp-Pro-dNal(2′)-Arg-Trp-Orn)-dVal-dPro-NH2 (SEQ ID NO: 65); Ac-Nle-c(Asp-Pro-dNal(2′)-Arg-Trp-dOrn)-dVal-dPro-NH2 (SEQ ID NO: 66); Ac-Nle-c(Glu-Pro-dNal(2′)-Arg-Trp-Orn)-dVal-dPro-NH2 (SEQ ID NO: 67); and Ac-Nle-c(Glu-Pro-dNal(2′)-Arg-Trp-dOrn)-dVal-dPro-NH2 (SEQ ID NO: 68), wherein c represents cyclization through R² and R⁶ or R⁷ via a lactam bond or a disulfide bond.

Clause 258. The use of clause 216, wherein the sequence of Formula (I) is selected from the group consisting of: Ac-Nle-c[Asp-dNal(2′)-Arg-Trp-Lys]-dVal-dPro-NH2 (SEQ ID NO: 69); Ac-Nle-c[Asp-Ala-dNal(2′)-Arg-Trp-Lys]-dVal-dPro-NH2 (SEQ ID NO: 70); Ac-Nle-c(Asp-dPro-dNal(2′)-Arg-Trp-Lys)-dVal-dPro-NH2 (SEQ ID NO: 71); Ac-Nle-c(Asp-dAla-dNal(2′)-Arg-Trp-Lys)-dVal-dPro-NH2 (SEQ ID NO: 72); Ac-Nle-c(Asp-dMet-dNal(2′)-Arg-Trp-Lys)-dVal-dPro-NH2 (SEQ ID NO: 73); Ac-Nle-c(Asp-Pro-Gly-dNal(2′)-Arg-Trp-Lys)-dVal-dPro-NH2 (SEQ ID NO: 74); Ac-Nle-c(Asp-Gly-Gly-dNal(2′)-Arg-Trp-Lys)-dVal-dPro-NH2 (SEQ ID NO: 75); Ac-Nle-c[Asp-Gly-Dnal(2′)-Arg-Trp-Lys]-dVal-dPro-NH2 (SEQ ID NO: 76); Ac-Nle-c[Asp-Leu-Dnal(2′)-Arg-Trp-Lys]-dVal-dPro-NH2 (SEQ ID NO: 77); Ac-Nle-c[Asp-Ile-Dnal(2′)-Arg-Trp-Lys]-dVal-dPro-NH2 (SEQ ID NO: 78); Ac-Nle-c[Asp-Val-Dnal(2′)-Arg-Trp-Lys]-dVal-dPro-NH2 (SEQ ID NO: 79); Ac-Nle-c[Asp-dLeu-dNal(2′)-Arg-Trp-Lys]-dVal-dPro-NH2 (SEQ ID NO: 80); Ac-Nle-c[Asp-dIIe-dNal(2′)-Arg-Trp-Lys]-dVal-dPro-NH2 (SEQ ID NO: 81); Ac-Nle-c[Asp-dVal-dNal(2′)-Arg-Trp-Lys]-dVal-dPro-NH2 (SEQ ID NO: 82); Ac-Nle-c[Asp-Trp-dNal(2′)-Arg-Trp-Lys]-dVal-dPro-NH2 (SEQ ID NO: 83); Ac-Nle-c[Asp-dTrp-dNal(2′)-Arg-Trp-Lys]-dVal-dPro-NH2 (SEQ ID NO: 84); Ac-Nle-c[Asp-transPro(guan)-dNal(2′)-Arg-Trp-Lys]-dVal-dPro-NH2 (SEQ ID NO: 85); and Ac-Nle-c[Asp-cisPro(guan)-dNal(2′)-Arg-Trp-Lys]-dVal-dPro-NH2 (SEQ ID NO: 86), wherein c represents cyclization through R² and R⁷ via a lactam bond.

Clause 259. The use of clause 216, wherein the sequence of Formula (I) is selected from the group consisting of: Ac-Nle-c(Asp-Pro-dNal(2′)-Trp-Lys)-dVal-dPro-NH2 (SEQ ID NO: 87); Ac-Nle-c(Asp-Pro-dNal(2′)-Lys-Trp-Lys)-dVal-dPro-NH2 (SEQ ID NO: 88); Ac-Nle-c(Asp-Pro-dNal(2′)-dLys-Trp-Lys)-dVal-dPro-NH2 (SEQ ID NO: 89); Ac-Nle-c(Asp-Pro-dNal(2′)-dArg-Trp-Lys)-dVal-dPro-NH2 (SEQ ID NO: 90); Ac-Nle-c(Asp-Pro-dNal(2′)-Orn-Trp-Lys)-dVal-dPro-NH2 (SEQ ID NO: 91); Ac-Nle-c(Asp-Pro-dNal(2′)-dOrn-Trp-Lys)-dVal-dPro-NH2 (SEQ ID NO: 92); Ac-Nle-c(Asp-Pro-dNal(2′)-His-Trp-Lys)-dVal-dPro-NH2 (SEQ ID NO: 93); Ac-Nle-c(Asp-Pro-dNal(2′)-Ala-Trp-Lys)-dVal-dPro-NH2 (SEQ ID NO: 94); Ac-Nle-c(Asp-Pro-dNal(2′)-Gly-Trp-Lys)-dVal-dPro-NH2 (SEQ ID NO: 95); Ac-Nle-c(Asp-Pro-dNal(2′)-Asp-Trp-Lys)-dVal-dPro-NH2 (SEQ ID NO: 96); Ac-Nle-c(Asp-Pro-dNal(2′)-Glu-Trp-Lys)-dVal-dPro-NH2 (SEQ ID NO: 97); Ac-Nle-c(Asp-Pro-dNal(2′)-dHis-Trp-Lys)-dVal-dPro-NH2 (SEQ ID NO: 98); Ac-Nle-c(Asp-Pro-dNal(2′)-dAla-Trp-Lys)-dVal-dPro-NH2 (SEQ ID NO: 99); Ac-Nle-c(Asp-Pro-dNal(2′)-dAsp-Trp-Lys)-dVal-dPro-NH2 (SEQ ID NO: 100); and Ac-Nle-c(Asp-Pro-dNal(2′)-dGlu-Trp-Lys)-dVal-dPro-NH2 (SEQ ID NO: 101), wherein c represents cyclization through R² and R⁷ via a lactam bond.

Clause 260. The use of clause 216, wherein the sequence of Formula (I) is selected from the group consisting of: Ac-Nle-c(Asp-Pro-dNal(2′)-Arg-Lys)-dVal-dPro-NH2 (SEQ ID NO: 102); Ac-Nle-c(Asp-Pro-dNal(2′)-Arg-Nal(1′)-Lys)-dVal-dPro-NH2 (SEQ ID NO: 103); Ac-Nle-c(Asp-Pro-dNal(2′)-Arg-Aia-Lys)-dVal-dPro-NH2 (SEQ ID NO: 104); Ac-Nle-c(Asp-Pro-dNal(2′)-Arg-Phe-Lys)-dVal-dPro-NH2 (SEQ ID NO: 105); Ac-Nle-c(Asp-Pro-dNal(2′)-Arg-Tyr-Lys)-dVal-dPro-NH2 (SEQ ID NO: 106); Ac-Nle-c(Asp-Pro-dNal(2′)-Arg-His-Lys)-dVal-dPro-NH2 (SEQ ID NO: 107); Ac-Nle-c(Asp-Pro-dNal(2′)-Arg-Ala-Lys)-dVal-dPro-NH2 (SEQ ID NO: 108); Ac-Nle-c(Asp-Pro-dNal(2′)-Arg-dNal(1′)-Lys)-dVal-dPro-NH2 (SEQ ID NO: 109); Ac-Nle-c(Asp-Pro-dNal(2′)-Arg-dPhe-Lys)-dVal-dPro-NH2 (SEQ ID NO: 110); Ac-Nle-c(Asp-Pro-dNal(2′)-Arg-dNal(2′)-Lys)-dVal-dPro-NH2 (SEQ ID NO: 111); Ac-Nle-c(Asp-Pro-dNal(2′)-Arg-dTyr-Lys)-dVal-dPro-NH2 (SEQ ID NO: 112); Ac-Nle-c(Asp-Pro-dNal(2′)-Arg-dHis-Lys)-dVal-dPro-NH2 (SEQ ID NO: 113); and Ac-Nle-c(Asp-Pro-dNal(2′)-Arg-dAla-Lys)-dVal-dPro-NH2 (SEQ ID NO: 114), wherein c represents cyclization through R² and R⁷ via a lactam bond.

Clause 261. The use of clause 216, wherein the sequence of Formula (I) is Ac-Nle-c(Asp-Pro-Bip-Arg-Trp-Lys)-dVal-dPro-NH2 (SEQ ID NO: 115), wherein c represents cyclization through R² and R⁷ via a lactam bond.

Clause 262. The use of clause 216, wherein the sequence of Formula (I) is selected from the group consisting of: Ac-Nle-c[Asp-Pro-dNal(2′)-Arg-Trp-Lys]-Val-Pro-NH2 (SEQ ID NO: 116); Ac-Nle-c[Asp-Pro-dNal(2′)-Arg-Trp-Lys]-dVal-dPro-OH (SEQ ID NO: 117); Ac-Nle-c[Asp-Pro-dNal(2′)-Arg-Trp-Lys]-dPro-dVal-OH (SEQ ID NO: 118); Ac-Nle-c[Asp-Pro-dNal(2′)-Arg-Trp-Lys]-dVal-Hyp-NH2 (SEQ ID NO: 119); Ac-Nle-c[Asp-Pro-dNal(2′)-Arg-Trp-Lys]-dVal-dHyp-NH2 (SEQ ID NO: 120); Ac-Nle-c[Asp-Pro-dNal(2′)-Arg-Trp-Lys]-Val-Hyp-NH2 (SEQ ID NO: 121); Ac-Nle-c[Asp-Pro-dNal(2′)-Arg-Trp-Lys]-Val-dHyp-NH2 (SEQ ID NO: 122); Ac-Nle-c[Asp-Pro-dNal(2′)-Arg-Trp-Lys]-Hyp-dVal-NH2 (SEQ ID NO: 123); Ac-Nle-c[Asp-Pro-dNal(2′)-Arg-Trp-Lys]-dHyp-dVal-NH2 (SEQ ID NO: 124); Ac-Nle-c[Asp-Pro-dNal(2′)-Arg-Trp-Lys]-Hyp-Val-NH2 (SEQ ID NO: 125); Ac-Nle-c[Asp-Pro-dNal(2′)-Arg-Trp-Lys]-dHyp-Val-NH2 (SEQ ID NO: 126); Ac-Nle-c[Asp-Pro-dNal(2′)-Arg-Trp-Lys]-dVal-dVal-NH2 (SEQ ID NO: 127); Ac-Nle-c[Asp-Pro-dNal(2′)-Arg-Trp-Lys]-dPro-dPro-NH2 (SEQ ID NO: 128); Ac-Nle-c[Asp-Pro-dNal(2′)-Arg-Trp-Lys]-dVal-NH2 (SEQ ID NO: 129); Ac-Nle-c[Asp-Pro-dNal(2′)-Arg-Trp-Lys]-dPro-NH2 (SEQ ID NO: 130); Ac-Nle-c[Asp-Pro-dNal(2′)-Arg-Trp-Lys]-Val-NH2 (SEQ ID NO: 131); Ac-Nle-c[Asp-Pro-dNal(2′)-Arg-Trp-Lys]-Pro-NH2 (SEQ ID NO: 132); Ac-Nle-c[Asp-Pro-dNal(2′)-Arg-Trp-Lys]-Ala-NH2 (SEQ ID NO: 133); Ac-Nle-c[Asp-Pro-dNal(2′)-Arg-Trp-Lys]-dAla-NH2 (SEQ ID NO: 134); Ac-Nle-c[Asp-Pro-dNal(2′)-Arg-Trp-Lys]-dHyp-NH2 (SEQ ID NO: 135); Ac-Nle-c[Asp-Pro-dNal(2′)-Arg-Trp-Lys]-Hyp-NH2 (SEQ ID NO: 136); Ac-Nle-c[Asp-Pro-dNal(2′)-Arg-Trp-Lys]-dAla-dAla-NH2 (SEQ ID NO: 137); Ac-Nle-c[Asp-Pro-dNal(2′)-Arg-Trp-Lys]-Ala-Ala-NH2 (SEQ ID NO: 138); Ac-Nle-c[Asp-Pro-dNal(2′)-Arg-Trp-Lys]-Gly-Gly-NH2 (SEQ ID NO: 139); Ac-Nle-c[Asp-Pro-dNal(2′)-Arg-Trp-Lys]-dVal-Asp-NH2 (SEQ ID NO: 140); Ac-Nle-c[Asp-Pro-dNal(2′)-Arg-Trp-Lys]-dVal-Arg-NH2 (SEQ ID NO: 141); Ac-Nle-c[Asp-Pro-dNal(2′)-Arg-Trp-Lys]-dVal-Asn-NH2 (SEQ ID NO: 142); Ac-Nle-c[Asp-Pro-dNal(2′)-Arg-Trp-Lys]-dVal-dAsp-NH2 (SEQ ID NO: 143); Ac-Nle-c[Asp-Pro-dNal(2′)-Arg-Trp-Lys]-dVal-dArg-NH2 (SEQ ID NO: 144); Ac-Nle-c[Asp-Pro-dNal(2′)-Arg-Trp-Lys]-dVal-dAsn-NH2 (SEQ ID NO: 145); Ac-Nle-c[Asp-Pro-dNal(2′)-Arg-Trp-Lys]-Asp-dPro-NH2 (SEQ ID NO: 146); Ac-Nle-c[Asp-Pro-dNal(2′)-Arg-Trp-Lys]-Arg-dPro-NH2 (SEQ ID NO: 147); Ac-Nle-c[Asp-Pro-dNal(2′)-Arg-Trp-Lys]-Asn-dPro-NH2 (SEQ ID NO: 148); Ac-Nle-c[Asp-Pro-dNal(2′)-Arg-Trp-Lys]-dAsp-dPro-NH2 (SEQ ID NO: 149); Ac-Nle-c[Asp-Pro-dNal(2′)-Arg-Trp-Lys]-dArg-dPro-NH2 (SEQ ID NO: 150); Ac-Nle-c[Asp-Pro-dNal(2′)-Arg-Trp-Lys]-dAsn-dPro-NH2 (SEQ ID NO: 151); Ac-Nle-c[Asp-Pro-dNal(2′)-Arg-Trp-Lys]-Asp-NH2 (SEQ ID NO: 152); Ac-Nle-c[Asp-Pro-dNal(2′)-Arg-Trp-Lys]-Arg-NH2 (SEQ ID NO: 153); Ac-Nle-c[Asp-Pro-dNal(2′)-Arg-Trp-Lys]-Asn-NH2 (SEQ ID NO: 154); Ac-Nle-c[Asp-Pro-dNal(2′)-Arg-Trp-Lys]-dAsp-NH2 (SEQ ID NO: 155); Ac-Nle-c[Asp-Pro-dNal(2′)-Arg-Trp-Lys]-dArg-NH2 (SEQ ID NO: 156); Ac-Nle-c[Asp-Pro-dNal(2′)-Arg-Trp-Lys]-dAsn-NH2 (SEQ ID NO: 157); Ac-Nle-c[Asp-Pro-dNal(2′)-Arg-Trp-Lys]-Lys-Pro-Val-NH2 (SEQ ID NO: 158); Ac-Nle-c[Asp-Pro-dNal(2′)-Arg-Trp-Lys]-Lys-dPro-dVal-NH2 (SEQ ID NO: 159); Ac-Nle-c[Asp-Pro-dNal(2′)-Arg-Trp-Lys]-dLys-dPro-dVal-NH2 (SEQ ID NO: 160); Ac-Nle-c[Asp-Pro-dNal(2′)-Arg-Trp-Lys]-Lys-dPro-NH2 (SEQ ID NO: 161); Ac-Nle-c[Asp-Pro-dNal(2′)-Arg-Trp-Lys]-dLys-dPro-NH2 (SEQ ID NO: 162); Ac-Nle-c[Asp-Pro-dNal(2′)-Arg-Trp-Lys]-Lys-Val-Pro-NH2 (SEQ ID NO: 163); Ac-Nle-c[Asp-Pro-dNal(2′)-Arg-Trp-Lys]-Lys-dVal-dPro-NH2 (SEQ ID NO: 164); Ac-Nle-c[Asp-Pro-dNal(2′)-Arg-Trp-Lys]-dLys-dVal-dPro-NH2 (SEQ ID NO: 165); Ac-Nle-c[Asp-Pro-dNal(2′)-Arg-Trp-Lys]-Arg-Pro-Val-NH2 (SEQ ID NO: 166); Ac-Nle-c[Asp-Pro-dNal(2′)-Arg-Trp-Lys]-Arg-dPro-dVal-NH2 (SEQ ID NO: 167); Ac-Nle-c[Asp-Pro-dNal(2′)-Arg-Trp-Lys]-dArg-dPro-dVal-NH2 (SEQ ID NO: 168); Ac-Nle-c[Asp-Pro-dNal(2′)-Arg-Trp-Lys]-Arg-Val-Pro-NH2 (SEQ ID NO: 169); Ac-Nle-c[Asp-Pro-dNal(2′)-Arg-Trp-Lys]-Arg-dVal-dPro-NH2 (SEQ ID NO: 170); Ac-Nle-c[Asp-Pro-dNal(2′)-Arg-Trp-Lys]-dArg-dVal-dPro-NH2 (SEQ ID NO: 171); Ac-Nle-c[Asp-Pro-dNal(2′)-Arg-Trp-Lys]-dTle-dVal-dPro-NH2 (SEQ ID NO: 172); Ac-Nle-c[Asp-Pro-dNal(2′)-Arg-Trp-Lys]-dTle-dPro-NH2 (SEQ ID NO: 173); Ac-Nle-c[Asp-Pro-dNal(2′)-Arg-Trp-Lys]-dTle-dVal-NH2 (SEQ ID NO: 174); Ac-Nle-c[Asp-Pro-dNal(2′)-Arg-Trp-Lys]-dTle-dTle-NH2 (SEQ ID NO: 175); Ac-Nle-c[Asp-Pro-dNal(2′)-Arg-Trp-Lys]-dTle-dTle-dPro-NH2 (SEQ ID NO: 176); Ac-Nle-c[Asp-Pro-dNal(2′)-Arg-Trp-Lys]-dTle-dTle-dVal-NH2 (SEQ ID NO: 177); Ac-Nle-c[Asp-Pro-dNal(2′)-Arg-Trp-Lys]-dPro-dTle-NH2 (SEQ ID NO: 178); and Ac-Nle-c[Asp-Pro-dNal(2′)-Arg-Trp-Lys]-dVal-dTle-NH2 (SEQ ID NO: 179), wherein c represents cyclization through R² and R⁷ via a lactam bond.

Clause 263. The use of clause 216, wherein the sequence of Formula (I) is selected from the group consisting of: Ac-Nle-c(Asp-Pro-dNal(2′)-Arg-Trp-Lys)-dVal-dVal-dPro-NH2 (SEQ ID NO: 180); Ac-Nle-c(Asp-Pro-dNal(2′)-Arg-Trp-Lys)-dPro-dVal-dPro-NH2 (SEQ ID NO: 181); Ac-Nle-c(Asp-Pro-dNal(2′)-Arg-Trp-Lys)-dVal-dVal-dVal-dPro-NH2 (SEQ ID NO: 182); Ac-Nle-c(Asp-Pro-dNal(2′)-Arg-Trp-Lys)-dVal-dPro-dVal-dPro-NH2 (SEQ ID NO: 183); and Ac-Nle-c(Asp-Pro-dNal(2′)-Arg-Trp-Lys)-dVal-dVal-dVal-dVal-dVal-dVal-dPro-NH2 (SEQ ID NO: 184), wherein c represents cyclization through R² and R⁷ via a lactam bond.

Clause 264. The use of clause 216, wherein X¹ is present and is acetylated norleucine, and R¹ is present and is norleucine.

Clause 265. The use of clause 264, wherein X² is present and is norleucine.

Clause 266. The use of clause 264, wherein X³ is present and is norleucine.

Clause 267. The use of clause 264, wherein the sequence of Formula (I) is selected from the group consisting of: Ac-Nle-Nle-c(Asp-Pro-dNal(2′)-Arg-Trp-Lys)-dVal-dPro-NH2 (SEQ ID NO: 185); Ac-Nle-Nle-Nle-c(Asp-Pro-dNal(2′)-Arg-Trp-Lys)-dVal-dPro-NH2 (SEQ ID NO: 186); and Ac-Nle-Nle-Nle-Nle-c(Asp-Pro-dNal(2′)-Arg-Trp-Lys)-dVal-dPro-NH2 (SEQ ID NO: 187), wherein c represents cyclization through R² and R⁷ via a lactam bond.

Clause 268. The use of clause 216, wherein the sequence of Formula (I) is selected from the group consisting of: Ac-Nle-c[Asp-Pro-Pro-dNal(2′)-Arg-Trp-Lys]-dVal-dPro-NH2 (SEQ ID NO: 195); Ac-Nle-c[Asp-Trp-Pro-dNal(2′)-Arg-Trp-Lys]-dVal-dPro-NH2 (SEQ ID NO: 196); Ac-Nle-c[Asp-Pro-dNal(2′)-Arg-dTrp-Lys]-dVal-dPro-NH2 (SEQ ID NO: 197); c[CO-cis-CH═CH—CO-Pro-D-Nal(2′)-Arg-Trp-Lys]-dVal-dPro-NH2 (SEQ ID NO: 198); Ac-Nle-c[Asp-Aba-D-Phe-Arg-Trp-Lys]-dVal-dPro-NH2 (SEQ ID NO: 199); Ac-Nle-c[Asp-β-Ala-D-Nal(2′)-Arg-Trp-Lys]-dVal-dPro-NH2 (SEQ ID NO: 200); Ac-Nle-c[Asp-Mamb-D-Nal(2′)-Arg-Trp-Lys]-dVal-dPro-NH2 (SEQ ID NO: 201); Ac-Nle-c[Asp-Acpc-D-Nal(2′)-Arg-Trp-Lys]-dVal-dPro-NH2 (SEQ ID NO: 202); Ac-c[Cys-Arg-D-Phe-Cys]-Trp-dVal-dPro-NH2 (SEQ ID NO: 203); Ac-Nle-c[Asp-Pro-dNal(2′)-Arg-Trp-Lys]-Trp-NH2 (SEQ ID NO: 204); Ac-Nle-c(Asp-Aba-dNal(2′)-Arg-Trp-Lys)-dVal-dPro-NH2 (SEQ ID NO: 205); Ac-Nle-c(Asp-Aia-dNal(2′)-Arg-Trp-Lys)-dVal-dPro-NH2 (SEQ ID NO: 206); Ac-Nle-c(Asp-Ata-dNal(2′)-Arg-Trp-Lys)-dVal-dPro-NH2 (SEQ ID NO: 207); Ac-Nle-c(Asp-Aia-dPhe-Arg-Trp-Lys)-dVal-dPro-NH2 (SEQ ID NO: 208); Ac-Nle-c(Asp-Ata-dPhe-Arg-Trp-Lys)-dVal-dPro-NH2 (SEQ ID NO: 209); Ac-Nle-c(Asp-Aba-p(CI)dPhe-Arg-Trp-Lys)-dVal-dPro-NH2 (SEQ ID NO: 210); Ac-Nle-c(Asp-Aia-p(CI)dPhe-Arg-Trp-Lys)-dVal-dPro-NH2 (SEQ ID NO: 211); and Ac-Nle-c(Asp-Ata-p(CI)dPhe-Arg-Trp-Lys)-dVal-dPro-NH2 (SEQ ID NO: 212), wherein c represents cyclization through R² and R⁷ or R⁸ via a lactam bond or through R² and R⁵ via a disulfide bond.

Clause 269. The use of clause 216, wherein the sequence of Formula (I) is selected from the group consisting of: Ac-Nle-c[Asp-Aic-dNal(2′)-Arg-Trp-Lys]-dVal-dPro-NH2 (SEQ ID NO: 240); Ac-Nle-c[Asp-Cpe-dNal(2′)-Arg-Trp-Lys]-dVal-dPro-NH2 (SEQ ID NO: 241); Ac-Nle-c[Asp-Che-dNal(2′)-Arg-Trp-Lys]-dVal-dPro-NH2 (SEQ ID NO: 242); Ac-Nle-c[Asp-Oic-dNal(2′)-Arg-Trp-Lys]-dVal-dPro-NH2 (SEQ ID NO: 243); Ac-Nle-c[Asp-loc-dNal(2′)-Arg-Trp-Lys]-dVal-dPro-NH2 (SEQ ID NO: 244); Ac-Nle-c[Asp-Tic-dNal(2′)-Arg-Trp-Lys]-dVal-dPro-NH2 (SEQ ID NO: 245); Ac-Nle-c[Asp-Pro-dNal(2′)-Arg-Trp-Pro-Lys]-dVal-dPro-NH2 (SEQ ID NO: 246); Ac-Nle-c[Asp-His-dNal(2′)-Pro-Trp-Lys]-dVal-dPro-NH2 (SEQ ID NO: 247); Ac-Nle-c[Asp-His-dNal(2′)-transPro(guan)-Trp-Lys]-dVal-dPro-NH2 (SEQ ID NO: 248); Ac-Nle-c[Asp-His-dNal(2′)-cisPro(guan)-Trp-Lys]-dVal-dPro-NH2 (SEQ ID NO: 249); Ac-Nle-c[Asp-Pro-dNal(2′)-Pro-Trp-Lys]-dVal-dPro-NH2 (SEQ ID NO: 250); Ac-Nle-c[Asp-Pro-dNal(2′)-transPro(guan)-Trp-Lys]-dVal-dPro-NH2 (SEQ ID NO: 251); Ac-Nle-c[Asp-Pro-dNal(2′)-cisPro(guan)-Trp-Lys]-dVal-dPro-NH2 (SEQ ID NO: 252); Ac-Nle-c(Asp-Pro-dNal(2′)-Arg-Aia-Lys)-dVal-dPro-NH2 (SEQ ID NO: 253); Ac-Nle-c(Asp-Pro-dNal(2′)-Arg-Aba-Lys)-dVal-dPro-NH2 (SEQ ID NO: 254); Ac-Nle-c(Asp-Pro-dNal(2′)-Arg-Ata-Lys)-dVal-dPro-NH2 (SEQ ID NO: 255); Ac-Nle-c[Asp-Glu-dNal(2′)-Arg-Trp-Lys]-dVal-dPro-NH2 (SEQ ID NO: 256); Ac-Nle-c[Asp-Glu-dNal(2′)-Arg-Trp-Gly-Lys]-dVal-dPro-NH2 (SEQ ID NO: 257); Ac-Nle-c[Asp-Pro-Glu-dNal(2′)-Arg-Trp-Lys]-dVal-dPro-NH2 (SEQ ID NO: 258); Ac-Nle-c[Asp-Pro-Glu-dNal(2′)-Arg-Trp-Gly-Lys]-dVal-dPro-NH2 (SEQ ID NO: 259); Ac-Nle-c[Asp-Pro-dNal(2′)-Arg-Trp-Gly-Lys]-dVal-dPro-NH2 (SEQ ID NO: 260); Ac-Nle-c[Asp-Pro-dNal(2′)-Arg-Trp-Gly-Lys]-dPro-dPro-Lys-Asp-NH2 (SEQ ID NO: 261); Ac-Nle-c[Asp-Pro-dNal(2′)-Arg-Trp-Gly-Lys]-dPro-dPro-dLys-dAsp-NH2 (SEQ ID NO: 262); Ac-Glu-c[Asp-Pro-dNal(2′)-Arg-Trp-Gly-Lys]-dPro-dPro-Lys-Asp-NH2 (SEQ ID NO: 263); and Ac-Nle-c[Asp-Pro-dNal(2′)-Arg-Trp-Lys]-dPro-dPro-dLys-dAsp-NH2 (SEQ ID NO: 264), wherein c represents cyclization through R² and R⁷ via a lactam bond.

Clause 270. The use of clause 216, wherein the sequence of Formula (I) is selected from the group consisting of: Ac-Nle-c[Asp-Glu-His-dNal(2′)-Arg-Trp-Lys]-dVal-dPro-NH2 (SEQ ID NO: 265); Ac-Nle-c[Asp-Glu-His-dNal(2′)-Arg-Trp-Gly-Lys]-dVal-dPro-NH2 (SEQ ID NO: 266); Ac-Nle-c(Asp-dAla-His-dNal(2′)-Arg-Trp-Lys)-dVal-dPro-NH2 (SEQ ID NO: 267); Ac-Arg-c(Asp-dAla-His-dNal(2′)-Arg-Trp-Lys)-dVal-dPro-NH2 (SEQ ID NO: 268); Ac-Arg-c(Asp-dAla-His-dPhe-Arg-Trp-Lys)-dVal-dPro-NH2 (SEQ ID NO: 269); Ac-Arg-c (Cys-dAla-His-dNal(2′)-Arg-Trp-Cys)-dVal-dPro-NH2 (SEQ ID NO: 270); Ac-dArg-c(Asp-dAla-His-dNal(2′)-Arg-Trp-Lys)-dVal-dPro-NH2 (SEQ ID NO: 271); Ac-Arg-c(Asp-dAla-dNal(2′)-Arg-Trp-Lys)-dVal-dPro-NH2 (SEQ ID NO: 272); Ac-dArg-c(Asp-dAla-dNal(2′)-Arg-Trp-Lys)-dVal-dPro-NH2 (SEQ ID NO: 273); Ac-Nle-c(Asp-Ala-His-dNal(2′)-Arg-Trp-Lys)-dVal-dPro-NH2 (SEQ ID NO: 274); Ac-Arg-c(Asp-Ala-His-dNal(2′)-Arg-Trp-Lys)-dVal-dPro-NH2 (SEQ ID NO: 275); Ac-dArg-c(Asp-Ala-His-dNal(2′)-Arg-Trp-Lys)-dVal-dPro-NH2 (SEQ ID NO: 276); Ac-Nle-c(Asp-Trp-Arg-dNal(2′)-Pro-Lys)-dVal-dPro-NH2 (SEQ ID NO: 277); Ac-Nle-c(Asp-Pro-Trp-dNal(2′)-Lys)-dVal-dPro-NH2 (SEQ ID NO: 278); Ac-Nle-c(Asp-Pro-Trp-Arg-dNal(2′)-Pro-Lys)-dVal-dPro-NH2 (SEQ ID NO: 279); Ac-Nle-c(Asp-Pro-Trp-Arg-dNal(2′)-Lys)-dVal-dPro-NH2 (SEQ ID NO: 280); Ac-Nle-c(Lys-Trp-Arg-dNal(2′)-Pro-Asp)-dVal-dPro-NH2 (SEQ ID NO: 281) Ac-Arg-c(Asp-dAla-His-dPhe-Arg-Trp-Lys)-NH2 (SEQ ID NO: 282); Ac-Arg-c (Cys-dAla-His-dNal(2′)-Arg-Trp-Cys)-NH2 (SEQ ID NO: 283); and Ac-Arg-c(Asp-dAla-His-dNal(2′)-Arg-Trp-Lys)-NH2 (SEQ ID NO: 284), wherein c represents cyclization through R² and any of R⁵—R⁸ via a lactam bond or a disulfide bond.

Clause 271. The use of clause 216, wherein the sequence of Formula (I) is: Ac-dVal-dPro-c(Asp-Trp-Arg-dNal(2′)-Pro-Lys)-Nle-NH2 (SEQ ID NO: 285); Ac-dVal-dPro-c (Lys-Trp-Arg-dNal(2′)-Pro-Asp)-Nle-NH2 (SEQ ID NO: 286); Ac-dVal-dPro-c(Asp-Trp-Arg-dNal(2′)-Pro-Lys)-Nle-Nle-NH2 (SEQ ID NO: 287); Ac-dVal-dPro-c (Lys-Trp-Arg-dNal(2′)-Pro-Asp)-Nle-Nle-NH2 (SEQ ID NO: 288); Ac-dVal-dPro-c(Asp-Trp-Arg-dNal(2′)-Pro-Lys)-dVal-dPro-NH2 (SEQ ID NO: 289); Ac-dVal-dPro-c (Lys-Trp-Arg-dNal(2′)-Pro-Asp)-dVal-dPro-NH2 (SEQ ID NO: 290), wherein c represents cyclization through R¹ and any one of R⁶ via a lactam bond.

Clause 272. The use of clause 216, wherein the non-naturally occurring melanocortin analog comprises any one of the sequences of SEQ ID NOs: 2-290.

Clause 273. The use of clause 216, wherein the non-naturally occurring melanocortin analog comprises any one of the sequences of SEQ ID NOs: 2-187, 196-212, and 240-290.

Clause 274. The use of clause 216, wherein the non-naturally occurring melanocortin analog comprises a sequence of SEQ ID NO: 3.

Clause 275. The use of any one of clauses 216-274, wherein the non-naturally occurring melanocortin analog is present in a composition.

Clause 276. The use of clause 275, wherein the composition further comprises a pharmaceutical salt.

Clause 277. The use of clause 275, wherein the composition further comprises a pharmaceutical carrier.

Clause 278. The use of clause 277, wherein the non-naturally occurring melanocortin analog is present in the composition in a concentration of 0.1 mg/ml to 500 mg/mL, relative to a total volume of the composition.

Clause 279. The use of clause 278, wherein the non-naturally occurring melanocortin analog comprises a sequence of SEQ ID NO: 3, and wherein the non-naturally occurring melanocortin analog is present in the composition in a concentration of 5 mg/ml to 100 mg/mL, relative to a total volume of the composition.

Clause 280. The use of clause 279, wherein the non-naturally occurring melanocortin analog is present in the composition in a concentration of about 50 mg/mL, relative to a total volume of the composition.

Clause 281. The use of any one of clauses 216-280, wherein the non-naturally occurring melanocortin analog is administered via intraperitoneal, intravenous, parenteral, subcutaneous, intramuscular, intracerebroventricular, intranasal, or oral administration.

Clause 282. The use of any one of clauses 275-281, wherein the composition comprising the non-naturally occurring melanocortin analog is administered to the subject parenterally.

Clause 283. The use of any one of clauses 275-282, wherein the composition comprising the non-naturally occurring melanocortin analog is administered to the subject subcutaneously.

Clause 284. The use of any one of clauses 216-283, wherein the non-naturally occurring melanocortin analog crosses the blood-brain-barrier of the subject.

Clause 285. The use of any one of clauses 216-284, wherein the therapeutically effective amount of the non-naturally occurring melanocortin analog is from 0.001 mg/kg to 25 mg/kg per body weight of the subject.

Clause 286. The use of any one of clauses 216-285, wherein the therapeutically effective amount of the non-naturally occurring melanocortin analog is from 0.5 mg/kg to 10 mg/kg per body weight of the subject.

Clause 287. The use of any one of clauses 216-286, wherein the non-naturally occurring melanocortin analog is administered at least once daily in an amount ranging from 0.001 mg/kg to 25 mg/kg per body weight of the subject.

Clause 288. The use of any one of clauses 216-287, wherein the non-naturally occurring melanocortin analog is administered at least once daily in an amount ranging from about 0.5 mg/kg to about 10 mg/kg per body weight of the subject.

Clause 289. The use of clause 287, wherein the non-naturally occurring melanocortin analog comprises a sequence of SEQ ID NO: 3, and the non-naturally occurring melanocortin analog is administered at least once daily in an amount ranging from 0.001 mg/kg to 25 mg/kg per body weight of the subject.

Clause 290. The use of clause 288, wherein the non-naturally occurring melanocortin analog comprises a sequence of SEQ ID NO: 3, and the non-naturally occurring melanocortin analog is administered at least once daily in an amount ranging from 0.5 mg/kg to 10 mg/kg per body weight of the subject.

Clause 291. The use of any one of clauses 216-290, wherein the non-naturally occurring melanocortin analog is administered to the subject for at least 1 day, 1 week, 1 month, 3 months, 6 months, 1 year, or 5 years.

Clause 292. The use of any one of clauses 216-290, wherein the non-naturally occurring melanocortin analog is administered to the subject for 1 day, 5 days, 7 days, 14 days, 21 days, 28 days, 35 days, 40 days, 45 days, 50 days, 60 days, 75 days, 90 days, 100 days, 110 days, or 120 days.

Clause 293. The use of clause 277, wherein the pharmaceutical carrier comprises water.

Clause 294. The use of any one of clauses 216-293, wherein the subject experiences loss of appetite, reduced appetite, decreased food consumption, and/or weight loss prior to the administration.

Clause 295. The use of clause 294, wherein the loss of appetite, reduced appetite, decreased food consumption, and/or weight loss is caused by cachexia.

Clause 296. The use of any one of clauses 216-295, wherein the use (i) stimulates appetite of the subject; (ii) increases food consumption by the subject; (iii) prevents or alleviates nausea, emesis, and/or anorexia in the subject; (iv) increases or maintains body weight or BMI of the subject; (v) prevents or reduces weight loss of the subject; (vi) increases or maintains muscle mass of the subject; (vii) prevents or reduces muscle mass loss of the subject; (viii) increases or maintains fat mass of the subject; and/or (ix) prevents or reduces fat mass loss of the subject.

Clause 297. The use of any one of clauses 216-296, wherein the use increases body weight or BMI of and/or increases food consumption by the subject.

Clause 298. The use of clause 296, wherein appetite of the subject is increased by at least 10% to 200% after the administration.

Clause 299. The use of clause 296, wherein appetite of the subject is increased by at least 25% to 100% after the administration.

Clause 300. The use of clause 298 or 299, wherein appetite is increased for at least 1 day, 1 week, 1 month, 3 months, 6 months, 1 year, or 5 years during the administration.

Clause 301. The use of clause 298 or 299, wherein appetite is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration.

Clause 302. The use of any one of clauses 298-301, wherein the increased appetite is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

Clause 303. The use of any one of clauses 296 and 298-302, wherein the appetite is assessed by a scale for measuring desire to eat, feeling of hunger, and/or satiety.

Clause 304. The use of clause 303, wherein the appetite is assessed between meals.

Clause 305. The use of any one of clauses 296 and 298-304, wherein the subject comprises two or more subjects, and wherein the appetite is an average appetite of the two or more subjects.

Clause 306. The use of clause 296 or 297, wherein food consumption of the subject is increased by at least 25% to 2,000% after the administration.

Clause 307. The use of clause 296 or 297, wherein food consumption of the subject is increased by at least 50% to 500% after the administration.

Clause 308. The use of clause 306 or 307, wherein food consumption is increased for at least 1 day, 1 week, 1 month, 3 months, 6 months, 1 year, or 5 years during the administration.

Clause 309. The use of clause 306 or 307, wherein food consumption is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration.

Clause 310. The use of any one of clauses 91-94, wherein the increased food consumption is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

Clause 311. The use of any one of clauses 296-297 and 306-310, wherein the food consumption is determined by total calories (kcal) consumed in 1 day or mass of food (grams or kilograms) consumed in 1 day.

Clause 312. The use of clause 296, wherein anorexia is determined by food consumption.

Clause 313. The use of clause 296 or 297, wherein body weight of the subject is increased by at least 5% to 200% after the administration.

Clause 314. The use of clause 296 or 297, wherein body weight of the subject is increased by at least 5% to 25% after the administration.

Clause 315. The use of clause 296 or 297, wherein body weight of the subject is increased by at least 20% to 100% after the administration.

Clause 316. The use of any one of clauses 313-315, wherein the increased body weight is maintained for at least 1 week, 2 weeks, 3 weeks, 1 month, 3 months, 6 months, 1 year, or 5 years, after the administration is stopped.

Clause 317. The use of clause 296, wherein muscle mass of the subject is increased by at least 1% to 100% after the administration.

Clause 318. The use of clause 296, wherein muscle mass of the subject is increased by at least 5% to 15% after the administration.

Clause 319. The use of clause 296, wherein muscle mass of the subject is increased by at least 25% to 50% after the administration.

Clause 320. The use of any one of clauses 102-104, wherein the increased muscle mass is maintained for at least 1 week, 2 weeks, 3 weeks, 1 month, 3 months, 6 months, 1 year, or 5 years, after the administration is stopped.

Clause 321. The use of any one of clauses 296 and 317-320, wherein the muscle mass is cardiac muscle mass, skeletal muscle mass, or both.

Clause 322. The use of clause 296, wherein fat mass of the subject is increased by at least 1% to 100% after the administration.

Clause 323. The use of clause 296, wherein fat mass of the subject is increased by at least 5% to 15% after the administration.

Clause 324. The use of clause 296, wherein fat mass of the subject is increased by at least 25% to 50% after the administration.

Clause 325. The use of any one of clauses 322-324, wherein the increased fat mass is maintained for at least 1 week, 2 weeks, 3 weeks, 1 month, 3 months, 6 months, 1 year, or 5 years, after the administration is stopped.

Clause 326. The use of any one of clauses 216-295, wherein the use stimulates appetite in the subject compared to a control subject, wherein the control subject is not administered the non-naturally occurring melanocortin analog.

Clause 327. The use of clause 326, wherein the control subject experiences loss of appetite, reduced appetite, decreased food consumption, and/or weight loss.

Clause 328. The use of clause 327, wherein the loss of appetite, reduced appetite, decreased food consumption, and/or weight loss is caused by cachexia.

Clause 329. The use of any one of clauses 326-328, wherein appetite of the subject is increased after the administration by at least 10% to 200%, relative to appetite of the control subject.

Clause 330. The use of any one of clauses 326-328, wherein appetite of the subject is increased after the administration by at least 25% to 100%, relative to appetite of the control subject.

Clause 331. The use of clause 329 or 330, wherein appetite is increased for at least 1 day, 1 week, 1 month, 3 months, 6 months, 1 year, or 5 years during the administration.

Clause 332. The use of clause 274 or 275, wherein appetite is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration.

Clause 333. The use of any one of clauses 329-332, wherein the increased appetite is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

Clause 334. The use of any one of clauses 329-333, wherein the appetite is assessed by a scale for measuring desire to eat, feeling of hunger, and/or level of satiety.

Clause 335. The use of clause 329, wherein the appetite is assessed between meals.

Clause 336. The use of any one of clauses 326-335, wherein the subject comprises two or more subjects, and wherein the appetite of the subject is an average appetite of the two or more subjects.

Clause 337. The use of any one of clauses 326-336, wherein the control subject comprises two or more control subjects, and wherein the appetite of the control subject is an average appetite of the two or more control subjects.

Clause 338. The use of any one of clauses 326-337, wherein food consumption of the subject is increased after the administration by at least 25% to 2,000%, relative to food consumption of the control subject.

Clause 339. The use of any one of clauses 326-328, wherein food consumption of the subject is increased after the administration by at least 50% to 500%, relative to food consumption of the control subject.

Clause 340. The use of clause 329 or 330, wherein food consumption is increased for at least 1 day, 1 week, 1 month, 3 months, 6 months, 1 year, or 5 years during the administration.

Clause 341. The use of clause 338 or 339, wherein food consumption is increased for at least 1 day, 5 days, 7 days, 14 days, 21 days, 28 days, 35 days, 40 days, 45 days, 50 days, 60 days, 75 days, 90 days, 100 days, 110 days, or 120 days during the administration.

Clause 342. The use of any one of clauses 338-341, wherein the increased food consumption is maintained for at least 1 day, 2 days, 3 days, 4 days, 5 days, 6 days, 1 week, 2 weeks, 3 weeks, 1 month, 3 months, 6 months, 1 year, or 5 years, after the administration is stopped.

Clause 343. The use of any one of clauses 338-342, wherein the food consumption is determined by total calories (kcal) consumed in 1 day or mass of food (grams or kilograms) consumed in 1 day.

Clause 344. The use of any one of clauses 326-328, wherein body weight of the subject is increased after the administration, and wherein weight gain of the subject after the administration is at least 5% to 200% greater than that of the control subject.

Clause 345. The use of any one of clauses 326-328, wherein body weight of the subject is increased after the administration, and wherein weight gain of the subject after the administration is at least 5% to 25% greater than that of the control subject.

Clause 346. The use of any one of clauses 326-328, wherein body weight of the subject is increased after the administration, and wherein weight gain of the subject after the administration is at least 20% to 100% greater than that of the control subject.

Clause 347. The use of any one of clauses 344-346, wherein the increased body weight is maintained for at least 1 week, 2 weeks, 3 weeks, 1 month, 3 months, 6 months, 1 year, or 5 years, after the administration is stopped.

Clause 348. The use of any one of clauses 326-328, wherein muscle mass of the subject is increased after the administration, and wherein muscle mass gain of the subject after the administration is at least 1% to 100% greater than that of the control subject.

Clause 349. The use of any one of clauses 326-328, wherein muscle mass of the subject is increased after the administration, and wherein muscle mass gain of the subject after the administration is at least 5% to 15% greater than that of the control subject.

Clause 350. The use of any one of clauses 326-328, wherein muscle mass of the subject is increased after the administration, and wherein muscle mass gain of the subject after the administration is at least 25% to 50% greater than that of the control subject.

Clause 351. The use of any one of clauses 248-350, wherein the increased muscle mass is maintained for at least 1 week, 2 weeks, 3 weeks, 1 month, 3 months, 6 months, 1 year, or 5 years, after the administration is stopped.

Clause 352. The use of any one of clauses 326-328, wherein fat mass of the subject is increased after the administration, and wherein fat mass gain of the subject after the administration is at least 1% to 100% greater than that of the control subject.

Clause 353. The use of any one of clauses 326-328, wherein fat mass of the subject is increased after the administration, and wherein fat mass gain of the subject after the administration is at least 5% to 15% greater than that of the control subject.

Clause 354. The use of any one of clauses 326-328, wherein fat mass of the subject is increased after the administration, and wherein fat mass gain of the subject after the administration is at least 25% to 50% greater than that of the control subject.

Clause 355. The use of any one of clauses 352-354, wherein the increased fat mass is maintained for at least 1 week, 2 weeks, 3 weeks, 1 month, 3 months, 6 months, 1 year, or 5 years, after the administration is stopped.

Clause 356. The use of any one of clauses 316-355, wherein the subject is a human.

Clause 357. The use of any one of clauses 316-355, wherein the subject is an animal.

Clause 358. The use of clause 356, wherein the subject has a body mass index (BMI) of 18.5 kg/m² to 25 kg/m².

Clause 359. The use of clause 356, wherein the subject has a BMI of less than

20 kg/m².

Clause 360. The use of clause 356, wherein the subject has a BMI of less than 18.5 kg/m².

Clause 361. The use of any one of clauses 216-295, wherein the subject is a metabolically unchallenged subject, and wherein the use increases body weight of and/or increases food consumption by the metabolically unchallenged subject compared to a control subject.

Clause 362. The use of clause 361, wherein the metabolically unchallenged subject and the control subject are the same subject.

Clause 363. The use of clause 361, wherein the metabolically unchallenged subject and the control subject are different subjects.

Clause 364. The use of any one of clauses 361-363, wherein the metabolically unchallenged subject experiences loss of appetite, reduced appetite, a loss of food consumption, and/or weight loss prior to the administration.

Clause 365. The use of any one of clauses 361-363, wherein the non-naturally occurring melanocortin analog is administered to the at least once daily in an amount ranging from 0.001 mg/kg to 25 mg/kg per body weight of the metabolically unchallenged subject.

Clause 366. The use of any one of clauses 361-365, wherein the non-naturally occurring melanocortin analog is administered at least once daily in an amount ranging from about 0.5 mg/kg to about 10 mg/kg per body weight of the metabolically unchallenged subject.

Clause 367. The use of any one of clauses 361-366, wherein the non-naturally occurring melanocortin analog is administered to the metabolically unchallenged subject for at least 1 day, 1 week, 1 month, 3 months, 6 months, 1 year, or 5 years.

Clause 368. The use of any one of clauses 361-366, wherein the non-naturally occurring melanocortin analog is administered to the metabolically unchallenged subject for 1 day, 7 days, 14 days, 21 days, 28 days, 35 days, 40 days, 45 days, 50 days, 60 days, 75 days, 90 days, 100 days, 110 days, or 120 days.

Clause 369. The use of any one of clauses 361-368, wherein body weight of the metabolically unchallenged subject is increased after the administration by at least 5% to 200%, relative to body weight of the control subject before the administration, wherein the control subject is a metabolically unchallenged subject.

Clause 370. The use of any one of clauses 361-368, wherein body weight of the metabolically unchallenged subject is increased after the administration by at least about 5% to about 25%, relative to body weight of the control subject before the administration, wherein the control subject is a metabolically unchallenged subject.

Clause 371. The use of any one of clauses 361-368, wherein body weight of the metabolically unchallenged subjects increased after the administration by at least about 20% to about 100%, relative to body weight of the control subject before the administration, wherein the control subject is a metabolically unchallenged subject.

Clause 372. The use of any one of clauses 361-368, wherein body weight of the metabolically unchallenged subject is increased after the administration by at least 5% to 200%, relative to body weight of the control subject, wherein the control subject is a metabolically unchallenged subject who experiences loss of appetite, reduced appetite, and/or weight loss.

Clause 373. The use of any one of clauses 361-368, wherein body weight of the subject is increased after the administration by at least about 5% to about 25%, relative to body weight of the control subject, wherein the control subject is a metabolically unchallenged subject who experiences loss of appetite, reduced appetite, and/or weight loss.

Clause 374. The use of any one of clauses 361-368, wherein body weight of the subject is increased after the administration by at least about 20% to about 100%, relative to body weight of the control subject, wherein the control subject is a metabolically unchallenged subject who experiences loss of appetite, reduced appetite, and/or weight loss.

Clause 375. The use of any one of clauses 369-374, wherein the increased body weight is maintained for at least 1 week, 2 weeks, 3 weeks, 1 month, 3 months, 6 months, 1 year, or 5 years, after the administration is stopped.

Clause 376. The use of any one of clauses 361-375, wherein food consumption of the metabolically unchallenged subject is increased after the administration by at least 50% to 2,000%, relative to food consumption of the metabolically unchallenged subject before the administration.

Clause 377. The use of any one of clauses 361-375, wherein food consumption of the metabolically unchallenged subject is increased after the administration by at least about 100%, relative to food consumption of the metabolically unchallenged subject before the administration.

Clause 378. The use of any one of clauses 361-375, wherein food consumption of the metabolically unchallenged subject is increased after the administration by at least about 1,000%, relative to food consumption of the metabolically unchallenged subject before the administration.

Clause 379. The use of any one of clauses 361-375, wherein food consumption of the metabolically unchallenged subject is increased after the administration by at least 50% to 2,000%, relative to food consumption of the control subject, wherein the control subject is a metabolically unchallenged subject who experiences loss of appetite, reduced appetite, and/or weight loss.

Clause 380. The use of any one of clauses 361-379, wherein food consumption is increased for at least 1 day, 1 week, 1 month, 3 months, 6 months, 1 year, or 5 years during the administration.

Clause 381. The use of any one of clauses 361-379, wherein food consumption is increased for at least 1 day, 7 days, 14 days, 21 days, 28 days, 35 days, 40 days, 45 days, 50 days, 60 days, 75 days, 90 days, 100 days, 110 days, or 120 days during the administration.

Clause 382. The use of any one of clauses 376-381, wherein the increased food consumption is maintained for at least 1 week, 2 weeks, 3 weeks, 1 month, 3 months, 6 months, 1 year, or 5 years, after the administration is stopped.

Clause 383. The use of any one of clauses 361-382, wherein food consumption is determined by total calories (kcal) consumed in 1 day or mass of food (grams or kilograms) consumed in 1 day.

Clause 384. The use of any one of clauses 361-382, wherein one or more side effects exhibited by the metabolically unchallenged subject after administration of the non-naturally occurring melanocortin analog are reduced compared to one or more side effects exhibited by a control subject that was administered a natural melanocortin.

Clause 385. The use of any one of clauses 361-384, the control subject is a metabolically unchallenged subject.

Clause 386. The use of any one of clauses 216-301, wherein the metabolically unchallenged subject has a disease or condition selected from the group consisting of a psychological disease or condition, an allergy, an intolerance, a gastrointestinal disease or condition, a side-effect from a medication, substance use, a viral infection, a bacterial infection, food poisoning, dehydration, fatigue, hormonal imbalance, pain, a cardiovascular disease or condition, anemia, an autoimmune disease or condition, a respiratory disease or condition, and an inflammatory disease or condition.

Clause 387. The use of clause 386, wherein the psychological disease or condition is selected from the group consisting of depression, bipolar disorder, schizophrenia, anorexia nervosa, anxiety, grief, stress, bulimia, post-traumatic stress disorder, phobia, aversions such as smell, taste, sight, texture, social anxiety, shock, obsessive-compulsive disorder, eating disorders dementia, Alzheimer's, Parkinson's, multiple sclerosis.

Clause 388. The use of clause 386, wherein the allergy or intolerance is selected from the group consisting of gluten, dairy, soy, nut and seed.

Clause 389. The use of clause 386, wherein the gastrointestinal disease or condition is selected from the group consisting of irritable bowel syndrome (IBS), celiac disease, and Crohn's disease.

Clause 390. The use of clause 386, wherein the disease or condition is advanced age.

Clause 391. The use of clause 386, wherein the medication is selected from the group consisting of antibiotics, codeine, morphine, sleeping pills, blood pressure medications, diuretics, anabolic steroids, cardiovascular medications, including digoxin, fluoxetine and hydralazine.

Clause 392. The use of clause 386, wherein the substance use is selected from the group consisting of cocaine, methamphetamines, heroin, alcohol.

Clause 393. The use of clause 386, wherein the disease or condition is substance withdrawal.

Clause 394. The use of clause 386, wherein the disease or condition is food poisoning.

Clause 395. The use of clause 386, wherein the disease or condition is dehydration.

Clause 396. The use of clause 386, wherein the viral infection, bacterial infection, or disease or condition caused by a viral infection or a bacterial infection is selected from the group consisting of upper respiratory infection, COVID-19, pneumonia, gastroenteritis, skin, meningitis, HIV, hepatitis, flu, common cold, urine infection.

Clause 397. The use of clause 386, wherein the disease or condition is selected from the group consisting of sleep deprivation, fatigue, chronic fatigue syndrome, nausea, loss of taste, smell, sight, and satiety.

Clause 398. The use of clause 386, wherein the disease or condition is pregnancy or hormonal therapy.

Clause 399. The use of clause 386, wherein the pain is caused by a condition or disease selected from the group consisting of fibromyalgia, migraines, nerve damage, postural orthostatic tachycardia syndrome, post-surgical, oral pain, and dental pain.

Clause 400. The use of clause 386, wherein the disease or condition is selected from the group consisting of heart disease, high blood pressure, anemia, lupus, rheumatoid arthritis, chronic lung disease, inflammatory conditions, and menopause.

Clause 401. The use of any one of clauses 361-400, wherein the metabolically unchallenged subject is a human.

Clause 402. The use of any one of clauses 361-400, wherein the metabolically unchallenged subject is an animal.

Clause 403. The use of any one of clauses 361-402, wherein the increased body weight or BMI comprises increased muscle mass, increased fat mass, or both.

Clause 404. The use of clause 403, wherein the muscle mass is skeletal muscle mass, cardiac muscle mass, or both.

Clause 405. The use of any one of clauses 361-402, wherein the increased body weight or BMI comprises increased lean mass.

Clause 406. The use of any one of clauses 216-360, wherein the subject has a cancer.

Clause 407. The use of clause 406, wherein the cancer is at least one selected from the group consisting of bone cancer, testicular cancer, gastric cancer, sarcoma, lymphoma, Hodgkin's lymphoma, leukemia, head and neck cancer, squamous cell head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colorectal cancer, oral cancer, skin cancer, and melanoma.

Clause 408. The use of clause 406 or 407, wherein the cancer is at least one selected from the group consisting of bone cancer, lung cancer, testicular cancer, breast cancer, ovarian cancer, cervical cancer, bladder cancer, and head and neck cancer.

Clause 409. The use of clause 406 or 407, wherein the cancer is at least one selected from the group consisting of non-small-cell lung cancer, colorectal cancer, stomach cancer, ovarian cancer, and pancreatic cancer.

Clause 410. The use of claim any one of clauses 406-409, further comprising administering an anticancer agent to the subject.

Clause 411. The use of clause 410, wherein the anticancer agent is at least one chemotherapeutic agent.

Clause 412. The use of clause 411, wherein the at least one chemotherapeutic agent comprises one or more chemotherapeutic agents selected from the group consisting of a platinum-coordination complex, an antimetabolite, a tubulin binding agent, an alkylating antineoplastic agent, and a cytotoxic antibiotic.

Clause 413. The use of any one of clauses 216-412, further comprising administering an appetite-regulating agent to the subject.

Clause 414. The use of any one of clauses 216-413, wherein the subject does not experience an adverse event following administration of the non-naturally occurring melanocortin analog.

Clause 415. The use of any one of clauses 216-414, wherein the subject does not experience an adverse event attributable to the non-naturally occurring melanocortin analog following administration.

Clause 416. The use of clause 296, wherein appetite of the subject is assessed by a scale for measuring satiety.

Clause 417. The use of clauses 303 or 416, wherein satiety decreased by at least 40% to 150% within 8 days after the administration.

Clause 418. The use of clauses 303 or 416, wherein satiety decreased by at least 7 points within 8 days after the administration.

Clause 419. The use of clauses 303 or 416, wherein satiety decreased below 15 within 8 days after the administration.

Clause 420. The use of clause 296 or 297, wherein body weight of the subject is increased by at least 2% to 10% between day 2 after the administration and day 7 after the administration.

Clause 421. The use of clause 296 or 297, wherein body weight of the subject is increased by at least 1.5 kg between day 2 after the administration and day 7 after the administration.

Clause 422. The use of clause 296 or 297, wherein BMI of the subject is increased by at least 1% to 10% between day 2 after the administration and day 7 after the administration.

Clause 423. The use of clause 296 or 297, wherein BMI of the subject is increased by at least 0.5 between day 2 after administration and day 7 after administration.

Clause 424. The use of any one of clauses 329-334, wherein appetite of the subject is assessed by a scale for measuring satiety.

Clause 425. The use of any one of clauses 334 or 424, wherein satiety of the subject is decreased after the administration by at least 40% to 150%, relative to satiety of the control subject.

Clause 426. The use of any one of clauses 334 or 424, wherein satiety of the subject is decreased after the administration by at least 14 points relative to satiety of the control subject.

Clause 427. The use of any one of clauses 326-328, wherein body weight of the subject is increased between day 2 after the administration and day 7 after the administration, and wherein weight gain of the subject between day 2 after the administration and day 7 after the administration is at least 20% to 150% greater than that of the control subject.

Clause 428. The use of any one of clauses 326-328, wherein body weight of the subject is increased between day 2 after the administration and day 7 after the administration, and wherein weight gain of the subject between day 2 after the administration and day 7 after administration is at least 0.5 kg greater than that of the control subject.

Clause 429. The use of any one of clauses 326-328, wherein BMI of the subject is increased between day 2 after the administration and day 7 after the administration, and wherein BMI increase of the subject between day 2 after the administration and day 7 after administration is at least 20% to 100% greater than that of the control subject.

Clause 430. The use of any one of clauses 326-328, wherein BMI of the subject is increased between day 2 after the administration and day 7 after the administration, and wherein BMI increase of the subject between day 2 after the administration and day 7 after administration is at least 0.25 greater than that of the control subject.

Clause 431. A use of a non-naturally occurring melanocortin analog in the manufacture of a medicament for stimulating appetite of a subject in need thereof, the use comprising: administering to the subject a therapeutically effective amount of the non-naturally occurring melanocortin analog, wherein the non-naturally occurring melanocortin analog comprises a sequence according to Formula (I), administering to the subject a therapeutically effective amount of a non-naturally occurring melanocortin analog comprising a sequence according to Formula (I),

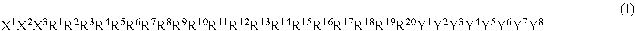

wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH═CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (β-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-α][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (loc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(CI)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);;
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
   wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine; and
wherein the non-naturally occurring melanocortin analog comprises one or more of the following features: (i) X² or X³ is present; (ii) R¹ is absent or is selected from the group consisting of dArg, dMet, dIIe, dLeu, dVal, dAla, Ala, Tle, dTle, DNle, Nva, Gly, dPro, dCys, dPhe, dTyr, dGln, dAsn, transPro(guan), cisPro(guan), dTyr, Tyr, and Dmt; (iii) R² is absent; (iv) R³ is selected from the group consisting of dPro, Pro-Gly, and dAla; (v) R⁴ is selected from the group consisting of His, Trp, and Phe; (vi) R⁵ is Phe; (vii) R⁷ is absent; (viii) R⁸ is present; (ix) R⁹ and/or R¹⁰ is present; (x) one or more of R¹¹—R²⁰ are present; and (xi) Y⁴ is present; and
the non-naturally occurring melanocortin analog is cyclized through a moiety selected from the group consisting of:
   a disulfide bond between R¹ or R² and R⁷ or X¹ when R¹ or R² is cysteine and R⁷ or X¹ is cysteine;
   a disulfide bond between R² and any one of R⁵—R²⁰ when R² and the any one of R⁵—R²⁰ are selected from the group consisting of cysteine, Pen, and dPen;
   a lactam bridge between R¹ and R⁷ when R¹ is norleucine and R⁷ is glutamic acid;
   a side-chain lactam bridge between R² and R⁷ when R² is glutamic acid, aspartic acid, or CO-cis-CH═CH—CO, and R⁷ is lysine or ornithine;
   a side-chain lactam bridge between R² and R⁸ when R² is glutamic acid or aspartic acid, R⁸ is lysine, and R⁷ is proline or tryptophan;
   a lactam closure between R¹ and R⁷ when R¹ is succinic acid or o-phthalic acid and R⁷ is lysine; and
   a lactam closure between R² and R⁷ when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus.

From the foregoing, it will be appreciated that specific embodiments of the present technology have been described in accordance with the present technology for purposes of illustration, but that various modifications may be made without deviating from the scope of the present technology. Accordingly, the present technology is not limited except as by the appended claims.

1. A method of stimulating appetite and increasing or maintaining muscle mass of a subject in need thereof, the method comprising:
administering to the subject, for at least 1 week, a therapeutically effective amount of a non-naturally occurring melanocortin analog comprising a sequence according to Formula (I), 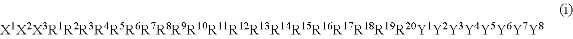
wherein:
R¹ is absent or is selected from the group consisting of cysteine, norleucine (Nle), acetylated norleucine (Ac-Nle), trans-4-guanidinyl-proline (transPro(guan)), cis-4-guanidinyl-proline (cisPro(guan)), acetylated cysteine, methylated D-phenylalanine, succinic acid, o-phthalic acid, tyrosine, D-tyrosine, di-methyl tyrosine (Dmt), aspartic acid, glutaric acid, CO-cis-CH═CH—CO, an n-pentanoyl group, an n-hexanoyl group, leucine, isoleucine, valine, norvaline, alanine, glycine, proline, methionine, lysine, phenylalanine, glutamic acid, asparagine, acetylated D-arginine, acetylated arginine, acetylated D-methionine, acetylated D-isoleucine, acetylated D-leucine, acetylated D-valine, acetylated alanine, acetylated D-alanine, acetylated tert-leucine (Tle), acetylated D-tert-leucine (dTle), acetylated norvaline (Nva), acetylated glycine, acetylated D-proline, acetylated D-phenylalanine, acetylated glutamic acid, acetylated D-tyrosine, acetylated D-glutamine, and acetylated D-asparagine;
R² is absent or is selected from the group consisting of proline, D-hydroxyproline (dHyp), hydroxyproline (Hyp), transPro(guan), cisPro(guan), aspartic acid, glutamic acid, glycine, lysine, alanine, D-alanine, tryptophan, cysteine, norleucine, arginine, succinic acid, glutaric acid, CO-cis-CH—CH—CO, an n-pentanoyl group, an n-hexanoyl group, methionine, phenylalanine, penicillamine (Pen), and D-penicillamine (dPen);
R³ is absent or is selected from the group consisting of histidine, histidine methylated at positions 1 or 3, D-proline, L-proline, hydroxyproline (Hyp), D-hydroxyproline (dHyp), transPro(guan), cisPro(guan), alanine, D-alanine, D-methionine, valine, D-valine, glutamic acid, prolylglycine (Pro-Gly), glycylglycine (Gly-Gly), tryptylarginine (Trp-Arg), glycine, phenylalanine, D-phenylalanine, succinic acid, D-leucine, leucine, D-isoleucine, isoleucine, tryptophan, arginine, 4-amino-1,2,4,5-tetrahydro-2-benzazepin-3-one (Aba), beta-alanine (b-Ala), 3-aminomethylbenzoic acid (Mamb), 1-aminocyclo-propane-1-carboxylic acid (Acpc), 2-aminotetraline-2-carboxylic acid (Atc), 7-amino-7,8-dihydro-4H-[1,2,3]triazolo-[1,5-a][1,4]diazepin-6 (5H)-one (Ata), 4-amino-1,4,5,6-tetrahydroazepino[4,3-b]indol-3 (2H)-one (Aia), 1-amino-4-phenylcyclohexane-carboxylic acid (APC), 4-aminophenylpiperidine-4-carboxylic acid (APPC), octohydroindole-2-carboxylic acid (Oic), 1-amino-1-cyclohexanecarboxylic acid (Che), tetrahydro-isoquinoline-3-carboxylic acid (Tic), indoline-2-carboxylic acid (Ioc), 2-aminoindone-2-carboxylic acid (Aic), and 1-amino-1-cyclopentane carboxylic (Cpe);
R⁴ is selected from the group consisting of histidine, D-phenylalanine, L-phenylalanine, D-Nal(2′), pCI-D-Phe, (o-Phe) Phe, aspartic acid, a disubstituted biphenyl(Bip), glycine, proline, cysteine, para-chloro-D-phenylalanine (p(C1)dPhe), para-bromo-D-phenylalanine (p(Br)dPhe), para-iodo-D-phenylalanine (p(I)dPhe), para-fluoro-D-phenylalanine (p(F)dPhe), and para-trifluoromethyl-D-phenylalanine (p(CF₃)dPhe);;
R⁵ is absent or is selected from the group consisting of arginine, homoarginine, ornithine, histidine, alanine, proline, transPro(guan), cisPro(guan), Pip, Nip, Tic, Phg, Sar, Azt, phenylalanine, D-Nal(2′), lysine, D-arginine, D-ornithine, D-histidine, D-alanine, D-lysine, glycine, aspartic acid, D-aspartic acid, glutamic acid, D-glutamic acid, cysteine, and p(I)dPhe;
R⁶ is absent or is selected from the group consisting of D-tryptophan, L-tryptophan, D-Nal(2′), L-Nal(2′), Tic, Bip, arginine, histidine, D-histidine, cysteine, Nal(1′), dNal(1′), Aia, phenylalanine, Aba, Ata, tyrosine, D-tyrosine, Pen, dPen, alanine, and D-alanine;
R⁷ is absent or is selected from the group consisting of glycine, glutamic acid, cysteine, lysine, D-cysteine, D-lysine, 2,3-diamino-propionic acid, methionine, proline, tryptophan, D-Nal(2′), ornithine, D-ornithine, Pen, dPen, and tetrahydro-isoquinoline-3-carboxylic acid (Tic);
R⁸ is absent or is lysine or arginine;
R⁹ is absent or is tryptophan;
R¹⁰ is absent or is lysine;
R¹¹—R²⁰ are each independently absent or selected from the group consisting of cysteine, norleucine, tyrosine, aspartic acid, leucine, isoleucine, valine, norvaline, alanine, glycine, proline, methionine, lysine, phenylalanine, glutamic acid, arginine, histidine, hydroxyproline, D-hydroxyproline, D-proline, prolylglycine (Pro-Gly), D-Nal(2′), L-Nal(2′), a disubstituted biphenyl, ornithine, and tryptophan;
wherein if R² is an n-pentanoyl group or an n-hexanoyl group, then R¹, X¹, X² and X³ are absent;
X¹ is absent or is selected from the group consisting of D-cysteine, L-cysteine, D-threonine, D-proline, L-proline, β-homo proline, D-alanine, L-alanine, β-alanine, D-arginine, L-arginine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, acetylated D-arginine, acetylated L-arginine, acetylated D-valine, and acetylated norleucine;
X² is absent or is selected from the group consisting of D-threonine, D-proline, L-proline, β-homo proline, D-alanine, L-alanine, β-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
X³ is absent or is selected from the group consisting of D-cysteine, L-cysteine, D-threonine, D-proline, L-proline, β-homo proline, D-alanine, L-alanine, D-valine, L-valine, β-valine, D-leucine, L-leucine, β-leucine, D-isoleucine, L-isoleucine, β-isoleucine, a piperazin-2-one ring, norvaline, glycine, methionine, lysine, phenylalanine, tyrosine, glutamic acid, asparagine, aspartic acid, and norleucine;
Y¹ is selected from the group consisting of D-alanine, L-alanine, D-valine, L-valine, D-leucine, L-leucine, D-isoleucine, L-isoleucine, D-tert-leucine, L-tert-leucine, L-proline, D-proline, Hyp, dHyp, glycine, aspartic acid, D-aspartic acid, arginine, D-arginine, asparagine, D-asparagine, lysine, D-lysine, and tryptophan;
Y² is absent or is selected from the group consisting of D-proline, L-proline, D-valine, L-valine, D-tert-leucine, L-tert-leucine, Hyp, dHyp, D-alanine, L-alanine, glycine, aspartic acid, D-aspartic acid, arginine, D-arginine, asparagine, and D-asparagine;
Y³ is absent or is selected from the group consisting of D-proline, L-proline, D-valine, and L-valine;
Y⁴ is absent or is D-proline or D-valine;
Y⁵ is absent or is D-proline or D-valine;
Y⁶ is absent or is D-proline or D-valine;
Y⁷ is absent or is D-proline or D-valine;
Y⁸ is absent or is D-proline or D-valine; and
wherein the non-naturally occurring melanocortin analog comprises one or more of the following features: (i) X² or X³ is present; (ii) R¹ is absent or is selected from the group consisting of dArg, dMet, dIIe, dLeu, dVal, dAla, Ala, Tle, dTle, DNle, Nva, Gly, dPro, dCys, dPhe, dTyr, dGln, dAsn, transPro(guan), cisPro(guan), dTyr, Tyr, and Dmt; (iii) R² is absent;
(iv) R³ is selected from the group consisting of dPro, Pro-Gly, and dAla; (v) R⁴ is selected from the group consisting of His, Trp, and Phe; (vi) R⁵ is Phe; (vii) R⁷ is absent; (viii) R⁸ is present;
(ix) R⁹ and/or R¹⁰ is present; (x) one or more of R¹¹—R²⁰ are present; and (xi) Y⁴ is present; and
the non-naturally occurring melanocortin analog is cyclized through a moiety selected from the group consisting of:
a disulfide bond between R¹ or R² and R⁷ or X¹ when R¹ or R² is cysteine and R⁷ or X¹ is cysteine;
a disulfide bond between R² and any one of R⁵—R²⁰ when R² and the any one of R⁵—R²⁰ are selected from the group consisting of cysteine, Pen, and dPen;
a lactam bridge between R¹ and R⁷ when R¹ is norleucine and R⁷ is glutamic acid;
a side-chain lactam bridge between R² and R⁷ when R² is glutamic acid, aspartic acid, or CO-cis-CH—CH—CO, and R⁷ is lysine or ornithine;
a side-chain lactam bridge between R² and R⁸ when R² is glutamic acid or aspartic acid, R⁸ is lysine, and R⁷ is proline or tryptophan;
a lactam closure between R¹ and R⁷ when R¹ is succinic acid or o-phthalic acid and R⁷ is lysine; and
a lactam closure between R² and R⁷when R² is succinic acid and R⁷ is 2,3-diamino-propionic acid;
X¹X²X³ represents an optionally present N-terminus; and
Y¹Y²Y³Y⁴Y⁵Y⁶Y⁷Y⁸ represents a C-terminus,
wherein following the administration, the muscle mass is increased or maintained for at least one week.

2. (canceled)

3. The method of claim 1, wherein the N-terminus, if present, is modified by an acetyl group and the C-terminus is modified by an amide group.

4.-16. (canceled)

17. The method of claim 3, wherein R¹ is norleucin,
and R² is aspartic acid.

18.-20. (canceled)

21. The method of claim 17, wherein:
Y¹ is D-valine and Y² is D-proline.

22.-27. (canceled)

28. The method of claim 1, wherein R¹, R², and R⁷ are present and R⁸—R²⁰ are absent, and the sequence of Formula (I) is cyclized through R² and R⁷ via a lactam bond.

29. The method of claim 28, wherein:
R¹ is acetylated norleucine;
R² is aspartic acid;
R³ is selected from the group consisting of proline, hydroxyproline, and D-hydroxyproline;
R⁴ is dNal(2′);
R⁵ is arginine;
R⁶ is;
R⁷ is lysine;
Y¹ is selected from the group consisting of D-valine, D-arginine, and D-lysine; and/or
Y², if present, is D-proline.

30. The method of claim 28, wherein the sequence of Formula (I) is:
(SEQ ID NO: 3)
Ac-Nle-_{c}(Asp-Pro-dNal2′-Arg-Trp-Lys)-dVal-dPro-NH₂;
or
(SEQ ID NO: 4)
Ac-Nle-_{c}(Asp-Hyp-dNal2′-Arg-Trp-Lys)-dVal-dPro-NH₂,
wherein c represents cyclization through R² and R⁷ via a lactam bond.

31.-58. (canceled)

59. The method of claim 1, wherein the non-naturally occurring melanocortin analog comprises a sequence selected from the group consisting of SEQ ID NO: 3, SEQ ID NO: 150, SEQ ID NO: 156, and SEQ ID NO: 160.

60. The method of claim 1, wherein the non-naturally occurring melanocortin analog is present in a composition.

61. (canceled)

62. The method of claim 60, wherein the composition further comprises a pharmaceutical carrier.

63. The method of claim 60, wherein the non-naturally occurring melanocortin analog is present in the composition in a concentration of 1 mg/mL to 50 mg/mL, relative to a total volume of the composition.

64. The method of claim 63, wherein the non-naturally occurring melanocortin analog comprises a sequence of SEQ ID NO: 3.

65. (canceled)

66. The method of claim 1, wherein the non-naturally occurring melanocortin analog is administered via intraperitoneal, intravenous, parenteral, subcutaneous, intramuscular, intracerebroventricular, intranasal, or oral administration.

67. The method of claim 66, wherein the parenteral administration is subcutaneous administration.

68. (canceled)

69. The method of claim 1, wherein the non-naturally occurring melanocortin analog crosses the blood-brain-barrier of the subject.

70. The method of claim 1, wherein the therapeutically effective amount of the non-naturally occurring melanocortin analog is from 0.5 mg/kg to 25 mg/kg per body weight of the subject.

71. (canceled)

72. The method of claim 1, wherein the non-naturally occurring melanocortin analog is administered at least once daily in an amount ranging from 0.5 mg/kg to 10 mg/kg per body weight of the subject.

73.-75. (canceled)

76. The method of claim 1, wherein the non-naturally occurring melanocortin analog is administered to the subject for at least 1 day, 1 week, 1 month, 3 months, 6 months, 1 year, or 5 years.

77.-102. (canceled)

103. The method of claim 1, wherein muscle mass of the subject is increased by at least 5% to 15% after the administration.

104. (canceled)

105. The method of claim 103, wherein the increased muscle mass is maintained for at least 1 week, 2 weeks, 3 weeks, 1 month, 3 months, 6 months, 1 year, or 5 years, after administration.

106.-431. (canceled)

432. The method of claim 1, wherein the non-naturally occurring melanocortin analog is administered to the subject once daily, twice daily, three times daily, once weekly, once every 2 weeks, or once per month.


SEQUENCE LISTING (398 sequences)

SEQ ID NO: 1 - protein, 4 aa, synthetic construct
HFRW

SEQ ID NO: 2 - protein, 9 aa, synthetic construct
  SITE 1: note = Acetylated Norleucine
  SITE 4: note = D-2'-naphthylalanine
  SITE 8: note = D-Valine
  SITE 9: note = D-Proline
  SITE 2..7: note = side chain cyclized via a lactam bond
XDPXRWKVP

SEQ ID NO: 3 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 4 - protein, 9 aa, synthetic construct
  SITE 1: note = Acetylated Norleucine
  SITE 3: note = Hydroxyproline
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 5 - skipped

SEQ ID NO: 6 - protein, 10 aa, synthetic construct
  SITE 1: note = Acetylated Norleucine
  SITE 5: note = D-2'-naphthylalanine
  SITE 9: note = D-Valine
  SITE 10: note = Amidated D-Proline
  SITE 2..8: note = side chain cyclized via a lactam bond
XDPHXRWKVP

SEQ ID NO: 7 - protein, 10 aa, synthetic construct
  SITE 1: note = Acetylated Norleucine
  SITE 5: note = D-2'-naphthylalanine
  SITE 9: note = D-Leucine
  SITE 10: note = Amidated D-Proline
  SITE 2..8: note = side chain cyclized via a lactam bond
XDFHXRWKLP

SEQ ID NO: 8 - protein, 10 aa, synthetic construct
  SITE 1: note = Acetylated Norleucine
  SITE 5: note = D-2'-naphthylalanine
  SITE 9: note = D-Isoleucine
  SITE 10: note = Amidated D-Proline
  SITE 2..8: note = side chain cyclized via a lactam bond
XDFHXRWKIP

SEQ ID NO: 9 - protein, 10 aa, synthetic construct
  SITE 1: note = Acetylated Norleucine
  SITE 4: note = D-2'-naphthylalanine
  SITE 9: note = D-Valine
  SITE 10: note = Amidated D-Proline
  SITE 2..8: note = side chain cyclized via a lactam bond
XDHXRWPKVP

SEQ ID NO: 10 - protein, 10 aa, synthetic construct
  SITE 1: note = Acetylated Norleucine
  SITE 3: note = Hydroxyproline
  SITE 4: note = D-2'-naphthylalanine
  SITE 9: note = D-Valine
  SITE 10: note = Amidated D-Proline
  SITE 2..8: note = side chain cyclized via a lactam bond
XDXXRWPKVP

SEQ ID NO: 11 - protein, 10 aa, synthetic construct
  SITE 1: note = Acetylated Norleucine
  SITE 5: note = D-2'-naphthylalanine
  SITE 9: note = D-Proline
  SITE 10: note = Amidated D-Valine
  SITE 2..8: note = side chain cyclized via a lactam bond
XDPHXRWKPV

SEQ ID NO: 12 - protein, 12 aa, synthetic construct
  SITE 1: note = Acetylated Norleucine
  SITE 7: note = D-2'-naphthylalanine
  SITE 11: note = D-Valine
  SITE 12: note = Amidated D-Proline
  SITE 2..10: note = side chain cyclized via a lactam bond
XDFFPHXRWKVP

SEQ ID NO: 13 - protein, 12 aa, synthetic construct
  SITE 1: note = Acetylated Norleucine
  SITE 7: note = D-2'-naphthylalanine
  SITE 11: note = D-Valine
  SITE 12: note = Amidated D-Proline
  SITE 4..10: note = side chain cyclized via a lactam bond
XFFDFHXRWKVP

SEQ ID NO: 14 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
XDPXRWKVP

SEQ ID NO: 15 - protein, 7 aa, synthetic construct
  SITE 1: note = Acetylated Norleucine
  SITE 3: note = D-2'-naphthylalanine
  SITE 6: note = D-Valine
  SITE 7: note = Amidated D-Proline
XPXRWVP

SEQ ID NO: 16 - protein, 9 aa, synthetic construct
  SITE 1: note = Acetylated D-Arginine
  SITE 4: note = D-2'-naphthylalanine
  SITE 8: note = D-Valine
  SITE 9: note = Amidated D-Proline
  SITE 2..7: note = side chain cyclized via a lactam bond
RDPXRWKVP

SEQ ID NO: 17 - protein, 9 aa, synthetic construct
  SITE 1: note = Acetylated D-Methionine
  SITE 4: note = D-2'-naphthylalanine
  SITE 8: note = D-Valine
  SITE 9: note = Amidated D-Proline
  SITE 2..7: note = side chain cyclized via a lactam bond
MDPXRWKVP

SEQ ID NO: 18 - protein, 9 aa, synthetic construct
  SITE 1: note = Acetylated D-Isoleucine
  SITE 4: note = D-2'-naphthylalanine
  SITE 8: note = D-Valine
  SITE 9: note = Amidated D-Proline
  SITE 2..7: note = side chain cyclized via a lactam bond
IDPXRWKVP

SEQ ID NO: 19 - protein, 9 aa, synthetic construct
  SITE 1: note = Acetylated D-Leucine
  SITE 4: note = D-2'-naphthylalanine
  SITE 8: note = D-Valine
  SITE 9: note = Amidated D-Proline
  SITE 2..7: note = side chain cyclized via a lactam bond
LDPXRWKVP

SEQ ID NO: 20 - protein, 9 aa, synthetic construct
  SITE 1: note = Acetylated D-Valine
  SITE 4: note = D-2'-naphthylalanine
  SITE 8: note = D-Valine
  SITE 9: note = Amidated D-Proline
  SITE 2..7: note = side chain cyclized via a lactam bond
VDPXRWKVP

SEQ ID NO: 21 - protein, 9 aa, synthetic construct
  SITE 1: note = Acetylated D-Alanine
  SITE 4: note = D-2'-naphthylalanine
  SITE 8: note = D-Valine
  SITE 9: note = Amidated D-Proline
  SITE 2..7: note = side chain cyclized via a lactam bond
ADPXRWKVP

SEQ ID NO: 22 - protein, 9 aa, synthetic construct
  SITE 1: note = Acetylated Residue
  SITE 4: note = D-2'-naphthylalanine
  SITE 8: note = D-Valine
  SITE 9: note = Amidated D-Proline
  SITE 2..7: note = side chain cyclized via a lactam bond
ADPXRWKVP

SEQ ID NO: 23 - protein, 9 aa, synthetic construct
  SITE 1: note = Acetylated Tert-Leucine
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 24 - protein, 9 aa, synthetic construct
  SITE 1: note = Acetylated D-Tert-Leucine
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 25 - protein, 9 aa, synthetic construct
  SITE 1: note = Acetylated D-Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 26 - protein, 9 aa, synthetic construct
  SITE 1: note = Acetylated Norvaline
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 27 - protein, 9 aa, synthetic construct
  SITE 1: note = Acetylated Residue
  SITE 4: note = D-2'-naphthylalanine
  SITE 8: note = D-Valine
  SITE 9: note = Amidated D-Proline
  SITE 2..7: note = side chain cyclized via a lactam bond
GDPXRWKVP

SEQ ID NO: 28 - protein, 9 aa, synthetic construct
  SITE 1: note = Acetylated D-Proline
  SITE 4: note = D-2'-naphthylalanine
  SITE 8: note = D-Valine
  SITE 9: note = Amidated D-Proline
  SITE 2..7: note = side chain cyclized via a lactam bond
PDPXRWKVP

SEQ ID NO: 29 - protein, 9 aa, synthetic construct
  SITE 1: note = Acetylated D-Cysteine
  SITE 4: note = D-2'-naphthylalanine
  SITE 8: note = D-Valine
  SITE 9: note = Amidated D-Proline
  SITE 2..7: note = side chain cyclized via a lactam bond
CDPXRWKVP

SEQ ID NO: 30 - protein, 9 aa, synthetic construct
  SITE 1: note = Acetylated D-Phenylalanine
  SITE 4: note = D-2'-naphthylalanine
  SITE 8: note = D-Valine
  SITE 9: note = Amidated D-Proline
  SITE 2..7: note = side chain cyclized via a lactam bond
FDPXRWKVP

SEQ ID NO: 31 - protein, 9 aa, synthetic construct
  SITE 1: note = Acetylated D-Tyrosine
  SITE 4: note = D-2'-naphthylalanine
  SITE 8: note = D-Valine
  SITE 9: note = Amidated D-Proline
  SITE 2..7: note = side chain cyclized via a lactam bond
YDPXRWKVP

SEQ ID NO: 32 - protein, 9 aa, synthetic construct
  SITE 1: note = Acetylated D-Glutamine
  SITE 4: note = D-2'-naphthylalanine
  SITE 8: note = D-Valine
  SITE 9: note = Amidated D-Proline
  SITE 2..7: note = side chain cyclized via a lactam bond
QDPXRWKVP

SEQ ID NO: 33 - protein, 9 aa, synthetic construct
  SITE 1: note = Acetylated D-Asparagine
  SITE 4: note = D-2'-naphthylalanine
  SITE 8: note = D-Valine
  SITE 9: note = Amidated D-Proline
  SITE 2..7: note = side chain cyclized via a lactam bond
NDPXRWKVP

SEQ ID NO: 34 - protein, 9 aa, synthetic construct
  SITE 1: note = Acetylated trans-4-guanidinyl-proline
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 35 - protein, 9 aa, synthetic construct
  SITE 1: note = Acetylated cis-4-guanidinyl-proline
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 36 - protein, 9 aa, synthetic construct
  SITE 1: note = D-Tyrosine
  SITE 4: note = D-2'-naphthylalanine
  SITE 8: note = D-Valine
  SITE 9: note = Amidated D-Proline
  SITE 2..7: note = side chain cyclized via a lactam bond
YDPXRWKVP

SEQ ID NO: 37 - protein, 9 aa, synthetic construct
  SITE 4: note = D-2'-naphthylalanine
  SITE 8: note = D-Valine
  SITE 9: note = Amidated D-Proline
  SITE 2..7: note = side chain cyclized via a lactam bond
YDPXRWKVP

SEQ ID NO: 38 - protein, 9 aa, synthetic construct
  SITE 1: note = Di-methyl tyrosine
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 39 - protein, 9 aa, synthetic construct
  SITE 1: note = Acetylated Norleucine
  SITE 2: note = D-Aspartic Acid
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XDPXRWKVP

SEQ ID NO: 40 - protein, 9 aa, synthetic construct
  SITE 1: note = Acetylated Norleucine
  SITE 4: note = D-2'-naphthylalanine
  SITE 7: note = D-Lysine
  SITE 8: note = D-Valine
  SITE 9: note = Amidated D-Proline
  VARIANT 2..7: note = cyclization may occur via a lactam bond or disulfide bond
XDPXRWKVP

SEQ ID NO: 41 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XCPXRWCVP

SEQ ID NO: 42 - protein, 9 aa, synthetic construct
  SITE 1: note = Acetylated Norleucine
  SITE 2: note = D-Cysteine
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XCPXRWCVP

SEQ ID NO: 43 - protein, 9 aa, synthetic construct
  SITE 1: note = Acetylated Norleucine
  SITE 4: note = D-2'-naphthylalanine
  SITE 7: note = D-Cysteine
  SITE 8: note = D-Valine
  SITE 9: note = Amidated D-Proline
  VARIANT 2..7: note = cyclization may occur via a lactam bond or disulfide bond
XCPXRWCVP

SEQ ID NO: 44 - protein, 9 aa, synthetic construct
  SITE 1: note = Acetylated Norleucine
  SITE 2: note = D-Cysteine
  SITE 4: note = D-2'-naphthylalanine
  SITE 7: note = D-Cysteine
  SITE 8: note = D-Valine
  SITE 9: note = Amidated D-Proline
  VARIANT 2..7: note = cyclization may occur via a lactam bond or disulfide bond
XCPXRWCVP

SEQ ID NO: 45 - protein, 8 aa, synthetic construct
  SITE 1: note = Acetylated Norleucine
  SITE 3: note = D-2'-naphthylalanine
  SITE 7: note = D-Valine
  SITE 8: note = Amidated D-Proline
  VARIANT 2..6: note = cyclization may occur via a lactam bond or disulfide bond
XCXRWCVP

SEQ ID NO: 46 - protein, 8 aa, synthetic construct
  SITE 1: note = Acetylated Norleucine
  SITE 2: note = D-Cysteine
  SITE 3: note = D-2'-naphthylalanine
  SITE 7: note = D-Valine
  SITE 8: note = Amidated D-Proline
  VARIANT 2..6: note = cyclization may occur via a lactam bond or disulfide bond
XCXRWCVP

SEQ ID NO: 47 - protein, 8 aa, synthetic construct
  SITE 1: note = Acetylated Norleucine
  SITE 3: note = D-2'-naphthylalanine
  SITE 6: note = D-Cysteine
  SITE 7: note = D-Valine
  SITE 8: note = Amidated D-Proline
  VARIANT 2..6: note = cyclization may occur via a lactam bond or disulfide bond
XCXRWCVP

SEQ ID NO: 48 - protein, 8 aa, synthetic construct
  SITE 1: note = Acetylated Norleucine
  SITE 2: note = D-Cysteine
  SITE 3: note = D-2'-naphthylalanine
  SITE 6: note = D-Cysteine
  SITE 7: note = D-Valine
  SITE 8: note = Amidated D-Proline
  VARIANT 2..6: note = cyclization may occur via a lactam bond or disulfide bond
XCXRWCVP

SEQ ID NO: 49 - protein, 9 aa, synthetic construct
  SITE 1: note = Acetylated Norleucine
  SITE 4: note = D-2'-naphthylalanine
  SITE 7: note = Penicillamine
  SITE 8: note = D-Valine
  SITE 9: note = Amidated D-Proline
  VARIANT 2..7: note = cyclization may occur via a lactam bond or disulfide bond
XCPXRWXVP

SEQ ID NO: 50 - protein, 9 aa, synthetic construct
  SITE 1: note = Acetylated Norleucine
  SITE 2: note = D-Cysteine
  SITE 4: note = D-2'-naphthylalanine
  SITE 7: note = Penicillamine
  SITE 8: note = D-Valine
  SITE 9: note = Amidated D-Proline
  VARIANT 2..7: note = cyclization may occur via a lactam bond or disulfide bond
XCPXRWXVP

SEQ ID NO: 51 - protein, 9 aa, synthetic construct
  SITE 1: note = Acetylated Norleucine
  SITE 2: note = Penicillamine
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XXPXRWCVP

SEQ ID NO: 52 - protein, 9 aa, synthetic construct
  SITE 1: note = Acetylated Norleucine
  SITE 2: note = Penicillamine
  SITE 4: note = D-2'-naphthylalanine
  SITE 7: note = D-Cysteine
  SITE 8: note = D-Valine
  SITE 9: note = Amidated D-Proline
  VARIANT 2..7: note = cyclization may occur via a lactam bond or disulfide bond
XXPXRWCVP

SEQ ID NO: 53 - protein, 9 aa, synthetic construct
  SITE 1: note = Acetylated Norleucine
  SITE 2: note = Penicillamine
  SITE 4: note = D-2'-naphthylalanine
  SITE 7: note = Penicillamine
  SITE 8: note = D-Valine
  SITE 9: note = Amidated D-Proline
  VARIANT 2..7: note = cyclization may occur via a lactam bond or disulfide bond
XXPXRWXVP

SEQ ID NO: 54 - protein, 9 aa, synthetic construct
  SITE 1: note = Acetylated Norleucine
  SITE 2: note = D-Penicillamine
  SITE 4: note = D-2'-naphthylalanine
  SITE 7: note = Penicillamine
  SITE 8: note = D-Valine
  SITE 9: note = Amidated D-Proline
  VARIANT 2..7: note = cyclization may occur via a lactam bond or disulfide bond
XXPXRWXVP

SEQ ID NO: 55 - protein, 9 aa, synthetic construct
  SITE 1: note = Acetylated Norleucine
  SITE 2: note = D-Penicillamine
  SITE 4: note = D-2'-naphthylalanine
  SITE 7: note = D-Penicillamine
  SITE 8: note = D-Valine
  SITE 9: note = Amidated D-Proline
  VARIANT 2..7: note = cyclization may occur via a lactam bond or disulfide bond
XXPXRWXVP

SEQ ID NO: 56 - protein, 9 aa, synthetic construct
  SITE 1: note = Acetylated Norleucine
  SITE 2: note = Penicillamine
  SITE 4: note = D-2'-naphthylalanine
  SITE 7: note = D-Penicillamine
  SITE 8: note = D-Valine
  SITE 9: note = Amidated D-Proline
  VARIANT 2..7: note = cyclization may occur via a lactam bond or disulfide bond
XXPXRWXVP

SEQ ID NO: 57 - protein, 8 aa, synthetic construct
  SITE 1: note = Acetylated Norleucine
  SITE 3: note = D-2'-naphthylalanine
  SITE 6: note = Penicillamine
  SITE 7: note = D-Valine
  SITE 8: note = Amidated D-Proline
  VARIANT 2..6: note = cyclization may occur via a lactam bond or disulfide bond
XCXRWXVP

SEQ ID NO: 58 - protein, 8 aa, synthetic construct
  SITE 1: note = Acetylated Norleucine
  SITE 2: note = D-Cysteine
  SITE 3: note = D-2'-naphthylalanine
  SITE 6: note = Penicillamine
  SITE 7: note = D-Valine
  SITE 8: note = Amidated D-Proline
  VARIANT 2..6: note = cyclization may occur via a lactam bond or disulfide bond
XCXRWXVP

SEQ ID NO: 59 - protein, 8 aa, synthetic construct
  SITE 1: note = Acetylated Norleucine
  SITE 2: note = Penicillamine
  SITE 3: note = D-2'-naphthylalanine
  SITE 7: note = D-Valine
  SITE 8: note = Amidated D-Proline
  VARIANT 2..6: note = cyclization may occur via a lactam bond or disulfide bond
XXXRWCVP

SEQ ID NO: 60 - protein, 8 aa, synthetic construct
  SITE 1: note = Acetylated Norleucine
  SITE 2: note = Penicillamine
  SITE 3: note = D-2'-naphthylalanine
  SITE 6: note = D-Cysteine
  SITE 7: note = D-Valine
  SITE 8: note = Amidated D-Proline
  VARIANT 2..6: note = cyclization may occur via a lactam bond or disulfide bond
XXXRWCVP

SEQ ID NO: 61 - protein, 8 aa, synthetic construct
  SITE 1: note = Acetylated Norleucine
  SITE 2: note = Penicillamine
  SITE 3: note = D-2'-naphthylalanine
  SITE 6: note = Penicillamine
  SITE 7: note = D-Valine
  SITE 8: note = Amidated D-Proline
  VARIANT 2..6: note = cyclization may occur via a lactam bond or disulfide bond
XXXRWXVP

SEQ ID NO: 62 - protein, 8 aa, synthetic construct
  SITE 1: note = Acetylated Norleucine
  SITE 2: note = D-Penicillamine
  SITE 3: note = D-2'-naphthylalanine
  SITE 6: note = Penicillamine
  SITE 7: note = D-Valine
  SITE 8: note = Amidated D-Proline
  VARIANT 2..6: note = cyclization may occur via a lactam bond or disulfide bond
XXXRWXVP

SEQ ID NO: 63 - protein, 8 aa, synthetic construct
  SITE 1: note = Acetylated Norleucine
  SITE 2: note = D-Penicillamine
  SITE 3: note = D-2'-naphthylalanine
  SITE 6: note = D-Penicillamine
  SITE 7: note = D-Valine
  SITE 8: note = Amidated D-Proline
  VARIANT 2..6: note = cyclization may occur via a lactam bond or disulfide bond
XXXRWXVP

SEQ ID NO: 64 - protein, 8 aa, synthetic construct
  SITE 1: note = Acetylated Norleucine
  SITE 2: note = Penicillamine
  SITE 3: note = D-2'-naphthylalanine
  SITE 6: note = D-Penicillamine
  SITE 7: note = D-Valine
  SITE 8: note = Amidated D-Proline
  VARIANT 2..6: note = cyclization may occur via a lactam bond or disulfide bond
XXXRWXVP

SEQ ID NO: 65 - protein, 9 aa, synthetic construct
  SITE 1: note = Acetylated Norleucine
  SITE 4: note = D-2'-naphthylalanine
  SITE 7: note = Ornithine
  SITE 8: note = D-Valine
  SITE 9: note = Amidated D-Proline
  VARIANT 2..7: note = cyclization may occur via a lactam bond or disulfide bond
XDPXRWXVP

SEQ ID NO: 66 - protein, 9 aa, synthetic construct
  SITE 1: note = Acetylated Norleucine
  SITE 4: note = D-2'-naphthylalanine
  SITE 7: note = D-Ornithine
  SITE 8: note = D-Valine
  SITE 9: note = Amidated D-Proline
  VARIANT 2..7: note = cyclization may occur via a lactam bond or disulfide bond
XDPXRWXVP

SEQ ID NO: 67 - protein, 9 aa, synthetic construct
  SITE 1: note = Acetylated Norleucine
  SITE 4: note = D-2'-naphthylalanine
  SITE 7: note = Ornithine
  SITE 8: note = D-Valine
  SITE 9: note = Amidated D-Proline
  VARIANT 2..7: note = cyclization may occur via a lactam bond or disulfide bond
XEPXRWXVP

SEQ ID NO: 68 - protein, 9 aa, synthetic construct
  SITE 1: note = Acetylated Norleucine
  SITE 4: note = D-2'-naphthylalanine
  SITE 7: note = D-Ornithine
  SITE 8: note = D-Valine
  SITE 9: note = Amidated D-Proline
  VARIANT 2..7: note = cyclization may occur via a lactam bond or disulfide bond
XEPXRWXVP

SEQ ID NO: 69 - protein, 8 aa, synthetic construct
  SITE 1: note = Acetylated Norleucine
  SITE 3: note = D-2'-naphthylalanine
  SITE 7: note = D-Valine
  SITE 8: note = Amidated D-Proline
  SITE 2..6: note = side chain cyclized via a lactam bond
XDXRWKVP

SEQ ID NO: 70 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDAXRWKVP

SEQ ID NO: 71 - protein, 9 aa, synthetic construct
  SITE 1: note = Acetylated Norleucine
  SITE 3: note = D-Proline
  SITE 4: note = D-2'-naphthylalanine
  SITE 8: note = D-Valine
  SITE 9: note = Amidated D-Proline
  SITE 2..7: note = side chain cyclized via a lactam bond
XDPXRWKVP

SEQ ID NO: 72 - protein, 9 aa, synthetic construct
  SITE 1: note = Acetylated Norleucine
  SITE 3: note = D-Alanine
  SITE 4: note = D-2'-naphthylalanine
  SITE 8: note = D-Valine
  SITE 9: note = Amidated D-Proline
  SITE 2..7: note = side chain cyclized via a lactam bond
XDAXRWKVP

SEQ ID NO: 73 - protein, 9 aa, synthetic construct
  SITE 1: note = Acetylated Norleucine
  SITE 3: note = D-Methionine
  SITE 4: note = D-2'-naphthylalanine
  SITE 8: note = D-Valine
  SITE 9: note = Amidated D-Proline
  SITE 2..7: note = side chain cyclized via a lactam bond
XDMXRWKVP

SEQ ID NO: 74 - protein, 10 aa, synthetic construct
  SITE 1: note = Acetylated Norleucine
  SITE 5: note = D-2'-naphthylalanine
  SITE 9: note = D-Valine
  SITE 10: note = Amidated D-Proline
  SITE 2..8: note = side chain cyclized via a lactam bond
XDPGXRWKVP

SEQ ID NO: 75 - protein, 10 aa, synthetic construct
  SITE 1: note = Acetylated Norleucine
  SITE 5: note = D-2'-naphthylalanine
  SITE 9: note = D-Valine
  SITE 10: note = Amidated D-Proline
  SITE 2..8: note = side chain cyclized via a lactam bond
XDGGXRWKVP

SEQ ID NO: 76 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDGXRWKVP

SEQ ID NO: 77 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDLXRWKVP

SEQ ID NO: 78 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDIXRWKVP

SEQ ID NO: 79 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDVXRWKVP

SEQ ID NO: 80 - protein, 9 aa, synthetic construct
  SITE 1: note = Acetylated Norleucine
  SITE 3: note = D-Leucine
  SITE 4: note = D-2'-naphthylalanine
  SITE 8: note = D-Valine
  SITE 9: note = Amidated D-Proline
  SITE 2..7: note = side chain cyclized via a lactam bond
XDLXRWKVP

SEQ ID NO: 81 - protein, 9 aa, synthetic construct
  SITE 1: note = Acetylated Norleucine
  SITE 3: note = D-Isoleucine
  SITE 4: note = D-2'-naphthylalanine
  SITE 8: note = D-Valine
  SITE 9: note = Amidated D-Proline
  SITE 2..7: note = side chain cyclized via a lactam bond
XDIXRWKVP

SEQ ID NO: 82 - protein, 9 aa, synthetic construct
  SITE 1: note = Acetylated Norleucine
  SITE 3: note = D-Valine
  SITE 4: note = D-2'-naphthylalanine
  SITE 8: note = D-Valine
  SITE 9: note = Amidated D-Proline
  SITE 2..7: note = side chain cyclized via a lactam bond
XDVXRWKVP

SEQ ID NO: 83 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDWXRWKVP

SEQ ID NO: 84 - protein, 9 aa, synthetic construct
  SITE 1: note = Acetylated Norleucine
  SITE 3: note = D-Tryptophan
  SITE 4: note = D-2'-naphthylalanine
  SITE 8: note = D-Valine
  SITE 9: note = Amidated D-Proline
  SITE 2..7: note = side chain cyclized via a lactam bond
XDWXRWKVP

SEQ ID NO: 85 - protein, 9 aa, synthetic construct
  SITE 1: note = Acetylated Norleucine
  SITE 3: note = trans-4-guanidinyl-proline
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 86 - protein, 9 aa, synthetic construct
  SITE 1: note = Acetylated Norleucine
  SITE 3: note = cis-4-guanidinyl-proline
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 87 - protein, 8 aa, synthetic construct
  SITE 1: note = Acetylated Norleucine
  SITE 4: note = D-2'-naphthylalanine
  SITE 7: note = D-Valine
  SITE 8: note = Amidated D-Proline
  SITE 2..6: note = side chain cyclized via a lactam bond
XDPXWKVP

SEQ ID NO: 88 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXKWKVP

SEQ ID NO: 89 - protein, 9 aa, synthetic construct
  SITE 1: note = Acetylated Norleucine
  SITE 4: note = D-2'-naphthylalanine
  SITE 5: note = D-Lysine
  SITE 8: note = D-Valine
  SITE 9: note = Amidated D-Proline
  SITE 2..7: note = side chain cyclized via a lactam bond
XDPXKWKVP

SEQ ID NO: 90 - protein, 9 aa, synthetic construct
  SITE 1: note = Acetylated Norleucine
  SITE 4: note = D-2'-naphthylalanine
  SITE 5: note = D-Arginine
  SITE 8: note = D-Valine
  SITE 9: note = Amidated D-Proline
  SITE 2..7: note = side chain cyclized via a lactam bond
XDPXRWKVP

SEQ ID NO: 91 - protein, 9 aa, synthetic construct
  SITE 1: note = Acetylated Norleucine
  SITE 4: note = D-2'-naphthylalanine
  SITE 5: note = Ornithine
  SITE 8: note = D-Valine
  SITE 9: note = Amidated D-Proline
  SITE 2..7: note = side chain cyclized via a lactam bond
XDPXXWKVP

SEQ ID NO: 92 - protein, 9 aa, synthetic construct
  SITE 1: note = Acetylated Norleucine
  SITE 4: note = D-2'-naphthylalanine
  SITE 5: note = D-Ornithine
  SITE 8: note = D-Valine
  SITE 9: note = Amidated D-Proline
  SITE 2..7: note = side chain cyclized via a lactam bond
XDPXXWKVP

SEQ ID NO: 93 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXHWKVP

SEQ ID NO: 94 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXAWKVP

SEQ ID NO: 95 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXGWKVP

SEQ ID NO: 96 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXDWKVP

SEQ ID NO: 97 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXEWKVP

SEQ ID NO: 98 - protein, 9 aa, synthetic construct
  SITE 1: note = Acetylated Norleucine
  SITE 4: note = D-2'-naphthylalanine
  SITE 5: note = D-Histidine
  SITE 8: note = D-Valine
  SITE 9: note = Amidated D-Proline
  SITE 2..7: note = side chain cyclized via a lactam bond
XDPXHWKVP

SEQ ID NO: 99 - protein, 9 aa, synthetic construct
  SITE 1: note = Acetylated Norleucine
  SITE 4: note = D-2'-naphthylalanine
  SITE 5: note = D-Alanine
  SITE 8: note = D-Valine
  SITE 9: note = Amidated D-Proline
  SITE 2..7: note = side chain cyclized via a lactam bond
XDPXAWKVP

SEQ ID NO: 100 - protein, 9 aa, synthetic construct
  SITE 1: note = Acetylated Norleucine
  SITE 4: note = D-2'-naphthylalanine
  SITE 5: note = D-Aspartic Acid
  SITE 8: note = D-Valine
  SITE 9: note = Amidated D-Proline
  SITE 2..7: note = side chain cyclized via a lactam bond
XDPXDWKVP

SEQ ID NO: 101 - protein, 9 aa, synthetic construct
  SITE 1: note = Acetylated Norleucine
  SITE 4: note = D-2'-naphthylalanine
  SITE 5: note = D-Glutamic Acid
  SITE 8: note = D-Valine
  SITE 9: note = Amidated D-Proline
  SITE 2..7: note = side chain cyclized via a lactam bond
XDPXEWKVP

SEQ ID NO: 102 - protein, 8 aa, synthetic construct
  SITE 1: note = Acetylated Norleucine
  SITE 4: note = D-2'-naphthylalanine
  SITE 7: note = D-Valine
  SITE 8: note = Amidated D-Proline
  SITE 2..6: note = side chain cyclized via a lactam bond
XDPXRKVP

SEQ ID NO: 103 - protein, 9 aa, synthetic construct
  SITE 1: note = Acetylated Norleucine
  SITE 4: note = D-2'-naphthylalanine
  SITE 6: note = 1'-naphthylalanine
  SITE 8: note = D-Valine
  SITE 9: note = Amidated D-Proline
  SITE 2..7: note = side chain cyclized via a lactam bond
XDPXRXKVP

SEQ ID NO: 104 - protein, 9 aa, synthetic construct
  SITE 1: note = Acetylated Norleucine
  SITE 4: note = D-2'-naphthylalanine
  SITE 6: note = 4-amino-1,4,5,6-tetrahydroazepino[4,3-b]indol-3(2H) -one
  SITE 8: note = D-Valine
  SITE 9: note = Amidated D-Proline
  SITE 2..7: note = side chain cyclized via a lactam bond
XDPXRXKVP

SEQ ID NO: 105 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXRFKVP

SEQ ID NO: 106 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXRYKVP

SEQ ID NO: 107 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXRHKVP

SEQ ID NO: 108 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXRAKVP

SEQ ID NO: 109 - protein, 9 aa, synthetic construct
  SITE 1: note = Acetylated Norleucine
  SITE 4: note = D-2'-naphthylalanine
  SITE 6: note = D-1'-Naphthylalanine
  SITE 8: note = D-Valine
  SITE 9: note = Amidated D-Proline
  SITE 2..7: note = side chain cyclized via a lactam bond
XDPXRXKVP

SEQ ID NO: 110 - protein, 9 aa, synthetic construct
  SITE 1: note = Acetylated Norleucine
  SITE 4: note = D-2'-naphthylalanine
  SITE 6: note = D-Phenylalanine
  SITE 8: note = D-Valine
  SITE 9: note = Amidated D-Proline
  SITE 2..7: note = side chain cyclized via a lactam bond
XDPXRFKVP

SEQ ID NO: 111 - protein, 9 aa, synthetic construct
  SITE 1: note = Acetylated Norleucine
  SITE 4: note = D-2'-naphthylalanine
  SITE 6: note = D-2'-naphthylalanine
  SITE 8: note = D-Valine
  SITE 9: note = Amidated D-Proline
  SITE 2..7: note = side chain cyclized via a lactam bond
XDPXRXKVP

SEQ ID NO: 112 - protein, 9 aa, synthetic construct
  SITE 1: note = Acetylated Norleucine
  SITE 4: note = D-2'-naphthylalanine
  SITE 6: note = D-Tyrosine
  SITE 8: note = D-Valine
  SITE 9: note = Amidated D-Proline
  SITE 2..7: note = side chain cyclized via a lactam bond
XDPXRYKVP

SEQ ID NO: 113 - protein, 9 aa, synthetic construct
  SITE 1: note = Acetylated Norleucine
  SITE 4: note = D-2'-naphthylalanine
  SITE 6: note = D-Histidine
  SITE 8: note = D-Valine
  SITE 9: note = Amidated D-Proline
  SITE 2..7: note = side chain cyclized via a lactam bond
XDPXRHKVP

SEQ ID NO: 114 - protein, 9 aa, synthetic construct
  SITE 1: note = Acetylated Norleucine
  SITE 4: note = D-2'-naphthylalanine
  SITE 6: note = D-Alanine
  SITE 8: note = D-Valine
  SITE 9: note = Amidated D-Proline
  SITE 2..7: note = side chain cyclized via a lactam bond
XDPXRAKVP

SEQ ID NO: 115 - protein, 9 aa, synthetic construct
  SITE 1: note = Acetylated Norleucine
  SITE 4: note = Biphenyl amino acid
  SITE 8: note = D-Valine
  SITE 9: note = Amidated D-Proline
  SITE 2..7: note = side chain cyclized via a lactam bond
XDPXRWKVP

SEQ ID NO: 116 - protein, 9 aa, synthetic construct
  SITE 1: note = Acetylated Norleucine
  SITE 4: note = D-2'-naphthylalanine
  SITE 9: note = Amidated Residue
  SITE 2..7: note = side chain cyclized via a lactam bond
XDPXRWKVP

SEQ ID NO: 117 - protein, 9 aa, synthetic construct
  SITE 1: note = Acetylated Norleucine
  SITE 4: note = D-2'-naphthylalanine
  SITE 8: note = D-Valine
  SITE 9: note = Hydroxylated D-Proline
  SITE 2..7: note = side chain cyclized via a lactam bond
XDPXRWKVP

SEQ ID NO: 118 - protein, 9 aa, synthetic construct
  SITE 1: note = Acetylated Norleucine
  SITE 4: note = D-2'-naphthylalanine
  SITE 8: note = D-Proline
  SITE 9: note = Hydroxylated D-Valine
  SITE 2..7: note = side chain cyclized via a lactam bond
XDPXRWKPV

SEQ ID NO: 119 - protein, 9 aa, synthetic construct
  SITE 1: note = Acetylated Norleucine
  SITE 4: note = D-2'-naphthylalanine
  SITE 8: note = D-Valine
  SITE 9: note = Amidated Hydroxyproline
  SITE 2..7: note = side chain cyclized via a lactam bond
XDPXRWKVX

SEQ ID NO: 120 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Hydroxyproline
  SITE 2..7: note = side chain cyclized via a lactam bond
XDPXRWKVX

SEQ ID NO: 121 - protein, 9 aa, synthetic construct
  SITE 1: note = Acetylated Norleucine
  SITE 4: note = D-2'-naphthylalanine
  SITE 9: note = Amidated Hydroxyproline
  SITE 2..7: note = side chain cyclized via a lactam bond
XDPXRWKVX

SEQ ID NO: 122 - protein, 9 aa, synthetic construct
  SITE 1: note = Acetylated Norleucine
  SITE 4: note = D-2'-naphthylalanine
  SITE 9: note = Amidated D-Hydroxyproline
  SITE 2..7: note = side chain cyclized via a lactam bond
XDPXRWKVX

SEQ ID NO: 123 - protein, 9 aa, synthetic construct
  SITE 1: note = Acetylated Norleucine
  SITE 4: note = D-2'-naphthylalanine
  SITE 8: note = Hydroxyproline
  SITE 9: note = Amidated D-Valine
  SITE 2..7: note = side chain cyclized via a lactam bond
XDPXRWKXV

SEQ ID NO: 124 - protein, 9 aa, synthetic construct
  SITE 1: note = Acetylated Norleucine
  SITE 4: note = D-2'-naphthylalanine
  SITE 8: note = D-Hydroxyproline
  SITE 9: note = Amidated D-Valine
  SITE 2..7: note = side chain cyclized via a lactam bond
XDPXRWKXV

SEQ ID NO: 125 - protein, 9 aa, synthetic construct
  SITE 1: note = Acetylated Norleucine
  SITE 4: note = D-2'-naphthylalanine
  SITE 8: note = Hydroxyproline
  SITE 9: note = Amidated Residue
  SITE 2..7: note = side chain cyclized via a lactam bond
XDPXRWKXV

SEQ ID NO: 126 - protein, 9 aa, synthetic construct
  SITE 1: note = Acetylated Norleucine
  SITE 4: note = D-2'-naphthylalanine
  SITE 8: note = D-Hydroxyproline
  SITE 9: note = Amidated Residue
  SITE 2..7: note = side chain cyclized via a lactam bond
XDPXRWKXV

SEQ ID NO: 127 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Valine
  SITE 2..7: note = side chain cyclized via a lactam bond
XDPXRWKVV

SEQ ID NO: 128 - protein, 9 aa, synthetic construct
  SITE 1: note = Acetylated Norleucine
  SITE 4: note = D-2'-naphthylalanine
  SITE 8: note = D-Proline
  SITE 9: note = Amidated D-Proline
  SITE 2..7: note = side chain cyclized via a lactam bond
XDPXRWKPP

SEQ ID NO: 129 - protein, 8 aa, synthetic construct
  SITE 1: note = Acetylated Norleucine
  SITE 4: note = D-2'-naphthylalanine
  SITE 8: note = Amidated D-Valine
  SITE 2..7: note = side chain cyclized via a lactam bond
XDPXRWKV

SEQ ID NO: 130 - protein, 8 aa, synthetic construct
  SITE 1: note = Acetylated Norleucine
  SITE 4: note = D-2'-naphthylalanine
  SITE 8: note = Amidated D-Proline
  SITE 2..7: note = side chain cyclized via a lactam bond
XDPXRWKP

SEQ ID NO: 131 - protein, 8 aa, synthetic construct
  SITE 1: note = Acetylated Norleucine
  SITE 4: note = D-2'-naphthylalanine
  SITE 8: note = Amidated Residue
  SITE 2..7: note = side chain cyclized via a lactam bond
XDPXRWKV

SEQ ID NO: 132 - protein, 8 aa, synthetic construct
  SITE 1: note = Acetylated Norleucine
  SITE 4: note = D-2'-naphthylalanine
  SITE 8: note = Amidated Residue
  SITE 2..7: note = side chain cyclized via a lactam bond
XDPXRWKP

SEQ ID NO: 133 - protein, 8 aa, synthetic construct
  SITE 1: note = Acetylated Norleucine
  SITE 4: note = D-2'-naphthylalanine
  SITE 8: note = Amidated Residue
  SITE 2..7: note = side chain cyclized via a lactam bond
XDPXRWKA

SEQ ID NO: 134 - protein, 8 aa, synthetic construct
  SITE 1: note = Acetylated Norleucine
  SITE 4: note = D-2'-naphthylalanine
  SITE 8: note = Amidated D-Alanine
  SITE 2..7: note = side chain cyclized via a lactam bond
XDPXRWKA

SEQ ID NO: 135 - protein, 8 aa, synthetic construct
  SITE 1: note = Acetylated Norleucine
  SITE 4: note = D-2'-naphthylalanine
  SITE 8: note = Amidated D-Hydroxyproline
  SITE 2..7: note = side chain cyclized via a lactam bond
XDPXRWKX

SEQ ID NO: 136 - protein, 8 aa, synthetic construct
  SITE 1: note = Acetylated Norleucine
  SITE 4: note = D-2'-naphthylalanine
  SITE 8: note = Amidated Hydroxyproline
  SITE 2..7: note = side chain cyclized via a lactam bond
XDPXRWKX

SEQ ID NO: 137 - protein, 9 aa, synthetic construct
  SITE 1: note = Acetylated Norleucine
  SITE 4: note = D-2'-naphthylalanine
  SITE 8: note = D-Alanine
  SITE 9: note = Amidated D-Alanine
  SITE 2..7: note = side chain cyclized via a lactam bond
XDPXRWKAA

SEQ ID NO: 138 - protein, 9 aa, synthetic construct
  SITE 1: note = Acetylated Norleucine
  SITE 4: note = D-2'-naphthylalanine
  SITE 9: note = Amidated Residue
  SITE 2..7: note = side chain cyclized via a lactam bond
XDPXRWKAA

SEQ ID NO: 139 - protein, 9 aa, synthetic construct
  SITE 1: note = Acetylated Norleucine
  SITE 4: note = D-2'-naphthylalanine
  SITE 9: note = Amidated Residue
  SITE 2..7: note = side chain cyclized via a lactam bond
XDPXRWKGG

SEQ ID NO: 140 - protein, 9 aa, synthetic construct
  SITE 1: note = Acetylated Norleucine
  SITE 4: note = D-2'-naphthylalanine
  SITE 8: note = D-Valine
  SITE 9: note = Amidated Residue
  SITE 2..7: note = side chain cyclized via a lactam bond
XDPXRWKVD

SEQ ID NO: 141 - protein, 9 aa, synthetic construct
  SITE 1: note = Acetylated Norleucine
  SITE 4: note = D-2'-naphthylalanine
  SITE 8: note = D-Valine
  SITE 9: note = Amidated Residue
  SITE 2..7: note = side chain cyclized via a lactam bond
XDPXRWKVR

SEQ ID NO: 142 - protein, 9 aa, synthetic construct
  SITE 1: note = Acetylated Norleucine
  SITE 4: note = D-2'-naphthylalanine
  SITE 8: note = D-Valine
  SITE 9: note = Amidated Residue
  SITE 2..7: note = side chain cyclized via a lactam bond
XDPXRWKVN

SEQ ID NO: 143 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Aspartic Acid
  SITE 2..7: note = side chain cyclized via a lactam bond
XDPXRWKVD

SEQ ID NO: 144 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Arginine
  SITE 2..7: note = side chain cyclized via a lactam bond
XDPXRWKVR

SEQ ID NO: 145 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Asparagine
  SITE 2..7: note = side chain cyclized via a lactam bond
XDPXRWKVN

SEQ ID NO: 146 - protein, 9 aa, synthetic construct
  SITE 1: note = Acetylated Norleucine
  SITE 4: note = D-2'-naphthylalanine
  SITE 9: note = Amidated D-Proline
  SITE 2..7: note = side chain cyclized via a lactam bond
XDPXRWKDP

SEQ ID NO: 147 - protein, 9 aa, synthetic construct
  SITE 1: note = Acetylated Norleucine
  SITE 4: note = D-2'-naphthylalanine
  SITE 9: note = Amidated D-Proline
  SITE 2..7: note = side chain cyclized via a lactam bond
XDPXRWKRP

SEQ ID NO: 148 - protein, 9 aa, synthetic construct
  SITE 1: note = Acetylated Norleucine
  SITE 4: note = D-2'-naphthylalanine
  SITE 9: note = Amidated D-Proline
  SITE 2..7: note = side chain cyclized via a lactam bond
XDPXRWKNP

SEQ ID NO: 149 - protein, 9 aa, synthetic construct
  SITE 1: note = Acetylated Norleucine
  SITE 4: note = D-2'-naphthylalanine
  SITE 8: note = D-Aspartic Acid
  SITE 9: note = Amidated D-Proline
  SITE 2..7: note = side chain cyclized via a lactam bond
XDPXRWKDP

SEQ ID NO: 150 - protein, 9 aa, synthetic construct
  SITE 1: note = Acetylated Norleucine
  SITE 4: note = D-2'-naphthylalanine
  SITE 8: note = D-Arginine
  SITE 9: note = Amidated D-Proline
  SITE 2..7: note = side chain cyclized via a lactam bond
XDPXRWKRP

SEQ ID NO: 151 - protein, 9 aa, synthetic construct
  SITE 1: note = Acetylated Norleucine
  SITE 4: note = D-2'-naphthylalanine
  SITE 8: note = D-Asparagine
  SITE 9: note = Amidated D-Proline
  SITE 2..7: note = side chain cyclized via a lactam bond
XDPXRWKNP

SEQ ID NO: 152 - protein, 8 aa, synthetic construct
  SITE 1: note = Acetylated Norleucine
  SITE 4: note = D-2'-naphthylalanine
  SITE 8: note = Amidated Residue
  SITE 2..7: note = side chain cyclized via a lactam bond
XDPXRWKD

SEQ ID NO: 153 - protein, 8 aa, synthetic construct
  SITE 1: note = Acetylated Norleucine
  SITE 4: note = D-2'-naphthylalanine
  SITE 8: note = Amidated Residue
  SITE 2..7: note = side chain cyclized via a lactam bond
XDPXRWKR

SEQ ID NO: 154 - protein, 8 aa, synthetic construct
  SITE 1: note = Acetylated Norleucine
  SITE 4: note = D-2'-naphthylalanine
  SITE 8: note = Amidated Residue
  SITE 2..7: note = side chain cyclized via a lactam bond
XDPXRWKN

SEQ ID NO: 155 - protein, 8 aa, synthetic construct
  SITE 1: note = Acetylated Norleucine
  SITE 4: note = D-2'-naphthylalanine
  SITE 8: note = Amidated D-Aspartic Acid
  SITE 2..7: note = side chain cyclized via a lactam bond
XDPXRWKD

SEQ ID NO: 156 - protein, 8 aa, synthetic construct
  SITE 1: note = Acetylated Norleucine
  SITE 4: note = D-2'-naphthylalanine
  SITE 8: note = Amidated D-Arginine
  SITE 2..7: note = side chain cyclized via a lactam bond
XDPXRWKR

SEQ ID NO: 157 - protein, 8 aa, synthetic construct
  SITE 1: note = Acetylated Norleucine
  SITE 4: note = D-2'-naphthylalanine
  SITE 8: note = Amidated D-Asparagine
  SITE 2..7: note = side chain cyclized via a lactam bond
XDPXRWKN

SEQ ID NO: 158 - protein, 10 aa, synthetic construct
  SITE 1: note = Acetylated Norleucine
  SITE 4: note = D-2'-naphthylalanine
  SITE 10: note = Amidated Residue
  SITE 2..7: note = side chain cyclized via a lactam bond
XDPXRWKKPV

SEQ ID NO: 159 - protein, 10 aa, synthetic construct
  SITE 1: note = Acetylated Norleucine
  SITE 4: note = D-2'-naphthylalanine
  SITE 9: note = D-Proline
  SITE 10: note = Amidated D-Valine
  SITE 2..7: note = side chain cyclized via a lactam bond
XDPXRWKKPV

SEQ ID NO: 160 - protein, 10 aa, synthetic construct
  SITE 1: note = Acetylated Norleucine
  SITE 4: note = D-2'-naphthylalanine
  SITE 8: note = D-Lysine
  SITE 9: note = D-Proline
  SITE 10: note = Amidated D-Valine
  SITE 2..7: note = side chain cyclized via a lactam bond
XDPXRWKKPV

SEQ ID NO: 161 - protein, 9 aa, synthetic construct
  SITE 1: note = Acetylated Norleucine
  SITE 4: note = D-2'-naphthylalanine
  SITE 9: note = Amidated D-Proline
  SITE 2..7: note = side chain cyclized via a lactam bond
XDPXRWKKP

SEQ ID NO: 162 - protein, 9 aa, synthetic construct
  SITE 1: note = Acetylated Norleucine
  SITE 4: note = D-2'-naphthylalanine
  SITE 8: note = D-Lysine
  SITE 9: note = Amidated D-Proline
  SITE 2..7: note = side chain cyclized via a lactam bond
XDPXRWKKP

SEQ ID NO: 163 - protein, 10 aa, synthetic construct
  SITE 1: note = Acetylated Norleucine
  SITE 4: note = D-2'-naphthylalanine
  SITE 10: note = Amidated Residue
  SITE 2..7: note = side chain cyclized via a lactam bond
XDPXRWKKVP

SEQ ID NO: 164 - protein, 10 aa, synthetic construct
  SITE 1: note = Acetylated Norleucine
  SITE 4: note = D-2'-naphthylalanine
  SITE 9: note = D-Valine
  SITE 10: note = Amidated D-Proline
  SITE 2..7: note = side chain cyclized via a lactam bond
XDPXRWKKVP

SEQ ID NO: 165 - protein, 10 aa, synthetic construct
  SITE 1: note = Acetylated Norleucine
  SITE 4: note = D-2'-naphthylalanine
  SITE 8: note = D-Lysine
  SITE 9: note = D-Valine
  SITE 10: note = Amidated D-Proline
  SITE 2..7: note = side chain cyclized via a lactam bond
XDPXRWKKVP

SEQ ID NO: 166 - protein, 10 aa, synthetic construct
  SITE 1: note = Acetylated Norleucine
  SITE 4: note = D-2'-naphthylalanine
  SITE 10: note = Amidated Residue
  SITE 2..7: note = side chain cyclized via a lactam bond
XDPXRWKRPV

SEQ ID NO: 167 - protein, 10 aa, synthetic construct
  SITE 1: note = Acetylated Norleucine
  SITE 4: note = D-2'-naphthylalanine
  SITE 9: note = D-Proline
  SITE 10: note = Amidated D-Valine
  SITE 2..7: note = side chain cyclized via a lactam bond
XDPXRWKRPV

SEQ ID NO: 168 - protein, 10 aa, synthetic construct
  SITE 1: note = Acetylated Norleucine
  SITE 4: note = D-2'-naphthylalanine
  SITE 8: note = D-Arginine
  SITE 9: note = D-Proline
  SITE 10: note = Amidated D-Valine
  SITE 2..7: note = side chain cyclized via a lactam bond
XDPXRWKRPV

SEQ ID NO: 169 - protein, 10 aa, synthetic construct
  SITE 1: note = Acetylated Norleucine
  SITE 4: note = D-2'-naphthylalanine
  SITE 10: note = Amidated Residue
  SITE 2..7: note = side chain cyclized via a lactam bond
XDPXRWKRVP

SEQ ID NO: 170 - protein, 10 aa, synthetic construct
  SITE 1: note = Acetylated Norleucine
  SITE 4: note = D-2'-naphthylalanine
  SITE 9: note = D-Valine
  SITE 10: note = Amidated D-Proline
  SITE 2..7: note = side chain cyclized via a lactam bond
XDPXRWKRVP

SEQ ID NO: 171 - protein, 10 aa, synthetic construct
  SITE 1: note = Acetylated Norleucine
  SITE 4: note = D-2'-naphthylalanine
  SITE 8: note = D-Arginine
  SITE 9: note = D-Valine
  SITE 10: note = Amidated D-Proline
  SITE 2..7: note = side chain cyclized via a lactam bond
XDPXRWKRVP

SEQ ID NO: 172 - protein, 10 aa, synthetic construct
  SITE 1: note = Acetylated Norleucine
  SITE 4: note = D-2'-naphthylalanine
  SITE 8: note = D-Tert-Leucine
  SITE 9: note = D-Valine
  SITE 10: note = Amidated D-Proline
  SITE 2..7: note = side chain cyclized via a lactam bond
XDPXRWKXVP

SEQ ID NO: 173 - protein, 9 aa, synthetic construct
  SITE 1: note = Acetylated Norleucine
  SITE 4: note = D-2'-naphthylalanine
  SITE 8: note = D-Tert-Leucine
  SITE 9: note = Amidated D-Proline
  SITE 2..7: note = side chain cyclized via a lactam bond
XDPXRWKXP

SEQ ID NO: 174 - protein, 9 aa, synthetic construct
  SITE 1: note = Acetylated Norleucine
  SITE 4: note = D-2'-naphthylalanine
  SITE 8: note = D-Tert-Leucine
  SITE 9: note = Amidated D-Valine
  SITE 2..7: note = side chain cyclized via a lactam bond
XDPXRWKXV

SEQ ID NO: 175 - protein, 9 aa, synthetic construct
  SITE 1: note = Acetylated Norleucine
  SITE 4: note = D-2'-naphthylalanine
  SITE 8: note = D-Tert-Leucine
  SITE 9: note = Amidated D-Tert-Leucine
  SITE 2..7: note = side chain cyclized via a lactam bond
XDPXRWKXX

SEQ ID NO: 176 - protein, 10 aa, synthetic construct
  SITE 1: note = Acetylated Norleucine
  SITE 4: note = D-2'-naphthylalanine
  SITE 8: note = D-Tert-Leucine
  SITE 9: note = D-Tert-Leucine
  SITE 10: note = Amidated D-Proline
  SITE 2..7: note = side chain cyclized via a lactam bond
XDPXRWKXXP

SEQ ID NO: 177 - protein, 10 aa, synthetic construct
  SITE 1: note = Acetylated Norleucine
  SITE 4: note = D-2'-naphthylalanine
  SITE 8: note = D-Tert-Leucine
  SITE 9: note = D-Tert-Leucine
  SITE 10: note = Amidated D-Valine
  SITE 2..7: note = side chain cyclized via a lactam bond
XDPXRWKXXV

SEQ ID NO: 178 - protein, 9 aa, synthetic construct
  SITE 1: note = Acetylated Norleucine
  SITE 4: note = D-2'-naphthylalanine
  SITE 8: note = D-Proline
  SITE 9: note = Amidated D-Tert-Leucine
  SITE 2..7: note = side chain cyclized via a lactam bond
XDPXRWKPX

SEQ ID NO: 179 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Tert-Leucine
  SITE 2..7: note = side chain cyclized via a lactam bond
XDPXRWKVX

SEQ ID NO: 180 - protein, 10 aa, synthetic construct
  SITE 1: note = Acetylated Norleucine
  SITE 4: note = D-2'-naphthylalanine
  SITE 8: note = D-Valine
  SITE 9: note = D-Valine
  SITE 10: note = Amidated D-Proline
  SITE 2..7: note = side chain cyclized via a lactam bond
XDPXRWKVVP

SEQ ID NO: 181 - protein, 10 aa, synthetic construct
  SITE 1: note = Acetylated Norleucine
  SITE 4: note = D-2'-naphthylalanine
  SITE 8: note = D-Proline
  SITE 9: note = D-Valine
  SITE 10: note = Amidated D-Proline
  SITE 2..7: note = side chain cyclized via a lactam bond
XDPXRWKPVP

SEQ ID NO: 182 - protein, 11 aa, synthetic construct
  SITE 1: note = Acetylated Norleucine
  SITE 4: note = D-2'-naphthylalanine
  SITE 8: note = D-Valine
  SITE 9: note = D-Valine
  SITE 10: note = D-Valine
  SITE 11: note = Amidated D-Proline
  SITE 2..7: note = side chain cyclized via a lactam bond
XDPXRWKVVVP

SEQ ID NO: 183 - protein, 11 aa, synthetic construct
  SITE 1: note = Acetylated Norleucine
  SITE 4: note = D-2'-naphthylalanine
  SITE 8: note = D-Valine
  SITE 9: note = D-Proline
  SITE 10: note = D-Valine
  SITE 11: note = Amidated D-Proline
  SITE 2..7: note = side chain cyclized via a lactam bond
XDPXRWKVPVP

SEQ ID NO: 184 - protein, 14 aa, synthetic construct
  SITE 1: note = Acetylated Norleucine
  SITE 4: note = D-2'-naphthylalanine
  SITE 8: note = D-Valine
  SITE 9: note = D-Valine
  SITE 10: note = D-Valine
  SITE 11: note = D-Valine
  SITE 12: note = D-Valine
  SITE 13: note = D-Valine
  SITE 14: note = Amidated D-Proline
  SITE 2..7: note = side chain cyclized via a lactam bond
XDPXRWKVVVVVVP

SEQ ID NO: 185 - protein, 10 aa, synthetic construct
  SITE 1: note = Acetylated Norleucine
  SITE 2: note = Norleucine
  SITE 5: note = D-2'-naphthylalanine
  SITE 9: note = D-Valine
  SITE 10: note = Amidated D-Proline
  SITE 3..8: note = side chain cyclized via a lactam bond
XXDPXRWKVP

SEQ ID NO: 186 - protein, 11 aa, synthetic construct
  SITE 1: note = Acetylated Norleucine
  SITE 2: note = Norleucine
  SITE 3: note = Norleucine
  SITE 6: note = D-2'-naphthylalanine
  SITE 10: note = D-Valine
  SITE 11: note = Amidated D-Proline
  SITE 4..9: note = side chain cyclized via a lactam bond
XXXDPXRWKVP

SEQ ID NO: 187 - protein, 12 aa, synthetic construct
  SITE 1: note = Acetylated Norleucine
  SITE 2: note = Norleucine
  SITE 3: note = Norleucine
  SITE 4: note = Norleucine
  SITE 7: note = D-2'-naphthylalanine
  SITE 11: note = D-Valine
  SITE 12: note = Amidated D-Proline
  SITE 5..10: note = side chain cyclized via a lactam bond
XXXXDPXRWKVP

SEQ ID NO: 188 - protein, 9 aa, synthetic construct
  SITE 1: note = Acetylated Norleucine
  SITE 4: note = D-Phenylalanine
  SITE 6: note = D-Tryptophan
  SITE 8: note = D-Valine
  SITE 9: note = Amidated D-Proline
  VARIANT 2..7: note = cyclization may occur via a lactam bond or disulfide bond
XDPFRWKVP

SEQ ID NO: 189 - protein, 10 aa, synthetic construct
  SITE 1: note = Acetylated Norleucine
  SITE 5: note = Para-iodo-D-phenylalanine
  SITE 7: note = Tetrahydro-isoquinoline-3-carboxylic acid
  SITE 9: note = D-Valine
  SITE 10: note = Amidated D-Proline
  VARIANT 2..8: note = cyclization may occur via a lactam bond or disulfide bond
XDHRXRXKVP

SEQ ID NO: 190 - protein, 10 aa, synthetic construct
  SITE 1: note = Acetylated Norleucine
  SITE 5: note = D-Biphenyl amino acid
  SITE 7: note = Tetrahydro-isoquinoline-3-carboxylic acid
  SITE 9: note = D-Valine
  SITE 10: note = Amidated D-Proline
  VARIANT 2..8: note = cyclization may occur via a lactam bond or disulfide bond
XDHRXRXKVP

SEQ ID NO: 191 - protein, 10 aa, synthetic construct
  SITE 1: note = Acetylated Norleucine
  SITE 5: note = Para-iodo-D-phenylalanine
  SITE 7: note = Tetrahydro-isoquinoline-3-carboxylic acid
  SITE 9: note = D-Valine
  SITE 10: note = Amidated D-Proline
  VARIANT 2..8: note = cyclization may occur via a lactam bond or disulfide bond
XDPRXRXKVP

SEQ ID NO: 192 - protein, 10 aa, synthetic construct
  SITE 1: note = Acetylated Norleucine
  SITE 5: note = D-Biphenyl amino acid
  SITE 7: note = Tetrahydro-isoquinoline-3-carboxylic acid
  SITE 9: note = D-Valine
  SITE 10: note = Amidated D-Proline
  VARIANT 2..8: note = cyclization may occur via a lactam bond or disulfide bond
XDPRXRXKVP

SEQ ID NO: 193 - protein, 10 aa, synthetic construct
  SITE 1: note = Acetylated Norleucine
  SITE 5: note = Para-iodo-D-phenylalanine
  SITE 7: note = Tetrahydro-isoquinoline-3-carboxylic acid
  SITE 9: note = D-Valine
  SITE 10: note = Amidated D-Proline
  VARIANT 2..8: note = cyclization may occur via a lactam bond or disulfide bond
XDRPXRXKVP

SEQ ID NO: 194 - protein, 10 aa, synthetic construct
  SITE 1: note = Acetylated Norleucine
  SITE 5: note = D-Biphenyl amino acid
  SITE 7: note = Tetrahydro-isoquinoline-3-carboxylic acid
  SITE 9: note = D-Valine
  SITE 10: note = Amidated D-Proline
  VARIANT 2..8: note = cyclization may occur via a lactam bond or disulfide bond
XDRPXRXKVP

SEQ ID NO: 195 - protein, 10 aa, synthetic construct
  SITE 1: note = Acetylated Norleucine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
XDPPXRWKVP

SEQ ID NO: 196 - protein, 10 aa, synthetic construct
  SITE 1: note = Acetylated Norleucine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
XDWPXRWKVP

SEQ ID NO: 197 - protein, 9 aa, synthetic construct
  SITE 1: note = Acetylated Norleucine
  SITE 4: note = D-2'-naphthylalanine
  SITE 6: note = D-Tryptophan
  SITE 8: note = D-Valine
  SITE 9: note = Amidated D-Proline
  VARIANT 2..7: note = cyclization may occur via a lactam bond or disulfide bond
XDPXRWKVP

SEQ ID NO: 198 - protein, 7 aa, synthetic construct
  SITE 1: note = CO-cis-CH=CH-CO moiety attached to Proline
  SITE 2: note = D-2'-naphthylalanine
  SITE 6: note = D-Valine
  SITE 7: note = Amidated D-Proline
  VARIANT 1..5: note = cyclization may occur via a lactam bond or disulfide bond
PXRWKVP

SEQ ID NO: 199 - protein, 9 aa, synthetic construct
  SITE 1: note = Acetylated Norleucine
  SITE 3: note = 4-amino-1,2,4,5-tetrahydro-2-benzazepin-3-one
  SITE 4: note = D-Phenylalanine
  SITE 8: note = D-Valine
  SITE 9: note = Amidated D-Proline
  VARIANT 2..7: note = cyclization may occur via a lactam bond or disulfide bond
XDXFRWKVP

SEQ ID NO: 200 - protein, 9 aa, synthetic construct
  SITE 1: note = Acetylated Norleucine
  SITE 3: note = beta-Alanine
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 201 - protein, 9 aa, synthetic construct
  SITE 1: note = Acetylated Norleucine
  SITE 3: note = 3-aminomethylbenzoic acid
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 202 - protein, 9 aa, synthetic construct
  SITE 1: note = Acetylated Norleucine
  SITE 3: note = 1-aminocyclo-propane-1-carboxylic acid
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 203 - protein, 7 aa, synthetic construct
  SITE 1: note = Acetylated Residue
  SITE 3: note = D-Phenylalanine
  SITE 6: note = D-Valine
  SITE 7: note = Amidated D-Proline
  VARIANT 1..4: note = cyclization may occur via a lactam bond or disulfide bond
CRFCWVP

SEQ ID NO: 204 - protein, 8 aa, synthetic construct
  SITE 1: note = Acetylated Norleucine
  SITE 4: note = D-2'-naphthylalanine
  SITE 8: note = Amidated Residue
  VARIANT 2..7: note = cyclization may occur via a lactam bond or disulfide bond
XDPXRWKW

SEQ ID NO: 205 - protein, 9 aa, synthetic construct
  SITE 1: note = Acetylated Norleucine
  SITE 3: note = 4-amino-1,2,4,5-tetrahydro-2-benzazepin-3-one
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 206 - protein, 9 aa, synthetic construct
  SITE 1: note = Acetylated Norleucine
  SITE 3: note = 4-amino-1,4,5,6-tetrahydroazepino[4,3-b]indol-3(2H) -one
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 207 - protein, 9 aa, synthetic construct
  SITE 1: note = Acetylated Norleucine
  SITE 3: note = 7-amino-7,8-dihydro-4H-[1,2,3]triazolo-[1,5-a][1,4] diazepin-6(5H)-one
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 208 - protein, 9 aa, synthetic construct
  SITE 1: note = Acetylated Norleucine
  SITE 3: note = 4-amino-1,4,5,6-tetrahydroazepino[4,3-b]indol-3(2H) -one
  SITE 4: note = D-Phenylalanine
  SITE 8: note = D-Valine
  SITE 9: note = Amidated D-Proline
  VARIANT 2..7: note = cyclization may occur via a lactam bond or disulfide bond
XDXFRWKVP

SEQ ID NO: 209 - protein, 9 aa, synthetic construct
  SITE 1: note = Acetylated Norleucine
  SITE 3: note = 7-amino-7,8-dihydro-4H-[1,2,3]triazolo-[1,5-a][1,4] diazepin-6(5H)-one
  SITE 4: note = D-Phenylalanine
  SITE 8: note = D-Valine
  SITE 9: note = Amidated D-Proline
  VARIANT 2..7: note = cyclization may occur via a lactam bond or disulfide bond
XDXFRWKVP

SEQ ID NO: 210 - protein, 9 aa, synthetic construct
  SITE 1: note = Acetylated Norleucine
  SITE 3: note = 4-amino-1,2,4,5-tetrahydro-2-benzazepin-3-one
  SITE 4: note = Para-chloro-D-phenylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 211 - protein, 9 aa, synthetic construct
  SITE 1: note = Acetylated Norleucine
  SITE 3: note = 4-amino-1,4,5,6-tetrahydroazepino[4,3-b]indol-3(2H) -one
  SITE 4: note = Para-chloro-D-phenylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 212 - protein, 9 aa, synthetic construct
  SITE 1: note = Acetylated Norleucine
  SITE 3: note = 7-amino-7,8-dihydro-4H-[1,2,3]triazolo-[1,5-a][1,4] diazepin-6(5H)-one
  SITE 4: note = Para-chloro-D-phenylalanine
  SITE 8: note = D-Valine
  SITE 9: note = Amidated D-Proline
  VARIANT 2..7: note = cyclization may occur via a lactam bond or disulfide bond
XDXXRWKVP

SEQ ID NO: 213 - protein, 9 aa, synthetic construct
  SITE 1: note = Acetylated Norleucine
  SITE 3: note = 2-aminotetraline-2-carboxylic acid
  SITE 4: note = D-Phenylalanine
  SITE 8: note = D-Valine
  SITE 9: note = Amidated D-Proline
  SITE 2..7: note = side chain cyclized via a lactam bond
XDXFRWKVP

SEQ ID NO: 214 - protein, 9 aa, synthetic construct
  SITE 1: note = Acetylated Norleucine
  SITE 3: note = 1-amino-4-phenylcyclohexane-carboxylic acid
  SITE 4: note = D-Phenylalanine
  SITE 8: note = D-Valine
  SITE 9: note = Amidated D-Proline
  SITE 2..7: note = side chain cyclized via a lactam bond
XDXFRWKVP

SEQ ID NO: 215 - protein, 9 aa, synthetic construct
  SITE 1: note = Acetylated Norleucine
  SITE 3: note = 4-aminophenylpiperidine-4-carboxylic acid
  SITE 4: note = D-Phenylalanine
  SITE 8: note = D-Valine
  SITE 9: note = Amidated D-Proline
  SITE 2..7: note = side chain cyclized via a lactam bond
XDXFRWKVP

SEQ ID NO: 216 - protein, 9 aa, synthetic construct
  SITE 1: note = Acetylated Norleucine
  SITE 4: note = Para-chloro-D-phenylalanine
  SITE 8: note = D-Valine
  SITE 9: note = Amidated D-Proline
  SITE 2..7: note = side chain cyclized via a lactam bond
XDPXRWKVP

SEQ ID NO: 217 - protein, 9 aa, synthetic construct
  SITE 1: note = Acetylated Norleucine
  SITE 4: note = Para-iodo-D-phenylalanine
  SITE 8: note = D-Valine
  SITE 9: note = Amidated D-Proline
  SITE 2..7: note = side chain cyclized via a lactam bond
XDPXRWKVP

SEQ ID NO: 218 - protein, 9 aa, synthetic construct
  SITE 1: note = Acetylated Norleucine
  SITE 4: note = Para-bromo-D-phenylalanine
  SITE 8: note = D-Valine
  SITE 9: note = Amidated D-Proline
  SITE 2..7: note = side chain cyclized via a lactam bond
XDPXRWKVP

SEQ ID NO: 219 - protein, 9 aa, synthetic construct
  SITE 1: note = Acetylated Norleucine
  SITE 4: note = Para-fluoro-D-phenylalanine
  SITE 8: note = D-Valine
  SITE 9: note = Amidated D-Proline
  SITE 2..7: note = side chain cyclized via a lactam bond
XDPXRWKVP

SEQ ID NO: 220 - protein, 9 aa, synthetic construct
  SITE 1: note = Acetylated Norleucine
  SITE 4: note = Para-trifluoromethyl-D-phenylalanine
  SITE 8: note = D-Valine
  SITE 9: note = Amidated D-Proline
  SITE 2..7: note = side chain cyclized via a lactam bond
XDPXRWKVP

SEQ ID NO: 221 - protein, 9 aa, synthetic construct
  SITE 1: note = Acetylated Norleucine
  SITE 4: note = Para-chloro-D-phenylalanine
  SITE 8: note = D-Valine
  SITE 9: note = Amidated D-Proline
  SITE 2..7: note = side chain cyclized via a lactam bond
XDHXRWKVP

SEQ ID NO: 222 - protein, 9 aa, synthetic construct
  SITE 1: note = Acetylated Norleucine
  SITE 4: note = Para-iodo-D-phenylalanine
  SITE 8: note = D-Valine
  SITE 9: note = Amidated D-Proline
  SITE 2..7: note = side chain cyclized via a lactam bond
XDHXRWKVP

SEQ ID NO: 223 - protein, 9 aa, synthetic construct
  SITE 1: note = Acetylated Norleucine
  SITE 4: note = Para-bromo-D-phenylalanine
  SITE 8: note = D-Valine
  SITE 9: note = Amidated D-Proline
  SITE 2..7: note = side chain cyclized via a lactam bond
XDHXRWKVP

SEQ ID NO: 224 - protein, 9 aa, synthetic construct
  SITE 1: note = Acetylated Norleucine
  SITE 4: note = Para-fluoro-D-phenylalanine
  SITE 8: note = D-Valine
  SITE 9: note = Amidated D-Proline
  SITE 2..7: note = side chain cyclized via a lactam bond
XDHXRWKVP

SEQ ID NO: 225 - protein, 9 aa, synthetic construct
  SITE 1: note = Acetylated Norleucine
  SITE 4: note = Para-trifluoromethyl-D-phenylalanine
  SITE 8: note = D-Valine
  SITE 9: note = Amidated D-Proline
  SITE 2..7: note = side chain cyclized via a lactam bond
XDHXRWKVP

SEQ ID NO: 226 - protein, 10 aa, synthetic construct
  SITE 1: note = Acetylated Norleucine
  SITE 5: note = D-Phenylalanine
  SITE 9: note = D-Valine
  SITE 10: note = Amidated D-Proline
  SITE 2..8: note = side chain cyclized via a lactam bond
XDPPFRWKVP

SEQ ID NO: 227 - protein, 10 aa, synthetic construct
  SITE 1: note = Acetylated Norleucine
  SITE 5: note = D-Phenylalanine
  SITE 9: note = D-Valine
  SITE 10: note = Amidated D-Proline
  SITE 2..8: note = side chain cyclized via a lactam bond
XDWPFRWKVP

SEQ ID NO: 228 - protein, 9 aa, synthetic construct
  SITE 1: note = Acetylated Norleucine
  SITE 4: note = D-Phenylalanine
  SITE 8: note = D-Valine
  SITE 9: note = Amidated D-Proline
  SITE 2..7: note = side chain cyclized via a lactam bond
XDHFPWKVP

SEQ ID NO: 229 - protein, 9 aa, synthetic construct
  SITE 1: note = Acetylated Norleucine
  SITE 4: note = D-Phenylalanine
  SITE 5: note = trans-4-guanidinyl-proline
  SITE 8: note = D-Valine
  SITE 9: note = Amidated D-Proline
  SITE 2..7: note = side chain cyclized via a lactam bond
XDHFXWKVP

SEQ ID NO: 230 - protein, 9 aa, synthetic construct
  SITE 1: note = Acetylated Norleucine
  SITE 4: note = D-Phenylalanine
  SITE 5: note = cis-4-guanidinyl-proline
  SITE 8: note = D-Valine
  SITE 9: note = Amidated D-Proline
  SITE 2..7: note = side chain cyclized via a lactam bond
XDHFXWKVP

SEQ ID NO: 231 - protein, 9 aa, synthetic construct
  SITE 1: note = Acetylated Norleucine
  SITE 4: note = D-Phenylalanine
  SITE 8: note = D-Valine
  SITE 9: note = Amidated D-Proline
  SITE 2..7: note = side chain cyclized via a lactam bond
XDPFPWKVP

SEQ ID NO: 232 - protein, 9 aa, synthetic construct
  SITE 1: note = Acetylated Norleucine
  SITE 4: note = D-Phenylalanine
  SITE 5: note = trans-4-guanidinyl-proline
  SITE 8: note = D-Valine
  SITE 9: note = Amidated D-Proline
  SITE 2..7: note = side chain cyclized via a lactam bond
XDPFXWKVP

SEQ ID NO: 233 - protein, 9 aa, synthetic construct
  SITE 1: note = Acetylated Norleucine
  SITE 4: note = D-Phenylalanine
  SITE 5: note = cis-4-guanidinyl-proline
  SITE 8: note = D-Valine
  SITE 9: note = Amidated D-Proline
  SITE 2..7: note = side chain cyclized via a lactam bond
XDPFXWKVP

SEQ ID NO: 234 - protein, 9 aa, synthetic construct
  SITE 1: note = Acetylated Norleucine
  SITE 4: note = D-Phenylalanine
  SITE 6: note = 4-amino-1,4,5,6-tetrahydroazepino[4,3-b]indol-3(2H) -one
  SITE 8: note = D-Valine
  SITE 9: note = Amidated D-Proline
  SITE 2..7: note = side chain cyclized via a lactam bond
XDPFRXKVP

SEQ ID NO: 235 - protein, 9 aa, synthetic construct
  SITE 1: note = Acetylated Norleucine
  SITE 4: note = D-Phenylalanine
  SITE 6: note = 4-amino-1,2,4,5-tetrahydro-2-benzazepin-3-one
  SITE 8: note = D-Valine
  SITE 9: note = Amidated D-Proline
  SITE 2..7: note = side chain cyclized via a lactam bond
XDPFRXKVP

SEQ ID NO: 236 - protein, 9 aa, synthetic construct
  SITE 1: note = Acetylated Norleucine
  SITE 4: note = D-Phenylalanine
  SITE 6: note = 7-amino-7,8-dihydro-4H-[1,2,3]triazolo-[1,5-a][1,4] diazepin-6(5H)-one
  SITE 8: note = D-Valine
  SITE 9: note = Amidated D-Proline
  SITE 2..7: note = side chain cyclized via a lactam bond
XDPFRXKVP

SEQ ID NO: 237 - protein, 9 aa, synthetic construct
  SITE 1: note = Acetylated Norleucine
  SITE 4: note = Para-trifluoromethyl-D-phenylalanine
  SITE 6: note = 4-amino-1,4,5,6-tetrahydroazepino[4,3-b]indol-3(2H) -one
  SITE 8: note = D-Valine
  SITE 9: note = Amidated D-Proline
  SITE 2..7: note = side chain cyclized via a lactam bond
XDPXRXKVP

SEQ ID NO: 238 - protein, 9 aa, synthetic construct
  SITE 1: note = Acetylated Norleucine
  SITE 4: note = Para-trifluoromethyl-D-phenylalanine
  SITE 6: note = 4-amino-1,2,4,5-tetrahydro-2-benzazepin-3-one
  SITE 8: note = D-Valine
  SITE 9: note = Amidated D-Proline
  SITE 2..7: note = side chain cyclized via a lactam bond
XDPXRXKVP

SEQ ID NO: 239 - protein, 9 aa, synthetic construct
  SITE 1: note = Acetylated Norleucine
  SITE 4: note = Para-trifluoromethyl-D-phenylalanine
  SITE 6: note = 7-amino-7,8-dihydro-4H-[1,2,3]triazolo-[1,5-a][1,4] diazepin-6(5H)-one
  SITE 8: note = D-Valine
  SITE 9: note = Amidated D-Proline
  SITE 2..7: note = side chain cyclized via a lactam bond
XDPXRXKVP

SEQ ID NO: 240 - protein, 9 aa, synthetic construct
  SITE 1: note = Acetylated Norleucine
  SITE 3: note = 2-aminoindone-2-carboxylic acid
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 241 - protein, 9 aa, synthetic construct
  SITE 1: note = Acetylated Norleucine
  SITE 3: note = 1-amino-1-cyclopentane carboxylic
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 242 - protein, 9 aa, synthetic construct
  SITE 1: note = Acetylated Norleucine
  SITE 3: note = 1-amino-1-cyclohexanecarboxylic acid
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 243 - protein, 9 aa, synthetic construct
  SITE 1: note = Acetylated Norleucine
  SITE 3: note = Octohydroindole-2-carboxylic acid
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 244 - protein, 9 aa, synthetic construct
  SITE 1: note = Acetylated Norleucine
  SITE 3: note = Indoline-2-carboxylic acid
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 245 - protein, 9 aa, synthetic construct
  SITE 1: note = Acetylated Norleucine
  SITE 3: note = Tetrahydro-isoquinoline-3-carboxylic acid
  SITE 4: note = D-2'-naphthylalanine
  SITE 8: note = D-Valine
  SITE 9: note = Amidated D-Proline
  SITE 2..7: note = side chain cyclized via a lactam bond
XDXXRWKVP

SEQ ID NO: 246 - protein, 10 aa, synthetic construct
  SITE 1: note = Acetylated Norleucine
  SITE 4: note = D-2'-naphthylalanine
  SITE 9: note = D-Valine
  SITE 10: note = Amidated D-Proline
  SITE 2..8: note = side chain cyclized via a lactam bond
XDPXRWPKVP

SEQ ID NO: 247 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDHXPWKVP

SEQ ID NO: 248 - protein, 9 aa, synthetic construct
  SITE 1: note = Acetylated Norleucine
  SITE 4: note = D-2'-naphthylalanine
  SITE 5: note = trans-4-guanidinyl-proline
  SITE 8: note = D-Valine
  SITE 9: note = Amidated D-Proline
  SITE 2..7: note = side chain cyclized via a lactam bond
XDHXXWKVP

SEQ ID NO: 249 - protein, 9 aa, synthetic construct
  SITE 1: note = Acetylated Norleucine
  SITE 4: note = D-2'-naphthylalanine
  SITE 5: note = cis-4-guanidinyl-proline
  SITE 8: note = D-Valine
  SITE 9: note = Amidated D-Proline
  SITE 2..7: note = side chain cyclized via a lactam bond
XDHXXWKVP

SEQ ID NO: 250 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDPXPWKVP

SEQ ID NO: 251 - protein, 9 aa, synthetic construct
  SITE 1: note = Acetylated Norleucine
  SITE 4: note = D-2'-naphthylalanine
  SITE 5: note = trans-4-guanidinyl-proline
  SITE 8: note = D-Valine
  SITE 9: note = Amidated D-Proline
  SITE 2..7: note = side chain cyclized via a lactam bond
XDPXXWKVP

SEQ ID NO: 252 - protein, 9 aa, synthetic construct
  SITE 1: note = Acetylated Norleucine
  SITE 4: note = D-2'-naphthylalanine
  SITE 5: note = cis-4-guanidinyl-proline
  SITE 8: note = D-Valine
  SITE 9: note = Amidated D-Proline
  SITE 2..7: note = side chain cyclized via a lactam bond
XDPXXWKVP

SEQ ID NO: 253 - protein, 9 aa, synthetic construct
  SITE 1: note = Acetylated Norleucine
  SITE 4: note = D-2'-naphthylalanine
  SITE 6: note = 4-amino-1,4,5,6-tetrahydroazepino[4,3-b]indol-3(2H) -one
  SITE 8: note = D-Valine
  SITE 9: note = Amidated D-Proline
  SITE 2..7: note = side chain cyclized via a lactam bond
XDPXRXKVP

SEQ ID NO: 254 - protein, 9 aa, synthetic construct
  SITE 1: note = Acetylated Norleucine
  SITE 4: note = D-2'-naphthylalanine
  SITE 6: note = 4-amino-1,2,4,5-tetrahydro-2-benzazepin-3-one
  SITE 8: note = D-Valine
  SITE 9: note = Amidated D-Proline
  SITE 2..7: note = side chain cyclized via a lactam bond
XDPXRXKVP

SEQ ID NO: 255 - protein, 9 aa, synthetic construct
  SITE 1: note = Acetylated Norleucine
  SITE 4: note = D-2'-naphthylalanine
  SITE 6: note = 7-amino-7,8-dihydro-4H-[1,2,3]triazolo-[1,5-a][1,4] diazepin-6(5H)-one
  SITE 8: note = D-Valine
  SITE 9: note = Amidated D-Proline
  SITE 2..7: note = side chain cyclized via a lactam bond
XDPXRXKVP

SEQ ID NO: 256 - protein, 9 aa, synthetic construct
  SITE 1: note = Acetylated Norleucine
  SITE 4: note = D-2'-naphthylalanine
  SITE 8: note = D-Valine
  SITE 9: note = Amidated D-Proline
  SITE 2..7: note = side chain cyclized via a lactam bond
XDEXRWKVP

SEQ ID NO: 257 - protein, 10 aa, synthetic construct
  SITE 1: note = Acetylated Norleucine
  SITE 4: note = D-2'-naphthylalanine
  SITE 9: note = D-Valine
  SITE 10: note = Amidated D-Proline
  SITE 2..8: note = side chain cyclized via a lactam bond
XDEXRWGKVP

SEQ ID NO: 258 - protein, 10 aa, synthetic construct
  SITE 1: note = Acetylated Norleucine
  SITE 5: note = D-2'-naphthylalanine
  SITE 9: note = D-Valine
  SITE 10: note = Amidated D-Proline
  SITE 2..8: note = side chain cyclized via a lactam bond
XDPEXRWKVP

SEQ ID NO: 259 - protein, 11 aa, synthetic construct
  SITE 1: note = Acetylated Norleucine
  SITE 5: note = D-2'-naphthylalanine
  SITE 10: note = D-Valine
  SITE 11: note = Amidated D-Proline
  SITE 2..9: note = side chain cyclized via a lactam bond
XDPEXRWGKVP

SEQ ID NO: 260 - protein, 10 aa, synthetic construct
  SITE 1: note = Acetylated Norleucine
  SITE 4: note = D-2'-naphthylalanine
  SITE 9: note = D-Valine
  SITE 10: note = Amidated D-Proline
  SITE 2..8: note = side chain cyclized via a lactam bond
XDPXRWGKVP

SEQ ID NO: 261 - protein, 12 aa, synthetic construct
  SITE 1: note = Acetylated Norleucine
  SITE 4: note = D-2'-naphthylalanine
  SITE 9: note = D-Proline
  SITE 10: note = D-Proline
  SITE 12: note = Amidated Residue
  SITE 2..8: note = side chain cyclized via a lactam bond
XDPXRWGKPPKD

SEQ ID NO: 262 - protein, 12 aa, synthetic construct
  SITE 1: note = Acetylated Norleucine
  SITE 4: note = D-2'-naphthylalanine
  SITE 9: note = D-Proline
  SITE 10: note = D-Proline
  SITE 11: note = D-Lysine
  SITE 12: note = Amidated D-Aspartic Acid
  SITE 2..8: note = side chain cyclized via a lactam bond
XDPXRWGKPPKD

SEQ ID NO: 263 - protein, 12 aa, synthetic construct
  SITE 1: note = Acetylated Residue
  SITE 4: note = D-2'-naphthylalanine
  SITE 9: note = D-Proline
  SITE 10: note = D-Proline
  SITE 12: note = Amidated Residue
  SITE 2..8: note = side chain cyclized via a lactam bond
EDPXRWGKPPKD

SEQ ID NO: 264 - protein, 11 aa, synthetic construct
  SITE 1: note = Acetylated Norleucine
  SITE 4: note = D-2'-naphthylalanine
  SITE 8: note = D-Proline
  SITE 9: note = D-Proline
  SITE 10: note = D-Lysine
  SITE 11: note = Amidated D-Aspartic Acid
  SITE 2..7: note = side chain cyclized via a lactam bond
XDPXRWKPPKD

SEQ ID NO: 265 - protein, 10 aa, synthetic construct
  SITE 1: note = Acetylated Norleucine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
XDEHXRWKVP

SEQ ID NO: 266 - protein, 11 aa, synthetic construct
  SITE 1: note = Acetylated Norleucine
  SITE 5: note = D-2'-naphthylalanine
  SITE 10: note = D-Valine
  SITE 11: note = Amidated D-Proline
  VARIANT 2..9: note = cyclization may occur via a lactam bond or disulfide bond
XDEHXRWGKVP

SEQ ID NO: 267 - protein, 10 aa, synthetic construct
  SITE 1: note = Acetylated Norleucine
  SITE 3: note = D-Alanine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
XDAHXRWKVP

SEQ ID NO: 268 - protein, 10 aa, synthetic construct
  SITE 1: note = Acetylated Residue
  SITE 3: note = D-Alanine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
RDAHXRWKVP

SEQ ID NO: 269 - protein, 10 aa, synthetic construct
  SITE 1: note = Acetylated Residue
  SITE 3: note = D-Alanine
  SITE 5: note = D-Phenylalanine
  SITE 9: note = D-Valine
  SITE 10: note = Amidated D-Proline
  VARIANT 2..8: note = cyclization may occur via a lactam bond or disulfide bond
RDAHFRWKVP

SEQ ID NO: 270 - protein, 10 aa, synthetic construct
  SITE 1: note = Acetylated Residue
  SITE 3: note = D-Alanine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
RCAHXRWCVP

SEQ ID NO: 271 - protein, 10 aa, synthetic construct
  SITE 1: note = Acetylated D-Arginine
  SITE 3: note = D-Alanine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
RDAHXRWKVP

SEQ ID NO: 272 - protein, 9 aa, synthetic construct
  SITE 1: note = Acetylated Residue
  SITE 3: note = D-Alanine
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
RDAXRWKVP

SEQ ID NO: 273 - protein, 9 aa, synthetic construct
  SITE 1: note = Acetylated D-Arginine
  SITE 3: note = D-Alanine
  SITE 4: note = D-2'-naphthylalanine
  SITE 8: note = D-Valine
  SITE 9: note = Amidated D-Proline
  VARIANT 2..7: note = cyclization may occur via a lactam bond or disulfide bond
RDAXRWKVP

SEQ ID NO: 274 - protein, 10 aa, synthetic construct
  SITE 1: note = Acetylated Norleucine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
XDAHXRWKVP

SEQ ID NO: 275 - protein, 10 aa, synthetic construct
  SITE 1: note = Acetylated Residue
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
RDAHXRWKVP

SEQ ID NO: 276 - protein, 10 aa, synthetic construct
  SITE 1: note = Acetylated D-Arginine
  SITE 5: note = D-2'-naphthylalanine
  SITE 9: note = D-Valine
  SITE 10: note = Amidated D-Proline
  VARIANT 2..8: note = cyclization may occur via a lactam bond or disulfide bond
RDAHXRWKVP

SEQ ID NO: 277 - protein, 9 aa, synthetic construct
  SITE 1: note = Acetylated Norleucine
  SITE 5: note = D-2'-naphthylalanine
  SITE 8: note = D-Valine
  SITE 9: note = Amidated D-Proline
  VARIANT 2..7: note = cyclization may occur via a lactam bond or disulfide bond
XDWRXPKVP

SEQ ID NO: 278 - protein, 8 aa, synthetic construct
  SITE 1: note = Acetylated Norleucine
  SITE 5: note = D-2'-naphthylalanine
  SITE 7: note = D-Valine
  SITE 8: note = Amidated D-Proline
  VARIANT 2..6: note = cyclization may occur via a lactam bond or disulfide bond
XDPWXKVP

SEQ ID NO: 279 - protein, 10 aa, synthetic construct
  SITE 1: note = Acetylated Norleucine
  SITE 6: note = D-2'-naphthylalanine
  SITE 9: note = D-Valine
  SITE 10: note = Amidated D-Proline
  VARIANT 2..8: note = cyclization may occur via a lactam bond or disulfide bond
XDPWRXPKVP

SEQ ID NO: 280 - protein, 9 aa, synthetic construct
  SITE 1: note = Acetylated Norleucine
  SITE 6: note = D-2'-naphthylalanine
  SITE 8: note = D-Valine
  SITE 9: note = Amidated D-Proline
  VARIANT 2..7: note = cyclization may occur via a lactam bond or disulfide bond
XDPWRXKVP

SEQ ID NO: 281 - protein, 9 aa, synthetic construct
  SITE 1: note = Acetylated Norleucine
  SITE 5: note = D-2'-naphthylalanine
  SITE 8: note = D-Valine
  SITE 9: note = Amidated D-Proline
  VARIANT 2..7: note = cyclization may occur via a lactam bond or disulfide bond
XKWRXPDVP

SEQ ID NO: 282 - protein, 8 aa, synthetic construct
  SITE 1: note = Acetylated Residue
  SITE 3: note = D-Alanine
  SITE 5: note = D-Phenylalanine
  SITE 8: note = Amidated Residue
  VARIANT 2..8: note = cyclization may occur via a lactam bond or disulfide bond
RDAHFRWK

SEQ ID NO: 283 - protein, 8 aa, synthetic construct
  SITE 1: note = Acetylated Residue
  SITE 3: note = D-Alanine
  SITE 5: note = D-2'-naphthylalanine
  SITE 8: note = Amidated Residue
  VARIANT 2..8: note = cyclization may occur via a lactam bond or disulfide bond
RCAHXRWC

SEQ ID NO: 284 - protein, 8 aa, synthetic construct
  SITE 1: note = Acetylated Residue
  SITE 3: note = D-Alanine
  SITE 5: note = D-2'-naphthylalanine
  SITE 8: note = Amidated Residue
  VARIANT 2..8: note = cyclization may occur via a lactam bond or disulfide bond
RDAHXRWK

SEQ ID NO: 285 - protein, 9 aa, synthetic construct
  SITE 1: note = Acetylated D-Valine
  SITE 2: note = D-Proline
  SITE 6: note = D-2'-naphthylalanine
  SITE 9: note = Amidated Norleucine
  SITE 3..8: note = side chain cyclized via a lactam bond
VPDWRXPKX

SEQ ID NO: 286 - protein, 9 aa, synthetic construct
  SITE 1: note = Acetylated D-Valine
  SITE 2: note = D-Proline
  SITE 6: note = D-2'-naphthylalanine
  SITE 9: note = Amidated Norleucine
  SITE 3..8: note = side chain cyclized via a lactam bond
VPKWRXPDX

SEQ ID NO: 287 - protein, 10 aa, synthetic construct
  SITE 1: note = Acetylated D-Valine
  SITE 2: note = D-Proline
  SITE 6: note = D-2'-naphthylalanine
  SITE 9: note = Norleucine
  SITE 10: note = Amidated Norleucine
  SITE 3..8: note = side chain cyclized via a lactam bond
VPDWRXPKXX

SEQ ID NO: 288 - protein, 10 aa, synthetic construct
  SITE 1: note = Acetylated D-Valine
  SITE 2: note = D-Proline
  SITE 6: note = D-2'-naphthylalanine
  SITE 9: note = Norleucine
  SITE 10: note = Amidated Norleucine
  SITE 3..8: note = side chain cyclized via a lactam bond
VPKWRXPDXX

SEQ ID NO: 289 - protein, 10 aa, synthetic construct
  SITE 1: note = Acetylated D-Valine
  SITE 2: note = D-Proline
  SITE 6: note = D-2'-naphthylalanine
  SITE 9: note = D-Valine
  SITE 10: note = Amidated D-Proline
  SITE 3..8: note = side chain cyclized via a lactam bond
VPDWRXPKVP

SEQ ID NO: 290 - protein, 10 aa, synthetic construct
  SITE 1: note = Acetylated D-Valine
  SITE 2: note = D-Proline
  SITE 6: note = D-2'-naphthylalanine
  SITE 9: note = D-Valine
  SITE 10: note = Amidated D-Proline
  SITE 3..8: note = side chain cyclized via a lactam bond
VPKWRXPDVP

SEQ ID NO: 291 - protein, 10 aa, synthetic construct
  SITE 1: note = Acetylated Norleucine
  SITE 9: note = D-Valine
  SITE 10: note = Amidated D-Proline
  SITE 2..8: note = side chain cyclized via a lactam bond
XDPFFRWKVP

SEQ ID NO: 292 - protein, 10 aa, synthetic construct
  SITE 1: note = Acetylated Residue
  SITE 10: note = Amidated Residue
  SITE 2..8: note = side chain cyclized via a lactam bond
LDPFFRWKVP

SEQ ID NO: 293 - protein, 10 aa, synthetic construct
  SITE 2..8: note = side chain cyclized via a lactam bond
LDPFFRWKVP

SEQ ID NO: 294 - protein, 8 aa, synthetic construct
LPFFRWVP

SEQ ID NO: 295 - protein, 8 aa, synthetic construct
IPFFRWVP

SEQ ID NO: 296 - protein, 8 aa, synthetic construct
VPFFRWVP

SEQ ID NO: 297 - protein, 10 aa, synthetic construct
LDPFFRWKVP

SEQ ID NO: 298 - protein, 10 aa, synthetic construct
IDPFFRWKVP

SEQ ID NO: 299 - protein, 10 aa, synthetic construct
VDPFFRWKVP

SEQ ID NO: 300 - protein, 9 aa, synthetic construct
  SITE 1: note = Acetylated Norleucine
  SITE 8: note = D-Valine
  SITE 9: note = Amidated D-Proline
  SITE 2..7: note = side chain cyclized via a lactam bond
XDPWRWKVP

SEQ ID NO: 301 - protein, 9 aa, synthetic construct
  SITE 1: note = Acetylated Residue
  SITE 9: note = Amidated Residue
  SITE 2..7: note = side chain cyclized via a lactam bond
LDPWRWKVP

SEQ ID NO: 302 - protein, 9 aa, synthetic construct
  SITE 2..7: note = side chain cyclized via a lactam bond
LDPWRWKVP

SEQ ID NO: 303 - protein, 7 aa, synthetic construct
LPWRWVP

SEQ ID NO: 304 - protein, 7 aa, synthetic construct
IPWRWVP

SEQ ID NO: 305 - protein, 7 aa, synthetic construct
VPWRWVP

SEQ ID NO: 306 - protein, 9 aa, synthetic construct
LDPWRWKVP

SEQ ID NO: 307 - protein, 9 aa, synthetic construct
IDPWRWKVP

SEQ ID NO: 308 - protein, 9 aa, synthetic construct
VDPWRWKVP

SEQ ID NO: 309 - protein, 9 aa, synthetic construct
  SITE 1: note = Acetylated Norleucine
  SITE 4: note = D-2'-naphthylalanine
  SITE 8: note = D-Proline
  SITE 9: note = Amidated D-Valine
  SITE 2..7: note = side chain cyclized via a lactam bond
XDPXRWKPV

SEQ ID NO: 310 - protein, 12 aa, synthetic construct
  SITE 1: note = Acetylated Residue
  SITE 3: note = Norleucine
  SITE 6: note = D-Phenylalanine
  SITE 12: note = Amidated Residue
SYXEHFRWGKPV

SEQ ID NO: 311 - protein, 7 aa, synthetic construct
  SITE 1: note = Acetylated Norleucine
  SITE 4: note = D-Phenylalanine
  SITE 7: note = Amidated Residue
  SITE 2..7: note = side chain cyclized via a lactam bond
XDHFRWK

SEQ ID NO: 312 - protein, 7 aa, synthetic construct
  SITE 1: note = Acetylated Norleucine
  SITE 4: note = D-Phenylalanine
  SITE 2..7: note = side chain cyclized via a lactam bond
XDHFRWK

SEQ ID NO: 313 - protein, 7 aa, synthetic construct
  SITE 1: note = Acetylated Norleucine
  SITE 4: note = D-2'-naphthylalanine
  SITE 7: note = Amidated Residue
  SITE 2..7: note = side chain cyclized via a lactam bond
XDHXRWK

SEQ ID NO: 314 - protein, 9 aa, synthetic construct
  SITE 1: note = Acetylated Norleucine
  SITE 4: note = D-2'-naphthylalanine
  SITE 9: note = Amidated Residue
  SITE 2..7: note = side chain cyclized via a lactam bond
XDPXRWKPV

SEQ ID NO: 315 - protein, 10 aa, synthetic construct
  SITE 1: note = Acetylated Norleucine
  SITE 4: note = D-2'-naphthylalanine
  SITE 8: note = D-Threonine
  SITE 9: note = D-Proline
  SITE 10: note = D-Threonine
  SITE 2..7: note = side chain cyclized via a lactam bond
XDPXRWKTPT

SEQ ID NO: 316 - protein, 9 aa, synthetic construct
  SITE 1: note = Acetylated Norleucine
  SITE 4: note = D-2'-naphthylalanine
  SITE 8: note = D-Proline
  SITE 9: note = Amidated D-Valine
  SITE 2..7: note = side chain cyclized via a lactam bond
XDHXRWKPV

SEQ ID NO: 317 - protein, 9 aa, synthetic construct
  SITE 1: note = Acetylated Norleucine
  SITE 2: note = D-Aspartic acid
  SITE 4: note = D-2'-naphthylalanine
  SITE 7: note = D-Lysine
  SITE 8: note = D-Proline
  SITE 9: note = Amidated D-Valine
  SITE 2..7: note = side chain cyclized via a lactam bond
XDPXRWKPV

SEQ ID NO: 318 - protein, 9 aa, synthetic construct
  SITE 1: note = Acetylated Norleucine
  SITE 4: note = D-2'-naphthylalanine
  SITE 8: note = beta-3-Proline
  SITE 9: note = Amidated beta-2-Valine
  SITE 2..7: note = side chain cyclized via a lactam bond
XDPXRWKXX

SEQ ID NO: 319 - protein, 9 aa, synthetic construct
  SITE 1: note = Acetylated Norleucine
  SITE 4: note = D-2'-naphthylalanine
  SITE 8: note = beta-3-Proline
  SITE 9: note = Amidated beta-3-Valine
  SITE 2..7: note = side chain cyclized via a lactam bond
XDPXRWKXX

SEQ ID NO: 320 - protein, 10 aa, synthetic construct
  SITE 1: note = Acetylated Norleucine
  SITE 4: note = D-2'-naphthylalanine
  SITE 8: note = D-Threonine
  SITE 9: note = D-Proline
  SITE 10: note = D-Threonine
  SITE 2..7: note = side chain cyclized via a lactam bond
XDPXRWKTPT

SEQ ID NO: 321 - protein, 10 aa, synthetic construct
  SITE 1: note = Acetylated Norleucine
  SITE 4: note = D-2'-naphthylalanine
  SITE 8: note = D-Threonine
  SITE 9: note = D-Proline
  SITE 10: note = D-Threonine
  SITE 2..7: note = side chain cyclized via a lactam bond
XDHXRWKTPT

SEQ ID NO: 322 - protein, 10 aa, synthetic construct
  SITE 1: note = Acetylated Norleucine
  SITE 2: note = D-Aspartic Acid
  SITE 4: note = D-2'-naphthylalanine
  SITE 7: note = D-Lysine
  SITE 8: note = D-Threonine
  SITE 9: note = D-Proline
  SITE 10: note = D-Threonine
  SITE 2..7: note = side chain cyclized via a lactam bond
XDPXRWKTPT

SEQ ID NO: 323 - protein, 10 aa, synthetic construct
  SITE 1: note = Acetylated Norleucine
  SITE 2: note = D-Aspartic Acid
  SITE 4: note = D-2'-naphthylalanine
  SITE 7: note = D-Lysine
  SITE 8: note = D-Threonine
  SITE 9: note = D-Proline
  SITE 10: note = D-Threonine
  SITE 2..7: note = side chain cyclized via a lactam bond
XDHXRWKTPT

SEQ ID NO: 324 - protein, 9 aa, synthetic construct
  SITE 1: note = Acetylated Norleucine
  SITE 4: note = D-2'-naphthylalanine
  SITE 8: note = D-Proline
  SITE 9: note = Amidated D-Valine
  SITE 2..7: note = side chain cyclized via a lactam bond
XDPXRWKPV

SEQ ID NO: 325 - protein, 9 aa, synthetic construct
  SITE 1: note = Acetylated Norleucine
  SITE 4: note = D-2'-naphthylalanine
  SITE 8: note = D-Proline
  SITE 9: note = Amidated D-Valine
  SITE 2..7: note = side chain cyclized via a lactam bond
XDHXRWKPV

SEQ ID NO: 326 - protein, 9 aa, synthetic construct
  SITE 1: note = Acetylated Norleucine
  SITE 2: note = D-Aspartic Acid
  SITE 4: note = D-2'-naphthylalanine
  SITE 7: note = D-Lysine
  SITE 8: note = D-Proline
  SITE 9: note = Amidated D-Valine
  SITE 2..7: note = side chain cyclized via a lactam bond
XDPXRWKPV

SEQ ID NO: 327 - protein, 10 aa, synthetic construct
  SITE 1: note = Acetylated Norleucine
  SITE 4: note = D-Phenylalanine
  SITE 8: note = D-Threonine
  SITE 9: note = D-Proline
  SITE 10: note = D-Threonine
  SITE 2..7: note = side chain cyclized via a lactam bond
XDPFRWKTPT

SEQ ID NO: 328 - protein, 10 aa, synthetic construct
  SITE 1: note = Acetylated Norleucine
  SITE 4: note = D-Phenylalanine
  SITE 8: note = D-Threonine
  SITE 9: note = D-Proline
  SITE 10: note = D-Threonine
  SITE 2..7: note = side chain cyclized via a lactam bond
XDHFRWKTPT

SEQ ID NO: 329 - protein, 10 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = D-Threonine
  SITE 9: note = D-Proline
  SITE 10: note = D-Threonine
  SITE 2..7: note = side chain cyclized via a lactam bond
XDPFRWKTPT

SEQ ID NO: 330 - protein, 10 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = D-Threonine
  SITE 9: note = D-Proline
  SITE 10: note = D-Threonine
  SITE 2..7: note = side chain cyclized via a lactam bond
XDHFRWKTPT

SEQ ID NO: 331 - protein, 9 aa, synthetic construct
  SITE 1: note = Acetylated Norleucine
  SITE 4: note = D-Phenylalanine
  SITE 8: note = D-Proline
  SITE 9: note = Amidated D-Valine
  SITE 2..7: note = side chain cyclized via a lactam bond
XDPFRWKPV

SEQ ID NO: 332 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = D-Proline
  SITE 9: note = Amidated D-Valine
  SITE 2..7: note = side chain cyclized via a lactam bond
XDHFRWKPV

SEQ ID NO: 333 - protein, 9 aa, synthetic construct
  SITE 1: note = Acetylated Norleucine
  SITE 4: note = D-2'-naphthylalanine
  SITE 8: note = D-Proline
  SITE 9: note = Amidated D-Valine
  SITE 2..7: note = side chain cyclized via a lactam bond
XDPXRWKPV

SEQ ID NO: 334 - protein, 9 aa, synthetic construct
  SITE 1: note = Acetylated Norleucine
  SITE 4: note = D-2'-naphthylalanine
  SITE 8: note = D-Proline
  SITE 9: note = Amidated D-Valine
  SITE 2..7: note = side chain cyclized via a lactam bond
XDHXRWKPV

SEQ ID NO: 335 - protein, 9 aa, synthetic construct
  SITE 1: note = Acetylated Norleucine
  SITE 4: note = D-Phenylalanine
  SITE 8: note = D-Valine
  SITE 9: note = Amidated D-Proline
  SITE 2..7: note = side chain cyclized via a lactam bond
XDPFRWKVP

SEQ ID NO: 336 - protein, 9 aa, synthetic construct
  SITE 1: note = Acetylated Norleucine
  SITE 4: note = D-Phenylalanine
  SITE 8: note = D-Valine
  SITE 9: note = Amidated D-Proline
  SITE 2..7: note = side chain cyclized via a lactam bond
XDHFRWKVP

SEQ ID NO: 337 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = D-Valine
  SITE 9: note = Amidated D-Proline
  SITE 2..7: note = side chain cyclized via a lactam bond
XDPFRWKVP

SEQ ID NO: 338 - protein, 9 aa, synthetic construct
  SITE 1: note = Acetylated Norleucine
  SITE 2: note = D-Aspartic Acid
  SITE 4: note = D-2'-naphthylalanine
  SITE 7: note = D-Lysine
  SITE 8: note = D-Valine
  SITE 9: note = Amidated D-Proline
  SITE 2..7: note = side chain cyclized via a lactam bond
XDHXRWKVP

SEQ ID NO: 339 - protein, 9 aa, synthetic construct
  SITE 1: note = Acetylated Norleucine
  SITE 4: note = D-Phenylalanine
  SITE 8: note = beta-Proline
  SITE 9: note = Amidated beta-Valine
  SITE 2..7: note = side chain cyclized via a lactam bond
XDPFRWKXX

SEQ ID NO: 340 - protein, 9 aa, synthetic construct
  SITE 1: note = Acetylated Norleucine
  SITE 4: note = D-Phenylalanine
  SITE 8: note = beta-Proline
  SITE 9: note = Amidated beta-Valine
  SITE 2..7: note = side chain cyclized via a lactam bond
XDHFRWKXX

SEQ ID NO: 341 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Proline
  SITE 9: note = Amidated beta-Valine
  SITE 2..7: note = side chain cyclized via a lactam bond
XDPFRWKXX

SEQ ID NO: 342 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Proline
  SITE 9: note = Amidated beta-Valine
  SITE 2..7: note = side chain cyclized via a lactam bond
XDHFRWKXX

SEQ ID NO: 343 - protein, 9 aa, synthetic construct
  SITE 1: note = Acetylated Norleucine
  SITE 4: note = D-Phenylalanine
  SITE 8: note = beta-Valine
  SITE 9: note = Amidated beta-Proline
  SITE 2..7: note = side chain cyclized via a lactam bond
XDPFRWKXX

SEQ ID NO: 344 - protein, 9 aa, synthetic construct
  SITE 1: note = Acetylated Norleucine
  SITE 4: note = D-Phenylalanine
  SITE 8: note = beta-Valine
  SITE 9: note = Amidated beta-Proline
  SITE 2..7: note = side chain cyclized via a lactam bond
XDHFRWKXX

SEQ ID NO: 345 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Valine
  SITE 9: note = Amidated beta-Proline
  SITE 2..7: note = side chain cyclized via a lactam bond
XDPFRWKXX

SEQ ID NO: 346 - protein, 9 aa, synthetic construct
  SITE 1: note = Acetylated Norleucine
  SITE 2: note = D-Aspartic Acid
  SITE 4: note = D-2'-naphthylalanine
  SITE 7: note = D-Lysine
  SITE 8: note = beta-Valine
  SITE 9: note = Amidated beta-Proline
  SITE 2..7: note = side chain cyclized via a lactam bond
XDHXRWKXX

SEQ ID NO: 347 - protein, 9 aa, synthetic construct
  SITE 1: note = Acetylated Norleucine
  SITE 4: note = D-Phenylalanine
  SITE 8: note = beta-Proline
  SITE 9: note = Amidated beta-Proline
  SITE 2..7: note = side chain cyclized via a lactam bond
XDPFRWKXX

SEQ ID NO: 348 - protein, 9 aa, synthetic construct
  SITE 1: note = Acetylated Norleucine
  SITE 4: note = D-Phenylalanine
  SITE 8: note = beta-Proline
  SITE 9: note = Amidated beta-Proline
  SITE 2..7: note = side chain cyclized via a lactam bond
XDHFRWKXX

SEQ ID NO: 349 - protein, 9 aa, synthetic construct
  SITE 1: note = Acetylated Norleucine
  SITE 2: note = D-Aspartic Acid
  SITE 4: note = D-2'-naphthylalanine
  SITE 7: note = D-Lysine
  SITE 8: note = beta-Proline
  SITE 9: note = Amidated beta-Proline
  SITE 2..7: note = side chain cyclized via a lactam bond
XDPXRWKXX

SEQ ID NO: 350 - protein, 9 aa, synthetic construct
  SITE 1: note = Acetylated Norleucine
  SITE 2: note = D-Aspartic Acid
  SITE 4: note = D-2'-naphthylalanine
  SITE 7: note = D-Lysine
  SITE 8: note = beta-Proline
  SITE 9: note = Amidated beta-Proline
  SITE 2..7: note = side chain cyclized via a lactam bond
XDHXRWKXX

SEQ ID NO: 351 - protein, 9 aa, synthetic construct
  SITE 1: note = Acetylated Norleucine
  SITE 4: note = D-Phenylalanine
  SITE 8: note = beta-Valine
  SITE 9: note = Amidated beta-Valine
  SITE 2..7: note = side chain cyclized via a lactam bond
XDPFRWKXX

SEQ ID NO: 352 - protein, 9 aa, synthetic construct
  SITE 1: note = Acetylated Norleucine
  SITE 4: note = D-Phenylalanine
  SITE 8: note = beta-Valine
  SITE 9: note = Amidated beta-Valine
  SITE 2..7: note = side chain cyclized via a lactam bond
XDHFRWKXX

SEQ ID NO: 353 - protein, 9 aa, synthetic construct
  SITE 1: note = Acetylated Norleucine
  SITE 2: note = D-Aspartic Acid
  SITE 7: note = D-Lysine
  SITE 8: note = beta-Valine
  SITE 9: note = Amidated beta-Valine
  SITE 2..7: note = side chain cyclized via a lactam bond
XDPFRWKXX

SEQ ID NO: 354 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Valine
  SITE 9: note = Amidated beta-Valine
  SITE 2..7: note = side chain cyclized via a lactam bond
XDHFRWKXX

SEQ ID NO: 355 - protein, 9 aa, synthetic construct
  SITE 1: note = Acetylated Norleucine
  SITE 4: note = D-Phenylalanine
  SITE 8: note = (3-methyl)-beta-Valine
  SITE 9: note = Amidated beta-Valine
  SITE 2..7: note = side chain cyclized via a lactam bond
XDPFRWKXX

SEQ ID NO: 356 - protein, 9 aa, synthetic construct
  SITE 1: note = Acetylated Norleucine
  SITE 4: note = D-Phenylalanine
  SITE 8: note = (3-methyl)-beta-Valine
  SITE 9: note = Amidated beta-Valine
  SITE 2..7: note = side chain cyclized via a lactam bond
XDHFRWKXX

SEQ ID NO: 357 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = (3-methyl)-beta-Valine
  SITE 9: note = Amidated beta-Valine
  SITE 2..7: note = side chain cyclized via a lactam bond
XDPFRWKXX

SEQ ID NO: 358 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = (3-methyl)-beta-Valine
  SITE 9: note = Amidated beta-Valine
  SITE 2..7: note = side chain cyclized via a lactam bond
XDHFRWKXX

SEQ ID NO: 359 - protein, 9 aa, synthetic construct
  SITE 1: note = Acetylated Norleucine
  SITE 4: note = D-Phenylalanine
  SITE 8: note = (3-methyl)-beta-Valine
  SITE 9: note = Amidated beta-Proline
  SITE 2..7: note = side chain cyclized via a lactam bond
XDPFRWKXX

SEQ ID NO: 360 - protein, 9 aa, synthetic construct
  SITE 1: note = Acetylated Norleucine
  SITE 4: note = D-Phenylalanine
  SITE 8: note = (3-methyl)-beta-Valine
  SITE 9: note = Amidated beta-Proline
  SITE 2..7: note = side chain cyclized via a lactam bond
XDHFRWKXX

SEQ ID NO: 361 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = (3-methyl)-beta-Valine
  SITE 9: note = Amidated beta-Proline
  SITE 2..7: note = side chain cyclized via a lactam bond
XDPFRWKXX

SEQ ID NO: 362 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = (3-methyl)-beta-Valine
  SITE 9: note = Amidated beta-Proline
  SITE 2..7: note = side chain cyclized via a lactam bond
XDHFRWKXX

SEQ ID NO: 363 - protein, 10 aa, synthetic construct
  SITE 1: note = Acetylated Norleucine
  SITE 4: note = D-Phenylalanine
  SITE 8: note = beta-Threonine
  SITE 9: note = beta-Proline
  SITE 10: note = beta-Threonine
  SITE 2..7: note = side chain cyclized via a lactam bond
XDPFRWKXXX

SEQ ID NO: 364 - protein, 10 aa, synthetic construct
  SITE 1: note = Acetylated Norleucine
  SITE 4: note = D-Phenylalanine
  SITE 8: note = beta-Threonine
  SITE 9: note = beta-Proline
  SITE 10: note = beta-Threonine
  SITE 2..7: note = side chain cyclized via a lactam bond
XDHFRWKXXX

SEQ ID NO: 365 - protein, 10 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Threonine
  SITE 9: note = beta-Proline
  SITE 10: note = beta-Threonine
  SITE 2..7: note = side chain cyclized via a lactam bond
XDPFRWKXXX

SEQ ID NO: 366 - protein, 10 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Threonine
  SITE 9: note = beta-Proline
  SITE 10: note = beta-Threonine
  SITE 2..7: note = side chain cyclized via a lactam bond
XDHFRWKXXX

SEQ ID NO: 367 - protein, 9 aa, synthetic construct
  SITE 1: note = Acetylated Norleucine
  SITE 4: note = D-Phenylalanine
  SITE 8: note = D-Proline
  SITE 9: note = Amidated D-Alanine
  SITE 2..7: note = side chain cyclized via a lactam bond
XDPFRWKPA

SEQ ID NO: 368 - protein, 9 aa, synthetic construct
  SITE 1: note = Acetylated Norleucine
  SITE 4: note = D-Phenylalanine
  SITE 8: note = D-Proline
  SITE 9: note = Amidated D-Alanine
  SITE 2..7: note = side chain cyclized via a lactam bond
XDHFRWKPA

SEQ ID NO: 369 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = D-Proline
  SITE 9: note = Amidated D-Alanine
  SITE 2..7: note = side chain cyclized via a lactam bond
XDPFRWKPA

SEQ ID NO: 370 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = D-Proline
  SITE 9: note = Amidated D-Alanine
  SITE 2..7: note = side chain cyclized via a lactam bond
XDHFRWKPA

SEQ ID NO: 371 - protein, 9 aa, synthetic construct
  SITE 1: note = Acetylated Norleucine
  SITE 4: note = D-Phenylalanine
  SITE 8: note = D-Alanine
  SITE 9: note = Amidated D-Proline
  SITE 2..7: note = side chain cyclized via a lactam bond
XDPFRWKAP

SEQ ID NO: 372 - protein, 9 aa, synthetic construct
  SITE 1: note = Acetylated Norleucine
  SITE 4: note = D-Phenylalanine
  SITE 8: note = D-Alanine
  SITE 9: note = Amidated D-Proline
  SITE 2..7: note = side chain cyclized via a lactam bond
XDHFRWKAP

SEQ ID NO: 373 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = D-Alanine
  SITE 9: note = Amidated D-Proline
  SITE 2..7: note = side chain cyclized via a lactam bond
XDPFRWKAP

SEQ ID NO: 374 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = D-Alanine
  SITE 9: note = Amidated D-Proline
  SITE 2..7: note = side chain cyclized via a lactam bond
XDHFRWKAP

SEQ ID NO: 375 - protein, 9 aa, synthetic construct
  SITE 1: note = Acetylated Norleucine
  SITE 4: note = D-Phenylalanine
  SITE 8: note = beta-Proline
  SITE 9: note = Amidated beta-Alanine
  SITE 2..7: note = side chain cyclized via a lactam bond
XDPFRWKXX

SEQ ID NO: 376 - protein, 9 aa, synthetic construct
  SITE 1: note = Acetylated Norleucine
  SITE 4: note = D-Phenylalanine
  SITE 8: note = beta-Proline
  SITE 9: note = Amidated beta-Alanine
  SITE 2..7: note = side chain cyclized via a lactam bond
XDHFRWKXX

SEQ ID NO: 377 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Proline
  SITE 9: note = Amidated beta-Alanine
  SITE 2..7: note = side chain cyclized via a lactam bond
XDPFRWKXX

SEQ ID NO: 378 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Proline
  SITE 9: note = Amidated beta-Alanine
  SITE 2..7: note = side chain cyclized via a lactam bond
XDHFRWKXX

SEQ ID NO: 379 - protein, 9 aa, synthetic construct
  SITE 1: note = Acetylated Norleucine
  SITE 4: note = D-Phenylalanine
  SITE 8: note = beta-Alanine
  SITE 9: note = Amidated beta-Proline
  SITE 2..7: note = side chain cyclized via a lactam bond
XDPFRWKXX

SEQ ID NO: 380 - protein, 9 aa, synthetic construct
  SITE 1: note = Acetylated Norleucine
  SITE 4: note = D-Phenylalanine
  SITE 8: note = beta-Alanine
  SITE 9: note = Amidated beta-Proline
  SITE 2..7: note = side chain cyclized via a lactam bond
XDHFRWKXX

SEQ ID NO: 381 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Alanine
  SITE 9: note = Amidated beta-Proline
  SITE 2..7: note = side chain cyclized via a lactam bond
XDPFRWKXX

SEQ ID NO: 382 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Alanine
  SITE 9: note = Amidated beta-Proline
  SITE 2..7: note = side chain cyclized via a lactam bond
XDHFRWKXX

SEQ ID NO: 383 - protein, 9 aa, synthetic construct
  SITE 1: note = Acetylated Norleucine
  SITE 4: note = D-Phenylalanine
  SITE 8: note = beta-Valine
  SITE 9: note = Amidated beta-Alanine
  SITE 2..7: note = side chain cyclized via a lactam bond
XDPFRWKXX

SEQ ID NO: 384 - protein, 9 aa, synthetic construct
  SITE 1: note = Acetylated Norleucine
  SITE 4: note = D-Phenylalanine
  SITE 8: note = beta-Valine
  SITE 9: note = Amidated beta-Alanine
  SITE 2..7: note = side chain cyclized via a lactam bond
XDHFRWKXX

SEQ ID NO: 385 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Valine
  SITE 9: note = Amidated beta-Alanine
  SITE 2..7: note = side chain cyclized via a lactam bond
XDPFRWKXX

SEQ ID NO: 386 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = beta-Valine
  SITE 9: note = Amidated beta-Alanine
  SITE 2..7: note = side chain cyclized via a lactam bond
XDHFRWKXX

SEQ ID NO: 387 - protein, 9 aa, synthetic construct
  SITE 1: note = Acetylated Norleucine
  SITE 4: note = D-Phenylalanine
  SITE 8: note = (3-methyl)-beta-Valine
  SITE 9: note = Amidated beta-Alanine
  SITE 2..7: note = side chain cyclized via a lactam bond
XDPFRWKXX

SEQ ID NO: 388 - protein, 9 aa, synthetic construct
  SITE 1: note = Acetylated Norleucine
  SITE 4: note = D-Phenylalanine
  SITE 8: note = (3-methyl)-beta-Valine
  SITE 9: note = Amidated beta-Alanine
  SITE 2..7: note = side chain cyclized via a lactam bond
XDHFRWKXX

SEQ ID NO: 389 - protein, 9 aa, synthetic construct
  SITE 1: note = Acetylated Norleucine
  SITE 2: note = D-Aspartic Acid
  SITE 4: note = D-Phenylalanine
  SITE 7: note = D-Lysine
  SITE 8: note = (3-methyl)-beta-Valine
  SITE 9: note = Amidated beta-Alanine
  SITE 2..7: note = side chain cyclized via a lactam bond
XDPFRWKXX

SEQ ID NO: 390 - protein, 9 aa, synthetic construct
  SITE 1: note = Acetylated Norleucine
  SITE 4: note = D-2'-naphthylalanine
  SITE 8: note = beta-2-Valine
  SITE 9: note = Amidated beta-3-Proline
  SITE 2..7: note = side chain cyclized via a lactam bond
XDPXRWKXX

SEQ ID NO: 391 - protein, 9 aa, synthetic construct
  SITE 1: note = Acetylated Norleucine
  SITE 4: note = D-2'-naphthylalanine
  SITE 8: note = beta-3-Valine
  SITE 9: note = Amidated beta-3-Proline
  SITE 2..7: note = side chain cyclized via a lactam bond
XDPXRWKXX

SEQ ID NO: 392 - protein, 9 aa, synthetic construct
  SITE 1: note = Acetylated Norleucine
  SITE 4: note = D-2'-naphthylalanine
  SITE 8: note = beta-3-Proline
  SITE 9: note = Amidated beta-3-Proline
  SITE 2..7: note = side chain cyclized via a lactam bond
XDPXRWKXX

SEQ ID NO: 393 - protein, 6 aa, synthetic construct
PFFRWK

SEQ ID NO: 394 - protein, 7 aa, synthetic construct
  SITE 1..7: note = side chain cyclized via a lactam bond
DPFFRWK

SEQ ID NO: 395 - protein, 5 aa, synthetic construct
PWRWK

SEQ ID NO: 396 - protein, 6 aa, synthetic construct
  SITE 1..6: note = side chain cyclized via a lactam bond
DPWRWK

SEQ ID NO: 397 - protein, 5 aa, synthetic construct
PWRWV

SEQ ID NO: 398 - protein, 7 aa, synthetic construct
  SITE 4: note = D-2'-naphthylalanine
  SITE 1..7: note = side chain cyclized via a lactam bond
DPHXRWK